SEQUENCE LISTING (18 sequences)

SEQ ID NO: 1 - protein, 189 aa, Homo sapiens
MALSFSLLMAVLVLSYKSICSLGCDLPQTHSLGNRRALILLAQMGRISHFSCLKDRHDFGFPEEEFDGHQFQKAQAISVLHEMIQQTFNLFSTEDSSAAWEQSLLEKFSTELYQQLNDLEACVIQEVGVEETPLMNEDSILAVRKYFQRITLYLTEKKYSPCAWEVVRAEIMRSLSFSTNLQKRLRRKD

SEQ ID NO: 2 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 3 - protein, 207 aa, Homo sapiens
MSTKPDMIQKCLWLEILMGIFIAGTLSLDCNLLNVHLRRVTWQNLRHLSSMSNSFPVECLRENIAFELPQEFLQYTQPMKRDIKKAFYEMSLQAFNIFSQHTFKYWKERHLKQIQIGLDQQAEYLNQCLEEDKNENEDMKEMKENEMKPSEARVPQLSSLELRRYFHRIDNFLKEKKYSDCAWEIVRVEIRRCLYYFYKFTALFRRK

SEQ ID NO: 4 - protein, 207 aa, Homo sapiens; identical to SEQ ID NO: 3

SEQ ID NO: 5 - protein, 195 aa, Homo sapiens
MALLFPLLAALVMTSYSPVGSLGCDLPQNHGLLSRNTLVLLHQMRRISPFLCLKDRRDFRFPQEMVKGSQLQKAHVMSVLHEMLQQIFSLFHTERSSAAWNMTLLDQLHTGLHQQLQHLETCLLQVVGEGESAGAISSPALTLRRYFQGIRVYLKEKKYSDCAWEVVRMEIMKSLFLSTNMQERLRSKDRDLGSS

SEQ ID NO: 6 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 7 - protein, 260 aa, synthetic construct
MIETYSQPSPRSVATGLPASMKIFMYLLTVFLITQMIGSVLFAVYLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVTLENGKQLTVKRQGLYYIYAQVTFCSNREPSSQRPFIVGLWLKPSSGSERILLKAANTHSSSQLCEQQSVHLGGVFELQPGASVFVNVTDPSQVSHGTGFTSFGLLKL

SEQ ID NO: 8 - protein, 252 aa, synthetic construct
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKDEDPQIAAHVVSEANSNAASVLQWAKKGYYTMKSNLVTLENGKQLTVKRQGLYYIYAQVTFCSNREPSSQRPFIVGLWLKPSSGSERILLKAANTHSSSQLCEQQSVHLGGVFELQPGASVFVNVTDPSQVSHGTGFTSFGLLKL

SEQ ID NO: 9 - protein, 260 aa, synthetic construct
MIETYSQPSPRSVATGLPASMKIFMYLLTVFLITQMIGSVLFAVYLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVTLENGKQLTVKRQGLYYIYAQVTFCSNREASSQAPFIVGLWLKPSSGSERILLKAANTHSSSQLCEQQSVHLGGVFELQPGASVFVNVTDPSQVSHGTGFTSFGLLKL

SEQ ID NO: 10 - protein, 252 aa, synthetic construct
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKDEDPQIAAHVVSEANSNAASVLQWAKKGYYTMKSNLVTLENGKQLTVKRQGLYYIYAQVTFCSNREASSQAPFIVGLWLKPSSGSERILLKAANTHSSSQLCEQQSVHLGGVFELQPGASVFVNVTDPSQVSHGTGFTSFGLLKL

SEQ ID NO: 11 - protein, 260 aa, synthetic construct
MIETYSQPSPRSVATGLPASMKIFMYLLTVFLITQMIGSVLFAVYLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVTLENGKQLTVKRQGLYYIYAQVTFCSNREASSQAPFIVGLWLKPSSGSERILLKAANTHSSSQLCEQQSIHLGGVFELQPGASVFVNVTDPSQVSHGTGFTSFGLLKL

SEQ ID NO: 12 - protein, 252 aa, synthetic construct
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKDEDPQIAAHVVSEANSNAASVLQWAKKGYYTMKSNLVTLENGKQLTVKRQGLYYIYAQVTFCSNREASSQAPFIVGLWLKPSSGSERILLKAANTHSSSQLCEQQSIHLGGVFELQPGASVFVNVTDPSQVSHGTGFTSFGLLKL

SEQ ID NO: 13 - protein, 260 aa, synthetic construct
MIETYSQPSPRSVATGLPASMKIFMYLLTVFLITQMIGSVLFAVYLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVTLENGKQLTVKRQGLYYIYAQVTFCSNREASSQAPFIVGLWLKPSSGSERILLKAANTHSSAKPCGQQSIHLGGVFELQPGASCFVNVTDPSQVSHGTGFTSFGLLKL

SEQ ID NO: 14 - protein, 252 aa, synthetic construct
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKDEDPQIAAHVVSEANSNAASVLQWAKKGYYTMKSNLVTLENGKQLTVKRQGLYYIYAQVTFCSNREASSQAPFIVGLWLKPSSGSERILLKAANTHSSAKPCGQQSIHLGGVFELQPGASVFVNVTDPSQVSHGTGFTSFGLLKL

SEQ ID NO: 15 - protein, 260 aa, synthetic construct
MIETYSQPSPRSVATGLPASMKIFMYLLTVFLITQMIGSVLFAVYLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVTLENGKQLTVKRQGLYYIYAQVTFCSNREPSSQRPFIVGLWLKPSSGSERILLKAANTHSSSQLCEQQSIHLGGVFELQPGASVFVNVTDPSQVSHGTGFTSFGLLKL

SEQ ID NO: 16 - protein, 252 aa, synthetic construct
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKDEDPQIAAHVVSEANSNAASVLQWAKKGYYTMKSNLVTLENGKQLTVKRQGLYYIYAQVTFCSNREPSSQRPFIVGLWLKPSSGSERILLKAANTHSSSQLCEQQSIHLGGVFELQPGASVFVNVTDPSQVSHGTGFTSFGLLKL

SEQ ID NO: 17 - protein, 260 aa, synthetic construct
MIETYSQPSPRSVATGLPASMKIFMYLLTVFLITQMIGSVLFAVYLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVTLENGKQLTVKRQGLYYIYAQVTFCSNREPSSQRPFIVGLWLKPSSGSERILLKAANTHSSAKPCGQQSIHLGGVFELQPGASVFVNVTDPSQVSHGTGFTSFGLLKL

SEQ ID NO: 18 - protein, 252 aa, synthetic construct
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKDEDPQIAAHVVSEANSNAASVLQWAKKGYYTMKSNLVTLENGKQLTVKRQGLYYIYAQVTFCSNREPSSQRPFIVGLWLKPSSGSERILLKAANTHSSAKPCGQQSIHLGGVFELQPGASVFVNVTDPSQVSHGTGFTSFGLLKL

(19) United States
(12) Patent Application Publication
(10) Pub. No.: US 2023/0270784 A1
(43) Pub. Date: Aug. 31, 2023

(54) ONCOLYTIC VIRUS BOOSTS T CELL RESPONSE FOR EFFECTIVE TIL THERAPY

(71) Applicant: Memgen, Inc., Houston, TX (US)

(72) Inventors: Amer A. BEG, Tampa, FL (US); Mark J. CANTWELL, Meadow Vista, CA (US)

(73) Assignee: Memgen, Inc., Houston, TX (US)

(21) Appl. No.: 18/062,921

(22) Filed: Dec. 7, 2022

Related U.S. Application Data
(60) Provisional application No. 63/286,728, filed on Dec. 7, 2021.

(51) Int. Cl. A61K 35/17; C12N 5/0783; A61P 35/00

(52) U.S. Cl. CPC A61K 35/17; C12N 5/0636; A61P 35/00; C12N 2501/24; C12N 2501/52

(57) ABSTRACT

Disclosed are and methods for expanding tumor infiltrating lymphocyte (TIL) populations and methods of the use of the expanded TIL population for treating cancer. In one aspect, disclosed herein are methods of generating tumor infiltrating lymphocytes comprising a) administering an effective amount of an oncolytic virus expressing one or more exogenous immunostimulatory molecules (such as, for example, CD40-L, MEM40, B7-1(CD80)/B7-2(CD86), OX40L, 4-1BBL, CD70, GITRL, LIGHT, TIM-4, ICAM-1, CD58 and/or SLAMF6) into a tumor cell; and b) harvesting the tumor infiltrating lymphocytes. In some aspects, the oncolytic virus can further express one or more type 1 interferon (IFN)(such as, for example, IFN-α, IFN-β, IFN-κ, IFN-δ, IFN-ε, IFN-τ, IFN-ω, and/or IFN-ζ). In some aspects, the TILs generated are obtained in the tumor microenvironment at the site of the administration of the oncolytic virus; however TILs can also be obtained at tumor microenvironments not infected with the oncolytic virus.

The present application claims the benefit of U.S. Provisional Application No. 63/106,215, filed Dec. 7, 2021, which is incorporated herein by reference in its entirety.

### BACKGROUND OF THE INVENTION

Recent advances in cancer immunotherapies have revolutionized treatment paradigms. However, many cancer patients derive little or no benefit from available treatments, including immune checkpoint inhibitors (ICI). A host of clinical studies have shown that pre-existing tumor-reactive T cells are critically important to achieve benefit from checkpoint inhibitors. Therefore, what is needed are new methods for augmenting the number and function of tumor-reactive T cells.

### SUMMARY

Disclosed are methods and compositions related to expanding tumor infiltrating lymphocyte (TIL) populations and using said TIL populations for the treatment of cancer.

In one aspect, disclosed herein are methods of generating tumor infiltrating lymphocytes comprising a) administering an effective amount of an oncolytic virus expressing one or more exogenous immunostimulatory molecules (such as, for example, CD40-L, MEM40, B7-1(CD80)/B7-2(CD86), OX40L, 4-1BBL, CD70, GITRL, LIGHT, TIM-4, ICAM-1, CD58 and/or SLAMF6) into a tumor cell; and b) harvesting the tumor infiltrating lymphocytes. In some aspects, the oncolytic virus can further express one or more type 1 interferon (IFN) (such as, for example, IFN-α, IFN-β, IFN-κ, IFN-δ, IFN-ε, IFN-τ, IFN-ω, and/or IFN-ζ). In some aspects, the TILs generated are obtained in the tumor microenvironment at the site of the administration of the oncolytic virus; however TILs can also be obtained at tumor microenvironments not infected with the oncolytic virus.

Also disclosed herein are methods of generating tumor infiltrating lymphocytes of any preceding aspect, wherein the oncolytic virus is administered via intra tumoral injection.

Also disclosed herein are methods of generating tumor infiltrating lymphocytes of any preceding aspect, further comprising expanding the harvested TILs ex vivo.

In one aspect, disclosed herein are methods of expanding a population of tumor infiltrating lymphocytes (TILs) or marrow infiltrating lymphocytes (MILs) comprising: a) harvesting TILs or MILs from a subject with a cancer; b) culturing the harvested TILs or MILs in the presence of antigen presenting cells infected with an oncolytic virus expressing one or more exogenous immunostimulatory molecules (such as, for example, CD40-L, MEM40, B7-1(CD80)/B7-2(CD86), OX40L, 4-1BBL, CD70, GITRL, LIGHT, TIM-4, ICAM-1, CD58 and/or SLAMF6). In some aspects, the oncolytic virus can further express one or more type 1 interferon (IFN) (such as, for example, IFN-α, IFN-β, IFN-κ, IFN-δ, IFN-ε, IFN-τ, IFN-ω, and/or IFN-ζ).

Also disclosed herein are methods of treating, reducing, inhibiting, decreasing, ameliorating, and/or preventing a cancer and/or metastasis in a subject comprising administering to a subject a therapeutically effective amount of the TILs or MILs of any of any preceding aspect. For example, disclosed herein are methods of treating, reducing, inhibiting, decreasing, ameliorating, and/or preventing a cancer and/or metastasis a cancer in a subject comprising: a) administering an effective amount of an oncolytic virus expressing one or more exogenous immunostimulatory molecules (such as, for example, CD40-L, MEM40, B7-1(CD80)/B7-2(CD86), OX40L, 4-1BBL, CD70, GITRL, LIGHT, TIM-4, ICAM-1, CD58 and/or SLAMF6) into a tumor cell; b) harvesting the tumor infiltrating lymphocytes (TILs) and/or marrow infiltrating lymphocytes (MILs); c) expanding the harvested TILs and/or MILs ex vivo; and d) administering a therapeutically effective amount of the expanded TILs and/or MILs to the subject. In some aspects, the cancerous or metastatic tumor being treated, reduced, inhibited, decreased, ameliorated, and/or prevented are abscopal to the tumor receiving any of the oncolytic viruses and/or TILs or MILs disclosed herein. In some aspects, the oncolytic virus can further express one or more type 1 interferon (IFN) (such as, for example, IFN-α, IFN-β, IFN-κ, IFN-δ, IFN-ε, IFN-τ, IFN-ω, and/or IFN-ζ).

Alternatively, disclosed herein are methods of treating, reducing, inhibiting, decreasing, ameliorating, and/or preventing a cancer and/or metastasis in a subject comprising a) harvesting tumor infiltrating lymphocytes (TILs) and/or marrow infiltrating lymphocytes (MILs) from a subject with a cancer; culturing the harvested TILs or MILs in the presence of antigen presenting cells infected with an oncolytic virus expressing one or more exogenous immunostimulatory molecules; and administering a therapeutically effective amount of the expanded TILs and/or MILs to the subject. In some aspects, the oncolytic virus can further express one or more type 1 interferon (IFN) (such as, for example, IFN-α, IFN-β, IFN-κ, IFN-δ, IFN-ε, IFN-τ, IFN-ω, and/or IFN-ζ).

In one aspect, disclosed herein are methods of treating, reducing, inhibiting, decreasing, ameliorating, and/or preventing a cancer and/or metastasis of any preceding aspect, further comprising administering to the subject an anti-cancer agent.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1A shows that B16-OVA cells were transduced with pLenti-Puro control lentivirus or lentivirus expressing mouse or human IFNβ followed by puromycin selection. Equal numbers of cells were plated 2 days after which supernatant was collected for ELISA to detect human and mouse IFNβ. n=6 Statistical analysis was done using two-way ANOVA for growth curves or a t test for OVA tetramer evaluation. Statistical significance is indicated by p-values as *p<0.05, **p<0.01, ***p<0.001. NS: not significant.

FIG. 1B shows that wild-type or IFNαR1 KO (IFNαR) C57BL/6 mice were subjected to two rounds of B16-OVA-pLenti, B16-OVA-mouse IFNβ or B16-OVA-human IFNβ vaccines on DO and 7 (cells were irradiated 100 Gy before injection). Percentage of OVA tetramer⁺ in MHCII⁻ and CD8⁺ cells in peripheral blood on D12 are shown. n=6 Statistical analysis was done using two-way ANOVA for growth curves or a t test for OVA tetramer evaluation. Statistical significance is indicated by p-values as *p<0.05, **p<0.01, ***p<0.001. NS: not significant.

FIG. 1C is the same as in (A) except mice were challenged with 3e5 live B16-OVA cells on D21. Tumor growth was determined as indicated. n=6 Statistical analysis was done using two-way ANOVA for growth curves or a t test for OVA tetramer evaluation. Statistical significance is indicated by p-values as *p<0.05, **p<0.01, ***p<0.001. NS: not significant.

FIG. 2A. Schematic representation of MEM40.

FIG. 2B. Schematic representation of MEM-288.

FIGS. 3A, 3B, 3C, and 3D show that (3A) A549 or (3C) B16-F10 cells were infected with control GFP virus or oncolytic Ad-MEM-188 or 288 as indicated at MOI=250 for 2 days. GFP and MEM40 expression was determined by flow cytometry; IFNβ secretion was determined by ELISA following infection with indicated OV in (3B) A549 cells or (3D) B16-F10 cells.

FIGS. 4A, 4B, and 4C show that (4A) 344SQ (344), (4B) B16-F10 mouse cell lines and (4C) A549 human cell line were infected with indicated OVs AD-GFP (GFP), MEM-188 (188) or MEM-288 (288) at different MOIs (1, 10, 100) for 2 days. Cell viability was determined by trypan blue staining assay 2 days after infection.

FIGS. 5A and 5B show that C57BL/6 mice were inoculated s.c. with 5e5 B16-OVA cells. On D12 and 16, these mice were subjected to two intratumoral injections of Ad-GFP, MEM-188 and MEM-288 (5A) 10e8 or (5B) 10e9 IU. Significance of tumor size difference is indicated compared to untreated control mice at the last time-point.

FIG. 5C shows the percentage of OVA-specific MHCII− and CD8+ cells in peripheral blood on D12 is shown after intratumoral injections of MEM-188 and MEM-288 at 10e9 IU in B16-OVA. Statistical analysis was done using two-way ANOVA for growth curves or a t test for OVAT evaluation. Statistical significance is indicated by p-values as *p<0.05, **p<0.01, ***p<0.001. NS: not significant. FIG. 5D shows the quantification of IFNγ ELISPOT result from splenic CD8 T cells. C57BL/6 WT, IFNαR1 KO and CD40 KO mice (3 per group) were inoculated s.c. with 5e5 B16-OVA cells on the flank and subjected to PBS (UT: untreated) or MEM-288 injection at 10e9 IU on D12 and 16 into tumors. Mouse spleens were subjected to magnetic bead isolation of CD8 T cells according to manufacturer's recommendations. Next, 2×10⁵/well CD8 cells and 1×10⁵/well of 50 Gy irradiated B16-OVA cells were plated in triplicate wells and incubated in 96-well plates at 37° C. for 24 h. Tumor cells were stimulated with IFNγ to increase MHC expression. T cells were also cultured alone or with Concavalin A (ConA) as negative and positive controls, respectively. Results are shown relative to ConA treatment of each group. Statistical analysis was done using t test. Statistical significance is indicated by p-values as *p<0.05, **p<0.01, ***p<0.001. NS: not significant.

FIG. 6A shows the treatment regimen in mice: C57BL/6 mice were inoculated s.c. with 5e5 B16-F10 cells on the primary site and with 2.5e5 B16-F10 cells on the contralateral site. As indicated, these mice were injected with MEM-288 at 10e9 IU on D12 and 16 into primary tumors and i.p. anti-PD-1 and CTLA-4 antibodies on D16, D19, D23 and 27.

FIGS. 6B and 6C shows that tumor growth was determined on the primary site (6B) and contralateral site (6C) as indicated. Statistical analysis was done using two-way ANOVA for growth curves. Statistical significance is indicated by p-values or as *p<0.05, **p<0.01, ***p<0.001. NS: not significant.

FIG. 6D shows Kaplan-Meier Survival Analysis showing overall survival of the mice. Statistical significance is indicated by p-values or as *p<0.05, **p<0.01, ***p<0.001. NS: not significant.

FIG. 7A sows 129 mice were inoculated s.c. with 5e5 344 cells on the flank and subjected to Ad-GFP, MEM-188 or 288 injections at 10e9 IU on D12 and D16 into the tumors. Tumor growth was determined on the primary site as indicated. Significance is indicated compared to control UT group (PBS injection). Statistical significance is indicated by p-values or as *p<0.05, **p<0.01, ***p<0.001. NS: not significant.

FIG. 7B shows typical H&E staining of tumors in lungs of s.c. 344 tumor bearing mice in (7A) on D38.

FIG. 7C shows quantification of tumor mets of individual mice from different groups is shown as indicated. Statistical significance is indicated by p-values or as *p<0.05, **p<0.01, ***p<0.001. NS: not significant.

FIG. 7D shows typical IHC staining of CD8 T cells in the lungs of mice in (7A).

FIG. 7E shows quantification of CD8 T cells density of individual mice from different groups is shown as indicated. Statistical significance is indicated by p-values or as *p<0.05, **p<0.01, ***p<0.001. NS: not significant.

FIGS. 8A and 8B show 129 mice were inoculated s.c. with 5e5 344 cells on the flank and subjected to MEM-288 injection at 10e9 IU on D12 and 16 into the tumors and i.p. anti-PD-1 antibody on D16, D19, D23 and 27. FIG. 8A shows IFNγ ELISPOT from the splenic CD8 T cells of the mice and FIG. 8B shows quantification of ELISPOT results from (A). Statistical analysis was done using t test. Statistical significance is indicated by p-values as *p<0.05, **p<0.01, ***p<0.001. NS: not significant.

FIG. 9A shows flow cytometry analysis scheme of B16-OVA to detect populations of Macrophages, Neutrophils, Monocytes and DCs as indicated.

FIG. 9B shows flow cytometry analysis scheme of B16-OVA to detect populations of B cells, CD4 T cells, CD8 T cells and OVA-specific CD8 T cells.

FIG. 10 shows C57BL/6 mice were inoculated s.c. with 5e5 B16-OVA or B16-OVA-ZsGreen cells on the flank. On D14, flow cytometry analysis of tumor B16-OVA to detect ZsGreen positive populations of macrophages and DCs identified as in FIG. 9A. Percentage of positive cells is indicated.

FIG. 11A shows C57BL/6 mice were inoculated s.c. with 5e5 B16-OVA or B16-OVA-ZsGreen cells on the flank. On D14, flow cytometry analysis was performed to detect ZsGreen⁺ tumor cells in CD45⁻ as indicated.

FIG. 11B shows B16-OVA-ZsGreen tumors were subjected to PBS or MEM-288 injection at 10e9 IU on D12 into tumors. Flow cytometry analysis of CD40L on ZsGreen⁺ CD45⁻ cells was performed from PBS and MEM-288 injected tumors.

FIG. 12A shows flow cytometry analysis of CD80 and CD86 expression in MHC-II^{high} macrophages and DCs (CD11b⁺ DC2) was performed on tumor in FIG. 11b. Injection of PBS and MEM-288 in tumors is indicated.

FIG. 12B shows mean florescence intensity (MFI) of results (A).

FIG. 13 shows DC populations in inguinal LNs. Two main populations of CD11c⁺ MHC-II⁺ DCs were detected. The MHC-II high population comprises of migratory DC1 and DC2. The MHC-II intermediate population comprises of resident DC1 and DC2. As expected, the migratory population has a higher proportion of CD103+DC1 while the resident population has a higher proportion of CD8a⁺ DC1.

FIG. 14 shows DC populations in LNs of WT, CD40 KO and IFNαR1 KO mice. Two main populations of CD11c⁺ MHC-II⁺ DCs were detected in all genotype mice. The MHC-II high population comprises of migratory DC1 and DC2. The MHC-II intermediate population comprises of resident DC1 and DC2.

FIGS. 15A and 15B show C57BL/6 mice were inoculated s.c. with 5e5 B16-OVA cells on the flank. Tumors were subjected to PBS (n=4), Adenovirus-GFP (n=3) or MEM-288 (n=4) injection at 10e9 IU on D12 and D16. On day 20, tumors were obtained to perform IHC to detect presence of CD8⁺ tumor infiltrating lymphocytes (TILs), CD8 T cell staining was scored on a 0-3 scale by a pathologist who was blinded to the groups. FIG. 15A shows typical images of tumors for the 3 treatment groups. FIG. 15B shows scored values of the 3 treatment groups.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings;

“Optional” or “optionally” means that the subsequently described event or circumstance may or may not occur, and that the description includes instances where said event or circumstance occurs and instances where it does not.

As used herein, the term “antigen presenting cell” (APC) refers to professional antigen presenting cell, which is selected from among dendritic cells, macrophages, and B cells. In some embodiments, the APC is a DC. In some embodiments, the APC is a mammalian cell. In some embodiments, the APC, such as DC, macrophage, or B cell is a human cell.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular type 1 IFN and immunostimulatory comprising oncolytic virus is disclosed and discussed and a number of modifications that can be made to a number of molecules including the type 1 IFN and immunostimulatory comprising oncolytic virus are discussed, specifically contemplated is each and every combination and permutation of type 1 IFN and immunostimulatory comprising oncolytic virus and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Engagement of CD40 expressed on DCs by CD40L ligand leads to acquisition of crucial cross-priming function to activate CD8 T cells. Consequently, therapeutic use of CD40 agonists have the potential for triggering strong antitumor T cell immunity. Recent clinical studies have shown a promising response rate of combination of a systemically delivered CD40 agonistic antibody with ICI and chemotherapy but which was also associated with significant toxicity. Type 1 IFNs can function as direct activators of the function of both dendritic cells (DCs) and T cells, in addition to their role in enhancing tumor immunogenicity by increasing expression of immune function genes in multiple cell types. This key immune stimulatory function of type 1 IFN has been recently exploited through use of stimulators of type 1 IFN expression, such as STING and TLR9 agonists. A synergistic relationship occurs between CD40 ligation and stimulators of type I IFN for robust CD8 T cell activation. However, the combined systemic administration of CD40 agonists and activators of type 1 IFNs have high toxicity.

In intralesional immunotherapy approaches, the tumor acts as the vaccine site leading to the activation of DCs and subsequent T cell stimulation to generate a systemic antitumoral immunity capable of controlling growth of distant non-treated tumors. Such approaches aimed at avoiding systemic toxicity have been recently tested with STING and TLR9 agonists. Herein is shown that combined intralesional activation of CD40 and type 1 IFN signaling leads to robust immune activation in the tumor microenvironment (TME) leading to high-level increase in a systemic T cell response.

Oncolytic viruses (OVs) have been developed for their ability to specifically replicate in cancer cells. Recent studies indicate that stimulation of host antitumor immunity is a key mechanism of action of OVs. OVs also allow the capacity to encode for transgenes which can be used to express powerful activators of the immune response to further enhance antitumor immunity. It is shown herein that an intralesionally delivered OV capable of activating an immunostimulatory molecule (such as, for example CD40) and/or type 1 IFN signaling in the TME can function as a potent activator of a systemic T cell response. To test this, a conditionally replicative type 5 adenovirus was developed that expresses a chimeric CD40L ligand (MEM40) and IFNβ in collaboration with Memgen, Inc. We further demonstrate that MEM-288 induces high level expression of CD40L and IFNβ and induces a robust systemic T cell response that is capable of significantly curtailing distant tumor growth in mouse melanoma and lung tumor models.

In one aspect, disclosed herein are methods of generating tumor infiltrating lymphocytes comprising a) administering an oncolytic virus expressing one or more type 1 interferon (IFN) (such as, for example, IFN-α, IFN-β, IFN-κ, IFN-δ, IFN-ε, IFN-τ, IFN-ω, and/or IFN-ζ) and/or one or more exogenous immunostimulatory molecules (such as, for example, CD40-L, MEM40, B7-1(CD80)/B7-2(CD86), OX40L, 4-1BBL, CD70, GITRL, LIGHT, TIM-4, ICAM-1, CD58 and/or SLAMF6) into a tumor cell; and b) harvesting the tumor infiltrating lymphocytes. For example, the disclosed oncolytic virus for use in the disclosed methods can express IFN-α and CD40-L; IFN-α and MEM40; IFN-α and B7-1(CD80)/B7-2(CD86); IFN-α and OX40L; IFN-α and 4-1BBL; IFN-α and CD70; IFN-α and GITRL; IFN-α and LIGHT; IFN-α and TIM-4; IFN-α and ICAM-1; IFN-α and CD58; IFN-α and SLAMF6; IFN-α, CD40-L, and MEM40; IFN-α, CD40-L, and B7-1(CD80)/B7-2(CD86); IFN-α, CD40-L, and OX40L; IFN-α, CD40-L, and 4-1BBL; IFN-α, CD40-L, and CD70; IFN-α, CD40-L, and GITRL; IFN-α, CD40-L, and LIGHT; IFN-α, CD40-L, and TIM-4; IFN-α, CD40-L, and ICAM-1; IFN-α, CD40-L, and CD58; IFN-α, CD40-L, and SLAMF6; IFN-α, CD40-L, MEM40, and B7-1(CD80)/B7-2(CD86); IFN-α, CD40-L, MEM40, and OX40L; IFN-α, CD40-L, MEM40, and 4-1BBL; IFN-α, CD40-L, MEM40, and CD70; IFN-α, CD40-L, MEM40, and GITRL; IFN-α, CD40-L, MEM40, and LIGHT; IFN-α, CD40-L, MEM40, and TIM-4; IFN-α, CD40-L, MEM40, and ICAM-1; IFN-α, CD40-L, MEM40, and CD58; IFN-α, CD40-L, MEM40, and SLAMF6; IFN-α, CD40-L, MEM40, B7-1(CD80)/B7-2(CD86), and OX40L; IFN-α, CD40-L, MEM40, B7-1(CD80)/B7-2(CD86), and 4-1BBL; IFN-α, CD40-L, MEM40, B7-1(CD80)/B7-2(CD86), and CD70; IFN-α, CD40-L, MEM40, B7-1(CD80)/B7-2(CD86), and GITRL; IFN-α, CD40-L, MEM40, B7-1(CD80)/B7-2(CD86), and LIGHT; IFN-α, CD40-L, MEM40, B7-1(CD80)/B7-2(CD86), and TIM-4; IFN-α, CD40-L, MEM40, B7-1(CD80)/B7-2(CD86), and ICAM-1; IFN-α, CD40-L, MEM40, B7-1(CD80)/B7-2(CD86), and CD58; IFN-α, CD40-L, MEM40, B7-1(CD80)/B7-2(CD86), and SLAMF6; IFN-α, CD40-L, MEM40, B7-1(CD80)/B7-2(CD86), OX40L, and 4-1BBL; IFN-α, CD40-L, MEM40, B7-1(CD80)B7-2(CD86), OX40L, and CD70; IFN-α, CD40-L, MEM40, B7-1(CD80)/B7-2(CD86), OX40L, and GITRL; IFN-α, CD40-L, MEM40, B7-1(CD80)/B7-2(CD86), OX40L, and LIGHT; IFN-α, CD40-L, MEM40, B7-1(CD80)/B7-2(CD86), OX40L, and TIM-4; IFN-α, CD40-L, MEM40, B7-1(CD80)/B7-2(CD86), OX40L, and ICAM-1; IFN-α, CD40-L, MEM40, B7-1(CD80)/B7-2(CD86), OX40L, and CD58; IFN-α, CD40-L, MEM40, B7-1(CD80)/B7-2(CD86), OX40L, and SLAMF6; IFN-α, CD40-L, MEM40, B7-1(CD80)/B7-2(CD86), OX40L, 4-1BBL, and CD70; IFN-α, CD40-L, MEM40, B7-1(CD80)/B7-2(CD86), OX40L, 4-1BBL, and GITRL; IFN-α, CD40-L, MEM40, B7-1(CD80)/B7-2(CD86), OX40L, 4-1BBL, and LIGHT; IFN-α, CD40-L, MEM40, B7-1(CD80)/B7-2(CD86), OX40L, 4-1BBL, and TIM-4; IFN-α, CD40-L, MEM40, B7-1(CD80)/B7-2(CD86), OX40L, 4-1BBL, and ICAM-1; IFN-α, CD40-L, MEM40, B7-1(CD80)/B7-2(CD86), OX40L, 4-1BBL, and CD58; IFN-α, CD40-L, MEM40, B7-1(CD80)/B7-2(CD86), OX40L, 4-1BBL, and SLAMF6; IFN-α, CD40-L, MEM40, B7-1(CD80)/B7-2(CD86), OX40L, 4-1BBL, CD70, and GITRL, IFN-α, CD40-L, MEM40, B7-1(CD80)/B7-2(CD86), OX40L, 4-1BBL, CD70, and LIGHT; IFN-α, CD40-L, MEM40, B7-1(CD80)/B7-2(CD86), OX40L, 4-1BBL, CD70, and TIM-4; IFN-α, CD40-L, MEM40, B7-1(CD80)/B7-2(CD86), OX40L, 4-1BBL, CD70, and ICAM-1; IFN-α, CD40-L, MEM40, B7-1(CD80)/B7-2(CD86), OX40L, 4-1BBL, CD70, and CD58; IFN-α, CD40-L, MEM40, B7-1(CD80)/B7-2(CD86), OX40L, 4-1BBL, CD70, and SLAMF6; IFN-α, CD40-L, MEM40, B7-1(CD80)/B7-2(CD86), OX40L, 4-1BBL, CD70, GITRL, and LIGHT; IFN-α, CD40-L, MEM40, B7-1(CD80)/B7-2(CD86), OX40L, 4-1BBL, CD70, GITRL, and TIM-4; IFN-α, CD40-L, MEM40, B7-1(CD80)/B7-2(CD86), OX40L, 4-1BBL, CD70, GITRL, and ICAM-1; IFN-α, CD40-L, MEM40, B7-1(CD80)/B7-2(CD86), OX40L, 4-1BBL, CD70, GITRL, and CD58; IFN-α, CD40-L, MEM40, B7-1(CD80)B7-2(CD86), OX40L, 4-1BBL, CD70, GITRL, and SLAMF6; IFN-α, CD40-L, MEM40, B7-1(CD80)/B7-2(CD86), OX40L, 4-1BBL, CD70, GITRL, LIGHT, and TIM-4; IFN-α, CD40-L, MEM40, B7-1(CD80)/B7-2(CD86), OX40L, 4-1BBL, CD70, GITRL, LIGHT and ICAM-1; IFN-α, CD40-L, MEM40, B7-1(CD80)/B7-2(CD86), OX40L, 4-1BBL, CD70, GITRL, LIGHT and CD58; IFN-α, CD40-L, MEM40, B7-1(CD80)/B7-2(CD86), OX40L, 4-1BBL, CD70, GITRL, LIGHT and SLAMF6; IFN-α, CD40-L, MEM40, B7-1(CD80)B7-2(CD86), OX40L, 4-1BBL, CD70, GITRL, LIGHT, TIM-4, and ICAM-1; IFN-α, CD40-L, MEM40, B7-1(CD80)/B7-2(CD86), OX40L, 4-1BBL, CD70, GITRL, LIGHT, TIM-4, and CD58; IFN-α, CD40-L, MEM40, B7-1(CD80)/B7-2(CD86), OX40L, 4-1BBL, CD70, GITRL, LIGHT, TIM-4, and SLAMF6; IFN-α, CD40-L, MEM40, B7-1(CD80)/B7-2(CD86), OX40L, 4-1BBL, CD70, GITRL, LIGHT, TIM-4, ICAM-1, and CD58; IFN-α, CD40-L, MEM40, B7-1(CD80)/B7-2(CD86), OX40L, 4-1BBL, CD70, GITRL, LIGHT, TIM-4, ICAM-1, and SLAMF6; IFN-α, CD40-L, MEM40, B7-1(CD80)/B7-2(CD86), OX40L, 4-1BBL, CD70, GITRL, LIGHT, TIM-4, ICAM-I, CD58, and SLAMF6; IFN-α, MEM40; IFN-α, MEM40, and B7-1(CD80)/B7-2(CD86); IFN-α, MEM40, and OX40L; IFN-α, MEM40, and 4-1BBL; IFN-α, MEM40, and CD70; IFN-α, MEM40, and GITRL; IFN-α, MEM40, and LIGHT; IFN-α, MEM40, and TIM-4; IFN-α, MEM40, and ICAM-1; IFN-α, MEM40, and CD58; IFN-α, MEM40, and SLAMF6; IFN-α, MEM40, and B7-1(CD80)/B7-2(CD86); IFN-α, MEM40, and OX40L; IFN-α, MEM40, and 4-1BBL; IFN-α, MEM40, and CD70; IFN-α, MEM40, and GITRL; IFN-α, MEM40, and LIGHT; IFN-α, MEM40, and TIM-4; IFN-α, MEM40, and ICAM-1; IFN-α, MEM40, and CD58; IFN-α, MEM40, and SLAMF6; IFN-α, MEM40, B7-1(CD80)/B7-2(CD86), and OX40L; IFN-α, MEM40, B7-1(CD80)/B7-2(CD86), and 4-1BBL; IFN-α, MEM40, B7-1(CD80)/B7-2(CD86), and CD70; IFN-α, MEM40, B7-1(CD80)/B7-2(CD86), and GITRL; IFN-α, MEM40, B7-1(CD80)/B7-2(CD86), and LIGHT; IFN-α, MEM40, B7-1(CD80)/B7-2(CD86), and TIM-4; IFN-α, MEM40, B7-1(CD80)/B7-2(CD86), and ICAM-1; IFN-α, MEM40, B7-1(CD80)/B7-2(CD86), and CD58; IFN-α, MEM40, B7-1(CD80)/B7-2(CD86), and SLAMF6; IFN-α, MEM40, B7-1(CD80)/B7-2(CD86), OX40L, and 4-1BBL; IFN-α, MEM40, B7-1(CD80)/B7-2(CD86), OX40L, and CD70; IFN-α, MEM40, B7-1(CD80)/B7-2(CD86), OX40L, and GITRL; IFN-α, MEM40, B7-1(CD80)/B7-2(CD86), OX40L, and LIGHT; IFN-α, MEM40, B7-1(CD80)/B7-2(CD86), OX40L, and TIM-4; IFN-α, MEM40, B7-1(CD80)/B7-2(CD86), OX40L, and ICAM-1; IFN-α, MEM40, B7-1(CD80)/B7-2(CD86), OX40L, and CD58; IFN-α, MEM40, B7-1(CD80)/B7-2(CD86), OX40L, and SLAMF6; IFN-α, MEM40, B7-1(CD80)/B7-2(CD86), OX40L, 4-1BBL, and CD70; IFN-α, MEM40, B7-1(CD80)/B7-2(CD86), OX40L, 4-1BBL, and GITRL; IFN-α, MEM40, B7-1(CD80)/B7-2(CD86), OX40L, 4-1BBL, and LIGHT; IFN-α, MEM40, B7-1(CD80)/B7-2(CD86), OX40L, 4-1BBL, and TIM-4; IFN-α, MEM40, B7-1(CD80)/B7-2(CD86), OX40L, 4-1BBL, and ICAM-1; IFN-α, MEM40, B7-1(CD80)/B7-2(CD86), OX40L, 4-1BBL, and CD58; IFN-α, MEM40, B7-1(CD80)/B7-2(CD86), OX40L, 4-1BBL, and SLAMF6; IFN-α, MEM40, B7-1(CD80)/B7-2(CD86), OX40L, 4-1BBL, CD70, and GITRL; IFN-α, MEM40, B7-1(CD80)/B7-2(CD86), OX40L, 4-1BBL, CD70, and LIGHT; IFN-α, MEM40, B7-1(CD80)/B7-2(CD86), OX40L, 4-1BBL, CD70, and TIM-4; IFN-α, MEM40, B7-1(CD80)/B7-2(CD86), OX40L, 4-1BBL, CD70, and ICAM-1; IFN-α, MEM40, B7-1(CD80)/B7-2(CD86), OX40L, 4-1BBL, CD70, and CD58; IFN-α, MEM40, B7-1(CD80)/B7-2(CD86), OX40L, 4-1BBL, CD70, and SLAMF6; IFN-α, MEM40, B7-1(CD80)/B7-2(CD86), OX40L, 4-1BBL, CD70, GITRL, and LIGHT; IFN-α, MEM40, B7-1(CD80)/B7-2(CD86), OX40L, 4-1BBL, CD70, GITRL, and TIM-4; IFN-α, MEM40, B7-1(CD80)/B7-2(CD86), OX40L, 4-1BBL, CD70, GITRL, and ICAM-1; IFN-α, MEM40, B7-1(CD80)/B7-2(CD86), OX40L, 4-1BBL, CD70, GITRL, and CD58; IFN-α, MEM40, B7-1(CD80)/B7-2(CD86), OX40L, 4-1BBL, CD70, GITRL, and SLAMF6; IFN-α, MEM40, B7-1(CD80)/B7-2(CD86), OX40L, 4-1BBL, CD70, GITRL, LIGHT, and TIM-4; IFN-α, MEM40, B7-1(CD80)/B7-2(CD86), OX40L, 4-1BBL, CD70, GITRL, LIGHT and ICAM-1; IFN-α, MEM40, B7-1(CD80)/B7-2(CD86), OX40L, 4-1BBL, CD70, GITRL, LIGHT and CD58; IFN-α, MEM40, B7-1(CD80)/B7-2(CD86), OX40L, 4-1BBL, CD70, GITRL, LIGHT and SLAMF6; IFN-α, MEM40, B7-1(CD80)/B7-2(CD86), OX40L, 4-1BBL, CD70, GITRL, LIGHT, TIM-4, and ICAM-1; IFN-α, MEM40, B7-1(CD80)/B7-2(CD86), OX40L, 4-1BBL, CD70, GITRL, LIGHT, TIM-4, and CD58; IFN-α, MEM40, B7-1(CD80)/B7-2(CD86), OX40L, 4-1BBL, CD70, GITRL, LIGHT, TIM-4, and SLAMF6; IFN-α, MEM40, B7-1(CD80)/B7-2(CD86), OX40L, 4-1BBL, CD70, GITRL, LIGHT, TIM-4, ICAM-1, and CD58; IFN-α, MEM40, B7-1(CD80)B7-2(CD86), OX40L, 4-1BBL, CD70, GITRL, LIGHT, TIM-4, ICAM-1, and SLAMF6; IFN-α, MEM40, B7-1(CD80)/B7-2(CD86), OX40L, 4-1BBL, CD70, GITRL, LIGHT, TIM-4, ICAM-1, CD58, and SLAMF6; IFN-α, and B7-1(CD80)/B7-2(CD86); IFN-α, B7-1(CD80)/B7-2(CD86), and OX40L; IFN-α, B7-1(CD80)/B7-2(CD86), and 4-1BBL; IFN-α, B7-1(CD80)/B7-2(CD86), and CD70; IFN-α, B7-1(CD80)/B7-2(CD86), and GITRL; IFN-α, B7-1(CD80)/B7-2(CD86), and LIGHT; IFN-α, B7-1(CD80)/B7-2(CD86), and TIM-4; IFN-α, B7-1(CD80)/B7-2(CD86), and ICAM-1; IFN-α, B7-1(CD80)/B7-2(CD86), and CD58; IFN-α, B7-1(CD80)/B7-2(CD86), and SLAMF6; IFN-α, B7-1(CD80)/B7-2(CD86), and OX40L; IFN-α, B7-1(CD80)/B7-2(CD86), and 4-1BBL; IFN-α, B7-1(CD80)/B7-2(CD86), and CD70; IFN-α, B7-1(CD80)/B7-2(CD86), and GITRL; IFN-α, B7-1(CD80)/B7-2(CD86), and LIGHT; IFN-α, B7-1(CD80)/B7-2(CD86), and TIM-4; IFN-α, B7-1(CD80)/B7-2(CD86), and ICAM-1; IFN-α, B7-1(CD80)/B7-2(CD86), and CD58; IFN-α, B7-1(CD80)/B7-2(CD86), and SLAMF6; IFN-α, B7-1(CD80)/B7-2(CD86), OX40L, and 4-1BBL; IFN-α, B7-1(CD80)/B7-2(CD86), OX40L, and CD70; IFN-α, B7-1(CD80)/B7-2(CD86), OX40L, and GITRL; IFN-α, B7-1(CD80)/B7-2(CD86), OX40L, and LIGHT; IFN-α, B7-1(CD80)B7-2(CD86), OX40L, and TIM-4; IFN-α, B7-1(CD80)/B7-2(CD86), OX40L, and ICAM-1; IFN-α, B7-1(CD80)/B7-2(CD86), OX40L, and CD58; IFN-α, B7-1(CD80)/B7-2(CD86), OX40L, and SLAMF6; IFN-α, B7-1(CD80)/B7-2(CD86), OX40L, 4-1BBL, and CD70; IFN-α, B7-1(CD80)B7-2(CD86), OX40L, 4-1BBL, and GITRL; IFN-α, B7-1(CD80)/B7-2(CD86), OX40L, 4-1BBL, and LIGHT; IFN-α, B7-1(CD80)/B7-2(CD86), OX40L, 4-1BBL, and TIM-4; IFN-α, B7-1(CD80)/B7-2(CD86), OX40L, 4-1BBL, and ICAM-1; IFN-α, B7-1(CD80)/B7-2(CD86), OX40L, 4-1BBL, and CD58; IFN-α, B7-1(CD80)/B7-2(CD86), OX40L, 4-1BBL, and SLAMF6; IFN-α, B7-1(CD80)/B7-2(CD86), OX40L, 4-1BBL, CD70, and GITRL; IFN-α, B7-1(CD80)/B7-2(CD86), OX40L, 4-1BBL, CD70, and LIGHT; IFN-α, B7-1(CD80)/B7-2(CD86), OX40L, 4-1BBL, CD70, and TIM-4; IFN-α, B7-1(CD80)/B7-2(CD86), OX40L, 4-1 BBL, CD70, and ICAM-1; IFN-α, B7-1(CD80)/B7-2(CD86), OX40L, 4-1BBL, CD70, and CD58; IFN-α, B7-1(CD80)/B7-2(CD86), OX40L, 4-1BBL, CD70, and SLAMF6; IFN-α, B7-1(CD80)/B7-2(CD86), OX40L, 4-1BBL, CD70, GITRL, and LIGHT; IFN-α, B7-1(CD80)/B7-2(CD86), OX40L, 4-1BBL, CD70, GITRL, and TIM-4; IFN-α, B7-1(CD80)/B7-2(CD86), OX40L, 4-1BBL, CD70, GITRL, and ICAM-1; IFN-α, B7-1(CD80)/B7-2(CD86), OX40L, 4-1BBL, CD70, GITRL, and CD58; IFN-α, B7-1(CD80)/B7-2(CD86), OX40L, 4-1BBL, CD70, GITRL, and SLAMF6; IFN-α, B7-1(CD80)B7-2(CD86), OX40L, 4-1BBL, CD70, GITRL, LIGHT, and TIM-4; IFN-α, B7-1(CD80)/B7-2(CD86), OX40L, 4-1BBL, CD70, GITRL, LIGHT and ICAM-1, IFN-α, B7-1(CD80)/B7-2(CD86), OX40L, 4-1BBL, CD70, GITRL, LIGHT and CD58; IFN-α, B7-1(CD80)/B7-2(CD86), OX40L, 4-1BBL, CD70, GITRL, LIGHT and SLAMF6; IFN-α, B7-1(CD80)/B7-2(CD86), OX40L, 4-1BBL, CD70, GITRL, LIGHT, TIM-4, and ICAM-1; IFN-α, B7-1(CD80)/B7-2(CD86), OX40L, 4-1BBL, CD70, GITRL, LIGHT, TIM-4, and CD58; IFN-α, B7-1(CD80)/B7-2(CD86), OX40L, 4-1BBL, CD70, GITRL, LIGHT, TIM-4, and SLAMF6; IFN-α, B7-1(CD80)/B7-2(CD86), OX40L, 4-1BBL, CD70, GITRL, LIGHT, TIM-4, ICAM-1, and CD58; IFN-α, B7-1(CD80)/B7-2(CD86), OX40L, 4-1BBL, CD70, GITRL, LIGHT, TIM-4, ICAM-1, and SLAMF6; IFN-α, B7-1(CD80)/B7-2(CD86), OX40L, 4-1BBL, CD70, GITRL, LIGHT, TIM-4, ICAM-1, CD58, and SLAMF6; IFN-α, and OX40L; IFN-α, OX40L, and 4-1BBL; IFN-α, OX40L, and CD70; IFN-α, OX40L, and GITRL; IFN-α, OX40L, and LIGHT; IFN-α, OX40L, and TIM-4; IFN-α, OX40L, and ICAM-1; IFN-α, OX40L, and CD58; IFN-α, OX40L, and SLAMF6; IFN-α, OX40L, and 4-1BBL; IFN-α, OX40L, and CD70; IFN-α, OX40L, and GITRL; IFN-α, OX40L, and LIGHT; IFN-α, OX40L, and TIM-4; IFN-α, OX40L, and ICAM-1; IFN-α, OX40L, and CD58; IFN-α, OX40L, and SLAMF6; IFN-α, OX40L, 4-1BBL, and CD70; IFN-α, OX40L, 4-1BBL, and GITRL; IFN-α, OX40L, 4-1BBL, and LIGHT; IFN-α, OX40L, 4-1BBL, and TIM-4; IFN-α, OX40L, 4-1BBL, and ICAM-1; IFN-α, OX40L, 4-1BBL, and CD58; IFN-α, OX40L, 4-1BBL, and SLAMF6; IFN-α, OX40L, 4-1BBL, CD70, and GITRL; IFN-α, OX40L, 4-1BBL, CD70, and LIGHT; IFN-α, OX40L, 4-1BBL, CD70, and TIM-4; IFN-α, OX40L, 4-1BBL, CD70, and ICAM-1; IFN-α, OX40L, 4-1BBL, CD70, and CD58; IFN-α, OX40L, 4-1BBL, CD70, and SLAMF6; IFN-α, OX40L, 4-1BBL, CD70, GITRL, and LIGHT; IFN-α, OX40L, 4-1BBL, CD70, GITRL, and TIM-4; IFN-α, OX40L, 4-1BBL, CD70, GITRL, and ICAM-1; IFN-α, OX40L, 4-1BBL, CD70, GITRL, and CD58; IFN-α, OX40L, 4-1BBL, CD70, GITRL, and SLAMF6; IFN-α, OX40L, 4-1BBL, CD70, GITRL, LIGHT, and TIM-4; IFN-α, OX40L, 4-1BBL, CD70, GITRL, LIGHT and ICAM-1; IFN-α, OX40L, 4-1BBL, CD70, GITRL, LIGHT and CD58; IFN-α, OX40L, 4-1BBL, CD70, GITRL, LIGHT and SLAMF6; IFN-α, OX40L, 4-1BBL, CD70, GITRL, LIGHT, TIM-4, and ICAM-1; IFN-α, OX40L, 4-1BBL, CD70, GITRL, LIGHT, TIM-4, and CD58; IFN-α, OX40L, 4-1BBL, CD70, GITRL, LIGHT, TIM-4, and SLAMF6; IFN-α, OX40L, 4-1BBL, CD70, GITRL, LIGHT, TIM-4, ICAM-1, and CD58; IFN-α, OX40L, 4-1BBL, CD70, GITRL, LIGHT, TIM-4, ICAM-1, and SLAMF6; IFN-α, OX40L, 4-1BBL, CD70, GITRL, LIGHT, TIM-4, ICAM-1, CD58, and SLAMF6; IFN-α, and 4-1BBL; IFN-α, 4-1BBL, and CD70; IFN-α, 4-1BBL, and GITRL; IFN-α, 4-1BBL, and LIGHT; IFN-α, 4-1BBL, and TIM-4; IFN-α, 4-1BBL, and ICAM-1; IFN-α, 4-1BBL, and CD58; IFN-α, 4-1BBL, and SLAMF6; IFN-α, 4-1BBL, and CD70; IFN-α, 4-1BBL, and GITRL; IFN-α, 4-1BBL, and LIGHT; IFN-α, 4-1BBL, and TIM-4; IFN-α, 4-1BBL, and ICAM-1; IFN-α, 4-1BBL, and CD58; IFN-α, 4-1BBL, and SLAMF6; IFN-α, 4-1BBL, CD70, and GITRL; IFN-α, 4-1BBL, CD70, and LIGHT; IFN-α, 4-1BBL, CD70, and TIM-4; IFN-α, 4-1BBL, CD70, and ICAM-1; IFN-α, 4-1BBL, CD70, and CD58; IFN-α, 4-1BBL, CD70, and SLAMF6; IFN-α, 4-1BBL, CD70, GITRL, and LIGHT; IFN-α, 4-1BBL, CD70, GITRL, and TIM-4; IFN-α, 4-1BBL, CD70, GITRL, and ICAM-1; IFN-α, 4-1BBL, CD70, GITRL, and CD58; IFN-α, 4-1BBL, CD70, GITRL, and SLAMF6; IFN-α, 4-1BBL, CD70, GITRL, LIGHT, and TIM-4; IFN-α, 4-1BBL, CD70, GITRL, LIGHT and ICAM-1; IFN-α, 4-1BBL, CD70, GITRL, LIGHT and CD58; IFN-α, 4-1BBL, CD70, GITRL, LIGHT and SLAMF6; IFN-α, 4-1BBL, CD70, GITRL, LIGHT, TIM-4, and ICAM-1; IFN-α, 4-1BBL, CD70, GITRL, LIGHT, TIM-4, and CD58; IFN-α, 4-1BBL, CD70, GITRL, LIGHT, TIM-4, and SLAMF6; IFN-α, 4-1BBL, CD70, GITRL, LIGHT, TIM-4, ICAM-1, and CD58; IFN-α, 4-1BBL, CD70, GITRL, LIGHT, TIM-4, ICAM-1, and SLAMF6; IFN-α, 4-1BBL, CD70, GITRL, LIGHT, TIM-4, ICAM-1, CD58, and SLAMF6; IFN-α and CD70; IFN-α, CD70, and GITRL; IFN-α, CD70, and LIGHT; IFN-α, CD70, and TIM-4; IFN-α, CD70, and ICAM-1; IFN-α, CD70, and CD58; IFN-α, CD70, and SLAMF6; IFN-α, CD70, and GITRL; IFN-α, CD70, and LIGHT; IFN-α, CD70, and TIM-4; IFN-α, CD70, and ICAM-1; IFN-α, CD70, and CD58; IFN-α, CD70, and SLAMF6; IFN-α, CD70, GITRL, and LIGHT; IFN-α, CD70, GITRL, and TIM-4; IFN-α, CD70, GITRL, and ICAM-1; IFN-α, CD70, GITRL, and CD58; IFN-α, CD70, GITRL, and SLAMF6; IFN-α, CD70, GITRL, LIGHT, and TIM-4; IFN-α, CD70, GITRL, LIGHT and ICAM-1; IFN-α, CD70, GITRL, LIGHT and CD58; IFN-α, CD70, GITRL, LIGHT and SLAMF6; IFN-α, CD70, GITRL, LIGHT, TIM-4, and ICAM-1; IFN-α, CD70, GITRL, LIGHT, TIM-4, and CD58; IFN-α, CD70, GITRL, LIGHT, TIM-4, and SLAMF6; IFN-α, CD70, GITRL, LIGHT, TIM-4, ICAM-1, and CD58; IFN-α, CD70, GITRL, LIGHT, TIM-4, ICAM-1, and SLAMF6; IFN-α, CD70, GITRL, LIGHT, TIM-4, ICAM-1, CD58, and SLAMF6; IFN-α and GITRL; IFN-α, GITRL, and LIGHT; IFN-α, GITRL, and TIM-4; IFN-α, GITRL, and ICAM-1; IFN-α, GITRL, and CD58; IFN-α, GITRL, and SLAMF6; IFN-α, GITRL, and LIGHT; IFN-α, GITRL, and TIM-4; IFN-α, GITRL, and ICAM-1; IFN-α, GITRL, and CD58; IFN-α, GITRL, and SLAMF6; IFN-α, GITRL, LIGHT, and TIM-4; IFN-α, GITRL, LIGHT and ICAM-1; IFN-α, GITRL, LIGHT and CD58; IFN-α, GITRL, LIGHT and SLAMF6; IFN-α, GITRL, LIGHT, TIM-4, and ICAM-1; IFN-α, GITRL, LIGHT, TIM-4, and CD58; IFN-α, GITRL, LIGHT, TIM-4, and SLAMF6; IFN-α, GITRL, LIGHT, TIM-4, ICAM-1, and CD58; IFN-α, GITRL, LIGHT, TIM-4, ICAM-1, and SLAMF6; IFN-α, GITRL, LIGHT, TIM-4, ICAM-1, CD58, and SLAMF6; IFN-α, and LIGHT; IFN-α, LIGHT, and TIM-4; IFN-α, LIGHT, and ICAM-1; IFN-α, LIGHT, and CD58; IFN-α, LIGHT, and SLAMF6; IFN-α, LIGHT, and TIM-4; IFN-α, LIGHT and ICAM-1; IFN-α, LIGHT and CD58; IFN-α, LIGHT and SLAMF6; IFN-α, LIGHT, TIM-4, and ICAM-1; IFN-α, LIGHT, TIM-4, and CD58; IFN-α, LIGHT, TIM-4, and SLAMF6; IFN-α, LIGHT, TIM-4, ICAM-1, and CD58; IFN-α, LIGHT, TIM-4, ICAM-1, and SLAMF6; IFN-α, LIGHT, TIM-4, ICAM-1, CD58, and SLAMF6; IFN-α, and TIM-4; IFN-α, TIM-4, and ICAM-1; IFN-α, TIM-4, and CD58; IFN-α, TIM-4, and SLAMF6; IFN-α, TIM-4, and ICAM-1; IFN-α, TIM-4, and CD58; IFN-α, TIM-4, and SLAMF6; IFN-α, TIM-4, ICAM-1, and CD58; IFN-α, TIM-4, ICAM-1, and SLAMF6; IFN-α, TIM-4, ICAM-1, CD58, and SLAMF6; IFN-α and ICAM-1; IFN-α, ICAM-1, and CD58; IFN-α, ICAM-1, and SLAMF6; IFN-α, ICAM-1, and CD58; IFN-α, ICAM-1, and SLAMF6; IFN-α, ICAM-1, CD58, and SLAMF6; IFN-α and CD58; IFN-α, CD58, and SLAMF6; IFN-α and SLAMF6; IFN-α, IFN-β, and CD40-L; IFN-α, IFN-β, and MEM40; IFN-α, IFN-β, and B7-1(CD80)/B7-2(CD86); IFN-α, IFN-β, and OX40L; IFN-α, IFN-β, and 4-1BBL; IFN-α, IFN-β, and CD70; IFN-α, IFN-β, and GITRL; IFN-α, IFN-β, and LIGHT; IFN-α, IFN-β, and TIM-4; IFN-α, IFN-0, and ICAM-1; IFN-α, IFN-β, and CD58; IFN-α, IFN-β, and SLAMF6; IFN-α, IFN-β, IFN-κ, and CD40-L; IFN-α, IFN-β, IFN-κ, and MEM40; IFN-α, IFN-β, IFN-κ, and B7-1(CD80)/B7-2(CD86); IFN-α, IFN-β, IFN-κ, and OX40L; IFN-α, IFN-β, IFN-κ, and 4-1BBL; IFN-α, IFN-β, IFN-κ, and CD70; IFN-α, IFN-β, IFN-κ, and GITRL; IFN-α, IFN-β, IFN-κ, and LIGHT; IFN-α, IFN-β, IFN-κ, and TIM-4; IFN-α, IFN-β, IFN-κ, and ICAM-1; IFN-α, IFN-β, and CD58; IFN-α, IFN-β, IFN-κ, and SLAMF6; IFN-α, IFN-β, IFN-κ, IFN-δ, and CD40-L; IFN-α, IFN-β, IFN-κ, IFN-δ, and MEM40; IFN-α, IFN-β, IFN-κ, IFN-δ, and B7-1(CD80)/B7-2(CD86); IFN-α, IFN-β, IFN-κ, IFN-δ, and OX40L; IFN-α, IFN-β, IFN-κ, IFN-δ, and 4-1BBL; IFN-α, IFN-β, IFN-κ, IFN-δ, and CD70; IFN-α, IFN-β, IFN-κ, IFN-δ, and GITRL; IFN-α, IFN-β, IFN-κ, IFN-δ, and LIGHT; IFN-α, IFN-β, IFN-κ, IFN-β, and TIM-4; IFN-α, IFN-β, IFN-κ, IFN-δ, and ICAM-1; IFN-α, IFN-β, IFN-δ, and CD58; IFN-α, IFN-β, IFN-κ, IFN-δ, and SLAMF6; IFN-α, IFN-β, IFN-κ, IFN-δ, IFN-ε, and CD40-L; IFN-α, IFN-β, IFN-κ, IFN-δ, IFN-ε, and MEM40; IFN-α, IFN-β, IFN-κ, IFN-δ, IFN-ε, and B7-1(CD80)/B7-2(CD86); IFN-α, IFN-β, IFN-κ, IFN-δ, IFN-ε, and OX40L; IFN-α, IFN-β, IFN-κ, IFN-δ, IFN-ε, and 4-1BBL; IFN-α, IFN-β, IFN-κ, IFN-δ, IFN-ε, and CD70; IFN-α, IFN-β, IFN-κ, IFN-δ, IFN-ε, and GITRL; IFN-α, IFN-β, IFN-κ, IFN-δ, IFN-ε, and LIGHT; IFN-α, IFN-β, IFN-κ, IFN-δ, IFN-ε, and TIM-4; IFN-α, IFN-β, IFN-κ, IFN-δ, IFN-ε, and ICAM-1; IFN-α, IFN-β, IFN-δ, IFN-ε, and CD58; IFN-α, IFN-β, IFN-κ, IFN-δ, IFN-ε, and SLAMF6; IFN-α, IFN-β, IFN-κ, IFN-δ, IFN-ε, IFN-τ, and CD40-L; IFN-α, IFN-β, IFN-κ, IFN-δ, IFN-ε, IFN-τ, and MEM40; IFN-α, IFN-β, IFN-κ, IFN-δ, IFN-ε, IFN-ε, and B7-1(CD80)/B7-2(CD86); IFN-α, IFN-β, IFN-κ, IFN-δ, IFN-ε, IFN-τ, and OX40L; IFN-α, IFN-β, IFN-κ, IFN-δ, IFN-ε, IFN-τ, and 4-1BBL; IFN-α, IFN-β, IFN-κ, IFN-δ, IFN-ε, IFN-ε, and CD70; IFN-α, IFN-β, IFN-κ, IFN-δ, IFN-ε, IFN-τ, and GITRL; IFN-α, IFN-β, IFN-κ, IFN-δ, IFN-ε, IFN-τ, and LIGHT; IFN-α, IFN-β, IFN-κ, IFN-δ, IFN-ε, IFN-τ, and TIM-4; IFN-α, IFN-β, IFN-κ, IFN-δ, IFN-ε, IFN-τ, and ICAM-1; IFN-α, IFN-β, IFN-δ, IFN-ε, IFN-τ, and CD58; IFN-α, IFN-β, IFN-κ, IFN-δ, IFN-ε, IFN-τ, and SLAMF6; IFN-α, IFN-β, IFN-κ, IFN-δ, IFN-ε, IFN-τ, IFN-ω, and CD40-L; IFN-α, IFN-β, IFN-κ, IFN-δ, IFN-ε, IFN-τ, IFN-ω, and MEM40; IFN-α, IFN-β, IFN-κ, IFN-δ, IFN-ε, IFN-τ, IFN-ω, and B7-1(CD80)/B7-2(CD86); IFN-α, IFN-β, IFN-κ, IFN-δ, IFN-ε, IFN-τ, IFN-ω, and OX40L; IFN-α, IFN-β, IFN-κ, IFN-δ, IFN-ε, IFN-ε, IFN-ω, and 4-1BBL; IFN-α, IFN-β, IFN-κ, IFN-δ, IFN-ε, IFN-τ, IFN-ω, and CD70; IFN-α, IFN-β, IFN-κ, IFN-δ, IFN-ε, IFN-τ, IFN-ω, and GITRL; IFN-α, IFN-β, IFN-κ, IFN-δ, IFN-ε, IFN-ε, IFN-ω, and LIGHT; IFN-α, IFN-β, IFN-κ, IFN-δ, IFN-ε, IFN-τ, IFN-ω, and TIM-4; IFN-α, IFN-β, IFN-κ, IFN-δ, IFN-ε, IFN-τ, IFN-ω, and ICAM-1; IFN-α, IFN-β, IFN-δ, IFN-ε, IFN-τ, IFN-ω, and CD58; IFN-α, IFN-β, IFN-κ, IFN-δ, IFN-ε, IFN-τ, IFN-ω, and SLAMF6; IFN-α, IFN-β, IFN-κ, IFN-δ, IFN-ε, IFN-τ, IFN-ω, IFN-ζ, and CD40-L; IFN-α, IFN-β, IFN-κ, IFN-δ, IFN-ε, IFN-τ, IFN-ω, IFN-ζ, and MEM40; IFN-α, IFN-β, IFN-κ, IFN-δ, IFN-ε, IFN-τ, IFN-ω, IFN-ζ, and B7-1(CD80)/B7-2(CD86); IFN-α, IFN-β, IFN-κ, IFN-δ, IFN-ε, IFN-τ, IFN-ω, IFN-ζ, and OX40L; IFN-α, IFN-β, IFN-κ, IFN-δ, IFN-ε, IFN-τ, IFN-ω, IFN-ζ, and 4-1BBL; IFN-α, IFN-β, IFN-κ, IFN-δ, IFN-ε, IFN-τ, IFN-ω, IFN-ζ, and CD70; IFN-α, IFN-β, IFN-κ, IFN-δ, IFN-ε, IFN-τ, IFN-ω, IFN-ζ, and GITRL; IFN-α, IFN-β, IFN-κ, IFN-δ, IFN-ε, IFN-τ, IFN-ω, IFN-ζ, and LIGHT, IFN-α, IFN-β, IFN-κ, IFN-δ, IFN-ε, IFN-τ, IFN-ω, IFN-ζ, and TIM-4; IFN-α, IFN-β, IFN-κ, IFN-δ, IFN-ε, IFN-τ, IFN-ω, IFN-ζ, and ICAM-1; IFN-α, IFN-β, IFN-δ, IFN-ε, IFN-ε, IFN-ω, IFN-ζ, and CD58; IFN-α, IFN-β, IFN-κ, IFN-δ, IFN-ε, IFN-τ, IFN-ω, IFN-ζ, and SLAMF6; IFN-β and CD40-L; IFN-β and MEM40; IFN-β and B7-1(CD80)/B7-2(CD86); IFN-β and OX40L; IFN-β and 4-1BBL; IFN-β and CD70; IFN-β and GITRL; IFN-β and LIGHT; IFN-β and TIM-4; IFN-β and ICAM-1; IFN-β and CD58; IFN-β and SLAMF6; IFN-κ and CD40-L; IFN-κ and MEM40; IFN-κ and B7-1(CD80)/B7-2(CD86); IFN-κ and OX40L; IFN-κ and 4-1BBL; IFN-κ and CD70; IFN-κ and GITRL; IFN-κ and LIGHT; IFN-κ and TIM-4; IFN-κ and ICAM-1; IFN-κ and CD58; IFN-α and SLAMF6; IFN-δ and CD40-L; IFN-δ and MEM40; IFN-δ and B7-1(CD80)/B7-2(CD86); IFN-δ and OX40L; IFN-δ and 4-1BBL; IFN-δ and CD70; IFN-δ and GITRL; IFN-δ and LIGHT; IFN-δ and TIM-4; IFN-δ and ICAM-1; IFN-δ and CD58; IFN-δ and SLAMF6; IFN-ε and CD40-L; IFN-ε and MEM40; IFN-ε and B7-1(CD80)/B7-2(CD86); IFN-ε and OX40L; IFN-ε and 4-1BBL; IFN-ε and CD70; IFN-ε and GITRL; IFN-ε and LIGHT; IFN-ε and TIM-4; IFN-ε and ICAM-1; IFN-ε and CD58; IFN-ε and SLAMF6; IFN-τ and CD40-L; IFN-τ and MEM40; IFN-τ and B7-1(CD80)/B7-2(CD86); IFN-τ and OX40L; IFN-τ and 4-1BBL; IFN-τ and CD70; IFN-τ and GITRL; IFN-τ and LIGHT; IFN-τ and TIM-4; IFN-τ and ICAM-1; IFN-τ and CD58; IFN-τ and SLAMF6; IFN-ω and CD40-L; IFN-ω and MEM40; IFN-ω and B7-1(CD80)/B7-2(CD86); IFN-ω and OX40L; IFN-ω and 4-1BBL; IFN-ω and CD70, IFN-ω and GITRL; IFN-ω and LIGHT; IFN-ω and TIM-4; IFN-ω and ICAM-1; IFN-ω and CD58; IFN-ω and SLAMF6; IFN-ζ and CD40-L; IFN-ζ and MEM40; IFN-ζ and B7-1(CD80)/B7-2(CD86); IFN-ζ and OX40L; IFN-ζ and 4-1BBL; IFN-ζ and CD70; IFN-ζ and GITRL; IFN-ζ and LIGHT; IFN-ζ and TIM-4; IFN-ζ and ICAM-1; IFN-ζ and CD58; IFN-ζ and SLAMF6; IFN-β, CD40-L, and MEM40; IFN-β, CD40-L, and B7-1(CD80)/B7-2(CD86); IFN-β, CD40-L, and OX40L; IFN-β, CD40-L, and 4-1BBL; IFN-β, CD40-L, and CD70; IFN-β, CD40-L, and GITRL; IFN-β, CD40-L, and LIGHT; IFN-β, CD40-L, and TIM-4; IFN-β, CD40-L, and ICAM-1; IFN-β, CD40-L, and CD58; IFN-β, CD40-L, and SLAMF6; IFN-β, CD40-L, MEM40, and B7-1(CD80)/B7-2(CD86); IFN-β, CD40-L, MEM40, and OX40L; IFN-β, CD40-L, MEM40, and 4-1BBL; IFN-β, CD40-L, MEM40, and CD70; IFN-β, CD40-L, MEM40, and GITRL; IFN-β, CD40-L, MEM40, and LIGHT; IFN-β, CD40-L, MEM40, and TIM-4; IFN-β, CD40-L, MEM40, and ICAM-1; IFN-β, CD40-L, MEM40, and CD58; IFN-β, CD40-L, MEM40, and SLAMF6; IFN-β, CD40-L, MEM40, B7-1(CD80)/B7-2(CD86), and OX40L; IFN-β, CD40-L, MEM40, B7-1(CD80)/B7-2(CD86), and 4-1BBL; IFN-β, CD40-L, MEM40, B7-1(CD80)B7-2(CD86), and CD70; IFN-β, CD40-L, MEM40, B7-1(CD80)/B7-2(CD86), and GITRL; IFN-β, CD40-L, MEM40, B7-1(CD80)/B7-2(CD86), and LIGHT; IFN-β, CD40-L, MEM40, B7-1(CD80)/B7-2(CD86), and TIM-4; IFN-β, CD40-L, MEM40, B7-1(CD80)/B7-2(CD86), and ICAM-1; IFN-β, CD40-L, MEM40, B7-1(CD80)/B7-2(CD86), and CD58; IFN-β, CD40-L, MEM40, B7-1(CD80)/B7-2(CD86), and SLAMF6; IFN-β, CD40-L, MEM40, B7-1(CD80)/B7-2(CD86), OX40L, and 4-1BBL; IFN-β, CD40-L, MEM40, B7-1(CD80)/B7-2(CD86), OX40L, and CD70; IFN-β, CD40-L, MEM40, B7-1(CD80)/B7-2(CD86), OX40L, and GITRL; IFN-β, CD40-L, MEM40, B7-1(CD80)/B7-2(CD86), OX40L, and LIGHT; IFN-β, CD40-L, MEM40, B7-1(CD80)/B7-2(CD86), OX40L, and TIM-4; IFN-β, CD40-L, MEM40, B7-1(CD80)/B7-2(CD86), OX40L, and ICAM-1; IFN-β, CD40-L, MEM40, B7-1(CD80)/B7-2(CD86), OX40L, and CD58; IFN-β, CD40-L, MEM40, B7-1(CD80)/B7-2(CD86), OX40L, and SLAMF6; IFN-β, CD40-L, MEM40, B7-1(CD80)/B7-2(CD86), OX40L, 4-1BBL, and CD70; IFN-β, CD40-L, MEM40, B7-1(CD80)/B7-2(CD86), OX40L, 4-1BBL, and GITRL; IFN-β, CD40-L, MEM40, B7-1(CD80)/B7-2(CD86), OX40L, 4-1BBL, and LIGHT; IFN-β, CD40-L, MEM40, B7-1(CD80)/B7-2(CD86), OX40L, 4-1BBL, and TIM-4; IFN-β, CD40-L, MEM40, B7-1(CD80)/B7-2(CD86), OX40L, 4-1BBL, and ICAM-1; IFN-β, CD40-L, MEM40, B7-1(CD80)/B7-2(CD86), OX40L, 4-1BBL, and CD58; IFN-β, CD40-L, MEM40, B7-1(CD80)/B7-2(CD86), OX40L, 4-1BBL, and SLAMF6; IFN-β, CD40-L, MEM40, B7-1(CD80)/B7-2(CD86), OX40L, 4-1BBL, CD70, and GITRL; IFN-β, CD40-L, MEM40, B7-1(CD80)/B7-2(CD86), OX40L, 4-1BBL, CD70, and LIGHT; IFN-β, CD40-L, MEM40, B7-1(CD80)/B7-2(CD86), OX40L, 4-1BBL, CD70, and TIM-4; IFN-β, CD40-L, MEM40, B7-1(CD80)/B7-2(CD86), OX40L, 4-1BBL, CD70, and ICAM-1; IFN-β, CD40-L, MEM40, B7-1(CD80)/B7-2(CD86), OX40L, 4-1BBL, CD70, and CD58; IFN-β, CD40-L, MEM40, B7-1(CD80)/B7-2(CD86), OX40L, 4-1BBL, CD70, and SLAMF6; IFN-β, CD40-L, MEM40, B7-1(CD80)/B7-2(CD86), OX40L, 4-1BBL, CD70, GITRL, and LIGHT; IFN-β, CD40-L, MEM40, B7-1(CD80)/B7-2(CD86), OX40L, 4-1BBL, CD70, GITRL, and TIM-4; IFN-β, CD40-L, MEM40, B7-1(CD80)/B7-2(CD86), OX40L, 4-1BBL, CD70, GITRL, and ICAM-1; IFN-β, CD40-L, MEM40, B7-1(CD80)/B7-2(CD86), OX40L, 4-1BBL, CD70, GITRL, and CD58; IFN-β, CD40-L, MEM40, B7-1(CD80)/B7-2(CD86), OX40L, 4-1BBL, CD70, GITRL, and SLAMF6; IFN-β, CD40-L, MEM40, B7-1(CD80)/B7-2(CD86), OX40L, 4-1BBL, CD70, GITRL, LIGHT, and TIM-4; IFN-β, CD40-L, MEM40, B7-1(CD80)/B7-2(CD86), OX40L, 4-1BBL, CD70, GITRL, LIGHT and ICAM-1, IFN-β, CD40-L, MEM40, B7-1(CD80)/B7-2(CD86), OX40L, 4-1BBL, CD70, GITRL, LIGHT and CD58; IFN-β, CD40-L, MEM40, B7-1(CD80)/B7-2(CD86), OX40L, 4-1BBL, CD70, GITRL, LIGHT and SLAMF6; IFN-β, CD40-L, MEM40, B7-1(CD80)/B7-2(CD86), OX40L, 4-1BBL, CD70, GITRL, LIGHT, TIM-4, and ICAM-1; IFN-β, CD40-L, MEM40, B7-1(CD80)/B7-2(CD86), OX40L, 4-1BBL, CD70, GITRL, LIGHT, TIM-4, and CD58; IFN-β, CD40-L, MEM40, B7-1(CD80)/B7-2(CD86), OX40L, 4-1BBL, CD70, GITRL, LIGHT, TIM-4, and SLAMF6; IFN-β, CD40-L, MEM40, B7-1(CD80)/B7-2(CD86), OX40L, 4-1BBL, CD70, GITRL, LIGHT, TIM-4, ICAM-1, and CD58; IFN-β, CD40-L, MEM40, B7-1(CD80)/B7-2(CD86), OX40L, 4-1BBL, CD70, GITRL, LIGHT, TIM-4, ICAM-1, and SLAMF6; IFN-β, CD40-L, MEM40, B7-1(CD80)/B7-2(CD86), OX40L, 4-1BBL, CD70, GITRL, LIGHT, TIM-4, ICAM-1, CD58, and SLAMF6; IFN-β, MEM40; IFN-β, MEM40, and B7-1(CD80)/B7-2(CD86); IFN-β, MEM40, and OX40L; IFN-β, MEM40, and 4-1BBL; IFN-β, MEM40, and CD70; IFN-β, MEM40, and GITRL; IFN-β, MEM40, and LIGHT; IFN-β, MEM40, and TIM-4; IFN-β, MEM40, and ICAM-1; IFN-β, MEM40, and CD58; IFN-β, MEM40, and SLAMF6; IFN-β, MEM40, and B7-1(CD80)/B7-2(CD86); IFN-β, MEM40, and OX40L; IFN-β, MEM40, and 4-1BBL; IFN-β, MEM40, and CD70; IFN-β, MEM40, and GITRL; IFN-β, MEM40, and LIGHT; IFN-β, MEM40, and TIM-4; IFN-β, MEM40, and ICAM-1; IFN-β, MEM40, and CD58; IFN-β, MEM40, and SLAMF6; IFN-β, MEM40, B7-1(CD80)/B7-2(CD86), and OX40L; IFN-β, MEM40, B7-1(CD80)/B7-2(CD86), and 4-1BBL; IFN-β, MEM40, B7-1(CD80)/B7-2(CD86), and CD70; IFN-β, MEM40, B7-1(CD80)/B7-2(CD86), and GITRL; IFN-β, MEM40, B7-1(CD80)/B7-2(CD86), and LIGHT; IFN-β, MEM40, B7-1(CD80)/B7-2(CD86), and TIM-4; IFN-β, MEM40, B7-1(CD80)/B7-2(CD86), and ICAM-1; IFN-β, MEM40, B7-1(CD80)/B7-2(CD86), and CD58; IFN-β, MEM40, B7-1(CD80)/B7-2(CD86), and SLAMF6; IFN-β, MEM40, B7-1(CD80)/B7-2(CD86), OX40L, and 4-1BBL; IFN-β, MEM40, B7-1(CD80)/B7-2(CD86), OX40L, and CD70; IFN-β, MEM40, B7-1(CD80)/B7-2(CD86), OX40L, and GITRL; IFN-β, MEM40, B7-1(CD80)/B7-2(CD86), OX40L, and LIGHT; IFN-β, MEM40, B7-1(CD80)/B7-2(CD86), OX40L, and TIM-4; IFN-β, MEM40, B7-1(CD80)/B7-2(CD86), OX40L, and ICAM-1; IFN-β, MEM40, B7-1(CD80)/B7-2(CD86), OX40L, and CD58; IFN-β, MEM40, B7-1(CD80)/B7-2(CD86), OX40L, and SLAMF6; IFN-β, MEM40, B7-1(CD80)/B7-2(CD86), OX40L, 4-1BBL, and CD70; IFN-β, MEM40, B7-1(CD80)/B7-2(CD86), OX40L, 4-1BBL, and GITRL; IFN-β, MEM40, B7-1(CD80)/B7-2(CD86), OX40L, 4-1BBL, and LIGHT; IFN-β, MEM40, B7-1(CD80)/B7-2(CD86), OX40L, 4-1BBL, and TIM-4; IFN-β, MEM40, B7-1(CD80)/B7-2(CD86), OX40L, 4-1BBL, and ICAM-1; IFN-β, MEM40, B7-1(CD80)/B7-2(CD86), OX40L, 4-1BBL, and CD58; IFN-β, MEM40, B7-1(CD80)/B7-2(CD86), OX40L, 4-1BBL, and SLAMF6; IFN-β, MEM40, B7-1(CD80)/B7-2(CD86), OX40L, 4-1BBL, CD70, and GITRL; IFN-β, MEM40, B7-1(CD80)/B7-2(CD86), OX40L, 4-1BBL, CD70, and LIGHT; IFN-β, MEM40, B7-1(CD80)/B7-2(CD86), OX40L, 4-1BBL, CD70, and TIM-4; IFN-β, MEM40, B7-1(CD80)/B7-2(CD86), OX40L, 4-1BBL, CD70, and ICAM-1; IFN-β, MEM40, B7-1(CD80)/B7-2(CD86), OX40L, 4-1BBL, CD70, and CD58; IFN-β, MEM40, B7-1(CD80)/B7-2(CD86), OX40L, 4-1BBL, CD70, and SLAMF6; IFN-β, MEM40, B7-1(CD80)/B7-2(CD86), OX40L, 4-1BBL, CD70, GITRL, and LIGHT; IFN-β, MEM40, B7-1(CD80)/B7-2(CD86), OX40L, 4-1BBL, CD70, GITRL, and TIM-4; IFN-β, MEM40, B7-1(CD80)/B7-2(CD86), OX40L, 4-1BBL, CD70, GITRL, and ICAM-1; IFN-β, MEM40, B7-1(CD80)/B7-2(CD86), OX40L, 4-1BBL, CD70, GITRL, and CD58; IFN-β, MEM40, B7-1(CD80)/B7-2(CD86), OX40L, 4-1BBL, CD70, GITRL, and SLAMF6; IFN-β, MEM40, B7-1(CD80)/B7-2(CD86), OX40L, 4-1BBL, CD70, GITRL, LIGHT, and TIM-4; IFN-β, MEM40, B7-1(CD80)/B7-2(CD86), OX40L, 4-1BBL, CD70, GITRL, LIGHT and ICAM-1; IFN-β, MEM40, B7-1(CD80)/B7-2(CD86), OX40L, 4-1 BBL, CD70, GITRL, LIGHT and CD58; IFN-β, MEM40, B7-1(CD80)/B7-2(CD86), OX40L, 4-1BBL, CD70, GITRL, LIGHT and SLAMF6; IFN-β, MEM40, B7-1(CD80)/B7-2(CD86), OX40L, 4-1BBL, CD70, GITRL, LIGHT, TIM-4, and ICAM-1; IFN-β, MEM40, B7-1(CD80)/B7-2(CD86), OX40L, 4-1BBL, CD70, GITRL, LIGHT, TIM-4, and CD58; IFN-β, MEM40, B7-1(CD80)/B7-2(CD86), OX40L, 4-1BBL, CD70, GITRL, LIGHT, TIM-4, and SLAMF6; IFN-β, MEM40, B7-1(CD80)/B7-2(CD86), OX40L, 4-1BBL, CD70, GITRL, LIGHT, TIM-4, ICAM-1, and CD58; IFN-β, MEM40, B7-1(CD80)/B7-2(CD86), OX40L, 4-1BBL, CD70, GITRL, LIGHT, TIM-4, ICAM-1, and SLAMF6; IFN-β, MEM40, B7-1(CD80)/B7-2(CD86), OX40L, 4-1BBL, CD70, GITRL, LIGHT, TIM-4, ICAM-1, CD58, and SLAMF6; IFN-β, and B7-1(CD80)/B7-2(CD86); IFN-β, B7-1(CD80)/B7-2(CD86), and OX40L; IFN-β, B7-1(CD80)/B7-2(CD86), and 4-1BBL; IFN-β, B7-1(CD80)/B7-2(CD86), and CD70; IFN-β, B7-1(CD80)/B7-2(CD86), and GITRL; IFN-β, B7-1(CD80)/B7-2(CD86), and LIGHT, IFN-β, B7-1(CD80)/B7-2(CD86), and TIM-4; IFN-β, B7-1(CD80)/B7-2(CD86), and ICAM-1; IFN-β, B7-1(CD80)/B7-2(CD86), and CD58; IFN-β, B7-1(CD80)/B7-2(CD86), and SLAMF6; IFN-β, B7-1(CD80)/B7-2(CD86), and OX40L; IFN-β, B7-1(CD80)/B7-2(CD86), and 4-1BBL; IFN-β, B7-1(CD80)/B7-2(CD86), and CD70; IFN-β, B7-1(CD80)/B7-2(CD86), and GITRL; IFN-β, B7-1(CD80)/B7-2(CD86), and LIGHT; IFN-β, B7-1(CD80)/B7-2(CD86), and TIM-4; IFN-β, B7-1(CD80)/B7-2(CD86), and ICAM-1; IFN-β, B7-1(CD80)/B7-2(CD86), and CD58; IFN-β, B7-1(CD80)/B7-2(CD86), and SLAMF6; IFN-β, B7-1(CD80)/B7-2(CD86), OX40L, and 4-1BBL; IFN-β, B7-1(CD80)/B7-2(CD86), OX40L, and CD70; IFN-β, B7-1(CD80)/B7-2(CD86), OX40L, and GITRL; IFN-β, B7-1(CD80)/B7-2(CD86), OX40L, and LIGHT; IFN-β, B7-1(CD80)/B7-2(CD86), OX40L, and TIM-4; IFN-β, B7-1(CD80)/B7-2(CD86), OX40L, and ICAM-1; IFN-β, B7-1(CD80)/B7-2(CD86), OX40L, and CD58; IFN-β, B7-1(CD80)/B7-2(CD86), OX40L, and SLAMF6; IFN-β, B7-1(CD80)/B7-2(CD86), OX40L, 4-1BBL, and CD70; IFN-β, B7-1(CD80)/B7-2(CD86), OX40L, 4-1BBL, and GITRL; IFN-β, B7-1(CD80)/B7-2(CD86), OX40L, 4-1BBL, and LIGHT; IFN-β, B7-1(CD80)/B7-2(CD86), OX40L, 4-1BBL, and TIM-4; IFN-β, B7-1(CD80)/B7-2(CD86), OX40L, 4-1BBL, and ICAM-1; IFN-β, B7-1(CD80)/B7-2(CD86), OX40L, 4-1BBL, and CD58; IFN-β, B7-1(CD80)/B7-2(CD86), OX40L, 4-1BBL, and SLAMF6; IFN-β, B7-1(CD80)/B7-2(CD86), OX40L, 4-1BBL, CD70, and GITRL; IFN-β, B7-1(CD80)/B7-2(CD86), OX40L, 4-1BBL, CD70, and LIGHT; IFN-β, B7-1(CD80)/B7-2(CD86), OX40L, 4-1BBL, CD70, and TIM-4; IFN-β, B7-1(CD80)/B7-2(CD86), OX40L, 4-1BBL, CD70, and ICAM-1; IFN-β, B7-1(CD80)/B7-2(CD86), OX40L, 4-1BBL, CD70, and CD58; IFN-β, B7-1(CD80)/B7-2(CD86), OX40L, 4-1BBL, CD70, and SLAMF6; IFN-β, B7-1(CD80)/B7-2(CD86), OX40L, 4-1BBL, CD70, GITRL, and LIGHT; IFN-β, B7-1(CD80)/B7-2(CD86), OX40L, 4-1BBL, CD70, GITRL, and TIM-4; IFN-β, B7-1(CD80)/B7-2(CD86), OX40L, 4-1BBL, CD70, GITRL, and ICAM-1; IFN-β, B7-1(CD80)/B7-2(CD86), OX40L, 4-1BBL, CD70, GITRL, and CD58; IFN-β, B7-1(CD80)/B7-2(CD86), OX40L, 4-1BBL, CD70, GITRL, and SLAMF6; IFN-β, B7-1(CD80)/B7-2(CD86), OX40L, 4-1BBL, CD70, GITRL, LIGHT, and TIM-4; IFN-β, B7-1(CD80)/B7-2(CD86), OX40L, 4-1BBL, CD70, GITRL, LIGHT and ICAM-1; IFN-β, B7-1(CD80)/B7-2(CD86), OX40L, 4-1BBL, CD70, GITRL, LIGHT and CD58; IFN-β, B7-1(CD80)/B7-2(CD86), OX40L, 4-1BBL, CD70, GITRL, LIGHT and SLAMF6; IFN-β, B7-1(CD80)/B7-2(CD86), OX40L, 4-1BBL, CD70, GITRL, LIGHT, TIM-4, and ICAM-1; IFN-β, B7-1(CD80)/B7-2(CD86), OX40L, 4-1BBL, CD70, GITRL, LIGHT, TIM-4, and CD58; IFN-β, B7-1(CD80)/B7-2(CD86), OX40L, 4-1BBL, CD70, GITRL, LIGHT, TIM-4, and SLAMF6; IFN-β, B7-1(CD80)/B7-2(CD86), OX40L, 4-1BBL, CD70, GITRL, LIGHT, TIM-4, ICAM-1, and CD58; IFN-β, B7-1(CD80)/B7-2(CD86), OX40L, 4-1BBL, CD70, GITRL, LIGHT, TIM-4, ICAM-1, and SLAMF6; IFN-β, B7-1(CD80)/B7-2(CD86), OX40L, 4-1BBL, CD70, GITRL, LIGHT, TIM-4, ICAM-1, CD58, and SLAMF6; IFN-0, and OX40L; IFN-β, OX40L, and 4-1BBL; IFN-β, OX40L, and CD70; IFN-β, OX40L, and GITRL; IFN-β, OX40L, and LIGHT; IFN-β, OX40L, and TIM-4; IFN-β, OX40L, and ICAM-1; IFN-β, OX40L, and CD58; IFN-β, OX40L, and SLAMF6; IFN-β, OX40L, and 4-1BBL; IFN-β, OX40L, and CD70; IFN-β, OX40L, and GITRL; IFN-β, OX40L, and LIGHT; IFN-β, OX40L, and TIM-4; IFN-β, OX40L, and ICAM-1; IFN-β, OX40L, and CD58; IFN-β, OX40L, and SLAMF6; IFN-β, OX40L, 4-1BBL, and CD70; IFN-β, OX40L, 4-1BBL, and GITRL; IFN-β, OX40L, 4-1BBL, and LIGHT; IFN-β, OX40L, 4-1BBL, and TIM-4; IFN-β, OX40L, 4-1BBL, and ICAM-1; IFN-β, OX40L, 4-1BBL, and CD58; IFN-β, OX40L, 4-1BBL, and SLAMF6; IFN-β, OX40L, 4-1BBL, CD70, and GITRL; IFN-β, OX40L, 4-1BBL, CD70, and LIGHT; IFN-β, OX40L, 4-1BBL, CD70, and TIM-4, IFN-β, OX40L, 4-1BBL, CD70, and ICAM-1; IFN-β, OX40L, 4-1BBL, CD70, and CD58; IFN-β, OX40L, 4-1BBL, CD70, and SLAMF6; IFN-β, OX40L, 4-1BBL, CD70, GITRL, and LIGHT; IFN-β, OX40L, 4-1BBL, CD70, GITRL, and TIM-4; IFN-β, OX40L, 4-1BBL, CD70, GITRL, and ICAM-1; IFN-β, OX40L, 4-1BBL, CD70, GITRL, and CD58; IFN-β, OX40L, 4-1BBL, CD70, GITRL, and SLAMF6; IFN-β, OX40L, 4-1BBL, CD70, GITRL, LIGHT, and TIM-4; IFN-β, OX40L, 4-1BBL, CD70, GITRL, LIGHT and ICAM-1; IFN-β, OX40L, 4-1BBL, CD70, GITRL, LIGHT and CD58; IFN-β, OX40L, 4-1BBL, CD70, GITRL, LIGHT and SLAMF6; IFN-β, OX40L, 4-1BBL, CD70, GITRL, LIGHT, TIM-4, and ICAM-1; IFN-β, OX40L, 4-1BBL, CD70, GITRL, LIGHT, TIM-4, and CD58; IFN-β, OX40L, 4-1BBL, CD70, GITRL, LIGHT, TIM-4, and SLAMF6; IFN-β, OX40L, 4-1BBL, CD70, GITRL, LIGHT, TIM-4, ICAM-1, and CD58; IFN-β, OX40L, 4-1BBL, CD70, GITRL, LIGHT, TIM-4, ICAM-1, and SLAMF6; IFN-β, OX40L, 4-1BBL, CD70, GITRL, LIGHT, TIM-4, ICAM-1, CD58, and SLAMF6; IFN-β, and 4-1BBL; IFN-β, 4-1BBL, and CD70; IFN-β, 4-1BBL, and GITRL; IFN-β, 4-1BBL, and LIGHT; IFN-β, 4-1BBL, and TIM-4; IFN-β, 4-1BBL, and ICAM-1; IFN-β, 4-1BBL, and CD58; IFN-β, 4-1BBL, and SLAMF6; IFN-β, 4-1BBL, and CD70; IFN-β, 4-1BBL, and GITRL; IFN-β, 4-1BBL, and LIGHT; IFN-β, 4-1BBL, and TIM-4; IFN-β, 4-1BBL, and ICAM-1; IFN-β, 4-1BBL, and CD58; IFN-β, 4-1BBL, and SLAMF6; IFN-β, 4-1BBL, CD70, and GITRL; IFN-β, 4-1BBL, CD70, and LIGHT; IFN-β, 4-1BBL, CD70, and TIM-4; IFN-β, 4-1BBL, CD70, and ICAM-1; IFN-β, 4-1BBL, CD70, and CD58; IFN-β, 4-1BBL, CD70, and SLAMF6; IFN-β, 4-1BBL, CD70, GITRL, and LIGHT; IFN-β, 4-1BBL, CD70, GITRL, and TIM-4; IFN-β, 4-1BBL, CD70, GITRL, and ICAM-1; IFN-β, 4-1BBL, CD70, GITRL, and CD58; IFN-β, 4-1BBL, CD70, GITRL, and SLAMF6; IFN-β, 4-1BBL, CD70, GITRL, LIGHT, and TIM-4; IFN-β, 4-1BBL, CD70, GITRL, LIGHT and ICAM-1; IFN-β, 4-1BBL, CD70, GITRL, LIGHT and CD58; IFN-β, 4-1BBL, CD70, GITRL, LIGHT and SLAMF6; IFN-β, 4-1BBL, CD70, GITRL, LIGHT, TIM-4, and ICAM-1; IFN-β, 4-1BBL, CD70, GITRL, LIGHT, TIM-4, and CD58; IFN-β, 4-1BBL, CD70, GITRL, LIGHT, TIM-4, and SLAMF6; IFN-β, 4-1BBL, CD70, GITRL, LIGHT, TIM-4, ICAM-1, and CD58; IFN-β, 4-1BBL, CD70, GITRL, LIGHT, TIM-4, ICAM-1, and SLAMF6; IFN-β, 4-1BBL, CD70, GITRL, LIGHT, TIM-4, ICAM-I, CD58, and SLAMF6; IFN-β and CD70; IFN-β, CD70, and GITRL; IFN-β, CD70, and LIGHT; IFN-β, CD70, and TIM-4; IFN-β, CD70, and ICAM-1; IFN-β, CD70, and CD58; IFN-β, CD70, and SLAMF6; IFN-β, CD70, and GITRL; IFN-β, CD70, and LIGHT; IFN-β, CD70, and TIM-4; IFN-0, CD70, and ICAM-1; IFN-β, CD70, and CD58; IFN-β, CD70, and SLAMF6; IFN-β, CD70, GITRL, and LIGHT; IFN-β, CD70, GITRL, and TIM-4; IFN-β, CD70, GITRL, and ICAM-1; IFN-β, CD70, GITRL, and CD58; IFN-β, CD70, GITRL, and SLAMF6; IFN-β, CD70, GITRL, LIGHT, and TIM-4; IFN-β, CD70, GITRL, LIGHT and ICAM-1; IFN-β, CD70, GITRL, LIGHT and CD58; IFN-β, CD70, GITRL, LIGHT and SLAMF6; IFN-β, CD70, GITRL, LIGHT, TIM-4, and ICAM-1; IFN-β, CD70, GITRL, LIGHT, TIM-4, and CD58; IFN-β, CD70, GITRL, LIGHT, TIM-4, and SLAMF6; IFN-β, CD70, GITRL, LIGHT, TIM-4, ICAM-1, and CD58; IFN-β, CD70, GITRL, LIGHT, TIM-4, ICAM-1, and SLAMF6; IFN-β, CD70, GITRL, LIGHT, TIM-4, ICAM-1, CD58, and SLAMF6; IFN-β and GITRL; IFN-β, GITRL, and LIGHT; IFN-β, GITRL, and TIM-4; IFN-β, GITRL, and ICAM-1; IFN-β, GITRL, and CD58; IFN-β, GITRL, and SLAMF6; IFN-β, GITRL, and LIGHT; IFN-β, GITRL, and TIM-4; IFN-β, GITRL, and ICAM-1; IFN-β, GITRL, and CD58; IFN-β, GITRL, and SLAMF6; IFN-β, GITRL, LIGHT, and TIM-4; IFN-β, GITRL, LIGHT and ICAM-1; IFN-β, GITRL, LIGHT and CD58; IFN-β, GITRL, LIGHT and SLAMF6; IFN-β, GITRL, LIGHT, TIM-4, and ICAM-1; IFN-β, GITRL, LIGHT, TIM-4, and CD58; IFN-β, GITRL, LIGHT, TIM-4, and SLAMF6; IFN-β, GITRL, LIGHT, TIM-4, ICAM-1, and CD58; IFN-β, GITRL, LIGHT, TIM-4, ICAM-1, and SLAMF6; IFN-β, GITRL, LIGHT, TIM-4, ICAM-1, CD58, and SLAMF6; IFN-β, and LIGHT; IFN-β, LIGHT, and TIM-4; IFN-β, LIGHT, and ICAM-1; IFN-β, LIGHT, and CD58; IFN-β, LIGHT, and SLAMF6; IFN-β, LIGHT, and TIM-4; IFN-β, LIGHT and ICAM-1; IFN-β, LIGHT and CD58; IFN-β, LIGHT and SLAMF6; IFN-β, LIGHT, TIM-4, and ICAM-1; IFN-β, LIGHT, TIM-4, and CD58; IFN-β, LIGHT, TIM-4, and SLAMF6; IFN-β, LIGHT, TIM-4, ICAM-1, and CD58; IFN-β, LIGHT, TIM-4, ICAM-1, and SLAMF6; IFN-β, LIGHT, TIM-4, ICAM-1, CD58, and SLAMF6; IFN-β, and TIM-4; IFN-β, TIM-4, and ICAM-1; IFN-β, TIM-4, and CD58; IFN-β, TIM-4, and SLAMF6; IFN-β, TIM-4, and ICAM-1; IFN-β, TIM-4, and CD58; IFN-β, TIM-4, and SLAMF6; IFN-β, TIM-4, ICAM-1, and CD58; IFN-β, TIM-4, ICAM-1, and SLAMF6; IFN-β, TIM-4, ICAM-1, CD58, and SLAMF6; IFN-β and ICAM-1; IFN-β, ICAM-1, and CD58; IFN-β, ICAM-1, and SLAMF6; IFN-β, ICAM-1, and CD58; IFN-β, ICAM-1, and SLAMF6; IFN-β, ICAM-1, CD58, and SLAMF6; IFN-β and CD58; IFN-β, CD58, and SLAMF6; IFN-β and SLAMF6; IFN-β, IFN-κ, and CD40-L; IFN-β, IFN-κ, and MEM40, IFN-β, IFN-κ, and B7-1(CD80)/B7-2(CD86); IFN-β, IFN-κ, and OX40L; IFN-β, IFN-κ, and 4-1BBL; IFN-β, IFN-κ, and CD70; IFN-β, IFN-κ, and GITRL; IFN-β, IFN-κ, and LIGHT; IFN-β, IFN-κ, and TIM-4; IFN-β, IFN-κ, and ICAM-1; IFN-0, and CD58; IFN-β, IFN-κ, and SLAMF6; IFN-β, IFN-κ, IFN-δ, and CD40-L; IFN-β, IFN-κ, IFN-δ, and MEM40; IFN-β, IFN-κ, IFN-δ, and B7-1(CD80)/B7-2(CD86); IFN-β, IFN-κ, IFN-δ, and OX40L; IFN-β, IFN-κ, IFN-δ, and 4-1BBL; IFN-β, IFN-κ, IFN-δ, and CD70; IFN-β, IFN-κ, IFN-δ, and GITRL; IFN-β, IFN-κ, IFN-δ, and LIGHT; IFN-β, IFN-κ, IFN-δ, and TIM-4; IFN-β, IFN-κ, IFN-δ, and ICAM-1; IFN-β, IFN-δ, and CD58; IFN-β, IFN-κ, IFN-δ, and SLAMF6; IFN-β, IFN-κ, IFN-δ, IFN-ε, and CD40-L, IFN-β, IFN-κ, IFN-δ, IFN-ε, and MEM40; IFN-β, IFN-κ, IFN-δ, IFN-ε, and B7-1(CD80)/B7-2(CD86); IFN-β, IFN-κ, IFN-δ, IFN-ε, and OX40L; IFN-β, IFN-κ, IFN-δ, IFN-ε, and 4-1BBL; IFN-β, IFN-κ, IFN-δ, IFN-ε, and CD70; IFN-β, IFN-κ, IFN-8, IFN-ε, and GITRL; IFN-β, IFN-κ, IFN-δ, IFN-ε, and LIGHT; IFN-β, IFN-κ, IFN-δ, IFN-ε, and TIM-4, IFN-β, IFN-κ, IFN-δ, IFN-ε, and ICAM-1; IFN-β, IFN-δ, IFN-ε, and CD58; IFN-β, IFN-κ, IFN-δ, IFN-ε, and SLAMF6; IFN-β, IFN-κ, IFN-δ, IFN-ε, IFN-τ, and CD40-L; IFN-β, IFN-κ, IFN-δ, IFN-ε, IFN-τ, and MEM40; IFN-β, IFN-κ, IFN-δ, IFN-ε, IFN-τ, and B7-1(CD80)/B7-2(CD86); IFN-β, IFN-κ, IFN-δ, IFN-ε, IFN-τ, and OX40L; IFN-β, IFN-κ, IFN-δ, IFN-ε, IFN-ε, and 4-1BBL; IFN-β, IFN-κ, IFN-δ, IFN-ε, IFN-τ, and CD70; IFN-β, IFN-κ, IFN-δ, IFN-ε, IFN-τ, and GITRL; IFN-β, IFN-κ, IFN-δ, IFN-ε, IFN-τ, and LIGHT; IFN-β, IFN-κ, IFN-δ, IFN-ε, IFN-τ, and TIM-4; IFN-β, IFN-κ, IFN-δ, IFN-ε, IFN-τ, and ICAM-1; IFN-β, IFN-δ, IFN-ε, IFN-τ, and CD58; IFN-β, IFN-κ, IFN-δ, IFN-ε, IFN-τ, and SLAMF6; IFN-β, IFN-κ, IFN-δ, IFN-ε, IFN-ε, IFN-ω, and CD40-L; IFN-β, IFN-κ, IFN-δ, IFN-ε, IFN-τ, IFN-ω, and MEM40; IFN-β, IFN-κ, IFN-δ, IFN-ε, IFN-τ, IFN-ω, and B7-1(CD80)/B7-2(CD86); IFN-β, IFN-κ, IFN-δ, IFN-ε, IFN-ε, IFN-ω, and OX40L, IFN-β, IFN-κ, IFN-δ, IFN-ε, IFN-τ, IFN-ω, and 4-1BBL; IFN-β, IFN-κ, IFN-δ, IFN-ε, IFN-τ, IFN-ω, and CD70; IFN-β, IFN-κ, IFN-δ, IFN-ε, IFN-τ, IFN-ω, and GITRL; IFN-β, IFN-κ, IFN-δ, IFN-ε, IFN-τ, IFN-ω, and LIGHT; IFN-β, IFN-κ, IFN-δ, IFN-ε, IFN-τ, IFN-ω, and TIM-4; IFN-β, IFN-κ, IFN-δ, IFN-ε, IFN-τ, IFN-ω, and ICAM-1; IFN-β, IFN-δ, IFN-ε, IFN-τ, IFN-ω, and CD58; IFN-β, IFN-κ, IFN-δ, IFN-ε, IFN-τ, IFN-ω, and SLAMF6; IFN-β, IFN-κ, IFN-δ, IFN-ε, IFN-τ, IFN-ω, IFN-ζ, and CD40-L; IFN-β, IFN-κ, IFN-δ, IFN-ε, IFN-τ, IFN-ω, IFN-ζ, and MEM40; IFN-β, IFN-κ, IFN-δ, IFN-ε, IFN-τ, IFN-ω, IFN-ζ, and B7-1(CD80)/B7-2(CD86); IFN-β, IFN-κ, IFN-δ, IFN-ε, IFN-τ, IFN-ω, IFN-ζ, and OX40L; IFN-β, IFN-κ, IFN-δ, IFN-ε, IFN-τ, IFN-ω, IFN-ζ, and 4-1BBL; IFN-β, IFN-κ, IFN-δ, IFN-ε, IFN-τ, IFN-ω, IFN-ζ, and CD70; IFN-β, IFN-κ, IFN-δ, IFN-ε, IFN-τ, IFN-ω, IFN-ζ, and GITRL; IFN-β, IFN-κ, IFN-δ, IFN-ε, IFN-τ, IFN-ω, IFN-ζ, and LIGHT; IFN-β, IFN-κ, IFN-δ, IFN-ε, IFN-τ, IFN-ω, IFN-ζ, and TIM-4; IFN-β, IFN-κ, IFN-δ, IFN-ε, IFN-τ, IFN-ω, IFN-ζ, and ICAM-1; IFN-β, IFN-δ, IFN-ε, IFN-τ, IFN-ω, IFN-ζ, and CD58; and/or IFN-β, IFN-κ, IFN-δ, IFN-ε, IFN-τ, IFN-ω, IFN-ζ, and SLAMF6. In one aspect, disclosed herein are methods of generating tumor infiltrating lymphocytes wherein the oncolytic virus further comprises IL-12 and/or IL-23. Alternatively, disclosed herein are methods of generating tumor infiltrating lymphocytes comprising a) administering an oncolytic virus expressing i) one or more type 1 interferon (IFN) (such as, for example, IFN-α, IFN-β, IFN-κ, IFN-δ, IFN-ε, IFN-τ, IFN-ω, and/or IFN-ζ) or one or more exogenous immunostimulatory molecules (such as, for example, CD40-L, MEM40, B7-1(CD80)/B7-2(CD86), OX40L, 4-1BBL, CD70, GITRL, LIGHT, TIM-4, ICAM-1, CD58 and/or SLAMF6) and/or ii) an interleukin selected from IL-12 and/or IL-23. For example, the oncolytic virus for use in the disclosed methods can comprise IFN-α and IL-12, IFN-β and IL-12, IFN-κ and IL-12, IFN-δ and IL-12, IFN-ε and IL-12, IFN-τ and IL-12, IFN-ω and IL-12, IFN-ζ and IL-12, CD40-L and IL-12, MEM40 and IL-12, B7-1(CD80)/B7-2(CD86) and IL-12, OX40L and IL-12, 4-1BBL and IL-12, CD70 and IL-12, GITRL and IL-12, LIGHT and IL-12, TIM-4 and IL-12, ICAM-1 and IL-12, CD58 and IL-12, SLAMF6 and IL-12, IFN-α and IL-23, IFN-β and IL-23, IFN-κ and IL-23, IFN-δ and IL-23, IFN-ε and IL-23, IFN-τ and IL-23, IFN-ω and IL-23, IFN-ζ and IL-23, CD40-L and IL-23, MEM40 and IL-23, B7-1(CD80)/B7-2(CD86) and IL-23, OX40L and IL-23, 4-1BBL and IL-23, CD70 and IL-23, GITRL and IL-23, LIGHT and IL-23, TIM-4 and IL-23, ICAM-1 and IL-23, CD58 and IL-23, SLAMF6 and IL-23, IFN-α, IL-12, and IL-23, IFN-β, IL-12, and IL-23, IFN-κ, IL-12, and IL-23, IFN-δ, IL-12, and IL-23, IFN-ε, IL-12, and IL-23, IFN-τ, IL-12, and IL-23, IFN-ω, IL-12, and IL-23, IFN-ζ, IL-12, and IL-23, CD40-L, IL-12, and IL-23, MEM40, IL-12, and IL-23, B7-1(CD80)/B7-2(CD86), IL-12, and IL-23, OX40L, IL-12, and IL-23, 4-1BBL, IL-12, and IL-23, CD70, IL-12, and IL-23, GITRL, IL-12, and IL-23, LIGHT, IL-12, and IL-23, TIM-4, IL-12, and IL-23, ICAM-1, IL-12, and IL-23, CD58, IL-12, and IL-23, and/or SLAMF6, IL-12, and IL-23.

It is understood and herein contemplated that the oncolytic virus can be administered to the tumor by any means known in the art including, but not limited to intratumoral injection. While the above methods result in dramatically increased numbers of TILs and MILs at the tumor site, it is also recognized that TILs and Mils can be generated and/or increased at tumor sites outside the tumor microenvironment being treated (i.e., an abscopal effect).

Just because TIL and MIL numbers increase at the target site of oncolytic virus administration and/or at abscopal tumor sites does not mean that further expansion cannot occur. In one aspect, it is understood and herein contemplated that said TILs in MILs can be further expanded if cultured ex vivo. Thus, also disclosed herein are methods of generating tumor infiltrating lymphocytes, further comprising expanding the harvested TILs ex vivo.

In one aspect, it is understood and herein contemplated that expansion of TILs and MILs does not have to be limited to in vivo oncolytic virus methods where tumors receiving an infection of an oncolytic virus and harvested, but rather the expansion of TILs can occur ex vivo by harvesting TILs and MILs in a cancer site and then culturing the harvested cells in the presence of antigen presenting cells that are infected with the oncolytic virus. Thus, the expansion of TILs and/or MILs occurs entirely ex vivo. Accordingly, disclosed herein are methods of expanding a population of tumor infiltrating lymphocytes (TILs) or marrow infiltrating lymphocytes (MILs) comprising: a) harvesting TILs or MILs from a subject with a cancer; b) culturing the harvested TILs or MILs in the presence of antigen presenting cells infected with an oncolytic virus expressing one or more type 1 interferon (IFN) (such as, for example, IFN-α, IFN-β, IFN-κ, IFN-δ, IFN-ε, IFN-τ, IFN-ω, and/or IFN-ζ) and/or one or more exogenous immunostimulatory molecules (such as, for example, CD40-L, MEM40, B7-1(CD80)/B7-2(CD86), OX40L, 4-1BBL, CD70, GITRL, LIGHT, TIM-4, ICAM-1, CD58 and/or SLAMF6). For example, the disclosed oncolytic virus for use in the disclosed methods can express IFN-α and CD40-L; IFN-α and MEM40; IFN-α and B7-1(CD80)/B7-2(CD86); IFN-α and OX40L; IFN-α and 4-1BBL; IFN-α and CD70; IFN-α and GITRL; IFN-α and LIGHT; IFN-α and TIM-4; IFN-α and ICAM-1; IFN-α and CD58; IFN-α and SLAMF6; IFN-α, CD40-L, and MEM40; IFN-α, CD40-L, and B7-1(CD80)/B7-2(CD86); IFN-α, CD40-L, and OX40L; IFN-α, CD40-L, and 4-1BBL; IFN-α, CD40-L, and CD70; IFN-α, CD40-L, and GITRL; IFN-α, CD40-L, and LIGHT; IFN-α, CD40-L, and TIM-4; IFN-α, CD40-L, and ICAM-1, IFN-α, CD40-L, and CD58; IFN-α, CD40-L, and SLAMF6; IFN-α, CD40-L, MEM40, and B7-1(CD80)/B7-2(CD86); IFN-α, CD40-L, MEM40, and OX40L; IFN-α, CD40-L, MEM40, and 4-1BBL; IFN-α, CD40-L, MEM40, and CD70; IFN-α, CD40-L, MEM40, and GITRL; IFN-α, CD40-L, MEM40, and LIGHT; IFN-α, CD40-L, MEM40, and TIM-4; IFN-α, CD40-L, MEM40, and ICAM-1; IFN-α, CD40-L, MEM40, and CD58; IFN-α, CD40-L, MEM40, and SLAMF6; IFN-α, CD40-L, MEM40, B7-1(CD80)/B7-2(CD86), and OX40L; IFN-α, CD40-L, MEM40, B7-1(CD80)/B7-2(CD86), and 4-1BBL; IFN-α, CD40-L, MEM40, B7-1(CD80)/B7-2(CD86), and CD70; IFN-α, CD40-L, MEM40, B7-1(CD80)/B7-2(CD86), and GITRL; IFN-α, CD40-L, MEM40, B7-1(CD80)/B7-2(CD86), and LIGHT; IFN-α, CD40-L, MEM40, B7-1(CD80)/B7-2(CD86), and TIM-4; IFN-α, CD40-L, MEM40, B7-1(CD80)/B7-2(CD86), and ICAM-1; IFN-α, CD40-L, MEM40, B7-1(CD80)/B7-2(CD86), and CD58; IFN-α, CD40-L, MEM40, B7-1(CD80)/B7-2(CD86), and SLAMF6; IFN-α, CD40-L, MEM40, B7-1(CD80)/B7-2(CD86), OX40L, and 4-1BBL; IFN-α, CD40-L, MEM40, B7-1(CD80)/B7-2(CD86), OX40L, and CD70; IFN-α, CD40-L, MEM40, B7-1(CD80)/B7-2(CD86), OX40L, and GITRL; IFN-α, CD40-L, MEM40, B7-1(CD80)B7-2(CD86), OX40L, and LIGHT; IFN-α, CD40-L, MEM40, B7-1(CD80)/B7-2(CD86), OX40L, and TIM-4; IFN-α, CD40-L, MEM40, B7-1(CD80)/B7-2(CD86), OX40L, and ICAM-1; IFN-α, CD40-L, MEM40, B7-1(CD80)/B7-2(CD86), OX40L, and CD58; IFN-α, CD40-L, MEM40, B7-1(CD80)/B7-2(CD86), OX40L, and SLAMF6; IFN-α, CD40-L, MEM40, B7-1(CD80)/B7-2(CD86), OX40L, 4-1BBL, and CD70; IFN-α, CD40-L, MEM40, B7-1(CD80)/B7-2(CD86), OX40L, 4-1BBL, and GITRL; IFN-α, CD40-L, MEM40, B7-1(CD80)/B7-2(CD86), OX40L, 4-1BBL, and LIGHT; IFN-α, CD40-L, MEM40, B7-1(CD80)/B7-2(CD86), OX40L, 4-1BBL, and TIM-4; IFN-α, CD40-L, MEM40, B7-1(CD80)/B7-2(CD86), OX40L, 4-1BBL, and ICAM-1; IFN-α, CD40-L, MEM40, B7-1(CD80)/B7-2(CD86), OX40L, 4-1BBL, and CD58; IFN-α, CD40-L, MEM40, B7-1(CD80)/B7-2(CD86), OX40L, 4-1BBL, and SLAMF6; IFN-α, CD40-L, MEM40, B7-1(CD80)/B7-2(CD86), OX40L, 4-1BBL, CD70, and GITRL; IFN-α, CD40-L, MEM40, B7-1(CD80)/B7-2(CD86), OX40L, 4-1BBL, CD70, and LIGHT; IFN-α, CD40-L, MEM40, B7-1(CD80)/B7-2(CD86), OX40L, 4-1BBL, CD70, and TIM-4; IFN-α, CD40-L, MEM40, B7-1(CD80)/B7-2(CD86), OX40L, 4-1BBL, CD70, and ICAM-1; IFN-α, CD40-L, MEM40, B7-1(CD80)/B7-2(CD86), OX40L, 4-1BBL, CD70, and CD58; IFN-α, CD40-L, MEM40, B7-1(CD80)/B7-2(CD86), OX40L, 4-1BBL, CD70, and SLAMF6; IFN-α, CD40-L, MEM40, B7-1(CD80)/B7-2(CD86), OX40L, 4-1BBL, CD70, GITRL, and LIGHT; IFN-α, CD40-L, MEM40, B7-1(CD80)/B7-2(CD86), OX40L, 4-1BBL, CD70, GITRL, and TIM-4; IFN-α, CD40-L, MEM40, B7-1(CD80)/B7-2(CD86), OX40L, 4-1BBL, CD70, GITRL, and ICAM-1; IFN-α, CD40-L, MEM40, B7-1(CD80)/B7-2(CD86), OX40L, 4-1BBL, CD70, GITRL, and CD58; IFN-α, CD40-L, MEM40, B7-1(CD80)/B7-2(CD86), OX40L, 4-1BBL, CD70, GITRL, and SLAMF6; IFN-α, CD40-L, MEM40, B7-1(CD80)/B7-2(CD86), OX40L, 4-1BBL, CD70, GITRL, LIGHT, and TIM-4; IFN-α, CD40-L, MEM40, B7-1(CD80)/B7-2(CD86), OX40L, 4-1BBL, CD70, GITRL, LIGHT and ICAM-1; IFN-α, CD40-L, MEM40, B7-1(CD80)/B7-2(CD86), OX40L, 4-1BBL, CD70, GITRL, LIGHT and CD58; IFN-α, CD40-L, MEM40, B7-1(CD80)/B7-2(CD86), OX40L, 4-1BBL, CD70, GITRL, LIGHT and SLAMF6; IFN-α, CD40-L, MEM40, B7-1(CD80)/B7-2(CD86), OX40L, 4-1BBL, CD70, GITRL, LIGHT, TIM-4, and ICAM-1; IFN-α, CD40-L, MEM40, B7-1(CD80)/B7-2(CD86), OX40L, 4-1BBL, CD70, GITRL, LIGHT, TIM-4, and CD58; IFN-α, CD40-L, MEM40, B7-1(CD80)/B7-2(CD86), OX40L, 4-1BBL, CD70, GITRL, LIGHT, TIM-4, and SLAMF6; IFN-α, CD40-L, MEM40, B7-1(CD80)/B7-2(CD86), OX40L, 4-1BBL, CD70, GITRL, LIGHT, TIM-4, ICAM-1, and CD58; IFN-α, CD40-L, MEM40, B7-1(CD80)/B7-2(CD86), OX40L, 4-1BBL, CD70, GITRL, LIGHT, TIM-4, ICAM-1, and SLAMF6; IFN-α, CD40-L, MEM40, B7-1(CD80)/B7-2(CD86), OX40L, 4-1BBL, CD70, GITRL, LIGHT, TIM-4, ICAM-1, CD58, and SLAMF6; IFN-α, MEM40; IFN-α, MEM40, and B7-1(CD80)/B7-2(CD86); IFN-α, MEM40, and OX40L; IFN-α, MEM40, and 4-1BBL; IFN-α, MEM40, and CD70; IFN-α, MEM40, and GITRL; IFN-α, MEM40, and LIGHT; IFN-α, MEM40, and TIM-4; IFN-α, MEM40, and ICAM-1; IFN-α, MEM40, and CD58; IFN-α, MEM40, and SLAMF6; IFN-α, MEM40, and B7-1(CD80)/B7-2(CD86); IFN-α, MEM40, and OX40L; IFN-α, MEM40, and 4-1BBL; IFN-α, MEM40, and CD70; IFN-α, MEM40, and GITRL; IFN-α, MEM40, and LIGHT; IFN-α, MEM40, and TIM-4, IFN-α, MEM40, and ICAM-I; IFN-α, MEM40, and CD58; IFN-α, MEM40, and SLAMF6; IFN-α, MEM40, B7-1(CD80)/B7-2(CD86), and OX40L; IFN-α, MEM40, B7-1(CD80)/B7-2(CD86), and 4-1BBL; IFN-α, MEM40, B7-1(CD80)/B7-2(CD86), and CD70; IFN-α, MEM40, B7-1(CD80)/B7-2(CD86), and GITRL; IFN-α, MEM40, B7-1(CD80)/B7-2(CD86), and LIGHT; IFN-α, MEM40, B7-1(CD80)/B7-2(CD86), and TIM-4; IFN-α, MEM40, B7-1(CD80)/B7-2(CD86), and ICAM-1; IFN-α, MEM40, B7-1(CD80)/B7-2(CD86), and CD58; IFN-α, MEM40, B7-1(CD80)/B7-2(CD86), and SLAMF6; IFN-α, MEM40, B7-1(CD80)/B7-2(CD86), OX40L, and 4-1BBL; IFN-α, MEM40, B7-1(CD80)/B7-2(CD86), OX40L, and CD70; IFN-α, MEM40, B7-1(CD80)/B7-2(CD86), OX40L, and GITRL; IFN-α, MEM40, B7-1(CD80)/B7-2(CD86), OX40L, and LIGHT; IFN-α, MEM40, B7-1(CD80)/B7-2(CD86), OX40L, and TIM-4; IFN-α, MEM40, B7-1(CD80)/B7-2(CD86), OX40L, and ICAM-1; IFN-α, MEM40, B7-1(CD80)/B7-2(CD86), OX40L, and CD58; IFN-α, MEM40, B7-1(CD80)/B7-2(CD86), OX40L, and SLAMF6; IFN-α, MEM40, B7-1(CD80)/B7-2(CD86), OX40L, 4-1BBL, and CD70; IFN-α, MEM40, B7-1(CD80)/B7-2(CD86), OX40L, 4-1BBL, and GITRL; IFN-α, MEM40, B7-1(CD80)/B7-2(CD86), OX40L, 4-1BBL, and LIGHT; IFN-α, MEM40, B7-1(CD80)/B7-2(CD86), OX40L, 4-1BBL, and TIM-4; IFN-α, MEM40, B7-1(CD80)/B7-2(CD86), OX40L, 4-1BBL, and ICAM-1; IFN-α, MEM40, B7-1(CD80)/B7-2(CD86), OX40L, 4-1BBL, and CD58; IFN-α, MEM40, B7-1(CD80)/B7-2(CD86), OX40L, 4-1BBL, and SLAMF6; IFN-α, MEM40, B7-1(CD80)/B7-2(CD86), OX40L, 4-1BBL, CD70, and GITRL; IFN-α, MEM40, B7-1(CD80)/B7-2(CD86), OX40L, 4-1BBL, CD70, and LIGHT; IFN-α, MEM40, B7-1(CD80)/B7-2(CD86), OX40L, 4-1BBL, CD70, and TIM-4; IFN-α, MEM40, B7-1(CD80)/B7-2(CD86), OX40L, 4-1BBL, CD70, and ICAM-1; IFN-α, MEM40, B7-1(CD80)/B7-2(CD86), OX40L, 4-1BBL, CD70, and CD58; IFN-α, MEM40, B7-1(CD80)/B7-2(CD86), OX40L, 4-1BBL, CD70, and SLAMF6; IFN-α, MEM40, B7-1(CD80)/B7-2(CD86), OX40L, 4-1BBL, CD70, GITRL, and LIGHT; IFN-α, MEM40, B7-1(CD80)/B7-2(CD86), OX40L, 4-1BBL, CD70, GITRL, and TIM-4; IFN-α, MEM40, B7-1(CD80)/B7-2(CD86), OX40L, 4-1BBL, CD70, GITRL, and ICAM-1; IFN-α, MEM40, B7-1(CD80)/B7-2(CD86), OX40L, 4-1BBL, CD70, GITRL, and CD58; IFN-α, MEM40, B7-1(CD80)/B7-2(CD86), OX40L, 4-1BBL, CD70, GITRL, and SLAMF6; IFN-α, MEM40, B7-1(CD80)/B7-2(CD86), OX40L, 4-1BBL, CD70, GITRL, LIGHT, and TIM-4; IFN-α, MEM40, B7-1(CD80)/B7-2(CD86), OX40L, 4-1BBL, CD70, GITRL, LIGHT and ICAM-1; IFN-α, MEM40, B7-1(CD80)/B7-2(CD86), OX40L, 4-1BBL, CD70, GITRL, LIGHT and CD58; IFN-α, MEM40, B7-1(CD80)/B7-2(CD86), OX40L, 4-1BBL, CD70, GITRL, LIGHT and SLAMF6; IFN-α, MEM40, B7-1(CD80)/B7-2(CD86), OX40L, 4-1 BBL, CD70, GITRL, LIGHT, TIM-4, and ICAM-1; IFN-α, MEM40, B7-1(CD80)/B7-2(CD86), OX40L, 4-1BBL, CD70, GITRL, LIGHT, TIM-4, and CD58; IFN-α, MEM40, B7-1(CD80)/B7-2(CD86), OX40L, 4-1BBL, CD70, GITRL, LIGHT, TIM-4, and SLAMF6; IFN-α, MEM40, B7-1(CD80)/B7-2(CD86), OX40L, 4-1BBL, CD70, GITRL, LIGHT, TIM-4, ICAM-1, and CD58; IFN-α, MEM40, B7-1(CD80)/B7-2(CD86), OX40L, 4-1BBL, CD70, GITRL, LIGHT, TIM-4, ICAM-1, and SLAMF6; IFN-α, MEM40, B7-1(CD80)/B7-2(CD86), OX40L, 4-1BBL, CD70, GITRL, LIGHT, TIM-4, ICAM-1, CD58, and SLAMF6; IFN-α, and B7-1(CD80)/B7-2(CD86); IFN-α, B7-1(CD80)/B7-2(CD86), and OX40L; IFN-α, B7-1(CD80)/B7-2(CD86), and 4-1BBL; IFN-α, B7-1(CD80)/B7-2(CD86), and CD70; IFN-α, B7-1(CD80)/B7-2(CD86), and GITRL; IFN-α, B7-1(CD80)/B7-2(CD86), and LIGHT; IFN-α, B7-1(CD80)/B7-2(CD86), and TIM-4; IFN-α, B7-1(CD80)/B7-2(CD86), and ICAM-1; IFN-α, B7-1(CD80)/B7-2(CD86), and CD58; IFN-α, B7-1(CD80)/B7-2(CD86), and SLAMF6; IFN-α, B7-1(CD80)/B7-2(CD86), and OX40L; IFN-α, B7-1(CD80)/B7-2(CD86), and 4-1BBL; IFN-α, B7-1(CD80)/B7-2(CD86), and CD70; IFN-α, B7-1(CD80)/B7-2(CD86), and GITRL; IFN-α, B7-1(CD80)/B7-2(CD86), and LIGHT; IFN-α, B7-1(CD80)/B7-2(CD86), and TIM-4; IFN-α, B7-1(CD80)/B7-2(CD86), and ICAM-1; IFN-α, B7-1(CD80)/B7-2(CD86), and CD58; IFN-α, B7-1(CD80)/B7-2(CD86), and SLAMF6; IFN-α, B7-1(CD80)/B7-2(CD86), OX40L, and 4-1BBL; IFN-α, B7-1(CD80)/B7-2(CD86), OX40L, and CD70; IFN-α, B7-1(CD80)/B7-2(CD86), OX40L, and GITRL; IFN-α, B7-1(CD80)/B7-2(CD86), OX40L, and LIGHT; IFN-α, B7-1(CD80)/B7-2(CD86), OX40L, and TIM-4; IFN-α, B7-1(CD80)/B7-2(CD86), OX40L, and ICAM-1; IFN-α, B7-1(CD80)/B7-2(CD86), OX40L, and CD58; IFN-α, B7-1(CD80)/B7-2(CD86), OX40L, and SLAMF6; IFN-α, B7-1(CD80)/B7-2(CD86), OX40L, 4-1BBL, and CD70; IFN-α, B7-1(CD80)/B7-2(CD86), OX40L, 4-1BBL, and GITRL; IFN-α, B7-1(CD80)/B7-2(CD86), OX40L, 4-1BBL, and LIGHT; IFN-α, B7-1(CD80)/B7-2(CD86), OX40L, 4-1BBL, and TIM-4; IFN-α, B7-1(CD80)/B7-2(CD86), OX40L, 4-1BBL, and ICAM-1; IFN-α, B7-1(CD80)/B7-2(CD86), OX40L, 4-1BBL, and CD58; IFN-α, B7-1(CD80)/B7-2(CD86), OX40L, 4-1BBL, and SLAMF6; IFN-α, B7-1(CD80)/B7-2(CD86), OX40L, 4-1BBL, CD70, and GITRL; IFN-α, B7-1(CD80)/B7-2(CD86), OX40L, 4-1BBL, CD70, and LIGHT; IFN-α, B7-1(CD80)/B7-2(CD86), OX40L, 4-1BBL, CD70, and TIM-4; IFN-α, B7-1(CD80)/B7-2(CD86), OX40L, 4-1BBL, CD70, and ICAM-1; IFN-α, B7-1(CD80)/B7-2(CD86), OX40L, 4-1BBL, CD70, and CD58; IFN-α, B7-1(CD80)/B7-2(CD86), OX40L, 4-1BBL, CD70, and SLAMF6; IFN-α, B7-1(CD80)/B7-2(CD86), OX40L, 4-1BBL, CD70, GITRL, and LIGHT; IFN-α, B7-1(CD80)/B7-2(CD86), OX40L, 4-1BBL, CD70, GITRL, and TIM-4; IFN-α, B7-1(CD80)/B7-2(CD86), OX40L, 4-1BBL, CD70, GITRL, and ICAM-1, IFN-α, B7-1(CD80)/B7-2(CD86), OX40L, 4-1BBL, CD70, GITRL, and CD58; IFN-α, B7-1(CD80)/B7-2(CD86), OX40L, 4-1BBL, CD70, GITRL, and SLAMF6; IFN-α, B7-1(CD80)/B7-2(CD86), OX40L, 4-1BBL, CD70, GITRL, LIGHT, and TIM-4; IFN-α, B7-1(CD80)/B7-2(CD86), OX40L, 4-1BBL, CD70, GITRL, LIGHT and ICAM-1; IFN-α, B7-1(CD80)/B7-2(CD86), OX40L, 4-1BBL, CD70, GITRL, LIGHT and CD58; IFN-α, B7-1(CD80)/B7-2(CD86), OX40L, 4-1BBL, CD70, GITRL, LIGHT and SLAMF6; IFN-α, B7-1(CD80)/B7-2(CD86), OX40L, 4-1BBL, CD70, GITRL, LIGHT, TIM-4, and ICAM-1; IFN-α, B7-1(CD80)/B7-2(CD86), OX40L, 4-1BBL, CD70, GITRL, LIGHT, TIM-4, and CD58; IFN-α, B7-1(CD80)/B7-2(CD86), OX40L, 4-1BBL, CD70, GITRL, LIGHT, TIM-4, and SLAMF6; IFN-α, B7-1(CD80)/B7-2(CD86), OX40L, 4-1BBL, CD70, GITRL, LIGHT, TIM-4, ICAM-1, and CD58; IFN-α, B7-1(CD80)/B7-2(CD86), OX40L, 4-1BBL, CD70, GITRL, LIGHT, TIM-4, ICAM-1, and SLAMF6; IFN-α, B7-1(CD80)/B7-2(CD86), OX40L, 4-1BBL, CD70, GITRL, LIGHT, TIM-4, ICAM-1, CD58, and SLAMF6; IFN-α, and OX40L; IFN-α, OX40L, and 4-1BBL; IFN-α, OX40L, and CD70; IFN-α, OX40L, and GITRL; IFN-α, OX40L, and LIGHT; IFN-α, OX40L, and TIM-4; IFN-α, OX40L, and ICAM-1; IFN-α, OX40L, and CD58; IFN-α, OX40L, and SLAMF6; IFN-α, OX40L, and 4-1BBL; IFN-α, OX40L, and CD70; IFN-α, OX40L, and GITRL; IFN-α, OX40L, and LIGHT; IFN-α, OX40L, and TIM-4; IFN-α, OX40L, and ICAM-1; IFN-α, OX40L, and CD58; IFN-α, OX40L, and SLAMF6; IFN-α, OX40L, 4-1BBL, and CD70; IFN-α, OX40L, 4-1BBL, and GITRL; IFN-α, OX40L, 4-1BBL, and LIGHT; IFN-α, OX40L, 4-1 BBL, and TIM-4, IFN-α, OX40L, 4-1BBL, and ICAM-1; IFN-α, OX40L, 4-1BBL, and CD58; IFN-α, OX40L, 4-1BBL, and SLAMF6; IFN-α, OX40L, 4-1BBL, CD70, and GITRL; IFN-α, OX40L, 4-1BBL, CD70, and LIGHT; IFN-α, OX40L, 4-1BBL, CD70, and TIM-4; IFN-α, OX40L, 4-1BBL, CD70, and ICAM-1; IFN-α, OX40L, 4-1BBL, CD70, and CD58; IFN-α, OX40L, 4-1BBL, CD70, and SLAMF6; IFN-α, OX40L, 4-1BBL, CD70, GITRL, and LIGHT; IFN-α, OX40L, 4-1BBL, CD70, GITRL, and TIM-4; IFN-α, OX40L, 4-1BBL, CD70, GITRL, and ICAM-1; IFN-α, OX40L, 4-1BBL, CD70, GITRL, and CD58; IFN-α, OX40L, 4-1BBL, CD70, GITRL, and SLAMF6; IFN-α, OX40L, 4-1BBL, CD70, GITRL, LIGHT, and TIM-4; IFN-α, OX40L, 4-1BBL, CD70, GITRL, LIGHT and ICAM-1; IFN-α, OX40L, 4-1BBL, CD70, GITRL, LIGHT and CD58; IFN-α, OX40L, 4-1BBL, CD70, GITRL, LIGHT and SLAMF6; IFN-α, OX40L, 4-1BBL, CD70, GITRL, LIGHT, TIM-4, and ICAM-1; IFN-α, OX40L, 4-1BBL, CD70, GITRL, LIGHT, TIM-4, and CD58; IFN-α, OX40L, 4-1BBL, CD70, GITRL, LIGHT, TIM-4, and SLAMF6; IFN-α, OX40L, 4-1BBL, CD70, GITRL, LIGHT, TIM-4, ICAM-1, and CD58; IFN-α, OX40L, 4-1BBL, CD70, GITRL, LIGHT, TIM-4, ICAM-1, and SLAMF6; IFN-α, OX40L, 4-1BBL, CD70, GITRL, LIGHT, TIM-4, ICAM-1, CD58, and SLAMF6; IFN-α, and 4-1BBL; IFN-α, 4-1BBL, and CD70; IFN-α, 4-1BBL, and GITRL; IFN-α, 4-1BBL, and LIGHT; IFN-α, 4-1BBL, and TIM-4; IFN-α, 4-1BBL, and ICAM-1; IFN-α, 4-1BBL, and CD58; IFN-α, 4-1BBL, and SLAMF6; IFN-α, 4-1BBL, and CD70; IFN-α, 4-1BBL, and GITRL; IFN-α, 4-1BBL, and LIGHT; IFN-α, 4-1BBL, and TIM-4; IFN-α, 4-1BBL, and ICAM-1; IFN-α, 4-1BBL, and CD58; IFN-α, 4-1BBL, and SLAMF6; IFN-α, 4-1BBL, CD70, and GITRL; IFN-α, 4-1BBL, CD70, and LIGHT; IFN-α, 4-1BBL, CD70, and TIM-4; IFN-α, 4-1 BBL, CD70, and ICAM-1; IFN-α, 4-1 BBL, CD70, and CD58; IFN-α, 4-1 BBL, CD70, and SLAMF6; IFN-α, 4-1BBL, CD70, GITRL, and LIGHT; IFN-α, 4-1BBL, CD70, GITRL, and TIM-4; IFN-α, 4-1BBL, CD70, GITRL, and ICAM-1; IFN-α, 4-1BBL, CD70, GITRL, and CD58; IFN-α, 4-1BBL, CD70, GITRL, and SLAMF6; IFN-α, 4-1BBL, CD70, GITRL, LIGHT, and TIM-4; IFN-α, 4-1BBL, CD70, GITRL, LIGHT and ICAM-1; IFN-α, 4-1BBL, CD70, GITRL, LIGHT and CD58; IFN-α, 4-1BBL, CD70, GITRL, LIGHT and SLAMF6; IFN-α, 4-1BBL, CD70, GITRL, LIGHT, TIM-4, and ICAM-1; IFN-α, 4-1BBL, CD70, GITRL, LIGHT, TIM-4, and CD58; IFN-α, 4-1BBL, CD70, GITRL, LIGHT, TIM-4, and SLAMF6; IFN-α, 4-1BBL, CD70, GITRL, LIGHT, TIM-4, ICAM-1, and CD58; IFN-α, 4-1BBL, CD70, GITRL, LIGHT, TIM-4, ICAM-1, and SLAMF6; IFN-α, 4-1BBL, CD70, GITRL, LIGHT, TIM-4, ICAM-1, CD58, and SLAMF6; IFN-α and CD70; IFN-α, CD70, and GITRL; IFN-α, CD70, and LIGHT; IFN-α, CD70, and TIM-4; IFN-α, CD70, and ICAM-1; IFN-α, CD70, and CD58; IFN-α, CD70, and SLAMF6; IFN-α, CD70, and GITRL; IFN-α, CD70, and LIGHT; IFN-α, CD70, and TIM-4; IFN-α, CD70, and ICAM-1; IFN-α, CD70, and CD58; IFN-α, CD70, and SLAMF6; IFN-α, CD70, GITRL, and LIGHT; IFN-α, CD70, GITRL, and TIM-4; IFN-α, CD70, GITRL, and ICAM-1; IFN-α, CD70, GITRL, and CD58; IFN-α, CD70, GITRL, and SLAMF6; IFN-α, CD70, GITRL, LIGHT, and TIM-4; IFN-α, CD70, GITRL, LIGHT and ICAM-1; IFN-α, CD70, GITRL, LIGHT and CD58; IFN-α, CD70, GITRL, LIGHT and SLAMF6; IFN-α, CD70, GITRL, LIGHT, TIM-4, and ICAM-1; IFN-α, CD70, GITRL, LIGHT, TIM-4, and CD58; IFN-α, CD70, GITRL, LIGHT, TIM-4, and SLAMF6; IFN-α, CD70, GITRL, LIGHT, TIM-4, ICAM-1, and CD58; IFN-α, CD70, GITRL, LIGHT, TIM-4, ICAM-1, and SLAMF6; IFN-α, CD70, GITRL, LIGHT, TIM-4, ICAM-1, CD58, and SLAMF6; IFN-α and GITRL; IFN-α, GITRL, and LIGHT; IFN-α, GITRL, and TIM-4; IFN-α, GITRL, and ICAM-1; IFN-α, GITRL, and CD58; IFN-α, GITRL, and SLAMF6; IFN-α, GITRL, and LIGHT; IFN-α, GITRL, and TIM-4; IFN-α, GITRL, and ICAM-1; IFN-α, GITRL, and CD58; IFN-α, GITRL, and SLAMF6; IFN-α, GITRL, LIGHT, and TIM-4; IFN-α, GITRL, LIGHT and ICAM-1; IFN-α, GITRL, LIGHT and CD58; IFN-α, GITRL, LIGHT and SLAMF6; IFN-α, GITRL, LIGHT, TIM-4, and ICAM-1; IFN-α, GITRL, LIGHT, TIM-4, and CD58; IFN-α, GITRL, LIGHT, TIM-4, and SLAMF6; IFN-α, GITRL, LIGHT, TIM-4, ICAM-1, and CD58; IFN-α, GITRL, LIGHT, TIM-4, ICAM-1, and SLAMF6; IFN-α, GITRL, LIGHT, TIM-4, ICAM-1, CD58, and SLAMF6; IFN-α, and LIGHT; IFN-α, LIGHT, and TIM-4; IFN-α, LIGHT, and ICAM-1; IFN-α, LIGHT, and CD58; IFN-α, LIGHT, and SLAMF6; IFN-α, LIGHT, and TIM-4; IFN-α, LIGHT and ICAM-1; IFN-α, LIGHT and CD58; IFN-α, LIGHT and SLAMF6; IFN-α, LIGHT, TIM-4, and ICAM-1; IFN-α, LIGHT, TIM-4, and CD58; IFN-α, LIGHT, TIM-4, and SLAMF6; IFN-α, LIGHT, TIM-4, ICAM-1, and CD58; IFN-α, LIGHT, TIM-4, ICAM-1, and SLAMF6; IFN-α, LIGHT, TIM-4, ICAM-1, CD58, and SLAMF6; IFN-α, and TIM-4; IFN-α, TIM-4, and ICAM-1; IFN-α, TIM-4, and CD58; IFN-α, TIM-4, and SLAMF6; IFN-α, TIM-4, and ICAM-1; IFN-α, TIM-4, and CD58; IFN-α, TIM-4, and SLAMF6; IFN-α, TIM-4, ICAM-1, and CD58; IFN-α, TIM-4, ICAM-1, and SLAMF6; IFN-α, TIM-4, ICAM-1, CD58, and SLAMF6; IFN-α and ICAM-1; IFN-α, ICAM-1, and CD58; IFN-α, ICAM-1, and SLAMF6; IFN-α, ICAM-1, and CD58; IFN-α, ICAM-1, and SLAMF6; IFN-α, ICAM-1, CD58, and SLAMF6; IFN-α and CD58; IFN-α, CD58, and SLAMF6; IFN-α and SLAMF6; IFN-α, IFN-β, and CD40-L; IFN-α, IFN-β, and MEM40; IFN-α, IFN-β, and B7-1(CD80)/B7-2(CD86); IFN-α, IFN-β, and OX40L; IFN-α, IFN-β, and 4-1BBL; IFN-α, IFN-β, and CD70; IFN-α, IFN-β, and GITRL; IFN-α, IFN-β, and LIGHT; IFN-α, IFN-β, and TIM-4; IFN-α, IFN-0, and ICAM-1; IFN-α, IFN-β, and CD58; IFN-α, IFN-β, and SLAMF6; IFN-α, IFN-β, IFN-κ, and CD40-L; IFN-α, IFN-β, IFN-κ, and MEM40; IFN-α, IFN-β, IFN-κ, and B7-1(CD80)/B7-2(CD86); IFN-α, IFN-β, IFN-κ, and OX40L; IFN-α, IFN-β, IFN-κ, and 4-1BBL; IFN-α, IFN-β, IFN-κ, and CD70; IFN-α, IFN-β, IFN-κ, and GITRL; IFN-α, IFN-β, IFN-κ, and LIGHT; IFN-α, IFN-β, IFN-κ, and TIM-4; IFN-α, IFN-β, IFN-κ, and ICAM-1; IFN-α, IFN-β, and CD58; IFN-α, IFN-β, IFN-κ, and SLAMF6; IFN-α, IFN-β, IFN-κ, IFN-δ, and CD40-L; IFN-α, IFN-β, IFN-κ, IFN-δ, and MEM40; IFN-α, IFN-β, IFN-κ, IFN-δ, and B7-1(CD80)/B7-2(CD86); IFN-α, IFN-β, IFN-κ, IFN-δ, and OX40L; IFN-α, IFN-β, IFN-κ, IFN-δ, and 4-1 BBL; IFN-α, IFN-β, IFN-κ, IFN-δ, and CD70; IFN-α, IFN-β, IFN-κ, IFN-δ, and GITRL; IFN-α, IFN-β, IFN-κ, IFN-δ, and LIGHT; IFN-α, IFN-β, IFN-κ, IFN-δ, and TIM-4; IFN-α, IFN-β, IFN-κ, IFN-δ, and ICAM-1; IFN-α, IFN-β, IFN-δ, and CD58; IFN-α, IFN-β, IFN-κ, IFN-δ, and SLAMF6; IFN-α, IFN-β, IFN-κ, IFN-δ, IFN-ε, and CD40-L; IFN-α, IFN-β, IFN-κ, IFN-δ, IFN-ε, and MEM40; IFN-α, IFN-β, IFN-κ, IFN-δ, IFN-ε, and B7-1(CD80)/B7-2(CD86); IFN-α, IFN-β, IFN-κ, IFN-δ, IFN-ε, and OX40L; IFN-α, IFN-β, IFN-κ, IFN-δ, IFN-ε, and 4-1BBL; IFN-α, IFN-β, IFN-κ, IFN-δ, IFN-ε, and CD70; IFN-α, IFN-β, IFN-κ, IFN-δ, IFN-ε, and GITRL; IFN-α, IFN-β, IFN-κ, IFN-δ, IFN-ε, and LIGHT; IFN-α, IFN-β, IFN-κ, IFN-δ, IFN-ε, and TIM-4; IFN-α, IFN-β, IFN-κ, IFN-δ, IFN-ε, and ICAM-1; IFN-α, IFN-β, IFN-δ, IFN-ε, and CD58; IFN-α, IFN-β, IFN-κ, IFN-δ, IFN-ε, and SLAMF6; IFN-α, IFN-β, IFN-κ, IFN-δ, IFN-ε, IFN-τ, and CD40-L; IFN-α, IFN-β, IFN-κ, IFN-δ, IFN-ε, IFN-τ, and MEM40; IFN-α, IFN-β, IFN-κ, IFN-δ, IFN-ε, IFN-ε, and B7-1(CD80)/B7-2(CD86); IFN-α, IFN-β, IFN-κ, IFN-δ, IFN-ε, IFN-τ, and OX40L; IFN-α, IFN-β, IFN-κ, IFN-δ, IFN-ε, IFN-τ, and 4-1BBL; IFN-α, IFN-β, IFN-κ, IFN-δ, IFN-ε, IFN-ε, and CD70; IFN-α, IFN-β, IFN-κ, IFN-δ, IFN-ε, IFN-τ, and GITRL; IFN-α, IFN-β, IFN-κ, IFN-δ, IFN-ε, IFN-τ, and LIGHT; IFN-α, IFN-β, IFN-κ, IFN-δ, IFN-ε, IFN-τ, and TIM-4; IFN-α, IFN-β, IFN-κ, IFN-δ, IFN-ε, IFN-τ, and ICAM-1; IFN-α, IFN-β, IFN-δ, IFN-ε, IFN-τ, and CD58; IFN-α, IFN-β, IFN-κ, IFN-δ, IFN-ε, IFN-τ, and SLAMF6; IFN-α, IFN-β, IFN-κ, IFN-δ, IFN-ε, IFN-τ, IFN-ω, and CD40-L; IFN-α, IFN-β, IFN-κ, IFN-δ, IFN-ε, IFN-τ, IFN-ω, and MEM40; IFN-α, IFN-β, IFN-κ, IFN-δ, IFN-ε, IFN-τ, IFN-ω, and B7-1(CD80)/B7-2(CD86); IFN-α, IFN-β, IFN-κ, IFN-δ, IFN-ε, IFN-τ, IFN-ω, and OX40L; IFN-α, IFN-β, IFN-κ, IFN-δ, IFN-ε, IFN-ε, IFN-ω, and 4-1BBL; IFN-α, IFN-β, IFN-κ, IFN-δ, IFN-ε, IFN-τ, IFN-ω, and CD70; IFN-α, IFN-β, IFN-κ, IFN-δ, IFN-ε, IFN-τ, IFN-ω, and GITRL; IFN-α, IFN-β, IFN-κ, IFN-δ, IFN-ε, IFN-ε, IFN-ω, and LIGHT; IFN-α, IFN-β, IFN-κ, IFN-δ, IFN-ε, IFN-τ, IFN-ω, and TIM-4; IFN-α, IFN-β, IFN-κ, IFN-δ, IFN-ε, IFN-τ, IFN-ω, and ICAM-1; IFN-α, IFN-β, IFN-δ, IFN-ε, IFN-τ, IFN-ω, and CD58; IFN-α, IFN-β, IFN-κ, IFN-δ, IFN-ε, IFN-τ, IFN-ω, and SLAMF6; IFN-α, IFN-β, IFN-κ, IFN-δ, IFN-ε, IFN-τ, IFN-ω, IFN-ζ, and CD40-L; IFN-α, IFN-β, IFN-κ, IFN-δ, IFN-ε, IFN-τ, IFN-ω, IFN-ζ, and MEM40; IFN-α, IFN-β, IFN-κ, IFN-δ, IFN-ε, IFN-τ, IFN-ω, IFN-ζ, and B7-1(CD80)/B7-2(CD86); IFN-α, IFN-β, IFN-κ, IFN-δ, IFN-ε, IFN-τ, IFN-ω, IFN-ζ, and OX40L; IFN-α, IFN-β, IFN-κ, IFN-δ, IFN-ε, IFN-τ, IFN-ω, IFN-ζ, and 4-1BBL; IFN-α, IFN-β, IFN-κ, IFN-δ, IFN-ε, IFN-τ, IFN-ω, IFN-ζ, and CD70; IFN-α, IFN-β, IFN-κ, IFN-δ, IFN-ε, IFN-τ, IFN-ω, IFN-ζ, and GITRL; IFN-α, IFN-β, IFN-κ, IFN-δ, IFN-ε, IFN-τ, IFN-ω, IFN-ζ, and LIGHT; IFN-α, IFN-β, IFN-κ, IFN-δ, IFN-ε, IFN-τ, IFN-ω, IFN-ζ, and TIM-4; IFN-α, IFN-β, IFN-κ, IFN-δ, IFN-ε, IFN-τ, IFN-ω, IFN-ζ, and ICAM-1; IFN-α, IFN-β, IFN-δ, IFN-ε, IFN-ε, IFN-ω, IFN-ζ, and CD58; IFN-α, IFN-β, IFN-κ, IFN-δ, IFN-ε, IFN-τ, IFN-ω, IFN-ζ, and SLAMF6; IFN-β and CD40-L; IFN-β and MEM40; IFN-β and B7-1(CD80)/B7-2(CD86); IFN-β and OX40L; IFN-β and 4-1BBL; IFN-β and CD70; IFN-β and GITRL; IFN-β and LIGHT; IFN-β and TIM-4; IFN-β and ICAM-1; IFN-β and CD58; IFN-β and SLAMF6; IFN-κ and CD40-L; IFN-κ and MEM40; IFN-κ and B7-1(CD80)/B7-2(CD86); IFN-κ and OX40L; IFN-κ and 4-1BBL; IFN-κ and CD70; IFN-κ and GITRL; IFN-κ and LIGHT; IFN-κ and TIM-4; IFN-κ and ICAM-1; IFN-κ and CD58; IFN-α and SLAMF6; IFN-δ and CD40-L; IFN-δ and MEM40; IFN-δ and B7-1(CD80)/B7-2(CD86); IFN-δ and OX40L; IFN-δ and 4-1BBL; IFN-δ and CD70; IFN-δ and GITRL; IFN-δ and LIGHT; IFN-δ and TIM-4; IFN-δ and ICAM-1; IFN-δ and CD58; IFN-δ and SLAMF6; IFN-ε and CD40-L, IFN-ε and MEM40; IFN-ε and B7-1(CD80)/B7-2(CD86); IFN-ε and OX40L; IFN-ε and 4-1BBL; IFN-ε and CD70; IFN-ε and GITRL; IFN-ε and LIGHT; IFN-ε and TIM-4; IFN-ε and ICAM-1; IFN-ε and CD58; IFN-ε and SLAMF6; IFN-τ and CD40-L; IFN-τ and MEM40; IFN-τ and B7-1(CD80)/B7-2(CD86); IFN-τ and OX40L; IFN-τ and 4-1BBL; IFN-τ and CD70; IFN-τ and GITRL; IFN-τ and LIGHT; IFN-τ and TIM-4; IFN-τ and ICAM-1; IFN-τ and CD58; IFN-τ and SLAMF6; IFN-ω and CD40-L; IFN-ω and MEM40; IFN-ω and B7-1(CD80)/B7-2(CD86); IFN-ω and OX40L; IFN-ω and 4-1BBL; IFN-ω and CD70; IFN-ω and GITRL; IFN-ω and LIGHT; IFN-ω and TIM-4; IFN-ω and ICAM-1; IFN-ω and CD58; IFN-ω and SLAMF6; IFN-ζ and CD40-L; IFN-ζ and MEM40; IFN-ζ and B7-1(CD80)/B7-2(CD86); IFN-ζ and OX40L; IFN-ζ and 4-1BBL; IFN-ζ and CD70; IFN-ζ and GITRL; IFN-ζ and LIGHT; IFN-ζ and TIM-4; IFN-ζ and ICAM-1; IFN-ζ and CD58; IFN-ζ and SLAMF6; IFN-β, CD40-L, and MEM40; IFN-β, CD40-L, and B7-1(CD80)/B7-2(CD86); IFN-β, CD40-L, and OX40L; IFN-β, CD40-L, and 4-1BBL; IFN-β, CD40-L, and CD70; IFN-β, CD40-L, and GITRL; IFN-β, CD40-L, and LIGHT; IFN-β, CD40-L, and TIM-4; IFN-β, CD40-L, and ICAM-1; IFN-β, CD40-L, and CD58; IFN-β, CD40-L, and SLAMF6; IFN-β, CD40-L, MEM40, and B7-1(CD80)/B7-2(CD86); IFN-β, CD40-L, MEM40, and OX40L; IFN-β, CD40-L, MEM40, and 4-1BBL; IFN-β, CD40-L, MEM40, and CD70; IFN-β, CD40-L, MEM40, and GITRL; IFN-β, CD40-L, MEM40, and LIGHT; IFN-β, CD40-L, MEM40, and TIM-4; IFN-β, CD40-L, MEM40, and ICAM-1; IFN-β, CD40-L, MEM40, and CD58; IFN-β, CD40-L, MEM40, and SLAMF6; IFN-β, CD40-L, MEM40, B7-1(CD80)/B7-2(CD86), and OX40L; IFN-β, CD40-L, MEM40, B7-1(CD80)/B7-2(CD86), and 4-1BBL; IFN-β, CD40-L, MEM40, B7-1(CD80)/B7-2(CD86), and CD70; IFN-β, CD40-L, MEM40, B7-1(CD80)/B7-2(CD86), and GITRL; IFN-β, CD40-L, MEM40, B7-1(CD80)/B7-2(CD86), and LIGHT; IFN-β, CD40-L, MEM40, B7-1(CD80)/B7-2(CD86), and TIM-4; IFN-β, CD40-L, MEM40, B7-1(CD80)/B7-2(CD86), and ICAM-1; IFN-β, CD40-L, MEM40, B7-1(CD80)/B7-2(CD86), and CD58; IFN-β, CD40-L, MEM40, B7-1(CD80)/B7-2(CD86), and SLAMF6; IFN-β, CD40-L, MEM40, B7-1(CD80)/B7-2(CD86), OX40L, and 4-1BBL; IFN-β, CD40-L, MEM40, B7-1(CD80)/B7-2(CD86), OX40L, and CD70; IFN-β, CD40-L, MEM40, B7-1(CD80)/B7-2(CD86), OX40L, and GITRL; IFN-β, CD40-L, MEM40, B7-1(CD80)/B7-2(CD86), OX40L, and LIGHT; IFN-β, CD40-L, MEM40, B7-1(CD80)/B7-2(CD86), OX40L, and TIM-4; IFN-β, CD40-L, MEM40, B7-1(CD80)/B7-2(CD86), OX40L, and ICAM-1; IFN-β, CD40-L, MEM40, B7-1(CD80)/B7-2(CD86), OX40L, and CD58; IFN-β, CD40-L, MEM40, B7-1(CD80)/B7-2(CD86), OX40L, and SLAMF6; IFN-β, CD40-L, MEM40, B7-1(CD80)/B7-2(CD86), OX40L, 4-1BBL, and CD70; IFN-β, CD40-L, MEM40, B7-1(CD80)/B7-2(CD86), OX40L, 4-1BBL, and GITRL; IFN-β, CD40-L, MEM40, B7-1(CD80)/B7-2(CD86), OX40L, 4-1BBL, and LIGHT; IFN-β, CD40-L, MEM40, B7-1(CD80)/B7-2(CD86), OX40L, 4-1BBL, and TIM-4; IFN-β, CD40-L, MEM40, B7-1(CD80)/B7-2(CD86), OX40L, 4-1BBL, and ICAM-1; IFN-β, CD40-L, MEM40, B7-1(CD80)/B7-2(CD86), OX40L, 4-1BBL, and CD58; IFN-β, CD40-L, MEM40, B7-1(CD80)/B7-2(CD86), OX40L, 4-1BBL, and SLAMF6; IFN-β, CD40-L, MEM40, B7-1(CD80)/B7-2(CD86), OX40L, 4-1BBL, CD70, and GITRL; IFN-β, CD40-L, MEM40, B7-1(CD80)/B7-2(CD86), OX40L, 4-1BBL, CD70, and LIGHT; IFN-β, CD40-L, MEM40, B7-1(CD80)/B7-2(CD86), OX40L, 4-1BBL, CD70, and TIM-4; IFN-β, CD40-L, MEM40, B7-1(CD80)/B7-2(CD86), OX40L, 4-1BBL, CD70, and ICAM-1; IFN-β, CD40-L, MEM40, B7-1(CD80)/B7-2(CD86), OX40L, 4-1BBL, CD70, and CD58; IFN-β, CD40-L, MEM40, B7-1(CD80)/B7-2(CD86), OX40L, 4-1BBL, CD70, and SLAMF6; IFN-β, CD40-L, MEM40, B7-1(CD80)/B7-2(CD86), OX40L, 4-1BBL, CD70, GITRL, and LIGHT; IFN-β, CD40-L, MEM40, B7-1(CD80)/B7-2(CD86), OX40L, 4-1BBL, CD70, GITRL, and TIM-4; IFN-β, CD40-L, MEM40, B7-1(CD80)/B7-2(CD86), OX40L, 4-1BBL, CD70, GITRL, and ICAM-1; IFN-β, CD40-L, MEM40, B7-1(CD80)/B7-2(CD86), OX40L, 4-1BBL, CD70, GITRL, and CD58; IFN-β, CD40-L, MEM40, B7-1(CD80)/B7-2(CD86), OX40L, 4-1BBL, CD70, GITRL, and SLAMF6; IFN-β, CD40-L, MEM40, B7-1(CD80)/B7-2(CD86), OX40L, 4-1BBL, CD70, GITRL, LIGHT, and TIM-4; IFN-β, CD40-L, MEM40, B7-1(CD80)/B7-2(CD86), OX40L, 4-1BBL, CD70, GITRL, LIGHT and ICAM-1; IFN-β, CD40-L, MEM40, B7-1(CD80)/B7-2(CD86), OX40L, 4-1BBL, CD70, GITRL, LIGHT and CD58; IFN-β, CD40-L, MEM40, B7-1(CD80)/B7-2(CD86), OX40L, 4-1BBL, CD70, GITRL, LIGHT and SLAMF6; IFN-β, CD40-L, MEM40, B7-1(CD80)/B7-2(CD86), OX40L, 4-1BBL, CD70, GITRL, LIGHT, TIM-4, and ICAM-1; IFN-β, CD40-L, MEM40, B7-1(CD80)/B7-2(CD86), OX40L, 4-1BBL, CD70, GITRL, LIGHT, TIM-4, and CD58; IFN-β, CD40-L, MEM40, B7-1(CD80)/B7-2(CD86), OX40L, 4-1BBL, CD70, GITRL, LIGHT, TIM-4, and SLAMF6; IFN-β, CD40-L, MEM40, B7-1(CD80)/B7-2(CD86), OX40L, 4-1BBL, CD70, GITRL, LIGHT, TIM-4, ICAM-1, and CD58; IFN-β, CD40-L, MEM40, B7-1(CD80)/B7-2(CD86), OX40L, 4-1BBL, CD70, GITRL, LIGHT, TIM-4, ICAM-1, and SLAMF6; IFN-β, CD40-L, MEM40, B7-1(CD80)/B7-2(CD86), OX40L, 4-1BBL, CD70, GITRL, LIGHT, TIM-4, ICAM-1, CD58, and SLAMF6; IFN-β, MEM40; IFN-β, MEM40, and B7-1(CD80)/B7-2(CD86); IFN-β, MEM40, and OX40L; IFN-β, MEM40, and 4-1BBL; IFN-β, MEM40, and CD70; IFN-β, MEM40, and GITRL; IFN-β, MEM40, and LIGHT; IFN-β, MEM40, and TIM-4; IFN-β, MEM40, and ICAM-1; IFN-β, MEM40, and CD58; IFN-β, MEM40, and SLAMF6; IFN-β, MEM40, and B7-1(CD80)/B7-2(CD86); IFN-β, MEM40, and OX40L; IFN-β, MEM40, and 4-1BBL; IFN-β, MEM40, and CD70; IFN-β, MEM40, and GITRL; IFN-β, MEM40, and LIGHT; IFN-β, MEM40, and TIM-4; IFN-β, MEM40, and ICAM-1; IFN-β, MEM40, and CD58; IFN-β, MEM40, and SLAMF6; IFN-β, MEM40, B7-1(CD80)B7-2(CD86), and OX40L; IFN-β, MEM40, B7-1(CD80)/B7-2(CD86), and 4-1BBL; IFN-β, MEM40, B7-1(CD80)/B7-2(CD86), and CD70; IFN-β, MEM40, B7-1(CD80)/B7-2(CD86), and GITRL; IFN-β, MEM40, B7-1(CD80)/B7-2(CD86), and LIGHT; IFN-β, MEM40, B7-1(CD80)/B7-2(CD86), and TIM-4; IFN-β, MEM40, B7-1(CD80)/B7-2(CD86), and ICAM-1; IFN-β, MEM40, B7-1(CD80)/B7-2(CD86), and CD58; IFN-β, MEM40, B7-1(CD80)/B7-2(CD86), and SLAMF6; IFN-β, MEM40, B7-1(CD80)/B7-2(CD86), OX40L, and 4-1BBL; IFN-β, MEM40, B7-1(CD80)/B7-2(CD86), OX40L, and CD70; IFN-β, MEM40, B7-1(CD80)/B7-2(CD86), OX40L, and GITRL; IFN-β, MEM40, B7-1(CD80)/B7-2(CD86), OX40L, and LIGHT; IFN-β, MEM40, B7-1(CD80)/B7-2(CD86), OX40L, and TIM-4; IFN-β, MEM40, B7-1(CD80)/B7-2(CD86), OX40L, and ICAM-1; IFN-β, MEM40, B7-1(CD80)/B7-2(CD86), OX40L, and CD58; IFN-β, MEM40, B7-1(CD80)/B7-2(CD86), OX40L, and SLAMF6; IFN-β, MEM40, B7-1(CD80)/B7-2(CD86), OX40L, 4-1BBL, and CD70; IFN-β, MEM40, B7-1(CD80)/B7-2(CD86), OX40L, 4-1BBL, and GITRL; IFN-β, MEM40, B7-1(CD80)/B7-2(CD86), OX40L, 4-1BBL, and LIGHT; IFN-β, MEM40, B7-1(CD80)/B7-2(CD86), OX40L, 4-1BBL, and TIM-4; IFN-β, MEM40, B7-1(CD80)/B7-2(CD86), OX40L, 4-1BBL, and ICAM-1; IFN-β, MEM40, B7-1(CD80)/B7-2(CD86), OX40L, 4-1BBL, and CD58; IFN-β, MEM40, B7-1(CD80)/B7-2(CD86), OX40L, 4-1BBL, and SLAMF6; IFN-β, MEM40, B7-1(CD80)/B7-2(CD86), OX40L, 4-1BBL, CD70, and GITRL; IFN-β, MEM40, B7-1(CD80)/B7-2(CD86), OX40L, 4-1BBL, CD70, and LIGHT; IFN-β, MEM40, B7-1(CD80)/B7-2(CD86), OX40L, 4-1BBL, CD70, and TIM-4; IFN-β, MEM40, B7-1(CD80)/B7-2(CD86), OX40L, 4-1BBL, CD70, and ICAM-1; IFN-β, MEM40, B7-1(CD80)/B7-2(CD86), OX40L, 4-1BBL, CD70, and CD58; IFN-β, MEM40, B7-1(CD80)/B7-2(CD86), OX40L, 4-1BBL, CD70, and SLAMF6; IFN-β, MEM40, B7-1(CD80)/B7-2(CD86), OX40L, 4-1BBL, CD70, GITRL, and LIGHT; IFN-β, MEM40, B7-1(CD80)/B7-2(CD86), OX40L, 4-1BBL, CD70, GITRL, and TIM-4; IFN-β, MEM40, B7-1(CD80)/B7-2(CD86), OX40L, 4-1BBL, CD70, GITRL, and ICAM-1; IFN-β, MEM40, B7-1(CD80)/B7-2(CD86), OX40L, 4-1BBL, CD70, GITRL, and CD58; IFN-β, MEM40, B7-1(CD80)/B7-2(CD86), OX40L, 4-1BBL, CD70, GITRL, and SLAMF6; IFN-β, MEM40, B7-1(CD80)/B7-2(CD86), OX40L, 4-1BBL, CD70, GITRL, LIGHT, and TIM-4; IFN-β, MEM40, B7-1(CD80)/B7-2(CD86), OX40L, 4-1BBL, CD70, GITRL, LIGHT and ICAM-1; IFN-β, MEM40, B7-1(CD80)/B7-2(CD86), OX40L, 4-1BBL, CD70, GITRL, LIGHT and CD58; IFN-β, MEM40, B7-1(CD80)/B7-2(CD86), OX40L, 4-1BBL, CD70, GITRL, LIGHT and SLAMF6; IFN-β, MEM40, B7-1(CD80)/B7-2(CD86), OX40L, 4-1BBL, CD70, GITRL, LIGHT, TIM-4, and ICAM-1; IFN-β, MEM40, B7-1(CD80)/B7-2(CD86), OX40L, 4-1BBL, CD70, GITRL, LIGHT, TIM-4, and CD58; IFN-β, MEM40, B7-1(CD80)/B7-2(CD86), OX40L, 4-1BBL, CD70, GITRL, LIGHT, TIM-4, and SLAMF6; IFN-β, MEM40, B7-1(CD80)/B7-2(CD86), OX40L, 4-1BBL, CD70, GITRL, LIGHT, TIM-4, ICAM-1, and CD58; IFN-β, MEM40, B7-1(CD80)/B7-2(CD86), OX40L, 4-1BBL, CD70, GITRL, LIGHT, TIM-4, ICAM-1, and SLAMF6; IFN-β, MEM40, B7-1(CD80)/B7-2(CD86), OX40L, 4-1BBL, CD70, GITRL, LIGHT, TIM-4, ICAM-1, CD58, and SLAMF6; IFN-β, and B7-1(CD80)/B7-2(CD86); IFN-β, B7-1(CD80)/B7-2(CD86), and OX40L; IFN-β, B7-1(CD80)/B7-2(CD86), and 4-1BBL; IFN-β, B7-1(CD80)/B7-2(CD86), and CD70; IFN-β, B7-1(CD80)/B7-2(CD86), and GITRL; IFN-β, B7-1(CD80)/B7-2(CD86), and LIGHT; IFN-β, B7-1(CD80)/B7-2(CD86), and TIM-4; IFN-β, B7-1(CD80)/B7-2(CD86), and ICAM-1; IFN-β, B7-1(CD80)/B7-2(CD86), and CD58; IFN-β, B7-1(CD80)/B7-2(CD86), and SLAMF6; IFN-β, B7-1(CD80)/B7-2(CD86), and OX40L; IFN-β, B7-1(CD80)/B7-2(CD86), and 4-1BBL; IFN-β, B7-1(CD80)/B7-2(CD86), and CD70; IFN-β, B7-1(CD80)/B7-2(CD86), and GITRL; IFN-β, B7-1(CD80)/B7-2(CD86), and LIGHT; IFN-β, B7-1(CD80)/B7-2(CD86), and TIM-4; IFN-β, B7-1(CD80)/B7-2(CD86), and ICAM-1; IFN-β, B7-1(CD80)/B7-2(CD86), and CD58; IFN-β, B7-1(CD80)/B7-2(CD86), and SLAMF6; IFN-β, B7-1(CD80)/B7-2(CD86), OX40L, and 4-1BBL; IFN-β, B7-1(CD80)/B7-2(CD86), OX40L, and CD70; IFN-β, B7-1(CD80)/B7-2(CD86), OX40L, and GITRL; IFN-β, B7-1(CD80)/B7-2(CD86), OX40L, and LIGHT; IFN-β, B7-1(CD80)/B7-2(CD86), OX40L, and TIM-4; IFN-β, B7-1(CD80)/B7-2(CD86), OX40L, and ICAM-1; IFN-β, B7-1(CD80)/B7-2(CD86), OX40L, and CD58; IFN-β, B7-1(CD80)/B7-2(CD86), OX40L, and SLAMF6; IFN-β, B7-1(CD80)/B7-2(CD86), OX40L, 4-1BBL, and CD70; IFN-β, B7-1(CD80)/B7-2(CD86), OX40L, 4-1BBL, and GITRL; IFN-β, B7-1(CD80)/B7-2(CD86), OX40L, 4-1BBL, and LIGHT; IFN-β, B7-1(CD80)/B7-2(CD86), OX40L, 4-1BBL, and TIM-4; IFN-β, B7-1(CD80)/B7-2(CD86), OX40L, 4-1BBL, and ICAM-1; IFN-β, B7-1(CD80)/B7-2(CD86), OX40L, 4-1BBL, and CD58; IFN-β, B7-1(CD80)/B7-2(CD86), OX40L, 4-1BBL, and SLAMF6; IFN-β, B7-1(CD80)/B7-2(CD86), OX40L, 4-1BBL, CD70, and GITRL; IFN-β, B7-1(CD80)/B7-2(CD86), OX40L, 4-1BBL, CD70, and LIGHT; IFN-β, B7-1(CD80)/B7-2(CD86), OX40L, 4-1BBL, CD70, and TIM-4; IFN-β, B7-1(CD80)/B7-2(CD86), OX40L, 4-1BBL, CD70, and ICAM-1; IFN-β, B7-1(CD80)/B7-2(CD86), OX40L, 4-1BBL, CD70, and CD58; IFN-β, B7-1(CD80)/B7-2(CD86), OX40L, 4-1BBL, CD70, and SLAMF6; IFN-β, B7-1(CD80)/B7-2(CD86), OX40L, 4-1BBL, CD70, GITRL, and LIGHT; IFN-β, B7-1(CD80)/B7-2(CD86), OX40L, 4-1BBL, CD70, GITRL, and TIM-4; IFN-β, B7-1(CD80)/B7-2(CD86), OX40L, 4-1BBL, CD70, GITRL, and ICAM-1; IFN-β, B7-1(CD80)/B7-2(CD86), OX40L, 4-1BBL, CD70, GITRL, and CD58; IFN-β, B7-1(CD80)/B7-2(CD86), OX40L, 4-1BBL, CD70, GITRL, and SLAMF6; IFN-β, B7-1(CD80)/B7-2(CD86), OX40L, 4-1BBL, CD70, GITRL, LIGHT, and TIM-4; IFN-β, B7-1(CD80)/B7-2(CD86), OX40L, 4-1BBL, CD70, GITRL, LIGHT and ICAM-1; IFN-β, B7-1(CD80)/B7-2(CD86), OX40L, 4-1BBL, CD70, GITRL, LIGHT and CD58; IFN-β, B7-1(CD80)/B7-2(CD86), OX40L, 4-1BBL, CD70, GITRL, LIGHT and SLAMF6; IFN-β, B7-1(CD80)/B7-2(CD86), OX40L, 4-1BBL, CD70, GITRL, LIGHT, TIM-4, and ICAM-1; IFN-β, B7-1(CD80)/B7-2(CD86), OX40L, 4-1BBL, CD70, GITRL, LIGHT, TIM-4, and CD58; IFN-β, B7-1(CD80)/B7-2(CD86), OX40L, 4-1BBL, CD70, GITRL, LIGHT, TIM-4, and SLAMF6; IFN-β, B7-1(CD80)/B7-2(CD86), OX40L, 4-1BBL, CD70, GITRL, LIGHT, TIM-4, ICAM-1, and CD58; IFN-β, B7-1(CD80)/B7-2(CD86), OX40L, 4-1BBL, CD70, GITRL, LIGHT, TIM-4, ICAM-1, and SLAMF6; IFN-β, B7-1(CD80)/B7-2(CD86), OX40L, 4-1BBL, CD70, GITRL, LIGHT, TIM-4, ICAM-1, CD58, and SLAMF6; IFN-0, and OX40L; IFN-β, OX40L, and 4-1BBL; IFN-β, OX40L, and CD70; IFN-β, OX40L, and GITRL; IFN-β, OX40L, and LIGHT; IFN-β, OX40L, and TIM-4; IFN-β, OX40L, and ICAM-1; IFN-β, OX40L, and CD58; IFN-β, OX40L, and SLAMF6; IFN-β, OX40L, and 4-1BBL; IFN-β, OX40L, and CD70; IFN-β, OX40L, and GITRL; IFN-β, OX40L, and LIGHT; IFN-β, OX40L, and TIM-4; IFN-β, OX40L, and ICAM-1; IFN-β, OX40L, and CD58; IFN-β, OX40L, and SLAMF6; IFN-β, OX40L, 4-1BBL, and CD70; IFN-β, OX40L, 4-1BBL, and GITRL; IFN-β, OX40L, 4-1BBL, and LIGHT; IFN-β, OX40L, 4-1BBL, and TIM-4; IFN-β, OX40L, 4-1BBL, and ICAM-1, IFN-β, OX40L, 4-1BBL, and CD58; IFN-β, OX40L, 4-1BBL, and SLAMF6; IFN-β, OX40L, 4-1BBL, CD70, and GITRL; IFN-β, OX40L, 4-1BBL, CD70, and LIGHT; IFN-β, OX40L, 4-1BBL, CD70, and TIM-4; IFN-β, OX40L, 4-1BBL, CD70, and ICAM-1; IFN-β, OX40L, 4-1BBL, CD70, and CD58; IFN-β, OX40L, 4-1BBL, CD70, and SLAMF6; IFN-β, OX40L, 4-1BBL, CD70, GITRL, and LIGHT; IFN-β, OX40L, 4-1BBL, CD70, GITRL, and TIM-4; IFN-β, OX40L, 4-1BBL, CD70, GITRL, and ICAM-1; IFN-β, OX40L, 4-1BBL, CD70, GITRL, and CD58; IFN-β, OX40L, 4-1BBL, CD70, GITRL, and SLAMF6; IFN-β, OX40L, 4-1BBL, CD70, GITRL, LIGHT, and TIM-4; IFN-β, OX40L, 4-1BBL, CD70, GITRL, LIGHT and ICAM-1; IFN-β, OX40L, 4-1BBL, CD70, GITRL, LIGHT and CD58; IFN-β, OX40L, 4-1BBL, CD70, GITRL, LIGHT and SLAMF6; IFN-β, OX40L, 4-1BBL, CD70, GITRL, LIGHT, TIM-4, and ICAM-1; IFN-β, OX40L, 4-1BBL, CD70, GITRL, LIGHT, TIM-4, and CD58; IFN-β, OX40L, 4-1BBL, CD70, GITRL, LIGHT, TIM-4, and SLAMF6; IFN-β, OX40L, 4-1BBL, CD70, GITRL, LIGHT, TIM-4, ICAM-1, and CD58; IFN-β, OX40L, 4-1BBL, CD70, GITRL, LIGHT, TIM-4, ICAM-1, and SLAMF6; IFN-β, OX40L, 4-1BBL, CD70, GITRL, LIGHT, TIM-4, ICAM-1, CD58, and SLAMF6; IFN-β, and 4-1BBL; IFN-β, 4-1BBL, and CD70; IFN-β, 4-1BBL, and GITRL; IFN-β, 4-1BBL, and LIGHT; IFN-β, 4-1BBL, and TIM-4; IFN-β, 4-1BBL, and ICAM-1; IFN-β, 4-1BBL, and CD58; IFN-β, 4-1BBL, and SLAMF6; IFN-β, 4-1BBL, and CD70; IFN-β, 4-1BBL, and GITRL; IFN-β, 4-1BBL, and LIGHT; IFN-β, 4-1BBL, and TIM-4; IFN-β, 4-1BBL, and ICAM-1; IFN-β, 4-1BBL, and CD58; IFN-β, 4-1BBL, and SLAMF6; IFN-β, 4-1BBL, CD70, and GITRL; IFN-β, 4-1BBL, CD70, and LIGHT; IFN-β, 4-1BBL, CD70, and TIM-4; IFN-β, 4-1BBL, CD70, and ICAM-1; IFN-β, 4-1BBL, CD70, and CD58; IFN-β, 4-1BBL, CD70, and SLAMF6; IFN-β, 4-1BBL, CD70, GITRL, and LIGHT; IFN-β, 4-1BBL, CD70, GITRL, and TIM-4; IFN-β, 4-1BBL, CD70, GITRL, and ICAM-1; IFN-β, 4-1BBL, CD70, GITRL, and CD58; IFN-β, 4-1BBL, CD70, GITRL, and SLAMF6; IFN-β, 4-1BBL, CD70, GITRL, LIGHT, and TIM-4; IFN-β, 4-1BBL, CD70, GITRL, LIGHT and ICAM-1; IFN-β, 4-1BBL, CD70, GITRL, LIGHT and CD58; IFN-β, 4-1BBL, CD70, GITRL, LIGHT and SLAMF6; IFN-β, 4-1BBL, CD70, GITRL, LIGHT, TIM-4, and ICAM-1; IFN-β, 4-1BBL, CD70, GITRL, LIGHT, TIM-4, and CD58; IFN-β, 4-1BBL, CD70, GITRL, LIGHT, TIM-4, and SLAMF6; IFN-β, 4-1BBL, CD70, GITRL, LIGHT, TIM-4, ICAM-1, and CD58; IFN-β, 4-1BBL, CD70, GITRL, LIGHT, TIM-4, ICAM-1, and SLAMF6; IFN-β, 4-1BBL, CD70, GITRL, LIGHT, TIM-4, ICAM-1, CD58, and SLAMF6; IFN-β and CD70; IFN-β, CD70, and GITRL; IFN-β, CD70, and LIGHT; IFN-β, CD70, and TIM-4; IFN-β, CD70, and ICAM-1; IFN-β, CD70, and CD58; IFN-β, CD70, and SLAMF6; IFN-β, CD70, and GITRL; IFN-β, CD70, and LIGHT; IFN-β, CD70, and TIM-4; IFN-0, CD70, and ICAM-1; IFN-β, CD70, and CD58; IFN-β, CD70, and SLAMF6; IFN-β, CD70, GITRL, and LIGHT; IFN-β, CD70, GITRL, and TIM-4; IFN-β, CD70, GITRL, and ICAM-1; IFN-β, CD70, GITRL, and CD58; IFN-β, CD70, GITRL, and SLAMF6; IFN-β, CD70, GITRL, LIGHT, and TIM-4; IFN-β, CD70, GITRL, LIGHT and ICAM-1; IFN-β, CD70, GITRL, LIGHT and CD58; IFN-β, CD70, GITRL, LIGHT and SLAMF6; IFN-β, CD70, GITRL, LIGHT, TIM-4, and ICAM-1; IFN-β, CD70, GITRL, LIGHT, TIM-4, and CD58; IFN-β, CD70, GITRL, LIGHT, TIM-4, and SLAMF6; IFN-β, CD70, GITRL, LIGHT, TIM-4, ICAM-1, and CD58; IFN-β, CD70, GITRL, LIGHT, TIM-4, ICAM-1, and SLAMF6; IFN-β, CD70, GITRL, LIGHT, TIM-4, ICAM-1, CD58, and SLAMF6; IFN-β and GITRL; IFN-β, GITRL, and LIGHT; IFN-β, GITRL, and TIM-4; IFN-β, GITRL, and ICAM-1; IFN-β, GITRL, and CD58; IFN-β, GITRL, and SLAMF6; IFN-β, GITRL, and LIGHT; IFN-β, GITRL, and TIM-4; IFN-β, GITRL, and ICAM-1; IFN-β, GITRL, and CD58; IFN-β, GITRL, and SLAMF6; IFN-β, GITRL, LIGHT, and TIM-4; IFN-β, GITRL, LIGHT and ICAM-1; IFN-β, GITRL, LIGHT and CD58; IFN-β, GITRL, LIGHT and SLAMF6; IFN-β, GITRL, LIGHT, TIM-4, and ICAM-1; IFN-β, GITRL, LIGHT, TIM-4, and CD58; IFN-β, GITRL, LIGHT, TIM-4, and SLAMF6; IFN-β, GITRL, LIGHT, TIM-4, ICAM-1, and CD58; IFN-β, GITRL, LIGHT, TIM-4, ICAM-I, and SLAMF6; IFN-β, GITRL, LIGHT, TIM-4, ICAM-1, CD58, and SLAMF6; IFN-β, and LIGHT; IFN-β, LIGHT, and TIM-4; IFN-β, LIGHT, and ICAM-1; IFN-β, LIGHT, and CD58; IFN-β, LIGHT, and SLAMF6; IFN-β, LIGHT, and TIM-4; IFN-β, LIGHT and ICAM-1; IFN-β, LIGHT and CD58; IFN-β, LIGHT and SLAMF6; IFN-β, LIGHT, TIM-4, and ICAM-1; IFN-β, LIGHT, TIM-4, and CD58; IFN-β, LIGHT, TIM-4, and SLAMF6; IFN-β, LIGHT, TIM-4, ICAM-1, and CD58; IFN-β, LIGHT, TIM-4, ICAM-1, and SLAMF6; IFN-β, LIGHT, TIM-4, ICAM-1, CD58, and SLAMF6; IFN-β, and TIM-4; IFN-β, TIM-4, and ICAM-1; IFN-β, TIM-4, and CD58; IFN-β, TIM-4, and SLAMF6; IFN-β, TIM-4, and ICAM-1; IFN-β, TIM-4, and CD58; IFN-β, TIM-4, and SLAMF6; IFN-β, TIM-4, ICAM-1, and CD58; IFN-β, TIM-4, ICAM-1, and SLAMF6; IFN-β, TIM-4, ICAM-1, CD58, and SLAMF6; IFN-β and ICAM-1; IFN-β, ICAM-1, and CD58; IFN-β, ICAM-1, and SLAMF6; IFN-β, ICAM-1, and CD58; IFN-β, ICAM-1, and SLAMF6; IFN-β, ICAM-1, CD58, and SLAMF6; IFN-β and CD58; IFN-β, CD58, and SLAMF6; IFN-β and SLAMF6; IFN-β, IFN-κ, and CD40-L; IFN-β, IFN-κ, and MEM40; IFN-β, IFN-κ, and B7-1(CD80)/B7-2(CD86); IFN-β, IFN-κ, and OX40L; IFN-β, IFN-κ, and 4-1BBL; IFN-β, IFN-κ, and CD70; IFN-β, IFN-κ, and GITRL; IFN-β, IFN-κ, and LIGHT; IFN-β, IFN-κ, and TIM-4; IFN-β, IFN-κ, and ICAM-1; IFN-0, and CD58; IFN-β, IFN-κ, and SLAMF6; IFN-β, IFN-κ, IFN-β, and CD40-L; IFN-β, IFN-κ, IFN-δ, and MEM40; IFN-β, IFN-κ, IFN-β, and B7-1(CD80)/B7-2(CD86); IFN-β, IFN-κ, IFN-δ, and OX40L; IFN-β, IFN-κ, IFN-δ, and 4-1BBL; IFN-β, IFN-κ, IFN-δ, and CD70; IFN-β, IFN-κ, IFN-δ, and GITRL; IFN-β, IFN-κ, IFN-δ, and LIGHT; IFN-β, IFN-κ, IFN-δ, and TIM-4; IFN-β, IFN-κ, IFN-δ, and ICAM-1; IFN-β, IFN-δ, and CD58; IFN-β, IFN-κ, IFN-δ, and SLAMF6; IFN-β, IFN-κ, IFN-δ, IFN-ε, and CD40-L; IFN-β, IFN-κ, IFN-δ, IFN-ε, and MEM40; IFN-β, IFN-κ, IFN-δ, IFN-ε, and B7-1(CD80)/B7-2(CD86); IFN-β, IFN-κ, IFN-δ, IFN-ε, and OX40L; IFN-β, IFN-κ, IFN-δ, IFN-ε, and 4-1BBL; IFN-β, IFN-κ, IFN-δ, IFN-ε, and CD70; IFN-β, IFN-κ, IFN-8, IFN-ε, and GITRL; IFN-β, IFN-κ, IFN-δ, IFN-ε, and LIGHT; IFN-β, IFN-κ, IFN-δ, IFN-ε, and TIM-4; IFN-β, IFN-κ, IFN-δ, IFN-ε, and ICAM-1; IFN-β, IFN-δ, IFN-ε, and CD58; IFN-β, IFN-κ, IFN-δ, IFN-ε, and SLAMF6; IFN-β, IFN-κ, IFN-δ, IFN-ε, IFN-τ, and CD40-L; IFN-β, IFN-κ, IFN-δ, IFN-ε, IFN-τ, and MEM40; IFN-β, IFN-κ, IFN-δ, IFN-ε, IFN-τ, and B7-1(CD80)/B7-2(CD86); IFN-β, IFN-κ, IFN-δ, IFN-ε, IFN-τ, and OX40L; IFN-β, IFN-κ, IFN-δ, IFN-ε, IFN-ε, and 4-1BBL; IFN-β, IFN-κ, IFN-δ, IFN-ε, IFN-τ, and CD70; IFN-β, IFN-κ, IFN-δ, IFN-ε, IFN-τ, and GITRL; IFN-β, IFN-κ, IFN-δ, IFN-ε, IFN-τ, and LIGHT; IFN-β, IFN-κ, IFN-δ, IFN-ε, IFN-τ, and TIM-4; IFN-β, IFN-κ, IFN-δ, IFN-ε, IFN-τ, and ICAM-1; IFN-β, IFN-δ, IFN-ε, IFN-τ, and CD58; IFN-β, IFN-κ, IFN-δ, IFN-ε, IFN-τ, and SLAMF6; IFN-β, IFN-κ, IFN-δ, IFN-ε, IFN-ε, IFN-ω, and CD40-L; IFN-β, IFN-κ, IFN-δ, IFN-ε, IFN-τ, IFN-ω, and MEM40; IFN-β, IFN-κ, IFN-δ, IFN-ε, IFN-τ, IFN-ω, and B7-1(CD80)/B7-2(CD86); IFN-β, IFN-κ, IFN-δ, IFN-ε, IFN-ε, IFN-ω, and OX40L; IFN-β, IFN-κ, IFN-δ, IFN-ε, IFN-τ, IFN-ω, and 4-1BBL; IFN-β, IFN-κ, IFN-δ, IFN-ε, IFN-τ, IFN-ω, and CD70; IFN-β, IFN-κ, IFN-δ, IFN-ε, IFN-τ, IFN-ω, and GITRL; IFN-β, IFN-κ, IFN-δ, IFN-ε, IFN-ζ, IFN-ω, and LIGHT; IFN-β, IFN-κ, IFN-δ, IFN-ε, IFN-ζ, IFN-ω, and TIM-4; IFN-β, IFN-κ, IFN-δ, IFN-ε, IFN-τ, IFN-ω, and ICAM-1; IFN-β, IFN-δ, IFN-ε, IFN-ζ, IFN-ω, and CD58; IFN-β, IFN-κ, IFN-δ, IFN-ε, IFN-τ, IFN-ω, and SLAMF6; IFN-β, IFN-κ, IFN-δ, IFN-ε, IFN-τ, IFN-ω, IFN-ζ, and CD40-L; IFN-β, IFN-κ, IFN-δ, IFN-ε, IFN-τ, IFN-ω, IFN-ζ, and MEM40; IFN-β, IFN-κ, IFN-δ, IFN-ε, IFN-τ, IFN-ω, IFN-ζ, and B7-1(CD80)/B7-2(CD86); IFN-β, IFN-κ, IFN-δ, IFN-ε, IFN-τ, IFN-ω, IFN-ζ, and OX40L; IFN-β, IFN-κ, IFN-δ, IFN-ε, IFN-τ, IFN-ω, IFN-ζ, and 4-1BBL; IFN-β, IFN-κ, IFN-δ, IFN-ε, IFN-τ, IFN-ω, IFN-ζ, and CD70; IFN-β, IFN-κ, IFN-δ, IFN-ε, IFN-τ, IFN-ω, IFN-ζ, and GITRL; IFN-β, IFN-κ, IFN-δ, IFN-ε, IFN-τ, IFN-ω, IFN-ζ, and LIGHT; IFN-β, IFN-κ, IFN-δ, IFN-ε, IFN-τ, IFN-ω, IFN-ζ, and TIM-4; IFN-β, IFN-κ, IFN-δ, IFN-ε, IFN-τ, IFN-ω, IFN-ζ, and ICAM-1; IFN-β, IFN-δ, IFN-ε, IFN-τ, IFN-ω, IFN-ζ, and CD58; and/or IFN-β, IFN-κ, IFN-δ, IFN-ε, IFN-τ, IFN-ω, IFN-ζ, and SLAMF6. In one aspect, disclosed herein are methods of expanding a population of tumor infiltrating lymphocytes (TILs) or marrow infiltrating lymphocytes (MILs) wherein the oncolytic virus further comprises IL-12 and/or IL-23. Alternatively, disclosed herein are methods of expanding a population of tumor infiltrating lymphocytes (TILs) or marrow infiltrating lymphocytes (MILs) comprising a) harvesting TILs or MILs from a subject with a cancer; b) culturing the harvested TILs or MILs in the presence of antigen presenting cells infected with an oncolytic virus expressing i) one or more type 1 interferon (IFN) (such as, for example, IFN-α, IFN-β, IFN-κ, IFN-δ, IFN-ε, IFN-τ, IFN-ω, and/or IFN-ζ) or one or more exogenous immunostimulatory molecules (such as, for example, CD40-L, MEM40, B7-1(CD80)/B7-2(CD86), OX40L, 4-1BBL, CD70, GITRL, LIGHT, TIM-4, ICAM-1, CD58 and/or SLAMF6) and/or ii) an interleukin selected from IL-12 and/or IL-23. For example, the oncolytic virus for use in the disclosed methods can comprise IFN-α and IL-12, IFN-β and IL-12, IFN-κ and IL-12, IFN-δ and IL-12, IFN-ε and IL-12, IFN-τ and IL-12, IFN-ω and IL-12, IFN-ζ and IL-12, CD40-L and IL-12, MEM40 and IL-12, B7-1(CD80)/B7-2(CD86) and IL-12, OX40L and IL-12, 4-1BBL and IL-12, CD70 and IL-12, GITRL and IL-12, LIGHT and IL-12, TIM-4 and IL-12, ICAM-1 and IL-12, CD58 and IL-12, SLAMF6 and IL-12, IFN-α and IL-23, IFN-β and IL-23, IFN-κ and IL-23, IFN-δ and IL-23, IFN-ε and IL-23, IFN-τ and IL-23, IFN-ω and IL-23, IFN-ζ and IL-23, CD40-L and IL-23, MEM40 and IL-23, B7-1(CD80)/B7-2(CD86) and IL-23, OX40L and IL-23, 4-1BBL and IL-23, CD70 and IL-23, GITRL and IL-23, LIGHT and IL-23, TIM-4 and IL-23, ICAM-1 and IL-23, CD58 and IL-23, SLAMF6 and IL-23, IFN-α, IL-12, and IL-23, IFN-β, IL-12, and IL-23, IFN-κ, IL-12, and IL-23, IFN-δ, IL-12, and IL-23, IFN-ε, IL-12, and IL-23, IFN-τ, IL-12, and IL-23, IFN-ω, IL-12, and IL-23, IFN-ζ, IL-12, and IL-23, CD40-L, IL-12, and IL-23, MEM40, IL-12, and IL-23, B7-1(CD80)/B7-2(CD86), IL-12, and IL-23, OX40L, IL-12, and IL-23, 4-1BBL, IL-12, and IL-23, CD70, IL-12, and IL-23, GITRL, IL-12, and IL-23, LIGHT, IL-12, and IL-23, TIM-4, IL-12, and IL-23, ICAM-1, IL-12, and IL-23, CD58, IL-12, and IL-23, and/or SLAMF6, IL-12, and IL-23.

In certain embodiments, the invention provides a viral vector, such as a lentivirus, that expresses one or more type 1 interferon (IFN) (such as, for example, IFN-α, IFN-β, IFN-κ, IFN-δ, IFN-ε, IFN-τ, IFN-ω, and/or IFN-ζ) and/or one or more exogenous immunostimulatory molecule (such as, for example, CD40-L, MEM40, cluster of differentiation (CD) 80 and/or CD 86 (also known as B7-1(CD80) and B7-2(CD86)), OX40L, 4-1BB ligand (4-1BBL), CD70, LIGHT, glucocorticoid-induced TNFR-related protein ligand (GITRL), LIGHT, T-cell immunoglobulin and mucin domain 4 (TIM-4), intracellular adhesion molecule-1 (ICAM-1), CD58, and/or signaling lymphocyte activation molecule (SLAM) family member 6 (SLAMF6)) or any combination thereof. For example, the oncolytic virus can express IFN-α and CD40-L; IFN-α and MEM40; IFN-α and B7-1(CD80)/B7-2(CD86); IFN-α and OX40L; IFN-α and 4-1BBL; IFN-α and CD70; IFN-α and GITRL; IFN-α and LIGHT; IFN-α and TIM-4; IFN-α and ICAM-1; IFN-α and CD58; IFN-α and SLAMF6; IFN-α, CD40-L, and MEM40; IFN-α, CD40-L, and B7-1(CD80)/B7-2(CD86); IFN-α, CD40-L, and OX40L; IFN-α, CD40-L, and 4-1BBL; IFN-α, CD40-L, and CD70; IFN-α, CD40-L, and GITRL; IFN-α, CD40-L, and LIGHT; IFN-α, CD40-L, and TIM-4; IFN-α, CD40-L, and ICAM-1; IFN-α, CD40-L, and CD58; IFN-α, CD40-L, and SLAMF6; IFN-α, CD40-L, MEM40, and B7-1(CD80)/B7-2(CD86); IFN-α, CD40-L, MEM40, and OX40L; IFN-α, CD40-L, MEM40, and 4-1BBL; IFN-α, CD40-L, MEM40, and CD70; IFN-α, CD40-L, MEM40, and GITRL; IFN-α, CD40-L, MEM40, and LIGHT; IFN-α, CD40-L, MEM40, and TIM-4; IFN-α, CD40-L, MEM40, and ICAM-1; IFN-α, CD40-L, MEM40, and CD58; IFN-α, CD40-L, MEM40, and SLAMF6; IFN-α, CD40-L, MEM40, B7-1(CD80)/B7-2(CD86), and OX40L; IFN-α, CD40-L, MEM40, B7-1(CD80)/B7-2(CD86), and 4-1BBL; IFN-α, CD40-L, MEM40, B7-1(CD80)/B7-2(CD86), and CD70; IFN-α, CD40-L, MEM40, B7-1(CD80)/B7-2(CD86), and GITRL; IFN-α, CD40-L, MEM40, B7-1(CD80)/B7-2(CD86), and LIGHT; IFN-α, CD40-L, MEM40, B7-1(CD80)/B7-2(CD86), and TIM-4; IFN-α, CD40-L, MEM40, B7-1(CD80)/B7-2(CD86), and ICAM-1; IFN-α, CD40-L, MEM40, B7-1(CD80)/B7-2(CD86), and CD58; IFN-α, CD40-L, MEM40, B7-1(CD80)/B7-2(CD86), and SLAMF6; IFN-α, CD40-L, MEM40, B7-1(CD80)/B7-2(CD86), OX40L, and 4-1BBL; IFN-α, CD40-L, MEM40, B7-1(CD80)/B7-2(CD86), OX40L, and CD70; IFN-α, CD40-L, MEM40, B7-1(CD80)/B7-2(CD86), OX40L, and GITRL; IFN-α, CD40-L, MEM40, B7-1(CD80)/B7-2(CD86), OX40L, and LIGHT; IFN-α, CD40-L, MEM40, B7-1(CD80)/B7-2(CD86), OX40L, and TIM-4; IFN-α, CD40-L, MEM40, B7-1(CD80)/B7-2(CD86), OX40L, and ICAM-1; IFN-α, CD40-L, MEM40, B7-1(CD80)/B7-2(CD86), OX40L, and CD58; IFN-α, CD40-L, MEM40, B7-1(CD80)/B7-2(CD86), OX40L, and SLAMF6; IFN-α, CD40-L, MEM40, B7-1(CD80)/B7-2(CD86), OX40L, 4-1BBL, and CD70; IFN-α, CD40-L, MEM40, B7-1(CD80)/B7-2(CD86), OX40L, 4-1BBL, and GITRL; IFN-α, CD40-L, MEM40, B7-1(CD80)/B7-2(CD86), OX40L, 4-1 BBL, and LIGHT; IFN-α, CD40-L, MEM40, B7-1(CD80)/B7-2(CD86), OX40L, 4-1BBL, and TIM-4; IFN-α, CD40-L, MEM40, B7-1(CD80)/B7-2(CD86), OX40L, 4-1BBL, and ICAM-1; IFN-α, CD40-L, MEM40, B7-1(CD80)/B7-2(CD86), OX40L, 4-1BBL, and CD58; IFN-α, CD40-L, MEM40, B7-1(CD80)/B7-2(CD86), OX40L, 4-1BBL, and SLAMF6; IFN-α, CD40-L, MEM40, B7-1(CD80)/B7-2(CD86), OX40L, 4-1BBL, CD70, and GITRL; IFN-α, CD40-L, MEM40, B7-1(CD80)/B7-2(CD86), OX40L, 4-1BBL, CD70, and LIGHT; IFN-α, CD40-L, MEM40, B7-1(CD80)/B7-2(CD86), OX40L, 4-1BBL, CD70, and TIM-4; IFN-α, CD40-L, MEM40, B7-1(CD80)/B7-2(CD86), OX40L, 4-1BBL, CD70, and ICAM-1; IFN-α, CD40-L, MEM40, B7-1(CD80)/B7-2(CD86), OX40L, 4-1BBL, CD70, and CD58; IFN-α, CD40-L, MEM40, B7-1(CD80)/B7-2(CD86), OX40L, 4-1BBL, CD70, and SLAMF6; IFN-α, CD40-L, MEM40, B7-1(CD80)/B7-2(CD86), OX40L, 4-1BBL, CD70, GITRL, and LIGHT; IFN-α, CD40-L, MEM40, B7-1(CD80)/B7-2(CD86), OX40L, 4-1BBL, CD70, GITRL, and TIM-4; IFN-α, CD40-L, MEM40, B7-1(CD80)/B7-2(CD86), OX40L, 4-1BBL, CD70, GITRL, and ICAM-1; IFN-α, CD40-L, MEM40, B7-1(CD80)/B7-2(CD86), OX40L, 4-1BBL, CD70, GITRL, and CD58; IFN-α, CD40-L, MEM40, B7-1(CD80)/B7-2(CD86), OX40L, 4-1BBL, CD70, GITRL, and SLAMF6; IFN-α, CD40-L, MEM40, B7-1(CD80)/B7-2(CD86), OX40L, 4-1BBL, CD70, GITRL, LIGHT, and TIM-4; IFN-α, CD40-L, MEM40, B7-1(CD80)/B7-2(CD86), OX40L, 4-1BBL, CD70, GITRL, LIGHT and ICAM-1; IFN-α, CD40-L, MEM40, B7-1(CD80)/B7-2(CD86), OX40L, 4-1BBL, CD70, GITRL, LIGHT and CD58; IFN-α, CD40-L, MEM40, B7-1(CD80)/B7-2(CD86), OX40L, 4-1BBL, CD70, GITRL, LIGHT and SLAMF6; IFN-α, CD40-L, MEM40, B7-1(CD80)/B7-2(CD86), OX40L, 4-1BBL, CD70, GITRL, LIGHT, TIM-4, and ICAM-1; IFN-α, CD40-L, MEM40, B7-1(CD80)/B7-2(CD86), OX40L, 4-1BBL, CD70, GITRL, LIGHT, TIM-4, and CD58; IFN-α, CD40-L, MEM40, B7-1(CD80)/B7-2(CD86), OX40L, 4-1BBL, CD70, GITRL, LIGHT, TIM-4, and SLAMF6; IFN-α, CD40-L, MEM40, B7-1(CD80)/B7-2(CD86), OX40L, 4-1BBL, CD70, GITRL, LIGHT, TIM-4, ICAM-1, and CD58; IFN-α, CD40-L, MEM40, B7-1(CD80)/B7-2(CD86), OX40L, 4-1BBL, CD70, GITRL, LIGHT, TIM-4, ICAM-1, and SLAMF6; IFN-α, CD40-L, MEM40, B7-1(CD80)/B7-2(CD86), OX40L, 4-1BBL, CD70, GITRL, LIGHT, TIM-4, ICAM-1, CD58, and SLAMF6; IFN-α, MEM40; IFN-α, MEM40, and B7-1(CD80)/B7-2(CD86); IFN-α, MEM40, and OX40L; IFN-α, MEM40, and 4-1BBL; IFN-α, MEM40, and CD70; IFN-α, MEM40, and GITRL; IFN-α, MEM40, and LIGHT; IFN-α, MEM40, and TIM-4; IFN-α, MEM40, and ICAM-1; IFN-α, MEM40, and CD58; IFN-α, MEM40, and SLAMF6; IFN-α, MEM40, and B7-1(CD80)/B7-2(CD86); IFN-α, MEM40, and OX40L; IFN-α, MEM40, and 4-1BBL; IFN-α, MEM40, and CD70; IFN-α, MEM40, and GITRL; IFN-α, MEM40, and LIGHT; IFN-α, MEM40, and TIM-4; IFN-α, MEM40, and ICAM-1; IFN-α, MEM40, and CD58; IFN-α, MEM40, and SLAMF6; IFN-α, MEM40, B7-1(CD80)/B7-2(CD86), and OX40L; IFN-α, MEM40, B7-1(CD80)/B7-2(CD86), and 4-1BBL; IFN-α, MEM40, B7-1(CD80)/B7-2(CD86), and CD70; IFN-α, MEM40, B7-1(CD80)/B7-2(CD86), and GITRL; IFN-α, MEM40, B7-1(CD80)/B7-2(CD86), and LIGHT; IFN-α, MEM40, B7-1(CD80)/B7-2(CD86), and TIM-4; IFN-α, MEM40, B7-1(CD80)/B7-2(CD86), and ICAM-1; IFN-α, MEM40, B7-1(CD80)/B7-2(CD86), and CD58; IFN-α, MEM40, B7-1(CD80)/B7-2(CD86), and SLAMF6; IFN-α, MEM40, B7-1(CD80)/B7-2(CD86), OX40L, and 4-1BBL; IFN-α, MEM40, B7-1(CD80)/B7-2(CD86), OX40L, and CD70; IFN-α, MEM40, B7-1(CD80)/B7-2(CD86), OX40L, and GITRL; IFN-α, MEM40, B7-1(CD80)/B7-2(CD86), OX40L, and LIGHT; IFN-α, MEM40, B7-1(CD80)/B7-2(CD86), OX40L, and TIM-4; IFN-α, MEM40, B7-1(CD80)/B7-2(CD86), OX40L, and ICAM-1; IFN-α, MEM40, B7-1(CD80)/B7-2(CD86), OX40L, and CD58; IFN-α, MEM40, B7-1(CD80)/B7-2(CD86), OX40L, and SLAMF6; IFN-α, MEM40, B7-1(CD80)/B7-2(CD86), OX40L, 4-1BBL, and CD70; IFN-α, MEM40, B7-1(CD80)/B7-2(CD86), OX40L, 4-1BBL, and GITRL; IFN-α, MEM40, B7-1(CD80)/B7-2(CD86), OX40L, 4-1BBL, and LIGHT; IFN-α, MEM40, B7-1(CD80)/B7-2(CD86), OX40L, 4-1BBL, and TIM-4; IFN-α, MEM40, B7-1(CD80)/B7-2(CD86), OX40L, 4-1BBL, and ICAM-1; IFN-α, MEM40, B7-1(CD80)/B7-2(CD86), OX40L, 4-1BBL, and CD58; IFN-α, MEM40, B7-1(CD80)/B7-2(CD86), OX40L, 4-1BBL, and SLAMF6; IFN-α, MEM40, B7-1(CD80)/B7-2(CD86), OX40L, 4-1BBL, CD70, and GITRL; IFN-α, MEM40, B7-1(CD80)/B7-2(CD86), OX40L, 4-1BBL, CD70, and LIGHT; IFN-α, MEM40, B7-1(CD80)/B7-2(CD86), OX40L, 4-1BBL, CD70, and TIM-4; IFN-α, MEM40, B7-1(CD80)/B7-2(CD86), OX40L, 4-1BBL, CD70, and ICAM-1; IFN-α, MEM40, B7-1(CD80)/B7-2(CD86), OX40L, 4-1BBL, CD70, and CD58; IFN-α, MEM40, B7-1(CD80)/B7-2(CD86), OX40L, 4-1BBL, CD70, and SLAMF6; IFN-α, MEM40, B7-1(CD80)/B7-2(CD86), OX40L, 4-1BBL, CD70, GITRL, and LIGHT; IFN-α, MEM40, B7-1(CD80)/B7-2(CD86), OX40L, 4-1BBL, CD70, GITRL, and TIM-4; IFN-α, MEM40, B7-1(CD80)/B7-2(CD86), OX40L, 4-1BBL, CD70, GITRL, and ICAM-1; IFN-α, MEM40, B7-1(CD80)/B7-2(CD86), OX40L, 4-1BBL, CD70, GITRL, and CD58; IFN-α, MEM40, B7-1(CD80)/B7-2(CD86), OX40L, 4-1BBL, CD70, GITRL, and SLAMF6; IFN-α, MEM40, B7-1(CD80)B7-2(CD86), OX40L, 4-1BBL, CD70, GITRL, LIGHT, and TIM-4; IFN-α, MEM40, B7-1(CD80)/B7-2(CD86), OX40L, 4-1BBL, CD70, GITRL, LIGHT and ICAM-1; IFN-α, MEM40, B7-1(CD80)/B7-2(CD86), OX40L, 4-1BBL, CD70, GITRL, LIGHT and CD58; IFN-α, MEM40, B7-1(CD80)/B7-2(CD86), OX40L, 4-1BBL, CD70, GITRL, LIGHT and SLAMF6; IFN-α, MEM40, B7-1(CD80)/B7-2(CD86), OX40L, 4-1BBL, CD70, GITRL, LIGHT, TIM-4, and ICAM-1; IFN-α, MEM40, B7-1(CD80)/B7-2(CD86), OX40L, 4-1BBL, CD70, GITRL, LIGHT, TIM-4, and CD58; IFN-α, MEM40, B7-1(CD80)/B7-2(CD86), OX40L, 4-1BBL, CD70, GITRL, LIGHT, TIM-4, and SLAMF6; IFN-α, MEM40, B7-1(CD80)/B7-2(CD86), OX40L, 4-1BBL, CD70, GITRL, LIGHT, TIM-4, ICAM-1, and CD58; IFN-α, MEM40, B7-1(CD80)/B7-2(CD86), OX40L, 4-1BBL, CD70, GITRL, LIGHT, TIM-4, ICAM-1, and SLAMF6; IFN-α, MEM40, B7-1(CD80)/B7-2(CD86), OX40L, 4-1BBL, CD70, GITRL, LIGHT, TIM-4, ICAM-1, CD58, and SLAMF6; IFN-α, and B7-1(CD80)B7-2(CD86); IFN-α, B7-1(CD80)/B7-2(CD86), and OX40L; IFN-α, B7-1(CD80)/B7-2(CD86), and 4-1BBL; IFN-α, B7-1(CD80)/B7-2(CD86), and CD70; IFN-α, B7-1(CD80)/B7-2(CD86), and GITRL; IFN-α, B7-1(CD80)/B7-2(CD86), and LIGHT; IFN-α, B7-1(CD80)/B7-2(CD86), and TIM-4; IFN-α, B7-1(CD80)/B7-2(CD86), and ICAM-1; IFN-α, B7-1(CD80)/B7-2(CD86), and CD58; IFN-α, B7-1(CD80)/B7-2(CD86), and SLAMF6; IFN-α, B7-1(CD80)/B7-2(CD86), and OX40L; IFN-α, B7-1(CD80)/B7-2(CD86), and 4-1BBL; IFN-α, B7-1(CD80)/B7-2(CD86), and CD70; IFN-α, B7-1(CD80)/B7-2(CD86), and GITRL; IFN-α, B7-1(CD80)/B7-2(CD86), and LIGHT; IFN-α, B7-1(CD80)/B7-2(CD86), and TIM-4; IFN-α, B7-1(CD80)/B7-2(CD86), and ICAM-1; IFN-α, B7-1(CD80)/B7-2(CD86), and CD58; IFN-α, B7-1(CD80)/B7-2(CD86), and SLAMF6; IFN-α, B7-1(CD80)/B7-2(CD86), OX40L, and 4-1BBL; IFN-α, B7-1(CD80)/B7-2(CD86), OX40L, and CD70; IFN-α, B7-1(CD80)/B7-2(CD86), OX40L, and GITRL; IFN-α, B7-1(CD80)/B7-2(CD86), OX40L, and LIGHT; IFN-α, B7-1(CD80)/B7-2(CD86), OX40L, and TIM-4, IFN-α, B7-1(CD80)/B7-2(CD86), OX40L, and ICAM-1; IFN-α, B7-1(CD80)/B7-2(CD86), OX40L, and CD58; IFN-α, B7-1(CD80)/B7-2(CD86), OX40L, and SLAMF6; IFN-α, B7-1(CD80)/B7-2(CD86), OX40L, 4-1BBL, and CD70; IFN-α, B7-1(CD80)/B7-2(CD86), OX40L, 4-1BBL, and GITRL; IFN-α, B7-1(CD80)/B7-2(CD86), OX40L, 4-1BBL, and LIGHT; IFN-α, B7-1(CD80)/B7-2(CD86), OX40L, 4-1BBL, and TIM-4; IFN-α, B7-1(CD80)/B7-2(CD86), OX40L, 4-1BBL, and ICAM-1; IFN-α, B7-1(CD80)/B7-2(CD86), OX40L, 4-1BBL, and CD58; IFN-α, B7-1(CD80)/B7-2(CD86), OX40L, 4-1BBL, and SLAMF6; IFN-α, B7-1(CD80)/B7-2(CD86), OX40L, 4-1BBL, CD70, and GITRL; IFN-α, B7-1(CD80)/B7-2(CD86), OX40L, 4-1BBL, CD70, and LIGHT; IFN-α, B7-1(CD80)/B7-2(CD86), OX40L, 4-1BBL, CD70, and TIM-4; IFN-α, B7-1(CD80)/B7-2(CD86), OX40L, 4-1BBL, CD70, and ICAM-1; IFN-α, B7-1(CD80)/B7-2(CD86), OX40L, 4-1BBL, CD70, and CD58; IFN-α, B7-1(CD80)/B7-2(CD86), OX40L, 4-1BBL, CD70, and SLAMF6; IFN-α, B7-1(CD80)/B7-2(CD86), OX40L, 4-1BBL, CD70, GITRL, and LIGHT; IFN-α, B7-1(CD80)/B7-2(CD86), OX40L, 4-1BBL, CD70, GITRL, and TIM-4; IFN-α, B7-1(CD80)/B7-2(CD86), OX40L, 4-1BBL, CD70, GITRL, and ICAM-1; IFN-α, B7-1(CD80)/B7-2(CD86), OX40L, 4-1BBL, CD70, GITRL, and CD58; IFN-α, B7-1(CD80)/B7-2(CD86), OX40L, 4-1BBL, CD70, GITRL, and SLAMF6; IFN-α, B7-1(CD80)/B7-2(CD86), OX40L, 4-1BBL, CD70, GITRL, LIGHT, and TIM-4; IFN-α, B7-1(CD80)/B7-2(CD86), OX40L, 4-1BBL, CD70, GITRL, LIGHT and ICAM-1; IFN-α, B7-1(CD80)/B7-2(CD86), OX40L, 4-1 BBL, CD70, GITRL, LIGHT and CD58; IFN-α, B7-1(CD80)/B7-2(CD86), OX40L, 4-1BBL, CD70, GITRL, LIGHT and SLAMF6; IFN-α, B7-1(CD80)/B7-2(CD86), OX40L, 4-1BBL, CD70, GITRL, LIGHT, TIM-4, and ICAM-1; IFN-α, B7-1(CD80)/B7-2(CD86), OX40L, 4-1BBL, CD70, GITRL, LIGHT, TIM-4, and CD58; IFN-α, B7-1(CD80)/B7-2(CD86), OX40L, 4-1BBL, CD70, GITRL, LIGHT, TIM-4, and SLAMF6; IFN-α, B7-1(CD80)/B7-2(CD86), OX40L, 4-1BBL, CD70, GITRL, LIGHT, TIM-4, ICAM-1, and CD58; IFN-α, B7-1(CD80)/B7-2(CD86), OX40L, 4-1BBL, CD70, GITRL, LIGHT, TIM-4, ICAM-1, and SLAMF6; IFN-α, B7-1(CD80)/B7-2(CD86), OX40L, 4-1BBL, CD70, GITRL, LIGHT, TIM-4, ICAM-1, CD58, and SLAMF6; IFN-α, and OX40L; IFN-α, OX40L, and 4-1BBL; IFN-α, OX40L, and CD70; IFN-α, OX40L, and GITRL; IFN-α, OX40L, and LIGHT; IFN-α, OX40L, and TIM-4; IFN-α, OX40L, and ICAM-1, IFN-α, OX40L, and CD58; IFN-α, OX40L, and SLAMF6; IFN-α, OX40L, and 4-1BBL; IFN-α, OX40L, and CD70; IFN-α, OX40L, and GITRL; IFN-α, OX40L, and LIGHT; IFN-α, OX40L, and TIM-4; IFN-α, OX40L, and ICAM-1; IFN-α, OX40L, and CD58; IFN-α, OX40L, and SLAMF6; IFN-α, OX40L, 4-1BBL, and CD70; IFN-α, OX40L, 4-1BBL, and GITRL; IFN-α, OX40L, 4-1BBL, and LIGHT; IFN-α, OX40L, 4-1BBL, and TIM-4; IFN-α, OX40L, 4-1BBL, and ICAM-1; IFN-α, OX40L, 4-1BBL, and CD58; IFN-α, OX40L, 4-1BBL, and SLAMF6; IFN-α, OX40L, 4-1BBL, CD70, and GITRL; IFN-α, OX40L, 4-1BBL, CD70, and LIGHT; IFN-α, OX40L, 4-1BBL, CD70, and TIM-4; IFN-α, OX40L, 4-1BBL, CD70, and ICAM-1; IFN-α, OX40L, 4-1BBL, CD70, and CD58; IFN-α, OX40L, 4-1BBL, CD70, and SLAMF6; IFN-α, OX40L, 4-1BBL, CD70, GITRL, and LIGHT; IFN-α, OX40L, 4-1BBL, CD70, GITRL, and TIM-4; IFN-α, OX40L, 4-1BBL, CD70, GITRL, and ICAM-1; IFN-α, OX40L, 4-1BBL, CD70, GITRL, and CD58; IFN-α, OX40L, 4-1BBL, CD70, GITRL, and SLAMF6; IFN-α, OX40L, 4-1BBL, CD70, GITRL, LIGHT, and TIM-4; IFN-α, OX40L, 4-1BBL, CD70, GITRL, LIGHT and ICAM-1; IFN-α, OX40L, 4-1BBL, CD70, GITRL, LIGHT and CD58; IFN-α, OX40L, 4-1BBL, CD70, GITRL, LIGHT and SLAMF6; IFN-α, OX40L, 4-1BBL, CD70, GITRL, LIGHT, TIM-4, and ICAM-1; IFN-α, OX40L, 4-1BBL, CD70, GITRL, LIGHT, TIM-4, and CD58; IFN-α, OX40L, 4-1BBL, CD70, GITRL, LIGHT, TIM-4, and SLAMF6; IFN-α, OX40L, 4-1BBL, CD70, GITRL, LIGHT, TIM-4, ICAM-1, and CD58; IFN-α, OX40L, 4-1BBL, CD70, GITRL, LIGHT, TIM-4, ICAM-1, and SLAMF6; IFN-α, OX40L, 4-1BBL, CD70, GITRL, LIGHT, TIM-4, ICAM-1, CD58, and SLAMF6; IFN-α, and 4-1BBL; IFN-α, 4-1BBL, and CD70, IFN-α, 4-1BBL, and GITRL; IFN-α, 4-1BBL, and LIGHT; IFN-α, 4-1BBL, and TIM-4; IFN-α, 4-1BBL, and ICAM-1; IFN-α, 4-1BBL, and CD58; IFN-α, 4-1BBL, and SLAMF6; IFN-α, 4-1BBL, and CD70; IFN-α, 4-1BBL, and GITRL; IFN-α, 4-1BBL, and LIGHT; IFN-α, 4-1BBL, and TIM-4; IFN-α, 4-1BBL, and ICAM-1; IFN-α, 4-1BBL, and CD58; IFN-α, 4-1BBL, and SLAMF6; IFN-α, 4-1BBL, CD70, and GITRL; IFN-α, 4-1 BBL, CD70, and LIGHT; IFN-α, 4-1BBL, CD70, and TIM-4 IFN-α, 4-1BBL, CD70, and ICAM-1; IFN-α, 4-1BBL, CD70, and CD58; IFN-α, 4-1BBL, CD70, and SLAMF6; IFN-α, 4-1BBL, CD70, GITRL, and LIGHT; IFN-α, 4-1BBL, CD70, GITRL, and TIM-4; IFN-α, 4-1BBL, CD70, GITRL, and ICAM-1; IFN-α, 4-1BBL, CD70, GITRL, and CD58; IFN-α, 4-1BBL, CD70, GITRL, and SLAMF6; IFN-α, 4-1BBL, CD70, GITRL, LIGHT, and TIM-4; IFN-α, 4-1BBL, CD70, GITRL, LIGHT and ICAM-1, IFN-α, 4-1BBL, CD70, GITRL, LIGHT and CD58; IFN-α, 4-1BBL, CD70, GITRL, LIGHT and SLAMF6; IFN-α, 4-1BBL, CD70, GITRL, LIGHT, TIM-4, and ICAM-1; IFN-α, 4-1BBL, CD70, GITRL, LIGHT, TIM-4, and CD58; IFN-α, 4-1BBL, CD70, GITRL, LIGHT, TIM-4, and SLAMF6; IFN-α, 4-1BBL, CD70, GITRL, LIGHT, TIM-4, ICAM-1, and CD58; IFN-α, 4-1BBL, CD70, GITRL, LIGHT, TIM-4, ICAM-1, and SLAMF6; IFN-α, 4-1 BBL, CD70, GITRL, LIGHT, TIM-4, ICAM-1, CD58, and SLAMF6; IFN-α and CD70; IFN-α, CD70, and GITRL; IFN-α, CD70, and LIGHT; IFN-α, CD70, and TIM-4; IFN-α, CD70, and ICAM-1; IFN-α, CD70, and CD58; IFN-α, CD70, and SLAMF6; IFN-α, CD70, and GITRL; IFN-α, CD70, and LIGHT; IFN-α, CD70, and TIM-4; IFN-α, CD70, and ICAM-1; IFN-α, CD70, and CD58; IFN-α, CD70, and SLAMF6; IFN-α, CD70, GITRL, and LIGHT; IFN-α, CD70, GITRL, and TIM-4; IFN-α, CD70, GITRL, and ICAM-1; IFN-α, CD70, GITRL, and CD58; IFN-α, CD70, GITRL, and SLAMF6; IFN-α, CD70, GITRL, LIGHT, and TIM-4; IFN-α, CD70, GITRL, LIGHT and ICAM-1; IFN-α, CD70, GITRL, LIGHT and CD58; IFN-α, CD70, GITRL, LIGHT and SLAMF6; IFN-α, CD70, GITRL, LIGHT, TIM-4, and ICAM-1; IFN-α, CD70, GITRL, LIGHT, TIM-4, and CD58; IFN-α, CD70, GITRL, LIGHT, TIM-4, and SLAMF6; IFN-α, CD70, GITRL, LIGHT, TIM-4, ICAM-1, and CD58; IFN-α, CD70, GITRL, LIGHT, TIM-4, ICAM-I, and SLAMF6; IFN-α, CD70, GITRL, LIGHT, TIM-4, ICAM-1, CD58, and SLAMF6; IFN-α and GITRL; IFN-α, GITRL, and LIGHT; IFN-α, GITRL, and TIM-4; IFN-α, GITRL, and ICAM-1; IFN-α, GITRL, and CD58; IFN-α, GITRL, and SLAMF6; IFN-α, GITRL, and LIGHT; IFN-α, GITRL, and TIM-4; IFN-α, GITRL, and ICAM-1; IFN-α, GITRL, and CD58; IFN-α, GITRL, and SLAMF6; IFN-α, GITRL, LIGHT, and TIM-4; IFN-α, GITRL, LIGHT and ICAM-1; IFN-α, GITRL, LIGHT and CD58; IFN-α, GITRL, LIGHT and SLAMF6; IFN-α, GITRL, LIGHT, TIM-4, and ICAM-1; IFN-α, GITRL, LIGHT, TIM-4, and CD58; IFN-α, GITRL, LIGHT, TIM-4, and SLAMF6; IFN-α, GITRL, LIGHT, TIM-4, ICAM-1, and CD58; IFN-α, GITRL, LIGHT, TIM-4, ICAM-1, and SLAMF6; IFN-α, GITRL, LIGHT, TIM-4, ICAM-1, CD58, and SLAMF6; IFN-α, and LIGHT; IFN-α, LIGHT, and TIM-4; IFN-α, LIGHT, and ICAM-1; IFN-α, LIGHT, and CD58; IFN-α, LIGHT, and SLAMF6; IFN-α, LIGHT, and TIM-4; IFN-α, LIGHT and ICAM-1; IFN-α, LIGHT and CD58; IFN-α, LIGHT and SLAMF6; IFN-α, LIGHT, TIM-4, and ICAM-1; IFN-α, LIGHT, TIM-4, and CD58; IFN-α, LIGHT, TIM-4, and SLAMF6; IFN-α, LIGHT, TIM-4, ICAM-I, and CD58; IFN-α, LIGHT, TIM-4, ICAM-1, and SLAMF6; IFN-α, LIGHT, TIM-4, ICAM-1, CD58, and SLAMF6; IFN-α, and TIM-4; IFN-α, TIM-4, and ICAM-1; IFN-α, TIM-4, and CD58; IFN-α, TIM-4, and SLAMF6; IFN-α, TIM-4, and ICAM-1; IFN-α, TIM-4, and CD58; IFN-α, TIM-4, and SLAMF6; IFN-α, TIM-4, ICAM-1, and CD58; IFN-α, TIM-4, ICAM-1, and SLAMF6; IFN-α, TIM-4, ICAM-1, CD58, and SLAMF6; IFN-α and ICAM-I; IFN-α, ICAM-1, and CD58; IFN-α, ICAM-1, and SLAMF6; IFN-α, ICAM-1, and CD58; IFN-α, ICAM-1, and SLAMF6; IFN-α, ICAM-1, CD58, and SLAMF6; IFN-α and CD58; IFN-α, CD58, and SLAMF6; IFN-α and SLAMF6; IFN-α, IFN-β, and CD40-L; IFN-α, IFN-β, and MEM40; IFN-α, IFN-β, and B7-1(CD80)/B7-2(CD86); IFN-α, IFN-β, and OX40L; IFN-α, IFN-β, and 4-1BBL; IFN-α, IFN-β, and CD70; IFN-α, IFN-β, and GITRL; IFN-α, IFN-β, and LIGHT; IFN-α, IFN-β, and TIM-4; IFN-α, IFN-0, and ICAM-1; IFN-α, IFN-β, and CD58; IFN-α, IFN-β, and SLAMF6; IFN-α, IFN-β, IFN-κ, and CD40-L; IFN-α, IFN-β, IFN-κ, and MEM40; IFN-α, IFN-β, IFN-κ, and B7-1(CD80)/B7-2(CD86); IFN-α, IFN-β, IFN-κ, and OX40L; IFN-α, IFN-β, IFN-κ, and 4-1BBL; IFN-α, IFN-β, IFN-κ, and CD70; IFN-α, IFN-β, IFN-κ, and GITRL; IFN-α, IFN-β, IFN-κ, and LIGHT; IFN-α, IFN-β, IFN-κ, and TIM-4; IFN-α, IFN-β, IFN-κ, and ICAM-1; IFN-α, IFN-β, and CD58; IFN-α, IFN-β, IFN-κ, and SLAMF6; IFN-α, IFN-β, IFN-κ, IFN-δ, and CD40-L; IFN-α, IFN-β, IFN-κ, IFN-ζ, and MEM40; IFN-α, IFN-β, IFN-κ, IFN-δ, and B7-1(CD80)/B7-2(CD86); IFN-α, IFN-β, IFN-κ, IFN-δ, and OX40L; IFN-α, IFN-β, IFN-κ, IFN-δ, and 4-1BBL; IFN-α, IFN-β, IFN-κ, IFN-δ, and CD70; IFN-α, IFN-β, IFN-κ, IFN-δ, and GITRL; IFN-α, IFN-β, IFN-κ, IFN-δ, and LIGHT; IFN-α, IFN-β, IFN-κ, IFN-δ, and TIM-4; IFN-α, IFN-β, IFN-κ, IFN-δ, and ICAM-1; IFN-α, IFN-β, IFN-δ, and CD58; IFN-α, IFN-β, IFN-κ, IFN-δ, and SLAMF6; IFN-α, IFN-β, IFN-κ, IFN-δ, IFN-ε, and CD40-L; IFN-α, IFN-β, IFN-κ, IFN-δ, IFN-ε, and MEM40; IFN-α, IFN-β, IFN-κ, IFN-δ, IFN-ε, and B7-1(CD80)/B7-2(CD86); IFN-α, IFN-β, IFN-κ, IFN-δ, IFN-ε, and OX40L; IFN-α, IFN-β, IFN-κ, IFN-δ, IFN-ε, and 4-1BBL; IFN-α, IFN-β, IFN-κ, IFN-δ, IFN-ε, and CD70; IFN-α, IFN-β, IFN-κ, IFN-δ, IFN-ε, and GITRL; IFN-α, IFN-β, IFN-κ, IFN-δ, IFN-ε, and LIGHT; IFN-α, IFN-β, IFN-κ, IFN-δ, IFN-ε, and TIM-4; IFN-α, IFN-β, IFN-κ, IFN-δ, IFN-ε, and ICAM-1; IFN-α, IFN-β, IFN-δ, IFN-ε, and CD58; IFN-α, IFN-β, IFN-κ, IFN-δ, IFN-ε, and SLAMF6; IFN-α, IFN-β, IFN-κ, IFN-δ, IFN-ε, IFN-τ, and CD40-L; IFN-α, IFN-β, IFN-κ, IFN-δ, IFN-ε, IFN-τ, and MEM40; IFN-α, IFN-β, IFN-κ, IFN-δ, IFN-ε, IFN-ε, and B7-1(CD80)/B7-2(CD86); IFN-α, IFN-β, IFN-κ, IFN-δ, IFN-ε, IFN-τ, and OX40L; IFN-α, IFN-β, IFN-κ, IFN-δ, IFN-ε, IFN-τ, and 4-1BBL; IFN-α, IFN-β, IFN-κ, IFN-δ, IFN-ε, IFN-ε, and CD70; IFN-α, IFN-β, IFN-κ, IFN-δ, IFN-ε, IFN-τ, and GITRL; IFN-α, IFN-β, IFN-κ, IFN-δ, IFN-ε, IFN-τ, and LIGHT, IFN-α, IFN-β, IFN-κ, IFN-δ, IFN-ε, IFN-τ, and TIM-4, IFN-α, IFN-β, IFN-κ, IFN-δ, IFN-ε, IFN-τ, and ICAM-1; IFN-α, IFN-β, IFN-δ, IFN-ε, IFN-τ, and CD58; IFN-α, IFN-β, IFN-κ, IFN-δ, IFN-ε, IFN-τ, and SLAMF6; IFN-α, IFN-β, IFN-κ, IFN-δ, IFN-ε, IFN-τ, IFN-ω, and CD40-L; IFN-α, IFN-β, IFN-κ, IFN-δ, IFN-ε, IFN-τ, IFN-ω, and MEM40; IFN-α, IFN-β, IFN-κ, IFN-δ, IFN-ε, IFN-τ, IFN-ω, and B7-1(CD80)/B7-2(CD86); IFN-α, IFN-β, IFN-κ, IFN-δ, IFN-ε, IFN-τ, IFN-ω, and OX40L; IFN-α, IFN-β, IFN-κ, IFN-δ, IFN-ε, IFN-ε, IFN-ω, and 4-1BBL; IFN-α, IFN-β, IFN-κ, IFN-δ, IFN-ε, IFN-τ, IFN-ω, and CD70; IFN-α, IFN-β, IFN-κ, IFN-δ, IFN-ε, IFN-τ, IFN-ω, and GITRL; IFN-α, IFN-β, IFN-κ, IFN-δ, IFN-ε, IFN-ε, IFN-ω, and LIGHT; IFN-α, IFN-β, IFN-κ, IFN-δ, IFN-ε, IFN-τ, IFN-ω, and TIM-4; IFN-α, IFN-β, IFN-κ, IFN-δ, IFN-ε, IFN-τ, IFN-ω, and ICAM-1; IFN-α, IFN-β, IFN-δ, IFN-ε, IFN-τ, IFN-ω, and CD58; IFN-α, IFN-β, IFN-κ, IFN-δ, IFN-ε, IFN-τ, IFN-ω, and SLAMF6; IFN-α, IFN-β, IFN-κ, IFN-δ, IFN-ε, IFN-τ, IFN-ω, IFN-ζ, and CD40-L; IFN-α, IFN-β, IFN-κ, IFN-δ, IFN-ε, IFN-τ, IFN-ω, IFN-ζ, and MEM40; IFN-α, IFN-β, IFN-κ, IFN-δ, IFN-ε, IFN-τ, IFN-ω, IFN-ζ, and B7-1(CD80)/B7-2(CD86); IFN-α, IFN-β, IFN-κ, IFN-δ, IFN-ε, IFN-τ, IFN-ω, IFN-ζ, and OX40L; IFN-α, IFN-β, IFN-κ, IFN-δ, IFN-ε, IFN-τ, IFN-ω, IFN-ζ, and 4-1BBL; IFN-α, IFN-β, IFN-κ, IFN-δ, IFN-ε, IFN-τ, IFN-ω, IFN-ζ, and CD70; IFN-α, IFN-β, IFN-κ, IFN-δ, IFN-ε, IFN-τ, IFN-ω, IFN-ζ, and GITRL; IFN-α, IFN-β, IFN-κ, IFN-δ, IFN-ε, IFN-τ, IFN-ω, IFN-ζ, and LIGHT; IFN-α, IFN-β, IFN-κ, IFN-δ, IFN-ε, IFN-τ, IFN-ω, IFN-ζ, and TIM-4; IFN-α, IFN-β, IFN-κ, IFN-δ, IFN-ε, IFN-τ, IFN-ω, IFN-ζ, and ICAM-1; IFN-α, IFN-β, IFN-δ, IFN-ε, IFN-ε, IFN-ω, IFN-ζ, and CD58; IFN-α, IFN-β, IFN-κ, IFN-δ, IFN-ε, IFN-τ, IFN-ω, IFN-ζ, and SLAMF6; IFN-β and CD40-L; IFN-β and MEM40; IFN-β and B7-1(CD80)B7-2(CD86); IFN-β and OX40L; IFN-β and 4-1BBL; IFN-β and CD70; IFN-β and GITRL; IFN-β and LIGHT; IFN-β and TIM-4; IFN-β and ICAM-1; IFN-β and CD58; IFN-β and SLAMF6; IFN-κ and CD40-L, IFN-κ and MEM40; IFN-κ and B7-1(CD80)/B7-2(CD86); IFN-κ and OX40L; IFN-κ and 4-1BBL; IFN-κ and CD70; IFN-κ and GITRL; IFN-κ and LIGHT; IFN-κ and TIM-4; IFN-κ and ICAM-1; IFN-κ and CD58; IFN-α and SLAMF6; IFN-δ and CD40-L; IFN-δ and MEM40; IFN-δ and B7-1(CD80)/B7-2(CD86); IFN-δ and OX40L; IFN-δ and 4-1BBL; IFN-δ and CD70; IFN-δ and GITRL; IFN-δ and LIGHT; IFN-δ and TIM-4, IFN-δ and ICAM-1; IFN-δ and CD58; IFN-δ and SLAMF6; IFN-ε and CD40-L; IFN-ε and MEM40; IFN-ε and B7-1(CD80)/B7-2(CD86); IFN-β and OX40L; IFN-ε and 4-1BBL; IFN-β and CD70; IFN-β and GITRL; IFN-ε and LIGHT; IFN-β and TIM-4; IFN-ε and ICAM-1; IFN-β and CD58; IFN-ε and SLAMF6; IFN-τ and CD40-L; IFN-τ and MEM40; IFN-τ and B7-1(CD80)/B7-2(CD86); IFN-τ and OX40L; IFN-τ and 4-1BBL; IFN-τ and CD70, IFN-τ and GITRL; IFN-τ and LIGHT; IFN-τ and TIM-4; IFN-τ and ICAM-1; IFN-τ and CD58; IFN-τ and SLAMF6; IFN-ω and CD40-L; IFN-ω and MEM40; IFN-ω and B7-1(CD80)/B7-2(CD86); IFN-ω and OX40L; IFN-ω and 4-1BBL; IFN-ω and CD70; IFN-ω and GITRL; IFN-ω and LIGHT; IFN-ω and TIM-4; IFN-ω and ICAM-1; IFN-ω and CD58; IFN-ω and SLAMF6; IFN-ζ and CD40-L; IFN-ζ and MEM40; IFN-ζ and B7-1(CD80)/B7-2(CD86); IFN-ζ and OX40L; IFN-ζ and 4-1BBL, IFN-ζ and CD70; IFN-ζ and GITRL; IFN-ζ and LIGHT; IFN-ζ and TIM-4; IFN-ζ and ICAM-1; IFN-ζ and CD58; IFN-ζ and SLAMF6; IFN-β, CD40-L, and MEM40; IFN-β, CD40-L, and B7-1(CD80)/B7-2(CD86); IFN-β, CD40-L, and OX40L; IFN-β, CD40-L, and 4-1BBL; IFN-β, CD40-L, and CD70; IFN-β, CD40-L, and GITRL; IFN-β, CD40-L, and LIGHT; IFN-β, CD40-L, and TIM-4; IFN-β, CD40-L, and ICAM-1; IFN-β, CD40-L, and CD58; IFN-β, CD40-L, and SLAMF6; IFN-β, CD40-L, MEM40, and B7-1(CD80)/B7-2(CD86); IFN-β, CD40-L, MEM40, and OX40L; IFN-β, CD40-L, MEM40, and 4-1BBL; IFN-β, CD40-L, MEM40, and CD70; IFN-β, CD40-L, MEM40, and GITRL; IFN-β, CD40-L, MEM40, and LIGHT; IFN-β, CD40-L, MEM40, and TIM-4; IFN-β, CD40-L, MEM40, and ICAM-1; IFN-β, CD40-L, MEM40, and CD58; IFN-β, CD40-L, MEM40, and SLAMF6; IFN-β, CD40-L, MEM40, B7-1(CD80)/B7-2(CD86), and OX40L; IFN-β, CD40-L, MEM40, B7-1(CD80)/B7-2(CD86), and 4-1BBL; IFN-β, CD40-L, MEM40, B7-1(CD80)/B7-2(CD86), and CD70; IFN-β, CD40-L, MEM40, B7-1(CD80)/B7-2(CD86), and GITRL; IFN-β, CD40-L, MEM40, B7-1(CD80)/B7-2(CD86), and LIGHT; IFN-β, CD40-L, MEM40, B7-1(CD80)/B7-2(CD86), and TIM-4; IFN-β, CD40-L, MEM40, B7-1(CD80)/B7-2(CD86), and ICAM-1; IFN-β, CD40-L, MEM40, B7-1(CD80)/B7-2(CD86), and CD58; IFN-β, CD40-L, MEM40, B7-1(CD80)/B7-2(CD86), and SLAMF6; IFN-β, CD40-L, MEM40, B7-1(CD80)/B7-2(CD86), OX40L, and 4-1BBL; IFN-β, CD40-L, MEM40, B7-1(CD80)/B7-2(CD86), OX40L, and CD70; IFN-β, CD40-L, MEM40, B7-1(CD80)/B7-2(CD86), OX40L, and GITRL; IFN-β, CD40-L, MEM40, B7-1(CD80)/B7-2(CD86), OX40L, and LIGHT; IFN-β, CD40-L, MEM40, B7-1(CD80)/B7-2(CD86), OX40L, and TIM-4; IFN-β, CD40-L, MEM40, B7-1(CD80)/B7-2(CD86), OX40L, and ICAM-1; IFN-β, CD40-L, MEM40, B7-1(CD80)/B7-2(CD86), OX40L, and CD58; IFN-β, CD40-L, MEM40, B7-1(CD80)/B7-2(CD86), OX40L, and SLAMF6; IFN-β, CD40-L, MEM40, B7-1(CD80)/B7-2(CD86), OX40L, 4-1BBL, and CD70; IFN-β, CD40-L, MEM40, B7-1(CD80)/B7-2(CD86), OX40L, 4-1BBL, and GITRL; IFN-β, CD40-L, MEM40, B7-1(CD80)/B7-2(CD86), OX40L, 4-1BBL, and LIGHT; IFN-β, CD40-L, MEM40, B7-1(CD80)/B7-2(CD86), OX40L, 4-1BBL, and TIM-4; IFN-β, CD40-L, MEM40, B7-1(CD80)/B7-2(CD86), OX40L, 4-1BBL, and ICAM-1; IFN-β, CD40-L, MEM40, B7-1(CD80)/B7-2(CD86), OX40L, 4-1BBL, and CD58; IFN-β, CD40-L, MEM40, B7-1(CD80)/B7-2(CD86), OX40L, 4-1BBL, and SLAMF6; IFN-β, CD40-L, MEM40, B7-1(CD80)/B7-2(CD86), OX40L, 4-1BBL, CD70, and GITRL; IFN-β, CD40-L, MEM40, B7-1(CD80)/B7-2(CD86), OX40L, 4-1BBL, CD70, and LIGHT; IFN-β, CD40-L, MEM40, B7-1(CD80)/B7-2(CD86), OX40L, 4-1BBL, CD70, and TIM-4; IFN-β, CD40-L, MEM40, B7-1(CD80)/B7-2(CD86), OX40L, 4-1BBL, CD70, and ICAM-1; IFN-β, CD40-L, MEM40, B7-1(CD80)/B7-2(CD86), OX40L, 4-1BBL, CD70, and CD58; IFN-β, CD40-L, MEM40, B7-1(CD80)/B7-2(CD86), OX40L, 4-1BBL, CD70, and SLAMF6; IFN-β, CD40-L, MEM40, B7-1(CD80)/B7-2(CD86), OX40L, 4-1BBL, CD70, GITRL, and LIGHT; IFN-β, CD40-L, MEM40, B7-1(CD80)/B7-2(CD86), OX40L, 4-1BBL, CD70, GITRL, and TIM-4; IFN-β, CD40-L, MEM40, B7-1(CD80)/B7-2(CD86), OX40L, 4-1BBL, CD70, GITRL, and ICAM-1; IFN-β, CD40-L, MEM40, B7-1(CD80)/B7-2(CD86), OX40L, 4-1BBL, CD70, GITRL, and CD58; IFN-β, CD40-L, MEM40, B7-1(CD80)/B7-2(CD86), OX40L, 4-1BBL, CD70, GITRL, and SLAMF6; IFN-β, CD40-L, MEM40, B7-1(CD80)/B7-2(CD86), OX40L, 4-1BBL, CD70, GITRL, LIGHT, and TIM-4; IFN-β, CD40-L, MEM40, B7-1(CD80)/B7-2(CD86), OX40L, 4-1BBL, CD70, GITRL, LIGHT and ICAM-1; IFN-β, CD40-L, MEM40, B7-1(CD80)/B7-2(CD86), OX40L, 4-1BBL, CD70, GITRL, LIGHT and CD58; IFN-β, CD40-L, MEM40, B7-1(CD80)/B7-2(CD86), OX40L, 4-1BBL, CD70, GITRL, LIGHT and SLAMF6; IFN-β, CD40-L, MEM40, B7-1(CD80)/B7-2(CD86), OX40L, 4-1BBL, CD70, GITRL, LIGHT, TIM-4, and ICAM-1; IFN-β, CD40-L, MEM40, B7-1(CD80)/B7-2(CD86), OX40L, 4-1BBL, CD70, GITRL, LIGHT, TIM-4, and CD58; IFN-β, CD40-L, MEM40, B7-1(CD80)/B7-2(CD86), OX40L, 4-1BBL, CD70, GITRL, LIGHT, TIM-4, and SLAMF6; IFN-β, CD40-L, MEM40, B7-1(CD80)/B7-2(CD86), OX40L, 4-1BBL, CD70, GITRL, LIGHT, TIM-4, ICAM-1, and CD58; IFN-β, CD40-L, MEM40, B7-1(CD80)/B7-2(CD86), OX40L, 4-1BBL, CD70, GITRL, LIGHT, TIM-4, ICAM-1, and SLAMF6; IFN-β, CD40-L, MEM40, B7-1(CD80)/B7-2(CD86), OX40L, 4-1BBL, CD70, GITRL, LIGHT, TIM-4, ICAM-1, CD58, and SLAMF6; IFN-β, MEM40; IFN-β, MEM40, and B7-1(CD80)/B7-2(CD86); IFN-β, MEM40, and OX40L; IFN-β, MEM40, and 4-1BBL; IFN-β, MEM40, and CD70; IFN-β, MEM40, and GITRL; IFN-β, MEM40, and LIGHT; IFN-β, MEM40, and TIM-4; IFN-β, MEM40, and ICAM-1; IFN-β, MEM40, and CD58; IFN-β, MEM40, and SLAMF6; IFN-β, MEM40, and B7-1(CD80)/B7-2(CD86); IFN-β, MEM40, and OX40L; IFN-β, MEM40, and 4-1BBL; IFN-β, MEM40, and CD70; IFN-β, MEM40, and GITRL; IFN-β, MEM40, and LIGHT; IFN-β, MEM40, and TIM-4; IFN-β, MEM40, and ICAM-1; IFN-β, MEM40, and CD58; IFN-β, MEM40, and SLAMF6; IFN-β, MEM40, B7-1(CD80)/B7-2(CD86), and OX40L; IFN-β, MEM40, B7-1(CD80)/B7-2(CD86), and 4-1BBL; IFN-β, MEM40, B7-1(CD80)/B7-2(CD86), and CD70; IFN-β, MEM40, B7-1(CD80)/B7-2(CD86), and GITRL; IFN-β, MEM40, B7-1(CD80)/B7-2(CD86), and LIGHT; IFN-β, MEM40, B7-1(CD80)/B7-2(CD86), and TIM-4; IFN-β, MEM40, B7-1(CD80)/B7-2(CD86), and ICAM-1; IFN-β, MEM40, B7-1(CD80)B7-2(CD86), and CD58; IFN-β, MEM40, B7-1(CD80)/B7-2(CD86), and SLAMF6; IFN-β, MEM40, B7-1(CD80)/B7-2(CD86), OX40L, and 4-1BBL; IFN-β, MEM40, B7-1(CD80)/B7-2(CD86), OX40L, and CD70; IFN-β, MEM40, B7-1(CD80)/B7-2(CD86), OX40L, and GITRL; IFN-β, MEM40, B7-1(CD80)/B7-2(CD86), OX40L, and LIGHT; IFN-β, MEM40, B7-1(CD80)/B7-2(CD86), OX40L, and TIM-4; IFN-β, MEM40, B7-1(CD80)/B7-2(CD86), OX40L, and ICAM-1; IFN-β, MEM40, B7-1(CD80)/B7-2(CD86), OX40L, and CD58; IFN-β, MEM40, B7-1(CD80)/B7-2(CD86), OX40L, and SLAMF6; IFN-β, MEM40, B7-1(CD80)/B7-2(CD86), OX40L, 4-1BBL, and CD70; IFN-β, MEM40, B7-1(CD80)/B7-2(CD86), OX40L, 4-1BBL, and GITRL; IFN-β, MEM40, B7-1(CD80)/B7-2(CD86), OX40L, 4-1BBL, and LIGHT; IFN-β, MEM40, B7-1(CD80)/B7-2(CD86), OX40L, 4-1BBL, and TIM-4; IFN-β, MEM40, B7-1(CD80)/B7-2(CD86), OX40L, 4-1BBL, and ICAM-1; IFN-β, MEM40, B7-1(CD80)/B7-2(CD86), OX40L, 4-1BBL, and CD58; IFN-β, MEM40, B7-1(CD80)/B7-2(CD86), OX40L, 4-1BBL, and SLAMF6; IFN-β, MEM40, B7-1(CD80)/B7-2(CD86), OX40L, 4-1BBL, CD70, and GITRL; IFN-β, MEM40, B7-1(CD80)/B7-2(CD86), OX40L, 4-1BBL, CD70, and LIGHT; IFN-β, MEM40, B7-1(CD80)/B7-2(CD86), OX40L, 4-1BBL, CD70, and TIM-4; IFN-β, MEM40, B7-1(CD80)/B7-2(CD86), OX40L, 4-1BBL, CD70, and ICAM-1; IFN-β, MEM40, B7-1(CD80)/B7-2(CD86), OX40L, 4-1BBL, CD70, and CD58; IFN-β, MEM40, B7-1(CD80)/B7-2(CD86), OX40L, 4-1BBL, CD70, and SLAMF6; IFN-β, MEM40, B7-1(CD80)/B7-2(CD86), OX40L, 4-1BBL, CD70, GITRL, and LIGHT, IFN-β, MEM40, B7-1(CD80)/B7-2(CD86), OX40L, 4-1BBL, CD70, GITRL, and TIM-4; IFN-β, MEM40, B7-1(CD80)/B7-2(CD86), OX40L, 4-1BBL, CD70, GITRL, and ICAM-1; IFN-β, MEM40, B7-1(CD80)/B7-2(CD86), OX40L, 4-1BBL, CD70, GITRL, and CD58; IFN-β, MEM40, B7-1(CD80)/B7-2(CD86), OX40L, 4-1BBL, CD70, GITRL, and SLAMF6; IFN-β, MEM40, B7-1(CD80)/B7-2(CD86), OX40L, 4-1BBL, CD70, GITRL, LIGHT, and TIM-4; IFN-β, MEM40, B7-1(CD80)/B7-2(CD86), OX40L, 4-1BBL, CD70, GITRL, LIGHT and ICAM-1; IFN-β, MEM40, B7-1(CD80)/B7-2(CD86), OX40L, 4-1BBL, CD70, GITRL, LIGHT and CD58; IFN-β, MEM40, B7-1(CD80)/B7-2(CD86), OX40L, 4-1BBL, CD70, GITRL, LIGHT and SLAMF6; IFN-β, MEM40, B7-1(CD80)/B7-2(CD86), OX40L, 4-1BBL, CD70, GITRL, LIGHT, TIM-4, and ICAM-1; IFN-β, MEM40, B7-1(CD80)/B7-2(CD86), OX40L, 4-1BBL, CD70, GITRL, LIGHT, TIM-4, and CD58; IFN-β, MEM40, B7-1(CD80)B7-2(CD86), OX40L, 4-1BBL, CD70, GITRL, LIGHT, TIM-4, and SLAMF6; IFN-β, MEM40, B7-1(CD80)/B7-2(CD86), OX40L, 4-1BBL, CD70, GITRL, LIGHT, TIM-4, ICAM-1, and CD58; IFN-β, MEM40, B7-1(CD80)/B7-2(CD86), OX40L, 4-1BBL, CD70, GITRL, LIGHT, TIM-4, ICAM-1, and SLAMF6; IFN-β, MEM40, B7-1(CD80)/B7-2(CD86), OX40L, 4-1BBL, CD70, GITRL, LIGHT, TIM-4, ICAM-1, CD58, and SLAMF6; IFN-β, and B7-1(CD80)/B7-2(CD86); IFN-β, B7-1(CD80)/B7-2(CD86), and OX40L; IFN-β, B7-1(CD80)/B7-2(CD86), and 4-1BBL; IFN-β, B7-1(CD80)/B7-2(CD86), and CD70; IFN-β, B7-1(CD80)/B7-2(CD86), and GITRL; IFN-β, B7-1(CD80)/B7-2(CD86), and LIGHT; IFN-β, B7-1(CD80)/B7-2(CD86), and TIM-4; IFN-β, B7-1(CD80)/B7-2(CD86), and ICAM-1; IFN-β, B7-1(CD80yB7-2(CD86), and CD58; IFN-β, B7-1(CD80)/B7-2(CD86), and SLAMF6; IFN-β, B7-1(CD80)/B7-2(CD86), and OX40L; IFN-β, B7-1(CD80)/B7-2(CD86), and 4-1BBL; IFN-β, B7-1(CD80)/B7-2(CD86), and CD70; IFN-β, B7-1(CD80)/B7-2(CD86), and GITRL; IFN-β, B7-1(CD80)/B7-2(CD86), and LIGHT; IFN-β, B7-1(CD80)/B7-2(CD86), and TIM-4; IFN-β, B7-1(CD80)/B7-2(CD86), and ICAM-1; IFN-β, B7-1(CD80)/B7-2(CD86), and CD58; IFN-β, B7-1(CD80)/B7-2(CD86), and SLAMF6; IFN-β, B7-1(CD80)/B7-2(CD86), OX40L, and 4-1BBL; IFN-β, B7-1(CD80)/B7-2(CD86), OX40L, and CD70; IFN-β, B7-1(CD80)/B7-2(CD86), OX40L, and GITRL; IFN-β, B7-1(CD80)/B7-2(CD86), OX40L, and LIGHT; IFN-β, B7-1(CD80)B7-2(CD86), OX40L, and TIM-4; IFN-β, B7-1(CD80)/B7-2(CD86), OX40L, and ICAM-1; IFN-β, B7-1(CD80)/B7-2(CD86), OX40L, and CD58; IFN-β, B7-1(CD80)B7-2(CD86), OX40L, and SLAMF6; IFN-β, B7-1(CD80)/B7-2(CD86), OX40L, 4-1BBL, and CD70; IFN-β, B7-1(CD80)/B7-2(CD86), OX40L, 4-1BBL, and GITRL; IFN-β, B7-1(CD80)/B7-2(CD86), OX40L, 4-1BBL, and LIGHT; IFN-β, B7-1(CD80)/B7-2(CD86), OX40L, 4-1BBL, and TIM-4; IFN-β, B7-1(CD80)/B7-2(CD86), OX40L, 4-1BBL, and ICAM-1; IFN-β, B7-1(CD80)/B7-2(CD86), OX40L, 4-1BBL, and CD58; IFN-β, B7-1(CD80)/B7-2(CD86), OX40L, 4-1BBL, and SLAMF6; IFN-β, B7-1(CD80)/B7-2(CD86), OX40L, 4-1BBL, CD70, and GITRL; IFN-β, B7-1(CD80)B7-2(CD86), OX40L, 4-1BBL, CD70, and LIGHT; IFN-β, B7-1(CD80)/B7-2(CD86), OX40L, 4-1BBL, CD70, and TIM-4; IFN-β, B7-1(CD80)/B7-2(CD86), OX40L, 4-1BBL, CD70, and ICAM-1; IFN-β, B7-1(CD80)/B7-2(CD86), OX40L, 4-1BBL, CD70, and CD58; IFN-β, B7-1(CD80)/B7-2(CD86), OX40L, 4-1BBL, CD70, and SLAMF6; IFN-β, B7-1(CD80)/B7-2(CD86), OX40L, 4-1BBL, CD70, GITRL, and LIGHT; IFN-β, B7-1(CD80)/B7-2(CD86), OX40L, 4-1BBL, CD70, GITRL, and TIM-4; IFN-β, B7-1(CD80)/B7-2(CD86), OX40L, 4-1BBL, CD70, GITRL, and ICAM-1; IFN-β, B7-1(CD80)/B7-2(CD86), OX40L, 4-1BBL, CD70, GITRL, and CD58; IFN-β, B7-1(CD80)/B7-2(CD86), OX40L, 4-1BBL, CD70, GITRL, and SLAMF6; IFN-β, B7-1(CD80)/B7-2(CD86), OX40L, 4-1BBL, CD70, GITRL, LIGHT, and TIM-4; IFN-β, B7-1(CD80)/B7-2(CD86), OX40L, 4-1BBL, CD70, GITRL, LIGHT and ICAM-1; IFN-β, B7-1(CD80)/B7-2(CD86), OX40L, 4-1BBL, CD70, GITRL, LIGHT and CD58; IFN-β, B7-1(CD80)B7-2(CD86), OX40L, 4-1BBL, CD70, GITRL, LIGHT and SLAMF6; IFN-β, B7-1(CD80)/B7-2(CD86), OX40L, 4-1BBL, CD70, GITRL, LIGHT, TIM-4, and ICAM-1; IFN-β, B7-1(CD80)/B7-2(CD86), OX40L, 4-1BBL, CD70, GITRL, LIGHT, TIM-4, and CD58; IFN-β, B7-1(CD80)/B7-2(CD86), OX40L, 4-1BBL, CD70, GITRL, LIGHT, TIM-4, and SLAMF6; IFN-β, B7-1(CD80)/B7-2(CD86), OX40L, 4-1BBL, CD70, GITRL, LIGHT, TIM-4, ICAM-1, and CD58; IFN-β, B7-1(CD80)/B7-2(CD86), OX40L, 4-1BBL, CD70, GITRL, LIGHT, TIM-4, ICAM-1, and SLAMF6; IFN-β, B7-1(CD80)/B7-2(CD86), OX40L, 4-1BBL, CD70, GITRL, LIGHT, TIM-4, ICAM-1, CD58, and SLAMF6; IFN-0, and OX40L; IFN-β, OX40L, and 4-1BBL; IFN-β, OX40L, and CD70; IFN-β, OX40L, and GITRL; IFN-β, OX40L, and LIGHT; IFN-β, OX40L, and TIM-4; IFN-β, OX40L, and ICAM-1, IFN-β, OX40L, and CD58; IFN-β, OX40L, and SLAMF6; IFN-β, OX40L, and 4-1BBL; IFN-β, OX40L, and CD70; IFN-β, OX40L, and GITRL; IFN-β, OX40L, and LIGHT; IFN-β, OX40L, and TIM-4; IFN-β, OX40L, and ICAM-1; IFN-β, OX40L, and CD58; IFN-β, OX40L, and SLAMF6; IFN-β, OX40L, 4-1BBL, and CD70; IFN-β, OX40L, 4-1BBL, and GITRL; IFN-β, OX40L, 4-1BBL, and LIGHT; IFN-β, OX40L, 4-1BBL, and TIM-4; IFN-β, OX40L, 4-1BBL, and ICAM-1; IFN-β, OX40L, 4-1BBL, and CD58; IFN-β, OX40L, 4-1BBL, and SLAMF6; IFN-β, OX40L, 4-1BBL, CD70, and GITRL; IFN-β, OX40L, 4-1BBL, CD70, and LIGHT; IFN-β, OX40L, 4-1BBL, CD70, and TIM-4, IFN-β, OX40L, 4-1BBL, CD70, and ICAM-1; IFN-β, OX40L, 4-1BBL, CD70, and CD58; IFN-β, OX40L, 4-1BBL, CD70, and SLAMF6; IFN-β, OX40L, 4-1BBL, CD70, GITRL, and LIGHT; IFN-β, OX40L, 4-1BBL, CD70, GITRL, and TIM-4; IFN-β, OX40L, 4-1BBL, CD70, GITRL, and ICAM-1; IFN-β, OX40L, 4-1BBL, CD70, GITRL, and CD58; IFN-β, OX40L, 4-1BBL, CD70, GITRL, and SLAMF6; IFN-β, OX40L, 4-1BBL, CD70, GITRL, LIGHT, and TIM-4; IFN-β, OX40L, 4-1BBL, CD70, GITRL, LIGHT and ICAM-1; IFN-β, OX40L, 4-1BBL, CD70, GITRL, LIGHT and CD58; IFN-β, OX40L, 4-1BBL, CD70, GITRL, LIGHT and SLAMF6; IFN-β, OX40L, 4-1BBL, CD70, GITRL, LIGHT, TIM-4, and ICAM-1; IFN-β, OX40L, 4-1BBL, CD70, GITRL, LIGHT, TIM-4, and CD58; IFN-β, OX40L, 4-1BBL, CD70, GITRL, LIGHT, TIM-4, and SLAMF6; IFN-β, OX40L, 4-1BBL, CD70, GITRL, LIGHT, TIM-4, ICAM-1, and CD58; IFN-β, OX40L, 4-1BBL, CD70, GITRL, LIGHT, TIM-4, ICAM-1, and SLAMF6; IFN-β, OX40L, 4-1BBL, CD70, GITRL, LIGHT, TIM-4, ICAM-1, CD58, and SLAMF6; IFN-β, and 4-1BBL; IFN-β, 4-1BBL, and CD70; IFN-β, 4-1BBL, and GITRL; IFN-β, 4-1BBL, and LIGHT; IFN-β, 4-1BBL, and TIM-4; IFN-β, 4-1BBL, and ICAM-1; IFN-β, 4-1BBL, and CD58; IFN-β, 4-1BBL, and SLAMF6; IFN-β, 4-1BBL, and CD70; IFN-β, 4-1BBL, and GITRL; IFN-β, 4-1BBL, and LIGHT; IFN-β, 4-1BBL, and TIM-4; IFN-β, 4-1BBL, and ICAM-1; IFN-β, 4-1BBL, and CD58; IFN-β, 4-1BBL, and SLAMF6; IFN-β, 4-1BBL, CD70, and GITRL; IFN-β, 4-1BBL, CD70, and LIGHT; IFN-β, 4-1BBL, CD70, and TIM-4; IFN-β, 4-1BBL, CD70, and ICAM-1; IFN-β, 4-1BBL, CD70, and CD58; IFN-β, 4-1BBL, CD70, and SLAMF6; IFN-β, 4-1BBL, CD70, GITRL, and LIGHT; IFN-β, 4-1BBL, CD70, GITRL, and TIM-4; IFN-β, 4-1BBL, CD70, GITRL, and ICAM-1; IFN-β, 4-1BBL, CD70, GITRL, and CD58; IFN-β, 4-1BBL, CD70, GITRL, and SLAMF6; IFN-β, 4-1BBL, CD70, GITRL, LIGHT, and TIM-4; IFN-β, 4-1BBL, CD70, GITRL, LIGHT and ICAM-1; IFN-β, 4-1BBL, CD70, GITRL, LIGHT and CD58; IFN-β, 4-1BBL, CD70, GITRL, LIGHT and SLAMF6; IFN-β, 4-1BBL, CD70, GITRL, LIGHT, TIM-4, and ICAM-1; IFN-β, 4-1BBL, CD70, GITRL, LIGHT, TIM-4, and CD58; IFN-β, 4-1BBL, CD70, GITRL, LIGHT, TIM-4, and SLAMF6; IFN-β, 4-1BBL, CD70, GITRL, LIGHT, TIM-4, ICAM-1, and CD58; IFN-β, 4-1BBL, CD70, GITRL, LIGHT, TIM-4, ICAM-I, and SLAMF6; IFN-β, 4-1BBL, CD70, GITRL, LIGHT, TIM-4, ICAM-1, CD58, and SLAMF6; IFN-β and CD70; IFN-β, CD70, and GITRL; IFN-β, CD70, and LIGHT; IFN-β, CD70, and TIM-4; IFN-β, CD70, and ICAM-1; IFN-β, CD70, and CD58; IFN-β, CD70, and SLAMF6; IFN-β, CD70, and GITRL; IFN-β, CD70, and LIGHT; IFN-β, CD70, and TIM-4; IFN-0, CD70, and ICAM-1; IFN-β, CD70, and CD58; IFN-β, CD70, and SLAMF6; IFN-β, CD70, GITRL, and LIGHT; IFN-β, CD70, GITRL, and TIM-4; IFN-β, CD70, GITRL, and ICAM-1; IFN-β, CD70, GITRL, and CD58; IFN-β, CD70, GITRL, and SLAMF6; IFN-β, CD70, GITRL, LIGHT, and TIM-4; IFN-β, CD70, GITRL, LIGHT and ICAM-1; IFN-β, CD70, GITRL, LIGHT and CD58; IFN-β, CD70, GITRL, LIGHT and SLAMF6; IFN-β, CD70, GITRL, LIGHT, TIM-4, and ICAM-1; IFN-β, CD70, GITRL, LIGHT, TIM-4, and CD58; IFN-β, CD70, GITRL, LIGHT, TIM-4, and SLAMF6; IFN-β, CD70, GITRL, LIGHT, TIM-4, ICAM-1, and CD58; IFN-β, CD70, GITRL, LIGHT, TIM-4, ICAM-1, and SLAMF6; IFN-β, CD70, GITRL, LIGHT, TIM-4, ICAM-1, CD58, and SLAMF6; IFN-β and GITRL; IFN-β, GITRL, and LIGHT; IFN-β, GITRL, and TIM-4; IFN-β, GITRL, and ICAM-1; IFN-β, GITRL, and CD58; IFN-β, GITRL, and SLAMF6; IFN-β, GITRL, and LIGHT; IFN-β, GITRL, and TIM-4; IFN-β, GITRL, and ICAM-1; IFN-β, GITRL, and CD58; IFN-β, GITRL, and SLAMF6; IFN-β, GITRL, LIGHT, and TIM-4; IFN-β, GITRL, LIGHT and ICAM-1; IFN-β, GITRL, LIGHT and CD58; IFN-β, GITRL, LIGHT and SLAMF6; IFN-β, GITRL, LIGHT, TIM-4, and ICAM-1; IFN-β, GITRL, LIGHT, TIM-4, and CD58; IFN-β, GITRL, LIGHT, TIM-4, and SLAMF6; IFN-β, GITRL, LIGHT, TIM-4, ICAM-1, and CD58; IFN-β, GITRL, LIGHT, TIM-4, ICAM-1, and SLAMF6; IFN-β, GITRL, LIGHT, TIM-4, ICAM-1, CD58, and SLAMF6; IFN-β, and LIGHT; IFN-β, LIGHT, and TIM-4; IFN-β, LIGHT, and ICAM-1; IFN-β, LIGHT, and CD58; IFN-β, LIGHT, and SLAMF6; IFN-β, LIGHT, and TIM-4; IFN-β, LIGHT and ICAM-1; IFN-β, LIGHT and CD58; IFN-β, LIGHT and SLAMF6; IFN-β, LIGHT, TIM-4, and ICAM-1; IFN-β, LIGHT, TIM-4, and CD58; IFN-β, LIGHT, TIM-4, and SLAMF6; IFN-β, LIGHT, TIM-4, ICAM-1, and CD58; IFN-β, LIGHT, TIM-4, ICAM-1, and SLAMF6; IFN-β, LIGHT, TIM-4, ICAM-1, CD58, and SLAMF6; IFN-β, and TIM-4; IFN-β, TIM-4, and ICAM-1; IFN-β, TIM-4, and CD58; IFN-β, TIM-4, and SLAMF6; IFN-β, TIM-4, and ICAM-1; IFN-β, TIM-4, and CD58; IFN-β, TIM-4, and SLAMF6; IFN-β, TIM-4, ICAM-1, and CD58; IFN-β, TIM-4, ICAM-1, and SLAMF6; IFN-β, TIM-4, ICAM-1, CD58, and SLAMF6; IFN-β and ICAM-1; IFN-β, ICAM-1, and CD58; IFN-β, ICAM-1, and SLAMF6; IFN-β, ICAM-1, and CD58; IFN-β, ICAM-1, and SLAMF6; IFN-β, ICAM-1, CD58, and SLAMF6; IFN-β and CD58; IFN-β, CD58, and SLAMF6; IFN-β and SLAMF6; IFN-β, IFN-κ, and CD40-L; IFN-β, IFN-κ, and MEM40; IFN-β, IFN-κ, and B7-1(CD80)/B7-2(CD86); IFN-β, IFN-κ, and OX40L, IFN-β, IFN-κ, and 4-1BBL; IFN-β, IFN-κ, and CD70; IFN-β, IFN-κ, and GITRL; IFN-β, IFN-κ, and LIGHT; IFN-β, IFN-κ, and TIM-4; IFN-β, IFN-κ, and ICAM-1; IFN-0, and CD58; IFN-β, IFN-κ, and SLAMF6; IFN-β, IFN-κ, IFN-β, and CD40-L; IFN-β, IFN-κ, IFN-δ, and MEM40; IFN-β, IFN-κ, IFN-β, and B7-1(CD80)/B7-2(CD86); IFN-β, IFN-κ, IFN-δ, and OX40L; IFN-β, IFN-κ, IFN-δ, and 4-1BBL; IFN-β, IFN-κ, IFN-δ, and CD70; IFN-β, IFN-κ, IFN-δ, and GITRL; IFN-β, IFN-κ, IFN-δ, and LIGHT; IFN-β, IFN-κ, IFN-δ, and TIM-4; IFN-β, IFN-κ, IFN-δ, and ICAM-1; IFN-β, IFN-δ, and CD58; IFN-β, IFN-κ, IFN-β, and SLAMF6; IFN-β, IFN-κ, IFN-δ, IFN-ε, and CD40-L; IFN-β, IFN-κ, IFN-δ, IFN-ε, and MEM40; IFN-β, IFN-κ, IFN-δ, IFN-ε, and B7-1(CD80)/B7-2(CD86); IFN-β, IFN-κ, IFN-δ, IFN-ε, and OX40L; IFN-β, IFN-κ, IFN-δ, IFN-ε, and 4-1BBL; IFN-β, IFN-κ, IFN-δ, IFN-ε, and CD70; IFN-β, IFN-κ, IFN-8, IFN-ε, and GITRL; IFN-β, IFN-κ, IFN-δ, IFN-ε, and LIGHT; IFN-β, IFN-κ, IFN-δ, IFN-ε, and TIM-4; IFN-β, IFN-κ, IFN-δ, IFN-ε, and ICAM-1; IFN-β, IFN-δ, IFN-ε, and CD58; IFN-β, IFN-κ, IFN-δ, IFN-ε, and SLAMF6; IFN-β, IFN-κ, IFN-δ, IFN-ε, IFN-τ, and CD40-L; IFN-β, IFN-κ, IFN-δ, IFN-ε, IFN-τ, and MEM40; IFN-β, IFN-κ, IFN-δ, IFN-ε, IFN-τ, and B7-1(CD80)/B7-2(CD86); IFN-β, IFN-κ, IFN-δ, IFN-ε, IFN-τ, and OX40L; IFN-β, IFN-κ, IFN-δ, IFN-ε, IFN-ε, and 4-1BBL; IFN-β, IFN-κ, IFN-δ, IFN-ε, IFN-τ, and CD70; IFN-β, IFN-κ, IFN-δ, IFN-ε, IFN-τ, and GITRL; IFN-β, IFN-κ, IFN-δ, IFN-ε, IFN-τ, and LIGHT; IFN-β, IFN-κ, IFN-δ, IFN-ε, IFN-τ, and TIM-4; IFN-β, IFN-κ, IFN-δ, IFN-ε, IFN-τ, and ICAM-1; IFN-β, IFN-δ, IFN-ε, IFN-τ, and CD58; IFN-β, IFN-κ, IFN-δ, IFN-ε, IFN-τ, and SLAMF6; IFN-β, IFN-κ, IFN-δ, IFN-ε, IFN-ε, IFN-ω, and CD40-L; IFN-β, IFN-κ, IFN-δ, IFN-ε, IFN-τ, IFN-ω, and MEM40; IFN-β, IFN-κ, IFN-δ, IFN-ε, IFN-τ, IFN-ω, and B7-1(CD80)/B7-2(CD86); IFN-β, IFN-κ, IFN-δ, IFN-ε, IFN-ε, IFN-ω, and OX40L; IFN-β, IFN-κ, IFN-δ, IFN-ε, IFN-τ, IFN-ω, and 4-1BBL; IFN-β, IFN-κ, IFN-δ, IFN-ε, IFN-τ, IFN-ω, and CD70; IFN-β, IFN-κ, IFN-δ, IFN-ε, IFN-τ, IFN-ω, and GITRL; IFN-β, IFN-κ, IFN-δ, IFN-ε, IFN-ζ, IFN-ω, and LIGHT; IFN-β, IFN-κ, IFN-δ, IFN-ε, IFN-ζ, IFN-ω, and TIM-4; IFN-β, IFN-κ, IFN-δ, IFN-ε, IFN-τ, IFN-ω, and ICAM-1; IFN-β, IFN-δ, IFN-ε, IFN-ζ, IFN-ω, and CD58; IFN-β, IFN-κ, IFN-δ, IFN-ε, IFN-ζ, IFN-ω, and SLAMF6; IFN-β, IFN-κ, IFN-δ, IFN-ε, IFN-τ, IFN-ω, IFN-ζ, and CD40-L; IFN-β, IFN-κ, IFN-δ, IFN-ε, IFN-τ, IFN-ω, IFN-ζ, and MEM40; IFN-β, IFN-κ, IFN-δ, IFN-ε, IFN-τ, IFN-ω, IFN-ζ, and B7-1(CD80)/B7-2(CD86); IFN-β, IFN-κ, IFN-δ, IFN-ε, IFN-τ, IFN-ω, IFN-ζ, and OX40L; IFN-β, IFN-κ, IFN-δ, IFN-ε, IFN-τ, IFN-ω, IFN-ζ, and 4-1BBL; IFN-β, IFN-κ, IFN-δ, IFN-ε, IFN-τ, IFN-ω, IFN-ζ, and CD70; IFN-β, IFN-κ, IFN-δ, IFN-ε, IFN-τ, IFN-ω, IFN-ζ, and GITRL; IFN-β, IFN-κ, IFN-δ, IFN-ε, IFN-τ, IFN-ω, IFN-ζ, and LIGHT; IFN-β, IFN-κ, IFN-δ, IFN-ε, IFN-τ, IFN-ω, IFN-ζ, and TIM-4; IFN-β, IFN-κ, IFN-δ, IFN-ε, IFN-τ, IFN-ω, IFN-ζ, and ICAM-1; IFN-β, IFN-δ, IFN-ε, IFN-τ, IFN-ω, IFN-ζ, and CD58; and/or IFN-β, IFN-κ, IFN-δ, IFN-ε, IFN-τ, IFN-ω, IFN-ζ, and SLAMF6. In one aspect, the viral vector further comprises IL-12 and/or IL-23. Alternatively, disclosed herein are viral vector, such as a lentivirus, that express i) one or more type 1 interferon (IFN) (such as, for example, IFN-α, IFN-β, IFN-κ, IFN-δ, IFN-ε, IFN-τ, IFN-ω, and/or IFN-ζ) or one or more exogenous immunostimulatory molecules (such as, for example, CD40-L, MEM40, B7-1(CD80)/B7-2(CD86), OX40L, 4-1BBL, CD70, GITRL, LIGHT, TIM-4, ICAM-1, CD58 and/or SLAMF6) and/or ii) an interleukin selected from IL-12 and/or IL-23. For example, the oncolytic virus for use in the disclosed methods can comprise IFN-α and IL-12, IFN-β and IL-12, IFN-κ and IL-12, IFN-δ and IL-12, IFN-ε and IL-12, IFN-τ and IL-12, IFN-ω and IL-12, IFN-ζ and IL-12, CD40-L and IL-12, MEM40 and IL-12, B7-1(CD80)/B7-2(CD86) and IL-12, OX40L and IL-12, 4-1BBL and IL-12, CD70 and IL-12, GITRL and IL-12, LIGHT and IL-12, TIM-4 and IL-12, ICAM-1 and IL-12, CD58 and IL-12, SLAMF6 and IL-12; IFN-α and IL-23, IFN-β and IL-23, IFN-κ and IL-23, IFN-δ and IL-23, IFN-ε and IL-23, IFN-τ and IL-23, IFN-ω and IL-23, IFN-ζ and IL-23, CD40-L and IL-23, MEM40 and IL-23, B7-1(CD80)/B7-2(CD86) and IL-23, OX40L and IL-23, 4-1BBL and IL-23, CD70 and IL-23, GITRL and IL-23, LIGHT and IL-23, TIM-4 and IL-23, ICAM-1 and IL-23, CD58 and IL-23, SLAMF6 and IL-23, IFN-α, IL-12, and IL-23, IFN-β, IL-12, and IL-23, IFN-κ, IL-12, and IL-23, IFN-δ, IL-12, and IL-23, IFN-ε, IL-12, and IL-23, IFN-τ, IL-12, and IL-23, IFN-ω, IL-12, and IL-23, IFN-ζ, IL-12, and IL-23, CD40-L, IL-12, and IL-23, MEM40, IL-12, and IL-23, B7-1(CD80)/B7-2(CD86), IL-12, and IL-23, OX40L, IL-12, and IL-23, 4-1BBL, IL-12, and IL-23, CD70, IL-12, and IL-23, GITRL, IL-12, and IL-23, LIGHT, IL-12, and IL-23, TIM-4, IL-12, and IL-23, ICAM-1, IL-12, and IL-23, CD58, IL-12, and IL-23, and/or SLAMF6, IL-12, and IL-23.

Additional embodiments of the invention provide oncolytic viruses expressing any combination of a type I IFN, such as, IFN-α, IFN-β, IFN-ε, IFN-κ, and IFN-ω, and an immunostimulatory molecule (such as, for example, CD40-L, MEM40, B7-1(CD80)/B7-2(CD86), OX40L, 4-1BBL, CD70, GITRL, LIGHT, TIM-4, ICAM-1, CD58 and/or SLAMF6), for example, via one or more heterologous nucleic acid sequences encoding a combination of IFN-β and CD40-L. For example, the disclosed oncolytic virus can express IFN-α and CD40-L; IFN-α and MEM40; IFN-α and B7-1(CD80)/B7-2(CD86); IFN-α and OX40L; IFN-α and 4-1BBL; IFN-α and CD70; IFN-α and GITRL; IFN-α and LIGHT; IFN-α and TIM-4; IFN-α and ICAM-1; IFN-α and CD58; IFN-α and SLAMF6; IFN-α, CD40-L, and MEM40; IFN-α, CD40-L, and B7-1(CD80)/B7-2(CD86); IFN-α, CD40-L, and OX40L; IFN-α, CD40-L, and 4-1BBL; IFN-α, CD40-L, and CD70; IFN-α, CD40-L, and GITRL; IFN-α, CD40-L, and LIGHT; IFN-α, CD40-L, and TIM-4; IFN-α, CD40-L, and ICAM-1; IFN-α, CD40-L, and CD58; IFN-α, CD40-L, and SLAMF6; IFN-α, CD40-L, MEM40, and B7-1(CD80)B7-2(CD86); IFN-α, CD40-L, MEM40, and OX40L; IFN-α, CD40-L, MEM40, and 4-1BBL; IFN-α, CD40-L, MEM40, and CD70; IFN-α, CD40-L, MEM40, and GITRL; IFN-α, CD40-L, MEM40, and LIGHT; IFN-α, CD40-L, MEM40, and TIM-4; IFN-α, CD40-L, MEM40, and ICAM-1; IFN-α, CD40-L, MEM40, and CD58; IFN-α, CD40-L, MEM40, and SLAMF6; IFN-α, CD40-L, MEM40, B7-1(CD80)/B7-2(CD86), and OX40L; IFN-α, CD40-L, MEM40, B7-1(CD80)/B7-2(CD86), and 4-1BBL; IFN-α, CD40-L, MEM40, B7-1(CD80)/B7-2(CD86), and CD70; IFN-α, CD40-L, MEM40, B7-1(CD80)/B7-2(CD86), and GITRL; IFN-α, CD40-L, MEM40, B7-1(CD80)/B7-2(CD86), and LIGHT; IFN-α, CD40-L, MEM40, B7-1(CD80)/B7-2(CD86), and TIM-4; IFN-α, CD40-L, MEM40, B7-1(CD80)/B7-2(CD86), and ICAM-1; IFN-α, CD40-L, MEM40, B7-1(CD80)/B7-2(CD86), and CD58; IFN-α, CD40-L, MEM40, B7-1(CD80)/B7-2(CD86), and SLAMF6; IFN-α, CD40-L, MEM40, B7-1(CD80)/B7-2(CD86), OX40L, and 4-1BBL; IFN-α, CD40-L, MEM40, B7-1(CD80)/B7-2(CD86), OX40L, and CD70; IFN-α, CD40-L, MEM40, B7-1(CD80)/B7-2(CD86), OX40L, and GITRL; IFN-α, CD40-L, MEM40, B7-1(CD80)/B7-2(CD86), OX40L, and LIGHT; IFN-α, CD40-L, MEM40, B7-1(CD80)/B7-2(CD86), OX40L, and TIM-4; IFN-α, CD40-L, MEM40, B7-1(CD80)/B7-2(CD86), OX40L, and ICAM-1; IFN-α, CD40-L, MEM40, B7-1(CD80)/B7-2(CD86), OX40L, and CD58; IFN-α, CD40-L, MEM40, B7-1(CD80)/B7-2(CD86), OX40L, and SLAMF6; IFN-α, CD40-L, MEM40, B7-1(CD80)/B7-2(CD86), OX40L, 4-1BBL, and CD70; IFN-α, CD40-L, MEM40, B7-1(CD80)/B7-2(CD86), OX40L, 4-1BBL, and GITRL; IFN-α, CD40-L, MEM40, B7-1(CD80)/B7-2(CD86), OX40L, 4-1BBL, and LIGHT; IFN-α, CD40-L, MEM40, B7-1(CD80)/B7-2(CD86), OX40L, 4-1BBL, and TIM-4; IFN-α, CD40-L, MEM40, B7-1(CD80)/B7-2(CD86), OX40L, 4-1BBL, and ICAM-1; IFN-α, CD40-L, MEM40, B7-1(CD80)/B7-2(CD86), OX40L, 4-1BBL, and CD58; IFN-α, CD40-L, MEM40, B7-1(CD80)/B7-2(CD86), OX40L, 4-1BBL, and SLAMF6; IFN-α, CD40-L, MEM40, B7-1(CD80)/B7-2(CD86), OX40L, 4-1BBL, CD70, and GITRL; IFN-α, CD40-L, MEM40, B7-1(CD80)/B7-2(CD86), OX40L, 4-1BBL, CD70, and LIGHT; IFN-α, CD40-L, MEM40, B7-1(CD80)/B7-2(CD86), OX40L, 4-1BBL, CD70, and TIM-4; IFN-α, CD40-L, MEM40, B7-1(CD80)/B7-2(CD86), OX40L, 4-1BBL, CD70, and ICAM-1; IFN-α, CD40-L, MEM40, B7-1(CD80)/B7-2(CD86), OX40L, 4-1BBL, CD70, and CD58; IFN-α, CD40-L, MEM40, B7-1(CD80)/B7-2(CD86), OX40L, 4-1BBL, CD70, and SLAMF6; IFN-α, CD40-L, MEM40, B7-1(CD80)/B7-2(CD86), OX40L, 4-1BBL, CD70, GITRL, and LIGHT; IFN-α, CD40-L, MEM40, B7-1(CD80)/B7-2(CD86), OX40L, 4-1BBL, CD70, GITRL, and TIM-4; IFN-α, CD40-L, MEM40, B7-1(CD80)/B7-2(CD86), OX40L, 4-1BBL, CD70, GITRL, and ICAM-1; IFN-α, CD40-L, MEM40, B7-1(CD80)/B7-2(CD86), OX40L, 4-1BBL, CD70, GITRL, and CD58; IFN-α, CD40-L, MEM40, B7-1(CD80)/B7-2(CD86), OX40L, 4-1BBL, CD70, GITRL, and SLAMF6; IFN-α, CD40-L, MEM40, B7-1(CD80)/B7-2(CD86), OX40L, 4-1BBL, CD70, GITRL, LIGHT, and TIM-4; IFN-α, CD40-L, MEM40, B7-1(CD80)/B7-2(CD86), OX40L, 4-1BBL, CD70, GITRL, LIGHT and ICAM-1; IFN-α, CD40-L, MEM40, B7-1(CD80)/B7-2(CD86), OX40L, 4-1BBL, CD70, GITRL, LIGHT and CD58; IFN-α, CD40-L, MEM40, B7-1(CD80)/B7-2(CD86), OX40L, 4-1BBL, CD70, GITRL, LIGHT and SLAMF6; IFN-α, CD40-L, MEM40, B7-1(CD80)/B7-2(CD86), OX40L, 4-1BBL, CD70, GITRL, LIGHT, TIM-4, and ICAM-1; IFN-α, CD40-L, MEM40, B7-1(CD80)/B7-2(CD86), OX40L, 4-1BBL, CD70, GITRL, LIGHT, TIM-4, and CD58; IFN-α, CD40-L, MEM40, B7-1(CD80)/B7-2(CD86), OX40L, 4-1BBL, CD70, GITRL, LIGHT, TIM-4, and SLAMF6; IFN-α, CD40-L, MEM40, B7-1(CD80)/B7-2(CD86), OX40L, 4-1BBL, CD70, GITRL, LIGHT, TIM-4, ICAM-1, and CD58; IFN-α, CD40-L, MEM40, B7-1(CD80)/B7-2(CD86), OX40L, 4-1BBL, CD70, GITRL, LIGHT, TIM-4, ICAM-1, and SLAMF6; IFN-α, CD40-L, MEM40, B7-1(CD80)/B7-2(CD86), OX40L, 4-1BBL, CD70, GITRL, LIGHT, TIM-4, ICAM-1, CD58, and SLAMF6; IFN-α, MEM40; IFN-α, MEM40, and B7-1(CD80)/B7-2(CD86); IFN-α, MEM40, and OX40L; IFN-α, MEM40, and 4-1BBL; IFN-α, MEM40, and CD70; IFN-α, MEM40, and GITRL; IFN-α, MEM40, and LIGHT; IFN-α, MEM40, and TIM-4; IFN-α, MEM40, and ICAM-1; IFN-α, MEM40, and CD58; IFN-α, MEM40, and SLAMF6; IFN-α, MEM40, and B7-1(CD80)/B7-2(CD86); IFN-α, MEM40, and OX40L; IFN-α, MEM40, and 4-1BBL; IFN-α, MEM40, and CD70; IFN-α, MEM40, and GITRL; IFN-α, MEM40, and LIGHT; IFN-α, MEM40, and TIM-4; IFN-α, MEM40, and ICAM-1; IFN-α, MEM40, and CD58; IFN-α, MEM40, and SLAMF6; IFN-α, MEM40, B7-1(CD80)/B7-2(CD86), and OX40L; IFN-α, MEM40, B7-1(CD80)B7-2(CD86), and 4-1BBL; IFN-α, MEM40, B7-1(CD80)/B7-2(CD86), and CD70; IFN-α, MEM40, B7-1(CD80)/B7-2(CD86), and GITRL; IFN-α, MEM40, B7-1(CD80)/B7-2(CD86), and LIGHT; IFN-α, MEM40, B7-1(CD80)/B7-2(CD86), and TIM-4; IFN-α, MEM40, B7-1(CD80)/B7-2(CD86), and ICAM-1; IFN-α, MEM40, B7-1(CD80)/B7-2(CD86), and CD58; IFN-α, MEM40, B7-1(CD80)/B7-2(CD86), and SLAMF6; IFN-α, MEM40, B7-1(CD80)/B7-2(CD86), OX40L, and 4-1BBL; IFN-α, MEM40, B7-1(CD80)/B7-2(CD86), OX40L, and CD70; IFN-α, MEM40, B7-1(CD80)/B7-2(CD86), OX40L, and GITRL; IFN-α, MEM40, B7-1(CD80)/B7-2(CD86), OX40L, and LIGHT; IFN-α, MEM40, B7-1(CD80)/B7-2(CD86), OX40L, and TIM-4; IFN-α, MEM40, B7-1(CD80)/B7-2(CD86), OX40L, and ICAM-1; IFN-α, MEM40, B7-1(CD80)/B7-2(CD86), OX40L, and CD58; IFN-α, MEM40, B7-1(CD80)/B7-2(CD86), OX40L, and SLAMF6; IFN-α, MEM40, B7-1(CD80)/B7-2(CD86), OX40L, 4-1BBL, and CD70; IFN-α, MEM40, B7-1(CD80)/B7-2(CD86), OX40L, 4-1BBL, and GITRL; IFN-α, MEM40, B7-1(CD80)/B7-2(CD86), OX40L, 4-1BBL, and LIGHT; IFN-α, MEM40, B7-1(CD80)/B7-2(CD86), OX40L, 4-1BBL, and TIM-4, IFN-α, MEM40, B7-1(CD80)/B7-2(CD86), OX40L, 4-1BBL, and ICAM-1; IFN-α, MEM40, B7-1(CD80)/B7-2(CD86), OX40L, 4-1BBL, and CD58; IFN-α, MEM40, B7-1(CD80)/B7-2(CD86), OX40L, 4-1BBL, and SLAMF6; IFN-α, MEM40, B7-1(CD80)/B7-2(CD86), OX40L, 4-1BBL, CD70, and GITRL; IFN-α, MEM40, B7-1(CD80)/B7-2(CD86), OX40L, 4-1BBL, CD70, and LIGHT; IFN-α, MEM40, B7-1(CD80)/B7-2(CD86), OX40L, 4-1BBL, CD70, and TIM-4; IFN-α, MEM40, B7-1(CD80)/B7-2(CD86), OX40L, 4-1BBL, CD70, and ICAM-1; IFN-α, MEM40, B7-1(CD80)/B7-2(CD86), OX40L, 4-1BBL, CD70, and CD58; IFN-α, MEM40, B7-1(CD80)/B7-2(CD86), OX40L, 4-1BBL, CD70, and SLAMF6; IFN-α, MEM40, B7-1(CD80)/B7-2(CD86), OX40L, 4-1BBL, CD70, GITRL, and LIGHT; IFN-α, MEM40, B7-1(CD80)/B7-2(CD86), OX40L, 4-1BBL, CD70, GITRL, and TIM-4; IFN-α, MEM40, B7-1(CD80)/B7-2(CD86), OX40L, 4-1BBL, CD70, GITRL, and ICAM-1; IFN-α, MEM40, B7-1(CD80)/B7-2(CD86), OX40L, 4-1BBL, CD70, GITRL, and CD58; IFN-α, MEM40, B7-1(CD80)/B7-2(CD86), OX40L, 4-1BBL, CD70, GITRL, and SLAMF6; IFN-α, MEM40, B7-1(CD80)/B7-2(CD86), OX40L, 4-1BBL, CD70, GITRL, LIGHT, and TIM-4; IFN-α, MEM40, B7-1(CD80)/B7-2(CD86), OX40L, 4-1BBL, CD70, GITRL, LIGHT and ICAM-1; IFN-α, MEM40, B7-1(CD80)/B7-2(CD86), OX40L, 4-1BBL, CD70, GITRL, LIGHT and CD58; IFN-α, MEM40, B7-1(CD80)/B7-2(CD86), OX40L, 4-1BBL, CD70, GITRL, LIGHT and SLAMF6; IFN-α, MEM40, B7-1(CD80)/B7-2(CD86), OX40L, 4-1 BBL, CD70, GITRL, LIGHT, TIM-4, and ICAM-1; IFN-α, MEM40, B7-1(CD80)/B7-2(CD86), OX40L, 4-1BBL, CD70, GITRL, LIGHT, TIM-4, and CD58; IFN-α, MEM40, B7-1(CD80)/B7-2(CD86), OX40L, 4-1BBL, CD70, GITRL, LIGHT, TIM-4, and SLAMF6; IFN-α, MEM40, B7-1(CD80)/B7-2(CD86), OX40L, 4-1BBL, CD70, GITRL, LIGHT, TIM-4, ICAM-1, and CD58; IFN-α, MEM40, B7-1(CD80)/B7-2(CD86), OX40L, 4-1BBL, CD70, GITRL, LIGHT, TIM-4, ICAM-1, and SLAMF6; IFN-α, MEM40, B7-1(CD80)/B7-2(CD86), OX40L, 4-1BBL, CD70, GITRL, LIGHT, TIM-4, ICAM-1, CD58, and SLAMF6; IFN-α, and B7-1(CD80)/B7-2(CD86); IFN-α, B7-1(CD80)/B7-2(CD86), and OX40L; IFN-α, B7-1(CD80)/B7-2(CD86), and 4-1BBL; IFN-α, B7-1(CD80)/B7-2(CD86), and CD70; IFN-α, B7-1(CD80)/B7-2(CD86), and GITRL; IFN-α, B7-1(CD80)/B7-2(CD86), and LIGHT; IFN-α, B7-1(CD80)/B7-2(CD86), and TIM-4; IFN-α, B7-1(CD80)/B7-2(CD86), and ICAM-1; IFN-α, B7-1(CD80)/B7-2(CD86), and CD58; IFN-α, B7-1(CD80)/B7-2(CD86), and SLAMF6; IFN-α, B7-1(CD80)/B7-2(CD86), and OX40L; IFN-α, B7-1(CD80)/B7-2(CD86), and 4-1BBL; IFN-α, B7-1(CD80)/B7-2(CD86), and CD70; IFN-α, B7-1(CD80)/B7-2(CD86), and GITRL; IFN-α, B7-1(CD80)/B7-2(CD86), and LIGHT; IFN-α, B7-1(CD80)/B7-2(CD86), and TIM-4; IFN-α, B7-1(CD80)/B7-2(CD86), and ICAM-1; IFN-α, B7-1(CD80)/B7-2(CD86), and CD58; IFN-α, B7-1(CD80)/B7-2(CD86), and SLAMF6; IFN-α, B7-1(CD80)/B7-2(CD86), OX40L, and 4-1BBL; IFN-α, B7-1(CD80)/B7-2(CD86), OX40L, and CD70; IFN-α, B7-1(CD80)/B7-2(CD86), OX40L, and GITRL; IFN-α, B7-1(CD80)/B7-2(CD86), OX40L, and LIGHT; IFN-α, B7-1(CD80)/B7-2(CD86), OX40L, and TIM-4; IFN-α, B7-1(CD80)/B7-2(CD86), OX40L, and ICAM-1; IFN-α, B7-1(CD80)/B7-2(CD86), OX40L, and CD58; IFN-α, B7-1(CD80)/B7-2(CD86), OX40L, and SLAMF6; IFN-α, B7-1(CD80)/B7-2(CD86), OX40L, 4-1BBL, and CD70; IFN-α, B7-1(CD80)/B7-2(CD86), OX40L, 4-1BBL, and GITRL; IFN-α, B7-1(CD80)/B7-2(CD86), OX40L, 4-1BBL, and LIGHT; IFN-α, B7-1(CD80)/B7-2(CD86), OX40L, 4-1BBL, and TIM-4; IFN-α, B7-1(CD80)/B7-2(CD86), OX40L, 4-1BBL, and ICAM-1; IFN-α, B7-1(CD80)/B7-2(CD86), OX40L, 4-1BBL, and CD58; IFN-α, B7-1(CD80)/B7-2(CD86), OX40L, 4-1BBL, and SLAMF6; IFN-α, B7-1(CD80)/B7-2(CD86), OX40L, 4-1BBL, CD70, and GITRL; IFN-α, B7-1(CD80)/B7-2(CD86), OX40L, 4-1BBL, CD70, and LIGHT; IFN-α, B7-1(CD80)/B7-2(CD86), OX40L, 4-1BBL, CD70, and TIM-4; IFN-α, B7-1(CD80)/B7-2(CD86), OX40L, 4-1BBL, CD70, and ICAM-1; IFN-α, B7-1(CD80)/B7-2(CD86), OX40L, 4-1BBL, CD70, and CD58; IFN-α, B7-1(CD80)/B7-2(CD86), OX40L, 4-1BBL, CD70, and SLAMF6; IFN-α, B7-1(CD80)/B7-2(CD86), OX40L, 4-1BBL, CD70, GITRL, and LIGHT; IFN-α, B7-1(CD80)/B7-2(CD86), OX40L, 4-1BBL, CD70, GITRL, and TIM-4; IFN-α, B7-1(CD80)/B7-2(CD86), OX40L, 4-1BBL, CD70, GITRL, and ICAM-1; IFN-α, B7-1(CD80)/B7-2(CD86), OX40L, 4-1BBL, CD70, GITRL, and CD58; IFN-α, B7-1(CD80)/B7-2(CD86), OX40L, 4-1BBL, CD70, GITRL, and SLAMF6; IFN-α, B7-1(CD80)/B7-2(CD86), OX40L, 4-1BBL, CD70, GITRL, LIGHT, and TIM-4; IFN-α, B7-1(CD80)/B7-2(CD86), OX40L, 4-1BBL, CD70, GITRL, LIGHT and ICAM-1; IFN-α, B7-1(CD80)/B7-2(CD86), OX40L, 4-1 BBL, CD70, GITRL, LIGHT and CD58; IFN-α, B7-1(CD80)/B7-2(CD86), OX40L, 4-1BBL, CD70, GITRL, LIGHT and SLAMF6; IFN-α, B7-1(CD80)/B7-2(CD86), OX40L, 4-1BBL, CD70, GITRL, LIGHT, TIM-4, and ICAM-1; IFN-α, B7-1(CD80)/B7-2(CD86), OX40L, 4-1BBL, CD70, GITRL, LIGHT, TIM-4, and CD58; IFN-α, B7-1(CD80)/B7-2(CD86), OX40L, 4-1BBL, CD70, GITRL, LIGHT, TIM-4, and SLAMF6; IFN-α, B7-1(CD80)/B7-2(CD86), OX40L, 4-1BBL, CD70, GITRL, LIGHT, TIM-4, ICAM-1, and CD58; IFN-α, B7-1(CD80)/B7-2(CD86), OX40L, 4-1BBL, CD70, GITRL, LIGHT, TIM-4, ICAM-1, and SLAMF6; IFN-α, B7-1(CD80)/B7-2(CD86), OX40L, 4-1BBL, CD70, GITRL, LIGHT, TIM-4, ICAM-1, CD58, and SLAMF6; IFN-α, and OX40L; IFN-α, OX40L, and 4-1BBL; IFN-α, OX40L, and CD70; IFN-α, OX40L, and GITRL; IFN-α, OX40L, and LIGHT; IFN-α, OX40L, and TIM-4; IFN-α, OX40L, and ICAM-1; IFN-α, OX40L, and CD58; IFN-α, OX40L, and SLAMF6; IFN-α, OX40L, and 4-1BBL; IFN-α, OX40L, and CD70; IFN-α, OX40L, and GITRL; IFN-α, OX40L, and LIGHT, IFN-α, OX40L, and TIM-4; IFN-α, OX40L, and ICAM-1; IFN-α, OX40L, and CD58; IFN-α, OX40L, and SLAMF6; IFN-α, OX40L, 4-1BBL, and CD70; IFN-α, OX40L, 4-1BBL, and GITRL; IFN-α, OX40L, 4-1BBL, and LIGHT; IFN-α, OX40L, 4-1BBL, and TIM-4, IFN-α, OX40L, 4-1BBL, and ICAM-1; IFN-α, OX40L, 4-1BBL, and CD58; IFN-α, OX40L, 4-1BBL, and SLAMF6; IFN-α, OX40L, 4-1BBL, CD70, and GITRL; IFN-α, OX40L, 4-1BBL, CD70, and LIGHT; IFN-α, OX40L, 4-1BBL, CD70, and TIM-4; IFN-α, OX40L, 4-1BBL, CD70, and ICAM-1; IFN-α, OX40L, 4-1BBL, CD70, and CD58; IFN-α, OX40L, 4-1BBL, CD70, and SLAMF6; IFN-α, OX40L, 4-1BBL, CD70, GITRL, and LIGHT; IFN-α, OX40L, 4-1BBL, CD70, GITRL, and TIM-4; IFN-α, OX40L, 4-1BBL, CD70, GITRL, and ICAM-1; IFN-α, OX40L, 4-1BBL, CD70, GITRL, and CD58; IFN-α, OX40L, 4-1BBL, CD70, GITRL, and SLAMF6; IFN-α, OX40L, 4-1BBL, CD70, GITRL, LIGHT, and TIM-4; IFN-α, OX40L, 4-1BBL, CD70, GITRL, LIGHT and ICAM-1; IFN-α, OX40L, 4-1BBL, CD70, GITRL, LIGHT and CD58; IFN-α, OX40L, 4-1BBL, CD70, GITRL, LIGHT and SLAMF6; IFN-α, OX40L, 4-1BBL, CD70, GITRL, LIGHT, TIM-4, and ICAM-1; IFN-α, OX40L, 4-1BBL, CD70, GITRL, LIGHT, TIM-4, and CD58; IFN-α, OX40L, 4-1BBL, CD70, GITRL, LIGHT, TIM-4, and SLAMF6; IFN-α, OX40L, 4-1BBL, CD70, GITRL, LIGHT, TIM-4, ICAM-1, and CD58; IFN-α, OX40L, 4-1BBL, CD70, GITRL, LIGHT, TIM-4, ICAM-1, and SLAMF6; IFN-α, OX40L, 4-1BBL, CD70, GITRL, LIGHT, TIM-4, ICAM-1, CD58, and SLAMF6; IFN-α, and 4-1BBL; IFN-α, 4-1BBL, and CD70; IFN-α, 4-1BBL, and GITRL; IFN-α, 4-1BBL, and LIGHT; IFN-α, 4-1BBL, and TIM-4; IFN-α, 4-1BBL, and ICAM-1; IFN-α, 4-1BBL, and CD58; IFN-α, 4-1BBL, and SLAMF6; IFN-α, 4-1BBL, and CD70; IFN-α, 4-1BBL, and GITRL; IFN-α, 4-1BBL, and LIGHT; IFN-α, 4-1BBL, and TIM-4; IFN-α, 4-1BBL, and ICAM-1; IFN-α, 4-1BBL, and CD58; IFN-α, 4-1BBL, and SLAMF6; IFN-α, 4-1BBL, CD70, and GITRL; IFN-α, 4-1 BBL, CD70, and LIGHT; IFN-α, 4-1BBL, CD70, and TIM-4; IFN-α, 4-1BBL, CD70, and ICAM-1; IFN-α, 4-1BBL, CD70, and CD58; IFN-α, 4-1BBL, CD70, and SLAMF6; IFN-α, 4-1BBL, CD70, GITRL, and LIGHT; IFN-α, 4-1BBL, CD70, GITRL, and TIM-4; IFN-α, 4-1BBL, CD70, GITRL, and ICAM-1; IFN-α, 4-1BBL, CD70, GITRL, and CD58; IFN-α, 4-1BBL, CD70, GITRL, and SLAMF6; IFN-α, 4-1BBL, CD70, GITRL, LIGHT, and TIM-4; IFN-α, 4-1BBL, CD70, GITRL, LIGHT and ICAM-1, IFN-α, 4-1BBL, CD70, GITRL, LIGHT and CD58; IFN-α, 4-1BBL, CD70, GITRL, LIGHT and SLAMF6; IFN-α, 4-1BBL, CD70, GITRL, LIGHT, TIM-4, and ICAM-1; IFN-α, 4-1BBL, CD70, GITRL, LIGHT, TIM-4, and CD58; IFN-α, 4-1BBL, CD70, GITRL, LIGHT, TIM-4, and SLAMF6; IFN-α, 4-1BBL, CD70, GITRL, LIGHT, TIM-4, ICAM-1, and CD58; IFN-α, 4-1BBL, CD70, GITRL, LIGHT, TIM-4, ICAM-1, and SLAMF6; IFN-α, 4-1 BBL, CD70, GITRL, LIGHT, TIM-4, ICAM-1, CD58, and SLAMF6; IFN-α and CD70; IFN-α, CD70, and GITRL; IFN-α, CD70, and LIGHT; IFN-α, CD70, and TIM-4; IFN-α, CD70, and ICAM-1; IFN-α, CD70, and CD58; IFN-α, CD70, and SLAMF6; IFN-α, CD70, and GITRL; IFN-α, CD70, and LIGHT; IFN-α, CD70, and TIM-4; IFN-α, CD70, and ICAM-1; IFN-α, CD70, and CD58; IFN-α, CD70, and SLAMF6; IFN-α, CD70, GITRL, and LIGHT; IFN-α, CD70, GITRL, and TIM-4; IFN-α, CD70, GITRL, and ICAM-1; IFN-α, CD70, GITRL, and CD58; IFN-α, CD70, GITRL, and SLAMF6; IFN-α, CD70, GITRL, LIGHT, and TIM-4; IFN-α, CD70, GITRL, LIGHT and ICAM-1; IFN-α, CD70, GITRL, LIGHT and CD58; IFN-α, CD70, GITRL, LIGHT and SLAMF6; IFN-α, CD70, GITRL, LIGHT, TIM-4, and ICAM-1; IFN-α, CD70, GITRL, LIGHT, TIM-4, and CD58; IFN-α, CD70, GITRL, LIGHT, TIM-4, and SLAMF6; IFN-α, CD70, GITRL, LIGHT, TIM-4, ICAM-1, and CD58; IFN-α, CD70, GITRL, LIGHT, TIM-4, ICAM-1, and SLAMF6; IFN-α, CD70, GITRL, LIGHT, TIM-4, ICAM-1, CD58, and SLAMF6; IFN-α and GITRL; IFN-α, GITRL, and LIGHT; IFN-α, GITRL, and TIM-4; IFN-α, GITRL, and ICAM-1; IFN-α, GITRL, and CD58; IFN-α, GITRL, and SLAMF6; IFN-α, GITRL, and LIGHT; IFN-α, GITRL, and TIM-4; IFN-α, GITRL, and ICAM-1; IFN-α, GITRL, and CD58; IFN-α, GITRL, and SLAMF6; IFN-α, GITRL, LIGHT, and TIM-4; IFN-α, GITRL, LIGHT and ICAM-1; IFN-α, GITRL, LIGHT and CD58; IFN-α, GITRL, LIGHT and SLAMF6; IFN-α, GITRL, LIGHT, TIM-4, and ICAM-1; IFN-α, GITRL, LIGHT, TIM-4, and CD58; IFN-α, GITRL, LIGHT, TIM-4, and SLAMF6; IFN-α, GITRL, LIGHT, TIM-4, ICAM-1, and CD58; IFN-α, GITRL, LIGHT, TIM-4, ICAM-1, and SLAMF6; IFN-α, GITRL, LIGHT, TIM-4, ICAM-1, CD58, and SLAMF6; IFN-α, and LIGHT; IFN-α, LIGHT, and TIM-4; IFN-α, LIGHT, and ICAM-1; IFN-α, LIGHT, and CD58; IFN-α, LIGHT, and SLAMF6; IFN-α, LIGHT, and TIM-4; IFN-α, LIGHT and ICAM-1; IFN-α, LIGHT and CD58; IFN-α, LIGHT and SLAMF6; IFN-α, LIGHT, TIM-4, and ICAM-1; IFN-α, LIGHT, TIM-4, and CD58; IFN-α, LIGHT, TIM-4, and SLAMF6; IFN-α, LIGHT, TIM-4, ICAM-I, and CD58; IFN-α, LIGHT, TIM-4, ICAM-1, and SLAMF6; IFN-α, LIGHT, TIM-4, ICAM-1, CD58, and SLAMF6; IFN-α, and TIM4; IFN-α, TIM-4, and ICAM-1; IFN-α, TIM-4, and CD58; IFN-α, TIM-4, and SLAMF6; IFN-α, TIM-4, and ICAM-1; IFN-α, TIM-4, and CD58; IFN-α, TIM-4, and SLAMF6; IFN-α, TIM-4, ICAM-1, and CD58; IFN-α, TIM-4, ICAM-1, and SLAMF6; IFN-α, TIM-4, ICAM-1, CD58, and SLAMF6; IFN-α and ICAM-I; IFN-α, ICAM-1, and CD58; IFN-α, ICAM-1, and SLAMF6; IFN-α, ICAM-1, and CD58; IFN-α, ICAM-1, and SLAMF6; IFN-α, ICAM-1, CD58, and SLAMF6; IFN-α and CD58; IFN-α, CD58, and SLAMF6; IFN-α and SLAMF6; IFN-α, IFN-β, and CD40-L; IFN-α, IFN-β, and MEM40; IFN-α, IFN-β, and B7-1(CD80)/B7-2(CD86); IFN-α, IFN-β, and OX40L; IFN-α, IFN-β, and 4-1BBL; IFN-α, IFN-β, and CD70; IFN-α, IFN-β, and GITRL; IFN-α, IFN-β, and LIGHT; IFN-α, IFN-β, and TIM-4; IFN-α, IFN-0, and ICAM-1; IFN-α, IFN-β, and CD58; IFN-α, IFN-β, and SLAMF6; IFN-α, IFN-β, IFN-κ, and CD40-L; IFN-α, IFN-β, IFN-κ, and MEM40; IFN-α, IFN-β, IFN-κ, and B7-1(CD80)/B7-2(CD86); IFN-α, IFN-β, IFN-κ, and OX40L; IFN-α, IFN-β, IFN-κ, and 4-1BBL; IFN-α, IFN-β, IFN-κ, and CD70; IFN-α, IFN-β, IFN-κ, and GITRL; IFN-α, IFN-β, IFN-κ, and LIGHT; IFN-α, IFN-β, IFN-κ, and TIM-4; IFN-α, IFN-β, IFN-κ, and ICAM-1; IFN-α, IFN-β, and CD58; IFN-α, IFN-β, IFN-κ, and SLAMF6; IFN-α, IFN-β, IFN-κ, IFN-δ, and CD40-L; IFN-α, IFN-β, IFN-κ, IFN-δ, and MEM40; IFN-α, IFN-β, IFN-κ, IFN-δ, and B7-1(CD80)/B7-2(CD86); IFN-α, IFN-β, IFN-κ, IFN-δ, and OX40L; IFN-α, IFN-β, IFN-κ, IFN-δ, and 4-1BBL; IFN-α, IFN-β, IFN-κ, IFN-δ, and CD70; IFN-α, IFN-β, IFN-κ, IFN-δ, and GITRL; IFN-α, IFN-β, IFN-κ, IFN-δ, and LIGHT; IFN-α, IFN-β, IFN-κ, IFN-δ, and TIM-4; IFN-α, IFN-β, IFN-κ, IFN-δ, and ICAM-1; IFN-α, IFN-β, IFN-δ, and CD58; IFN-α, IFN-β, IFN-κ, IFN-δ, and SLAMF6; IFN-α, IFN-β, IFN-κ, IFN-δ, IFN-ε, and CD40-L; IFN-α, IFN-β, IFN-κ, IFN-δ, IFN-ε, and MEM40; IFN-α, IFN-β, IFN-κ, IFN-δ, IFN-ε, and B7-1(CD80)/B7-2(CD86); IFN-α, IFN-β, IFN-κ, IFN-δ, IFN-ε, and OX40L; IFN-α, IFN-β, IFN-κ, IFN-δ, IFN-ε, and 4-1BBL; IFN-α, IFN-β, IFN-κ, IFN-δ, IFN-ε, and CD70; IFN-α, IFN-β, IFN-κ, IFN-δ, IFN-ε, and GITRL; IFN-α, IFN-β, IFN-κ, IFN-δ, IFN-ε, and LIGHT; IFN-α, IFN-β, IFN-κ, IFN-δ, IFN-ε, and TIM-4; IFN-α, IFN-β, IFN-κ, IFN-δ, IFN-ε, and ICAM-1; IFN-α, IFN-β, IFN-δ, IFN-ε, and CD58; IFN-α, IFN-β, IFN-κ, IFN-δ, IFN-ε, and SLAMF6; IFN-α, IFN-β, IFN-κ, IFN-δ, IFN-ε, IFN-τ, and CD40-L; IFN-α, IFN-β, IFN-κ, IFN-δ, IFN-ε, IFN-τ, and MEM40; IFN-α, IFN-β, IFN-κ, IFN-δ, IFN-ε, IFN-ε, and B7-1(CD80)/B7-2(CD86); IFN-α, IFN-β, IFN-κ, IFN-δ, IFN-ε, IFN-τ, and OX40L; IFN-α, IFN-β, IFN-κ, IFN-δ, IFN-ε, IFN-τ, and 4-1BBL; IFN-α, IFN-β, IFN-κ, IFN-δ, IFN-ε, IFN-ε, and CD70; IFN-α, IFN-β, IFN-κ, IFN-δ, IFN-ε, IFN-τ, and GITRL; IFN-α, IFN-β, IFN-κ, IFN-δ, IFN-ε, IFN-τ, and LIGHT, IFN-α, IFN-β, IFN-κ, IFN-δ, IFN-ε, IFN-τ, and TIM-4, IFN-α, IFN-β, IFN-κ, IFN-δ, IFN-ε, IFN-τ, and ICAM-1; IFN-α, IFN-β, IFN-δ, IFN-ε, IFN-τ, and CD58; IFN-α, IFN-β, IFN-κ, IFN-δ, IFN-ε, IFN-τ, and SLAMF6; IFN-α, IFN-β, IFN-κ, IFN-δ, IFN-ε, IFN-τ, IFN-ω, and CD40-L; IFN-α, IFN-β, IFN-κ, IFN-δ, IFN-ε, IFN-τ, IFN-ω, and MEM40; IFN-α, IFN-β, IFN-κ, IFN-δ, IFN-ε, IFN-τ, IFN-ω, and B7-1(CD80)/B7-2(CD86); IFN-α, IFN-β, IFN-κ, IFN-δ, IFN-ε, IFN-τ, IFN-ω, and OX40L; IFN-α, IFN-β, IFN-κ, IFN-δ, IFN-ε, IFN-ε, IFN-ω, and 4-1BBL; IFN-α, IFN-β, IFN-κ, IFN-δ, IFN-ε, IFN-τ, IFN-ω, and CD70; IFN-α, IFN-β, IFN-κ, IFN-δ, IFN-ε, IFN-τ, IFN-ω, and GITRL; IFN-α, IFN-β, IFN-κ, IFN-δ, IFN-ε, IFN-ε, IFN-ω, and LIGHT; IFN-α, IFN-β, IFN-κ, IFN-δ, IFN-ε, IFN-τ, IFN-ω, and TIM-4; IFN-α, IFN-β, IFN-κ, IFN-δ, IFN-ε, IFN-τ, IFN-ω, and ICAM-1; IFN-α, IFN-β, IFN-δ, IFN-ε, IFN-τ, IFN-ω, and CD58; IFN-α, IFN-β, IFN-κ, IFN-δ, IFN-ε, IFN-τ, IFN-ω, and SLAMF6; IFN-α, IFN-β, IFN-κ, IFN-δ, IFN-ε, IFN-τ, IFN-ω, IFN-ζ, and CD40-L; IFN-α, IFN-β, IFN-κ, IFN-δ, IFN-ε, IFN-τ, IFN-ω, IFN-ζ, and MEM40; IFN-α, IFN-β, IFN-κ, IFN-δ, IFN-ε, IFN-τ, IFN-ω, IFN-ζ, and B7-1(CD80)/B7-2(CD86); IFN-α, IFN-β, IFN-κ, IFN-δ, IFN-ε, IFN-τ, IFN-ω, IFN-ζ, and OX40L; IFN-α, IFN-β, IFN-κ, IFN-δ, IFN-ε, IFN-τ, IFN-ω, IFN-ζ, and 4-1BBL; IFN-α, IFN-β, IFN-κ, IFN-δ, IFN-ε, IFN-τ, IFN-ω, IFN-ζ, and CD70; IFN-α, IFN-β, IFN-κ, IFN-δ, IFN-ε, IFN-τ, IFN-ω, IFN-ζ, and GITRL; IFN-α, IFN-β, IFN-κ, IFN-δ, IFN-ε, IFN-τ, IFN-ω, IFN-ζ, and LIGHT; IFN-α, IFN-β, IFN-κ, IFN-δ, IFN-ε, IFN-τ, IFN-ω, IFN-ζ, and TIM-4; IFN-α, IFN-β, IFN-κ, IFN-δ, IFN-ε, IFN-τ, IFN-ω, IFN-ζ, and ICAM-1; IFN-α, IFN-β, IFN-δ, IFN-ε, IFN-τ, IFN-ω, IFN-ζ, and CD58; IFN-α, IFN-β, IFN-κ, IFN-δ, IFN-ε, IFN-τ, IFN-ω, IFN-ζ, and SLAMF6; IFN-β and CD40-L; IFN-β and MEM40; IFN-β and B7-1(CD80)/B7-2(CD86); IFN-β and OX40L; IFN-β and 4-1BBL; IFN-β and CD70; IFN-β and GITRL; IFN-β and LIGHT; IFN-β and TIM-4; IFN-β and ICAM-1; IFN-β and CD58; IFN-β and SLAMF6; IFN-κ and CD40-L; IFN-κ and MEM40; IFN-κ and B7-1(CD80)/B7-2(CD86); IFN-κ and OX40L; IFN-κ and 4-1BBL; IFN-κ and CD70; IFN-κ and GITRL; IFN-κ and LIGHT; IFN-κ and TIM-4; IFN-κ and ICAM-1; IFN-κ and CD58; IFN-α and SLAMF6; IFN-δ and CD40-L; IFN-δ and MEM40; IFN-8 and B7-1(CD80)/B7-2(CD86); IFN-δ and OX40L; IFN-δ and 4-1BBL; IFN-δ and CD70; IFN-δ and GITRL; IFN-δ and LIGHT; IFN-δ and TIM-4; IFN-δ and ICAM-1; IFN-δ and CD58; IFN-δ and SLAMF6; IFN-ε and CD40-L; IFN-ε and MEM40; IFN-ε and B7-1(CD80)/B7-2(CD86); IFN-ε and OX40L; IFN-ε and 4-1BBL; IFN-ε and CD70; IFN-ε and GITRL; IFN-ε and LIGHT; IFN-ε and TIM-4; IFN-ε and ICAM-1; IFN-ε and CD58; IFN-ε and SLAMF6; IFN-τ and CD40-L; IFN-τ and MEM40; IFN-τ and B7-1(CD80)/B7-2(CD86); IFN-τ and OX40L; IFN-τ and 4-1BBL; IFN-τ and CD70; IFN-τ and GITRL; IFN-τ and LIGHT; IFN-τ and TIM-4; IFN-τ and ICAM-1; IFN-τ and CD58; IFN-τ and SLAMF6; IFN-ω and CD40-L; IFN-ω and MEM40; IFN-ω and B7-1(CD80)/B7-2(CD86); IFN-ω and OX40L; IFN-ω and 4-1BBL; IFN-ω and CD70; IFN-ω and GITRL; IFN-ω and LIGHT; IFN-ω and TIM-4; IFN-ω and ICAM-1; IFN-ω and CD58; IFN-ω and SLAMF6; IFN-ζ and CD40-L; IFN-ζ and MEM40; IFN-ζ and B7-1(CD80)/B7-2(CD86); IFN-ζ and OX40L; IFN-ζ and 4-1BBL; IFN-ζ and CD70; IFN-ζ and GITRL; IFN-ζ and LIGHT; IFN-ζ and TIM-4; IFN-ζ and ICAM-1; IFN-ζ and CD58; IFN-ζ and SLAMF6; IFN-β, CD40-L, and MEM40; IFN-β, CD40-L, and B7-1(CD80)/B7-2(CD86); IFN-β, CD40-L, and OX40L; IFN-β, CD40-L, and 4-1BBL; IFN-β, CD40-L, and CD70; IFN-β, CD40-L, and GITRL; IFN-β, CD40-L, and LIGHT; IFN-β, CD40-L, and TIM-4; IFN-β, CD40-L, and ICAM-1; IFN-β, CD40-L, and CD58; IFN-β, CD40-L, and SLAMF6; IFN-β, CD40-L, MEM40, and B7-1(CD80)/B7-2(CD86); IFN-β, CD40-L, MEM40, and OX40L; IFN-β, CD40-L, MEM40, and 4-1BBL; IFN-β, CD40-L, MEM40, and CD70; IFN-β, CD40-L, MEM40, and GITRL; IFN-β, CD40-L, MEM40, and LIGHT; IFN-β, CD40-L, MEM40, and TIM-4; IFN-β, CD40-L, MEM40, and ICAM-1; IFN-β, CD40-L, MEM40, and CD58; IFN-β, CD40-L, MEM40, and SLAMF6; IFN-β, CD40-L, MEM40, B7-1(CD80)/B7-2(CD86), and OX40L; IFN-β, CD40-L, MEM40, B7-1(CD80)/B7-2(CD86), and 4-1BBL; IFN-β, CD40-L, MEM40, B7-1(CD80)/B7-2(CD86), and CD70; IFN-β, CD40-L, MEM40, B7-1(CD80)/B7-2(CD86), and GITRL; IFN-β, CD40-L, MEM40, B7-1(CD80)/B7-2(CD86), and LIGHT; IFN-β, CD40-L, MEM40, B7-1(CD80)/B7-2(CD86), and TIM-4; IFN-β, CD40-L, MEM40, B7-1(CD80)/B7-2(CD86), and ICAM-1; IFN-β, CD40-L, MEM40, B7-1(CD80)/B7-2(CD86), and CD58; IFN-β, CD40-L, MEM40, B7-1(CD80)/B7-2(CD86), and SLAMF6; IFN-β, CD40-L, MEM40, B7-1(CD80)/B7-2(CD86), OX40L, and 4-1BBL; IFN-β, CD40-L, MEM40, B7-1(CD80)/B7-2(CD86), OX40L, and CD70; IFN-β, CD40-L, MEM40, B7-1(CD80)/B7-2(CD86), OX40L, and GITRL; IFN-β, CD40-L, MEM40, B7-1(CD80)/B7-2(CD86), OX40L, and LIGHT; IFN-β, CD40-L, MEM40, B7-1(CD80)/B7-2(CD86), OX40L, and TIM-4; IFN-β, CD40-L, MEM40, B7-1(CD80)/B7-2(CD86), OX40L, and ICAM-1; IFN-β, CD40-L, MEM40, B7-1(CD80)/B7-2(CD86), OX40L, and CD58; IFN-β, CD40-L, MEM40, B7-1(CD80)/B7-2(CD86), OX40L, and SLAMF6; IFN-β, CD40-L, MEM40, B7-1(CD80)/B7-2(CD86), OX40L, 4-1BBL, and CD70; IFN-β, CD40-L, MEM40, B7-1(CD80)/B7-2(CD86), OX40L, 4-1BBL, and GITRL; IFN-β, CD40-L, MEM40, B7-1(CD80)/B7-2(CD86), OX40L, 4-1BBL, and LIGHT; IFN-β, CD40-L, MEM40, B7-1(CD80)/B7-2(CD86), OX40L, 4-1BBL, and TIM-4; IFN-β, CD40-L, MEM40, B7-1(CD80)/B7-2(CD86), OX40L, 4-1BBL, and ICAM-1; IFN-β, CD40-L, MEM40, B7-1(CD80)/B7-2(CD86), OX40L, 4-1BBL, and CD58; IFN-β, CD40-L, MEM40, B7-1(CD80)/B7-2(CD86), OX40L, 4-1BBL, and SLAMF6; IFN-β, CD40-L, MEM40, B7-1(CD80)/B7-2(CD86), OX40L, 4-1BBL, CD70, and GITRL; IFN-β, CD40-L, MEM40, B7-1(CD80)/B7-2(CD86), OX40L, 4-1BBL, CD70, and LIGHT; IFN-β, CD40-L, MEM40, B7-1(CD80)/B7-2(CD86), OX40L, 4-1BBL, CD70, and TIM-4; IFN-β, CD40-L, MEM40, B7-1(CD80)/B7-2(CD86), OX40L, 4-1BBL, CD70, and ICAM-1; IFN-β, CD40-L, MEM40, B7-1(CD80)/B7-2(CD86), OX40L, 4-1BBL, CD70, and CD58; IFN-β, CD40-L, MEM40, B7-1(CD80)/B7-2(CD86), OX40L, 4-1BBL, CD70, and SLAMF6; IFN-β, CD40-L, MEM40, B7-1(CD80)/B7-2(CD86), OX40L, 4-1BBL, CD70, GITRL, and LIGHT; IFN-β, CD40-L, MEM40, B7-1(CD80)/B7-2(CD86), OX40L, 4-1BBL, CD70, GITRL, and TIM-4; IFN-β, CD40-L, MEM40, B7-1(CD80)/B7-2(CD86), OX40L, 4-1BBL, CD70, GITRL, and ICAM-1; IFN-β, CD40-L, MEM40, B7-1(CD80)/B7-2(CD86), OX40L, 4-1BBL, CD70, GITRL, and CD58; IFN-β, CD40-L, MEM40, B7-1(CD80)/B7-2(CD86), OX40L, 4-1BBL, CD70, GITRL, and SLAMF6; IFN-β, CD40-L, MEM40, B7-1(CD80)/B7-2(CD86), OX40L, 4-1BBL, CD70, GITRL, LIGHT, and TIM-4; IFN-β, CD40-L, MEM40, B7-1(CD80)/B7-2(CD86), OX40L, 4-1BBL, CD70, GITRL, LIGHT and ICAM-1; IFN-β, CD40-L, MEM40, B7-1(CD80)/B7-2(CD86), OX40L, 4-1BBL, CD70, GITRL, LIGHT and CD58; IFN-β, CD40-L, MEM40, B7-1(CD80)/B7-2(CD86), OX40L, 4-1BBL, CD70, GITRL, LIGHT and SLAMF6; IFN-β, CD40-L, MEM40, B7-1(CD80)/B7-2(CD86), OX40L, 4-1BBL, CD70, GITRL, LIGHT, TIM-4, and ICAM-1; IFN-β, CD40-L, MEM40, B7-1(CD80)/B7-2(CD86), OX40L, 4-1BBL, CD70, GITRL, LIGHT, TIM-4, and CD58; IFN-β, CD40-L, MEM40, B7-1(CD80)/B7-2(CD86), OX40L, 4-1BBL, CD70, GITRL, LIGHT, TIM-4, and SLAMF6; IFN-β, CD40-L, MEM40, B7-1(CD80)/B7-2(CD86), OX40L, 4-1BBL, CD70, GITRL, LIGHT, TIM-4, ICAM-1, and CD58; IFN-β, CD40-L, MEM40, B7-1(CD80)/B7-2(CD86), OX40L, 4-1BBL, CD70, GITRL, LIGHT, TIM-4, ICAM-1, and SLAMF6; IFN-β, CD40-L, MEM40, B7-1(CD80)/B7-2(CD86), OX40L, 4-1BBL, CD70, GITRL, LIGHT, TIM-4, ICAM-1, CD58, and SLAMF6; IFN-β, MEM40; IFN-β, MEM40, and B7-1(CD80)/B7-2(CD86); IFN-β, MEM40, and OX40L; IFN-β, MEM40, and 4-1BBL; IFN-β, MEM40, and CD70; IFN-β, MEM40, and GITRL; IFN-β, MEM40, and LIGHT; IFN-β, MEM40, and TIM-4; IFN-β, MEM40, and ICAM-1; IFN-β, MEM40, and CD58; IFN-β, MEM40, and SLAMF6; IFN-β, MEM40, and B7-1(CD80)/B7-2(CD86); IFN-β, MEM40, and OX40L; IFN-β, MEM40, and 4-1BBL; IFN-β, MEM40, and CD70; IFN-β, MEM40, and GITRL; IFN-β, MEM40, and LIGHT; IFN-β, MEM40, and TIM-4; IFN-β, MEM40, and ICAM-1; IFN-β, MEM40, and CD58; IFN-β, MEM40, and SLAMF6; IFN-β, MEM40, B7-1(CD80)B7-2(CD86), and OX40L; IFN-β, MEM40, B7-1(CD80)/B7-2(CD86), and 4-1BBL; IFN-β, MEM40, B7-1(CD80)/B7-2(CD86), and CD70; IFN-β, MEM40, B7-1(CD80)/B7-2(CD86), and GITRL; IFN-β, MEM40, B7-1(CD80)/B7-2(CD86), and LIGHT; IFN-β, MEM40, B7-1(CD80)/B7-2(CD86), and TIM-4; IFN-β, MEM40, B7-1(CD80)/B7-2(CD86), and ICAM-1; IFN-β, MEM40, B7-1(CD80)/B7-2(CD86), and CD58; IFN-β, MEM40, B7-1(CD80)/B7-2(CD86), and SLAMF6; IFN-β, MEM40, B7-1(CD80)/B7-2(CD86), OX40L, and 4-1BBL; IFN-β, MEM40, B7-1(CD80)/B7-2(CD86), OX40L, and CD70; IFN-β, MEM40, B7-1(CD80)/B7-2(CD86), OX40L, and GITRL; IFN-β, MEM40, B7-1(CD80)/B7-2(CD86), OX40L, and LIGHT; IFN-β, MEM40, B7-1(CD80)/B7-2(CD86), OX40L, and TIM-4; IFN-β, MEM40, B7-1(CD80)/B7-2(CD86), OX40L, and ICAM-1; IFN-β, MEM40, B7-1(CD80)/B7-2(CD86), OX40L, and CD58; IFN-β, MEM40, B7-1(CD80)/B7-2(CD86), OX40L, and SLAMF6; IFN-β, MEM40, B7-1(CD80)/B7-2(CD86), OX40L, 4-1BBL, and CD70; IFN-β, MEM40, B7-1(CD80)/B7-2(CD86), OX40L, 4-1BBL, and GITRL, IFN-β, MEM40, B7-1(CD80)/B7-2(CD86), OX40L, 4-1BBL, and LIGHT; IFN-β, MEM40, B7-1(CD80)/B7-2(CD86), OX40L, 4-1BBL, and TIM-4; IFN-β, MEM40, B7-1(CD80)/B7-2(CD86), OX40L, 4-1BBL, and ICAM-1; IFN-β, MEM40, B7-1(CD80)/B7-2(CD86), OX40L, 4-1BBL, and CD58; IFN-β, MEM40, B7-1(CD80)/B7-2(CD86), OX40L, 4-1BBL, and SLAMF6; IFN-β, MEM40, B7-1(CD80)/B7-2(CD86), OX40L, 4-1BBL, CD70, and GITRL; IFN-β, MEM40, B7-1(CD80)/B7-2(CD86), OX40L, 4-1BBL, CD70, and LIGHT; IFN-β, MEM40, B7-1(CD80)/B7-2(CD86), OX40L, 4-1BBL, CD70, and TIM-4; IFN-β, MEM40, B7-1(CD80)/B7-2(CD86), OX40L, 4-1BBL, CD70, and ICAM-1; IFN-β, MEM40, B7-1(CD80)/B7-2(CD86), OX40L, 4-1BBL, CD70, and CD58; IFN-β, MEM40, B7-1(CD80)/B7-2(CD86), OX40L, 4-1BBL, CD70, and SLAMF6; IFN-β, MEM40, B7-1(CD80)/B7-2(CD86), OX40L, 4-1BBL, CD70, GITRL, and LIGHT; IFN-β, MEM40, B7-1(CD80)/B7-2(CD86), OX40L, 4-1BBL, CD70, GITRL, and TIM-4; IFN-β, MEM40, B7-1(CD80)/B7-2(CD86), OX40L, 4-1BBL, CD70, GITRL, and ICAM-1; IFN-β, MEM40, B7-1(CD80)/B7-2(CD86), OX40L, 4-1BBL, CD70, GITRL, and CD58; IFN-β, MEM40, B7-1(CD80)/B7-2(CD86), OX40L, 4-1BBL, CD70, GITRL, and SLAMF6; IFN-β, MEM40, B7-1(CD80)/B7-2(CD86), OX40L, 4-1BBL, CD70, GITRL, LIGHT, and TIM-4; IFN-β, MEM40, B7-1(CD80)/B7-2(CD86), OX40L, 4-1BBL, CD70, GITRL, LIGHT and ICAM-1; IFN-β, MEM40, B7-1(CD80)/B7-2(CD86), OX40L, 4-1BBL, CD70, GITRL, LIGHT and CD58; IFN-β, MEM40, B7-1(CD80)/B7-2(CD86), OX40L, 4-1BBL, CD70, GITRL, LIGHT and SLAMF6; IFN-β, MEM40, B7-1(CD80)/B7-2(CD86), OX40L, 4-1BBL, CD70, GITRL, LIGHT, TIM-4, and ICAM-1; IFN-β, MEM40, B7-1(CD80)/B7-2(CD86), OX40L, 4-1BBL, CD70, GITRL, LIGHT, TIM-4, and CD58; IFN-β, MEM40, B7-1(CD80)B7-2(CD86), OX40L, 4-1BBL, CD70, GITRL, LIGHT, TIM-4, and SLAMF6; IFN-β, MEM40, B7-1(CD80)/B7-2(CD86), OX40L, 4-1BBL, CD70, GITRL, LIGHT, TIM-4, ICAM-1, and CD58; IFN-β, MEM40, B7-1(CD80)/B7-2(CD86), OX40L, 4-1BBL, CD70, GITRL, LIGHT, TIM-4, ICAM-1, and SLAMF6; IFN-β, MEM40, B7-1(CD80)/B7-2(CD86), OX40L, 4-1BBL, CD70, GITRL, LIGHT, TIM-4, ICAM-1, CD58, and SLAMF6; IFN-β, and B7-1(CD80)/B7-2(CD86); IFN-β, B7-1(CD80)/B7-2(CD86), and OX40L; IFN-β, B7-1(CD80)/B7-2(CD86), and 4-1BBL; IFN-β, B7-1(CD80)/B7-2(CD86), and CD70; IFN-β, B7-1(CD80)/B7-2(CD86), and GITRL; IFN-β, B7-1(CD80)/B7-2(CD86), and LIGHT; IFN-β, B7-1(CD80)/B7-2(CD86), and TIM-4; IFN-β, B7-1(CD80)/B7-2(CD86), and ICAM-1; IFN-β, B7-1(CD80)/B7-2(CD86), and CD58; IFN-β, B7-1(CD80)/B7-2(CD86), and SLAMF6; IFN-β, B7-1(CD80)/B7-2(CD86), and OX40L; IFN-β, B7-1(CD80)/B7-2(CD86), and 4-1BBL; IFN-β, B7-1(CD80)/B7-2(CD86), and CD70; IFN-β, B7-1(CD80)/B7-2(CD86), and GITRL; IFN-β, B7-1(CD80)/B7-2(CD86), and LIGHT; IFN-β, B7-1(CD80)/B7-2(CD86), and TIM-4; IFN-β, B7-1(CD80)/B7-2(CD86), and ICAM-1; IFN-β, B7-1(CD80)/B7-2(CD86), and CD58; IFN-β, B7-1(CD80)/B7-2(CD86), and SLAMF6; IFN-β, B7-1(CD80)/B7-2(CD86), OX40L, and 4-1BBL; IFN-β, B7-1(CD80)/B7-2(CD86), OX40L, and CD70; IFN-β, B7-1(CD80)/B7-2(CD86), OX40L, and GITRL; IFN-β, B7-1(CD80)/B7-2(CD86), OX40L, and LIGHT; IFN-β, B7-1(CD80)/B7-2(CD86), OX40L, and TIM-4; IFN-β, B7-1(CD80)/B7-2(CD86), OX40L, and ICAM-1; IFN-β, B7-1(CD80)/B7-2(CD86), OX40L, and CD58; IFN-β, B7-1(CD80)/B7-2(CD86), OX40L, and SLAMF6; IFN-β, B7-1(CD80)/B7-2(CD86), OX40L, 4-1BBL, and CD70; IFN-β, B7-1(CD80)/B7-2(CD86), OX40L, 4-1BBL, and GITRL; IFN-β, B7-1(CD80)/B7-2(CD86), OX40L, 4-1BBL, and LIGHT; IFN-β, B7-1(CD80)/B7-2(CD86), OX40L, 4-1BBL, and TIM-4; IFN-β, B7-1(CD80)/B7-2(CD86), OX40L, 4-1BBL, and ICAM-1; IFN-β, B7-1(CD80)/B7-2(CD86), OX40L, 4-1BBL, and CD58; IFN-β, B7-1(CD80)/B7-2(CD86), OX40L, 4-1BBL, and SLAMF6; IFN-β, B7-1(CD80)/B7-2(CD86), OX40L, 4-1BBL, CD70, and GITRL; IFN-β, B7-1(CD80)/B7-2(CD86), OX40L, 4-1BBL, CD70, and LIGHT; IFN-β, B7-1(CD80)/B7-2(CD86), OX40L, 4-1BBL, CD70, and TIM-4; IFN-β, B7-1(CD80)/B7-2(CD86), OX40L, 4-1BBL, CD70, and ICAM-1; IFN-β, B7-1(CD80)/B7-2(CD86), OX40L, 4-1BBL, CD70, and CD58; IFN-β, B7-1(CD80)/B7-2(CD86), OX40L, 4-1BBL, CD70, and SLAMF6; IFN-β, B7-1(CD80)/B7-2(CD86), OX40L, 4-1BBL, CD70, GITRL, and LIGHT; IFN-β, B7-1(CD80)/B7-2(CD86), OX40L, 4-1BBL, CD70, GITRL, and TIM-4; IFN-β, B7-1(CD80)/B7-2(CD86), OX40L, 4-1BBL, CD70, GITRL, and ICAM-1; IFN-β, B7-1(CD80)/B7-2(CD86), OX40L, 4-1BBL, CD70, GITRL, and CD58; IFN-β, B7-1(CD80)/B7-2(CD86), OX40L, 4-1BBL, CD70, GITRL, and SLAMF6; IFN-β, B7-1(CD80)/B7-2(CD86), OX40L, 4-1BBL, CD70, GITRL, LIGHT, and TIM-4; IFN-β, B7-1(CD80)/B7-2(CD86), OX40L, 4-1BBL, CD70, GITRL, LIGHT and ICAM-1; IFN-β, B7-1(CD80)/B7-2(CD86), OX40L, 4-1BBL, CD70, GITRL, LIGHT and CD58; IFN-β, B7-1(CD80)/B7-2(CD86), OX40L, 4-1BBL, CD70, GITRL, LIGHT and SLAMF6; IFN-β, B7-1(CD80)/B7-2(CD86), OX40L, 4-1BBL, CD70, GITRL, LIGHT, TIM-4, and ICAM-1; IFN-β, B7-1(CD80)/B7-2(CD86), OX40L, 4-1BBL, CD70, GITRL, LIGHT, TIM-4, and CD58; IFN-β, B7-1(CD80)/B7-2(CD86), OX40L, 4-1BBL, CD70, GITRL, LIGHT, TIM-4, and SLAMF6; IFN-β, B7-1(CD80)/B7-2(CD86), OX40L, 4-1BBL, CD70, GITRL, LIGHT, TIM-4, ICAM-1, and CD58; IFN-β, B7-1(CD80)/B7-2(CD86), OX40L, 4-1BBL, CD70, GITRL, LIGHT, TIM-4, ICAM-1, and SLAMF6; IFN-β, B7-1(CD80)/B7-2(CD86), OX40L, 4-1BBL, CD70, GITRL, LIGHT, TIM-4, ICAM-1, CD58, and SLAMF6; IFN-0, and OX40L; IFN-β, OX40L, and 4-1BBL; IFN-β, OX40L, and CD70; IFN-β, OX40L, and GITRL; IFN-β, OX40L, and LIGHT; IFN-β, OX40L, and TIM-4; IFN-β, OX40L, and ICAM-1; IFN-β, OX40L, and CD58; IFN-β, OX40L, and SLAMF6; IFN-β, OX40L, and 4-1BBL; IFN-β, OX40L, and CD70; IFN-β, OX40L, and GITRL; IFN-β, OX40L, and LIGHT; IFN-β, OX40L, and TIM-4; IFN-β, OX40L, and ICAM-1; IFN-β, OX40L, and CD58; IFN-β, OX40L, and SLAMF6; IFN-β, OX40L, 4-1BBL, and CD70; IFN-β, OX40L, 4-1BBL, and GITRL; IFN-β, OX40L, 4-1BBL, and LIGHT; IFN-β, OX40L, 4-1BBL, and TIM-4; IFN-β, OX40L, 4-1BBL, and ICAM-1, IFN-β, OX40L, 4-1BBL, and CD58; IFN-β, OX40L, 4-1BBL, and SLAMF6; IFN-β, OX40L, 4-1BBL, CD70, and GITRL; IFN-β, OX40L, 4-1BBL, CD70, and LIGHT; IFN-β, OX40L, 4-1BBL, CD70, and TIM-4; IFN-β, OX40L, 4-1BBL, CD70, and ICAM-1; IFN-β, OX40L, 4-1BBL, CD70, and CD58; IFN-β, OX40L, 4-1BBL, CD70, and SLAMF6; IFN-β, OX40L, 4-1BBL, CD70, GITRL, and LIGHT; IFN-β, OX40L, 4-1BBL, CD70, GITRL, and TIM-4; IFN-β, OX40L, 4-1BBL, CD70, GITRL, and ICAM-1; IFN-β, OX40L, 4-1BBL, CD70, GITRL, and CD58; IFN-β, OX40L, 4-1BBL, CD70, GITRL, and SLAMF6; IFN-β, OX40L, 4-1BBL, CD70, GITRL, LIGHT, and TIM-4; IFN-β, OX40L, 4-1BBL, CD70, GITRL, LIGHT and ICAM-1; IFN-β, OX40L, 4-1BBL, CD70, GITRL, LIGHT and CD58; IFN-β, OX40L, 4-1BBL, CD70, GITRL, LIGHT and SLAMF6; IFN-β, OX40L, 4-1BBL, CD70, GITRL, LIGHT, TIM-4, and ICAM-1; IFN-β, OX40L, 4-1BBL, CD70, GITRL, LIGHT, TIM-4, and CD58; IFN-β, OX40L, 4-1BBL, CD70, GITRL, LIGHT, TIM-4, and SLAMF6; IFN-β, OX40L, 4-1BBL, CD70, GITRL, LIGHT, TIM-4, ICAM-1, and CD58; IFN-β, OX40L, 4-1BBL, CD70, GITRL, LIGHT, TIM-4, ICAM-1, and SLAMF6; IFN-β, OX40L, 4-1BBL, CD70, GITRL, LIGHT, TIM-4, ICAM-1, CD58, and SLAMF6; IFN-β, and 4-1BBL; IFN-β, 4-1BBL, and CD70; IFN-β, 4-1BBL, and GITRL; IFN-β, 4-1BBL, and LIGHT; IFN-β, 4-1BBL, and TIM-4; IFN-β, 4-1BBL, and ICAM-1; IFN-β, 4-1BBL, and CD58; IFN-β, 4-1BBL, and SLAMF6; IFN-β, 4-1BBL, and CD70; IFN-β, 4-1BBL, and GITRL; IFN-β, 4-1BBL, and LIGHT; IFN-β, 4-1BBL, and TIM-4; IFN-β, 4-1BBL, and ICAM-1; IFN-β, 4-1BBL, and CD58; IFN-β, 4-1BBL, and SLAMF6; IFN-β, 4-1BBL, CD70, and GITRL; IFN-β, 4-1BBL, CD70, and LIGHT; IFN-β, 4-1BBL, CD70, and TIM-4; IFN-β, 4-1BBL, CD70, and ICAM-1; IFN-β, 4-1BBL, CD70, and CD58; IFN-β, 4-1BBL, CD70, and SLAMF6; IFN-β, 4-1BBL, CD70, GITRL, and LIGHT; IFN-β, 4-1BBL, CD70, GITRL, and TIM-4; IFN-β, 4-1BBL, CD70, GITRL, and ICAM-1; IFN-β, 4-1BBL, CD70, GITRL, and CD58; IFN-β, 4-1BBL, CD70, GITRL, and SLAMF6; IFN-β, 4-1BBL, CD70, GITRL, LIGHT, and TIM-4; IFN-β, 4-1BBL, CD70, GITRL, LIGHT and ICAM-1; IFN-β, 4-1BBL, CD70, GITRL, LIGHT and CD58; IFN-β, 4-1BBL, CD70, GITRL, LIGHT and SLAMF6; IFN-β, 4-1BBL, CD70, GITRL, LIGHT, TIM-4, and ICAM-1; IFN-β, 4-1BBL, CD70, GITRL, LIGHT, TIM-4, and CD58; IFN-β, 4-1BBL, CD70, GITRL, LIGHT, TIM-4, and SLAMF6; IFN-β, 4-1BBL, CD70, GITRL, LIGHT, TIM-4, ICAM-1, and CD58; IFN-β, 4-1BBL, CD70, GITRL, LIGHT, TIM-4, ICAM-1, and SLAMF6; IFN-β, 4-1BBL, CD70, GITRL, LIGHT, TIM-4, ICAM-1, CD58, and SLAMF6; IFN-β and CD70; IFN-β, CD70, and GITRL; IFN-β, CD70, and LIGHT; IFN-β, CD70, and TIM-4; IFN-β, CD70, and ICAM-1; IFN-β, CD70, and CD58; IFN-β, CD70, and SLAMF6; IFN-β, CD70, and GITRL; IFN-β, CD70, and LIGHT; IFN-β, CD70, and TIM-4; IFN-0, CD70, and ICAM-1; IFN-β, CD70, and CD58; IFN-β, CD70, and SLAMF6; IFN-β, CD70, GITRL, and LIGHT; IFN-β, CD70, GITRL, and TIM-4; IFN-β, CD70, GITRL, and ICAM-1; IFN-β, CD70, GITRL, and CD58; IFN-β, CD70, GITRL, and SLAMF6; IFN-β, CD70, GITRL, LIGHT, and TIM-4; IFN-β, CD70, GITRL, LIGHT and ICAM-1; IFN-β, CD70, GITRL, LIGHT and CD58; IFN-β, CD70, GITRL, LIGHT and SLAMF6; IFN-β, CD70, GITRL, LIGHT, TIM-4, and ICAM-1; IFN-β, CD70, GITRL, LIGHT, TIM-4, and CD58; IFN-β, CD70, GITRL, LIGHT, TIM-4, and SLAMF6; IFN-β, CD70, GITRL, LIGHT, TIM-4, ICAM-1, and CD58; IFN-β, CD70, GITRL, LIGHT, TIM-4, ICAM-1, and SLAMF6; IFN-β, CD70, GITRL, LIGHT, TIM-4, ICAM-1, CD58, and SLAMF6; IFN-β and GITRL; IFN-β, GITRL, and LIGHT; IFN-β, GITRL, and TIM-4; IFN-β, GITRL, and ICAM-1; IFN-β, GITRL, and CD58; IFN-β, GITRL, and SLAMF6; IFN-β, GITRL, and LIGHT; IFN-β, GITRL, and TIM-4; IFN-β, GITRL, and ICAM-1; IFN-β, GITRL, and CD58; IFN-β, GITRL, and SLAMF6; IFN-β, GITRL, LIGHT, and TIM-4; IFN-β, GITRL, LIGHT and ICAM-1; IFN-β, GITRL, LIGHT and CD58; IFN-β, GITRL, LIGHT and SLAMF6; IFN-β, GITRL, LIGHT, TIM-4, and ICAM-1; IFN-β, GITRL, LIGHT, TIM-4, and CD58; IFN-β, GITRL, LIGHT, TIM-4, and SLAMF6; IFN-β, GITRL, LIGHT, TIM-4, ICAM-1, and CD58; IFN-β, GITRL, LIGHT, TIM-4, ICAM-1, and SLAMF6; IFN-β, GITRL, LIGHT, TIM-4, ICAM-1, CD58, and SLAMF6; IFN-β, and LIGHT; IFN-β, LIGHT, and TIM-4; IFN-β, LIGHT, and ICAM-1; IFN-β, LIGHT, and CD58; IFN-β, LIGHT, and SLAMF6; IFN-β, LIGHT, and TIM-4; IFN-β, LIGHT and ICAM-1; IFN-β, LIGHT and CD58; IFN-β, LIGHT and SLAMF6; IFN-β, LIGHT, TIM-4, and ICAM-1; IFN-β, LIGHT, TIM-4, and CD58; IFN-β, LIGHT, TIM-4, and SLAMF6; IFN-β, LIGHT, TIM-4, ICAM-1, and CD58; IFN-β, LIGHT, TIM-4, ICAM-1, and SLAMF6; IFN-β, LIGHT, TIM-4, ICAM-1, CD58, and SLAMF6; IFN-β, and TIM-4; IFN-β, TIM-4, and ICAM-1; IFN-β, TIM-4, and CD58; IFN-β, TIM-4, and SLAMF6; IFN-β, TIM-4, and ICAM-1; IFN-β, TIM-4, and CD58; IFN-β, TIM-4, and SLAMF6; IFN-β, TIM-4, ICAM-1, and CD58; IFN-β, TIM-4, ICAM-1, and SLAMF6; IFN-β, TIM-4, ICAM-1, CD58, and SLAMF6; IFN-β and ICAM-1; IFN-β, ICAM-1, and CD58; IFN-β, ICAM-1, and SLAMF6; IFN-β, ICAM-1, and CD58; IFN-β, ICAM-1, and SLAMF6; IFN-β, ICAM-1, CD58, and SLAMF6; IFN-β and CD58; IFN-β, CD58, and SLAMF6; IFN-β and SLAMF6; IFN-β, IFN-κ, and CD40-L; IFN-β, IFN-κ, and MEM40; IFN-β, IFN-κ, and B7-1(CD80)/B7-2(CD86); IFN-β, IFN-κ, and OX40L; IFN-β, IFN-κ, and 4-1BBL; IFN-β, IFN-κ, and CD70; IFN-β, IFN-κ, and GITRL; IFN-β, IFN-κ, and LIGHT; IFN-β, IFN-κ, and TIM-4; IFN-β, IFN-κ, and ICAM-1; IFN-0, and CD58; IFN-β, IFN-κ, and SLAMF6; IFN-β, IFN-κ, IFN-δ, and CD40-L; IFN-β, IFN-κ, IFN-δ, and MEM40; IFN-β, IFN-κ, IFN-δ, and B7-1(CD80)/B7-2(CD86); IFN-β, IFN-κ, IFN-δ, and OX40L; IFN-β, IFN-κ, IFN-δ, and 4-1BBL; IFN-β, IFN-κ, IFN-δ, and CD70; IFN-β, IFN-κ, IFN-δ, and GITRL; IFN-β, IFN-κ, IFN-δ, and LIGHT; IFN-β, IFN-κ, IFN-δ, and TIM-4; IFN-β, IFN-κ, IFN-δ, and ICAM-1; IFN-β, IFN-δ, and CD58; IFN-β, IFN-κ, IFN-δ, and SLAMF6; IFN-β, IFN-κ, IFN-δ, IFN-ε, and CD40-L; IFN-β, IFN-κ, IFN-δ, IFN-ε, and MEM40; IFN-β, IFN-κ, IFN-δ, IFN-ε, and B7-1(CD80)/B7-2(CD86); IFN-β, IFN-κ, IFN-δ, IFN-ε, and OX40L; IFN-β, IFN-κ, IFN-δ, IFN-ε, and 4-1BBL; IFN-β, IFN-κ, IFN-δ, IFN-ε, and CD70; IFN-β, IFN-κ, IFN-δ, IFN-ε, and GITRL; IFN-β, IFN-κ, IFN-δ, IFN-ε, and LIGHT; IFN-β, IFN-κ, IFN-δ, IFN-ε, and TIM-4; IFN-β, IFN-κ, IFN-δ, IFN-ε, and ICAM-1; IFN-β, IFN-δ, IFN-ε, and CD58; IFN-β, IFN-κ, IFN-δ, IFN-ε, and SLAMF6; IFN-β, IFN-κ, IFN-δ, IFN-ε, IFN-τ, and CD40-L; IFN-β, IFN-κ, IFN-δ, IFN-ε, IFN-τ, and MEM40; IFN-β, IFN-κ, IFN-δ, IFN-ε, IFN-τ, and B7-1(CD80)/B7-2(CD86); IFN-β, IFN-κ, IFN-δ, IFN-ε, IFN-τ, and OX40L; IFN-β, IFN-κ, IFN-δ, IFN-ε, IFN-ε, and 4-1BBL; IFN-β, IFN-κ, IFN-δ, IFN-ε, IFN-τ, and CD70; IFN-β, IFN-κ, IFN-δ, IFN-ε, IFN-τ, and GITRL; IFN-β, IFN-κ, IFN-δ, IFN-ε, IFN-τ, and LIGHT; IFN-β, IFN-κ, IFN-δ, IFN-ε, IFN-τ, and TIM-4; IFN-β, IFN-κ, IFN-δ, IFN-ε, IFN-τ, and ICAM-L; IFN-β, IFN-δ, IFN-ε, IFN-τ, and CD58; IFN-β, IFN-κ, IFN-δ, IFN-ε, IFN-τ, and SLAMF6; IFN-β, IFN-κ, IFN-δ, IFN-ε, IFN-ε, IFN-ω, and CD40-L; IFN-β, IFN-κ, IFN-δ, IFN-ε, IFN-τ, IFN-ω, and MEM40; IFN-β, IFN-κ, IFN-δ, IFN-ε, IFN-τ, IFN-ω, and B7-1(CD80)/B7-2(CD86); IFN-β, IFN-κ, IFN-δ, IFN-ε, IFN-ε, IFN-ω, and OX40L; IFN-β, IFN-κ, IFN-δ, IFN-ε, IFN-τ, IFN-ω, and 4-1BBL, IFN-β, IFN-κ, IFN-δ, IFN-ε, IFN-τ, IFN-ω, and CD70; IFN-β, IFN-κ, IFN-δ, IFN-ε, IFN-τ, IFN-ω, and GITRL; IFN-β, IFN-κ, IFN-δ, IFN-ε, IFN-τ, IFN-ω, and LIGHT; IFN-β, IFN-κ, IFN-δ, IFN-ε, IFN-τ, IFN-ω, and TIM-4; IFN-β, IFN-κ, IFN-δ, IFN-ε, IFN-τ, IFN-ω, and ICAM-1; IFN-β, IFN-δ, IFN-ε, IFN-τ, IFN-ω, and CD58; IFN-β, IFN-κ, IFN-δ, IFN-ε, IFN-τ, IFN-ω, and SLAMF6; IFN-β, IFN-κ, IFN-δ, IFN-ε, IFN-τ, IFN-ω, IFN-ζ, and CD40-L; IFN-β, IFN-κ, IFN-δ, IFN-ε, IFN-τ, IFN-ω, IFN-ζ, and MEM40; IFN-β, IFN-κ, IFN-δ, IFN-ε, IFN-τ, IFN-ω, IFN-ζ, and B7-1(CD80)/B7-2(CD86); IFN-β, IFN-κ, IFN-δ, IFN-ε, IFN-τ, IFN-ω, IFN-ζ, and OX40L; IFN-β, IFN-κ, IFN-δ, IFN-ε, IFN-τ, IFN-ω, IFN-ζ, and 4-1BBL; IFN-β, IFN-κ, IFN-δ, IFN-ε, IFN-τ, IFN-ω, IFN-ζ, and CD70; IFN-β, IFN-κ, IFN-δ, IFN-ε, IFN-τ, IFN-ω, IFN-ζ, and GITRL; IFN-β, IFN-κ, IFN-δ, IFN-ε, IFN-τ, IFN-ω, IFN-ζ, and LIGHT; IFN-β, IFN-κ, IFN-δ, IFN-ε, IFN-τ, IFN-ω, IFN-ζ, and TIM-4; IFN-β, IFN-κ, IFN-δ, IFN-ε, IFN-τ, IFN-ω, IFN-ζ, and ICAM-1; IFN-β, IFN-δ, IFN-ε, IFN-τ, IFN-ω, IFN-ζ, and CD58; and/or IFN-β, IFN-κ, IFN-δ, IFN-ε, IFN-τ, IFN-ω, IFN-ζ, and SLAMF6. In one aspect, the oncolytic viruses further comprising IL-12 and/or IL-23. Alternatively, disclosed herein are oncolytic viruses that express i) one or more type 1 interferon (IFN) (such as, for example, IFN-α, IFN-β, IFN-κ, IFN-δ, IFN-ε, IFN-τ, IFN-ω, and/or IFN-ζ) or one or more exogenous immunostimulatory molecules (such as, for example, CD40-L, MEM40, B7-1(CD80)/B7-2(CD86), OX40L, 4-1BBL, CD70, GITRL, LIGHT, TIM-4, ICAM-1, CD58 and/or SLAMF6) and/or ii) an interleukin selected from IL-12 and/or IL-23. For example, the oncolytic virus for use in the disclosed methods can comprise IFN-α and IL-12, IFN-β and IL-12, IFN-κ and IL-12, IFN-δ and IL-12, IFN-ε and IL-12, IFN-τ and IL-12, IFN-ω and IL-12, IFN-ζ and IL-12, CD40-L and IL-12, MEM40 and IL-12, B7-1(CD80)/B7-2(CD86) and IL-12, OX40L and IL-12, 4-1BBL and IL-12, CD70 and IL-12, GITRL and IL-12, LIGHT and IL-12, TIM-4 and IL-12, ICAM-1 and IL-12, CD58 and IL-12, SLAMF6 and IL-12, IFN-α and IL-23, IFN-β and IL-23, IFN-κ and IL-23, IFN-δ and IL-23, IFN-ε and IL-23, IFN-τ and IL-23, IFN-ω and IL-23, IFN-ζ and IL-23, CD40-L and IL-23, MEM40 and IL-23, B7-1(CD80)/B7-2(CD86) and IL-23, OX40L and IL-23, 4-1BBL and IL-23, CD70 and IL-23, GITRL and IL-23, LIGHT and IL-23, TIM-4 and IL-23, ICAM-1 and IL-23, CD58 and IL-23, SLAMF6 and IL-23, IFN-α, IL-12, and IL-23, IFN-β, IL-12, and IL-23, IFN-κ, IL-12, and IL-23, IFN-δ, IL-12, and IL-23, IFN-ε, IL-12, and IL-23, IFN-τ, IL-12, and IL-23, IFN-ω, IL-12, and IL-23, IFN-ζ, IL-12, and IL-23, CD40-L, IL-12, and IL-23, MEM40, IL-12, and IL-23, B7-1(CD80)/B7-2(CD86), IL-12, and IL-23, OX40L, IL-12, and IL-23, 4-1BBL, IL-12, and IL-23, CD70, IL-12, and IL-23, GITRL, IL-12, and IL-23, LIGHT, IL-12, and IL-23, TIM-4, IL-12, and IL-23, ICAM-1, IL-12, and IL-23, CD58, IL-12, and IL-23, and/or SLAMF6, IL-12, and IL-23. Any oncolytic virus may be utilized. In some embodiments, the oncolytic virus can be adenovirus, retrovirus, herpes virus, picomavirus (including coxsackievirus, poliovirus, and Seneca Valley virus), paramyxovirus (including measles virus and Newcastle disease virus (NDV)), parvovirus, rhabdovirus (including vesicular stomatitis virus (VSV)), or vaccinia virus. Preferably, the oncolytic virus is replication competent.

Further embodiments of the invention provide methods of treating a malignancy in a subject by administering the oncolytic viruses disclosed herein, in combination by administering a checkpoint inhibitor to the subject. As used herein “checkpoint inhibitors” comprise any agent that disrupts immune checkpoints including, but not limited to antibodies that block PD-1 (such as, for example, Pembrolizumab, Cemiplimab, Nivolumab (BMS-936558 or MDX1106), CT-011, and/or MK-3475), PD-L1 (such as, for example, Avelumab, Atezolizumab, Durvalumab, MDX-1105 (BMS-936559), MPDL3280A, and/or MSB0010718C), PD-L2 (such as, for example, rHIgM12B7), CTLA-4 (such as, for example, Ipilimumab (MDX-010) and/or Tremelimumab (CP-675,206)), IDO, B7-H3 (such as, for example, MGA271, MGC-018 and/or FPA-150), B7-H4 (such as, for example, MGC-018 and/or FPA-150), TIM3 (such as, for example, MBG453, Sym023, and/or TSR-022), LAG-3 (such as, for example, BMS-986016, LAG525, REGEN3767, Tebotelimab, BI 754,091, eftilagimod alpha, and/or FS 118), A2aR (such as, for example, EOS100850 and/or AB928), CD73 (such as, for example, CPI-006), NKG2A (such as, for example, monolizumab), PVRIG/PVRL2 (such as, for example, COM701), CEACAM1 (such as, for example, CM24), CEACAM 5/6 (such as, for example, NEO201), FAK (such as, for example Defactinib), CCL2/CCR2 (such as, for example PF-04136309), LIF (such as, for example MSC-1), phosphatidylserine (such as, for example Bavituximab), CLEVER-1 (such as, for example FP-1305), Ang2 (such as, for example Trebananib), SEMA4D (such as, for example Pepinemab), IL-8 (such as, for example, BMS-986253), IL-1 and IL-1R3 (such as, for example, CAN04 and/or Canakinumab), CSF-1 (such as, for example, Lacnotuzumab, LY3022855, emactuzumab, and/or pexidartinib), and/or CD47 (such as, for example, Hu5F9-G4, ALX148, TTI-662, and/or RRx-001).

IFN-α can be a human IFN-α represented by the sequence of SEQ ID NO: 1; IFN-α can be a mammalian IFN-α, such as, mouse, rat, rabbit, pig, feline, canine, or bovine IFN-α. Additional embodiments of IFN-α from other mammals are known to a skilled artisan and such embodiments are within the purview of the invention. In certain embodiments, IFN-α has between 80.00% and, up to, including 99.99% (e.g., 80, 81, 82, 83, 84, 85, 86, 87, 88, 89, 90, 91, 92, 93, 94, 95, 96, 97, 98, or 99%) sequence identity to the human wild-type IFN-α, for example, IFN-α of SEQ ID NO: 1.

IFN-β can be a human IFN-β represented by the sequence of SEQ ID NO: 2; IFN-β can be a mammalian IFN-β, such as, mouse, rat, rabbit, pig, feline, canine, or bovine IFN-β. Additional embodiments of IFN-β from other mammals are known to a skilled artisan and such embodiments are within the purview of the invention. In certain embodiments, IFN-β has between 80.00% and, up to, including 99.99% (e.g., 80, 81, 82, 83, 84, 85, 86, 87, 88, 89, 90, 91, 92, 93, 94, 95, 96, 97, 98, or 99%) sequence identity to the human wild-type IFN-β, for example, IFN-β of SEQ ID NO: 2. In a specific embodiment, IFN-β lacks the first 22 amino acids of SEQ ID NO: 2 and optionally, further has a substitution of cysteine 17 of the resultant 165 amino acid peptide with serine.

IFN-ε can be a human IFN-ε represented by the sequence of SEQ ID NO: 3; IFN-ε can be a mammalian IFN-ε, such as, mouse, rat, rabbit, pig, feline, canine, or bovine IFN-ε. Additional embodiments of IFN-ε from other mammals are known to a skilled artisan and such embodiments are within the purview of the invention. In certain embodiments, IFN-ε has between 80.00% and, up to, including 99.99% (e.g., 80, 81, 82, 83, 84, 85, 86, 87, 88, 89, 90, 91, 92, 93, 94, 95, 96, 97, 98, or 99%) sequence identity to the human wild-type IFN-ε, for example, IFN-ε of SEQ ID NO: 3.

IFN-κ can be a human IFN-κ represented by the sequence of SEQ ID NO: 4; IFN-κ can be a mammalian IFN-κ, such as, mouse, rat, rabbit, pig, feline, canine, or bovine IFN-κ. Additional embodiments of IFN-κ from other mammals are known to a skilled artisan and such embodiments are within the purview of the invention. In certain embodiments, IFN-κ has between 80.00% and, up to, including 99.99% (e.g., 80, 81, 82, 83, 84, 85, 86, 87, 88, 89, 90, 91, 92, 93, 94, 95, 96, 97, 98, or 99%) sequence identity to the human wild-type IFN-κ, for example, IFN-κ of SEQ ID NO: 4.

IFN-ω can be a human IFN-ω represented by the sequence of SEQ ID NO: 5; IFN-ω can be a mammalian IFN-ω, such as, mouse, rat, rabbit, pig, feline, canine, or bovine IFN-ω. Additional embodiments of IFN-ω from other mammals are known to a skilled artisan and such embodiments are within the purview of the invention. In certain embodiments, IFN-ω has between 80.00% and, up to, including 99.99% (e.g., 80, 81, 82, 83, 84, 85, 86, 87, 88, 89, 90, 91, 92, 93, 94, 95, 96, 97, 98, or 99%) sequence identity to the human wild-type IFN-ω, for example, IFN-ω of SEQ ID NO: 5.

CD40-L can be a human CD40-L represented by the sequence of SEQ ID NO: 6, CD40-L can be a mammalian CD40-L, such as, mouse, rat, rabbit, pig, feline, canine, or bovine CD40-L. Additional embodiments of CD40-L from other mammals are known to a skilled artisan and such embodiments are within the purview of the invention. In certain embodiments, CD40-L has between 80.00% and, up to, including 99.99% (e.g., 80, 81, 82, 83, 84, 85, 86, 87, 88, 89, 90, 91, 92, 93, 94, 95, 96, 97, 98, or 99%) sequence identity to the human wild-type CD40-L, for example, CD40-L of SEQ ID NO: 6.

The CD40-L can be a chimeric CD40-L or a non-chimeric CD40-L polypeptide. In some embodiments, the CD40-L expressed in an oncolytic virus of the invention, is a chimeric CD40-L. Such chimeric CD40-L polypeptides comprise CD40-L domains or subdomains from at least two different species, for example, human and mouse CD40-L. A chimeric CD40-L provides higher stimulation of the immune response compared to a naturally occurring CD40-L. Examples of chimeric CD40-L suitable for use in the current invention are disclosed in U.S. Pat. Nos. 7,495,090, 7,928,213, and 8,138,310. Each of these patents is incorporated herein by reference in their entirety.

In certain embodiments of chimeric CD40-L, at least one domain or subdomain of CD40-L that contains a cleavage site of human CD40-L is replaced with a corresponding domain or subdomain of non-human CD40-L, preferably murine CD40-L. In addition, a chimeric CD40-L can comprise a domain or subdomain of human CD40-L that binds to a CD40-L receptor. Domains I to IV of a human CD40-L (SEQ ID NO: 6) correspond to amino acid portions 1-14, 14-45, 46-110, and 111-261 of SEQ ID NO: 6. A skilled artisan can determine domains I to IV of a non-human CD40-L based on sequence alignment of the non-human CD40-L with the human CD40-L. Certain domain positions of non-human CD40-L are provided in Table 1 of U.S. Pat. No. 7,495,090, which is herein incorporated by reference in its entirety.

In some embodiments, the chimeric CD40-L comprises a first subdomain of non-human CD40-L, wherein the subdomain replaces a cleavage site of human CD40-L, and a second subdomain of human CD40-L that binds to a CD40-L receptor.

The first subdomain can comprise a subdomain of domain IV of a non-human CD40-L. In addition, the first subdomain can further comprise domain III, or a subdomain or domain III, of a non-human CD40-L. In certain embodiments, the first subdomain replaces a portion of a cleavage site of human CD40-L. In further embodiments, in addition to domain IV or a subdomain of domain IV, and optionally, domain III or a subdomain of domain III, a chimeric CD40-L further comprises domain II or a subdomain of domain II, of a non-human CD40-L. Furthermore, the first subdomain of a chimeric CD40-L can comprise domain I or a subdomain or domain I, of non-human CD40-L. Thus, in certain chimeric CD40-L, the first subdomain comprises domains or subdomains of domain I, II, III and IV, of a non-human CD40-L. In preferred embodiments, the non-human CD40-L is a murine CD40-L.

In preferred embodiments, the chimeric human/mouse CD40 ligand has 92% amino acid sequence homology with human CD40L (SEQ ID NO: 12) (See, U.S. Pat. No. 7,495,090, herein incorporated by reference and referred to herein as “MEM40”). “CD40 ligand” and “CD40-L” may be used interchangeably herein, and may also be referred to as “CD154”. Specifically, domains I, II and III—the regions that contain the intracellular, intra-membrane, and proximal extracellular domains, respectively, of this molecule—have been fully humanized. In domain IV, which contains the CD40 binding portion of the molecule, only those murine domains necessary for optimum CD40 ligand expression in cells are retained, MEM40 is fully humanized at the 3′ end of the molecule where antibody binding neutralizes the activity of the murine CD 154 (CD40 ligand) when administered to humans.

Non-limiting examples of chimeric CD40-L useful in the current invention comprise the following sequences:

SEQ ID NO: 7
MIETYSQPSPRSVATGLPASMKIFMYLLTVFLITQMIGSVLFAVYLHRRLDKVEEEVNLH
EDFVFIKKLKRCNKGEGSLSLLNCEEMRRQFEDLVKDITLNKEEKKENSFEMQRGDEDPQ
IAAHVVSEANSNAASVLQWAKKGYYTMKSNLVTLENGKQLTVKRQGLYYIYAQVTFCSNR
EPSSQRPFIVGLWLKPSSGSERILLKAANTHSSSQLCEQQSVHLGGVFELQPGASVFVNV
TDPSQVSHGTGFTSFGLLKL
SEQ ID NO: 8:
MIETYNQTSPRSAATGLPISMKIFMYLLTVFLITQMIGSALFAVYLHRRLDKIEDERNLH
EDFVFMKTIQRCNTGERSLSLLNCEEIKSQFEGFVKDIMLNKEETKKDEDPQIAAHVVSE
ANSNAASVLQWAKKGYYTMKSNLVTLENGKQLTVKRQGLYYIYAQVTFCSNREPSSQRPF
IVGLWLKPSSGSERILLKAANTHSSSQLCEQQSVHLGGVFELQPGASVFVNVTDPSQVSH
GTGFTSFGLLKL
SEQ ID NO: 9:
MIETYSQPSPRSVATGLPASMKIFMYLLTVFLITQMIGSVLFAVYLHRRLDKVEEEVNLH
EDFVFIKKLKRCNKGEGSLSLLNCEEMRRQFEDLVKDITLNKEEKKENSFEMQRGDEDPQ
IAAHVVSEANSNAASVLQWAKKGYYTMKSNLVTLENGKQLTVKRQGLYYIYAQVTFCSNR
EASSQAPFIVGLWLKPSSGSERILLKAANTHSSSQLCEQQSVHLGGVFELQPGASVFVNV
TDPSQVSHGTGFTSFGLLKL
SEQ ID NO: 10
MIETYNQTSPRSAATGLPISMKIFMYLLTVFLITQMIGSALFAVYLHRRLDKIEDERNLH
EDFVFMKTIQRCNTGERSLSLLNCEEIKSQFEGFVKDIMLNKEETKKDEDPQIAAHVVSE
ANSNAASVLQWAKKGYYTMKSNLVTLENGKQLTVKRQGLYYIYAQVTFCSNREASSQAPF
IVGLWLKPSSGSERILLKAANTHSSSQLCEQQSVHLGGVFELQPGASVFVNVTDPSQVSH
GTGFTSFGLLKL
SEQ ID NO: 11
MIETYSQPSPRSVATGLPASMKIFMYLLTVFLITQMIGSVLFAVYLHRRLDKVEEEVNLH
EDFVFIKKLKRCNKGEGSLSLLNCEEMRRQFEDLVKDITLNKEEKKENSFEMQRGDEDPQ
IAAHVVSEANSNAASVLQWAKKGYYTMKSNLVTLENGKQLTVKRQGLYYIYAQVTFCSNR
EASSQAPFIVGLWLKPSSGSERILLKAANTHSSSQLCEQQSIHLGGVFELQPGASVFVNV
TDPSQVSHGTGFTSFGLLKL
SEQ ID NO: 12
MIETYNQTSPRSAATGLPISMKIFMYLLTVFLITQMIGSALFAVYLHRRLDKIEDERNLH
EDFVFMKTIQRCNTGERSLSLLNCEEIKSQFEGFVKDIMLNKEETKKDEDPQIAAHVVSE
ANSNAASVLQWAKKGYYTMKSNLVTLENGKQLTVKRQGLYYIYAQVTFCSNREASSQAPF
IVGLWLKPSSGSERILLKAANTHSSSQLCEQQSIHLGGVFELQPGASVFVNVTDPSQVSH
GTGFTSFGLLKL
SEQ ID NO: 13
MIETYSQPSPRSVATGLPASMKIFMYLLTVFLITQMIGSVLFAVYLHRRLDKVEEEVNLH
EDFVFIKKLKRCNKGEGSLSLLNCEEMRRQFEDLVKDITLNKEEKKENSFEMQRGDEDPQ
IAAHVVSEANSNAASVLQWAKKGYYTMKSNLVTLENGKQLTVKRQGLYYIYAQVTFCSNR
EASSQAPFIVGLWLKPSSGSERILLKAANTHSSAKPCGQQSIHLGGVFELQPGASCFVNV
TDPSQVSHGTGFTSFGLLKL
SEQ ID NO: 14
MIETYNQTSPRSAATGLPISMKIFMYLLTVFLITQMIGSALFAVYLHRRLDKIEDERNLH
EDFVFMKTIQRCNTGERSLSLLNCEEIKSQFEGFVKDIMLNKEETKKDEDPQIAAHVVSE
ANSNAASVLQWAKKGYYTMKSNLVTLENGKQLTVKRQGLYYIYAQVTFCSNREASSQAPF
IVGLWLKPSSGSERILLKAANTHSSAKPCGQQSIHLGGVFELQPGASVFVNVTDPSQVSH
GTGFTSFGLLKL
SEQ ID NO: 15
MIETYSQPSPRSVATGLPASMKIFMYLLTVFLITQMIGSVLFAVYLHRRLDKVEEEVNLH
EDFVFIKKLKRCNKGEGSLSLLNCEEMRRQFEDLVKDITLNKEEKKENSFEMQRGDEDPQ
IAAHVVSEANSNAASVLQWAKKGYYTMKSNLVTLENGKQLTVKRQGLYYIYAQVTFCSNR
EPSSQRPFIVGLWLKPSSGSERILLKAANTHSSSQLCEQQSIHLGGVFELQPGASVFVNV
TDPSQVSHGTGFTSFGLLKL
SEQ ID NO: 16
MIETYNQTSPRSAATGLPISMKIFMYLLTVFLITQMIGSALFAVYLHRRLDKIEDERNLH
EDFVFMKTIQRCNTGERSLSLLNCEEIKSQFEGFVKDIMLNKEETKKDEDPQIAAHVVSE
ANSNAASVLQWAKKGYYTMKSNLVTLENGKQLTVKRQGLYYIYAQVTFCSNREPSSQRPF
IVGLWLKPSSGSERILLKAANTHSSSQLCEQQSIHLGGVFELQPGASVFVNVTDPSQVSH
GTGFTSFGLLKL
SEQ ID NO: 17
MIETYSQPSPRSVATGLPASMKIFMYLLTVFLITQMIGSVLFAVYLHRRLDKVEEEVNLH
EDFVFIKKLKRCNKGEGSLSLLNCEEMRRQFEDLVKDITLNKEEKKENSFEMQRGDEDPQ
IAAHVVSEANSNAASVLQWAKKGYYTMKSNLVTLENGKQLTVKRQGLYYIYAQVTFCSNR
EPSSQRPFIVGLWLKPSSGSERILLKAANTHSSAKPCGQQSIHLGGVFELQPGASVFVNV
TDPSQVSHGTGFTSFGLLKL
SEQ ID NO: 18
MIETYNQTSPRSAATGLPISMKIFMYLLTVFLITQMIGSALFAVYLHRRLDKIEDERNLH
EDFVFMKTIQRCNTGERSLSLLNCEEIKSQFEGFVKDIMLNKEETKKDEDPQIAAHVVSE
ANSNAASVLQWAKKGYYTMKSNLVTLENGKQLTVKRQGLYYIYAQVTFCSNREPSSQRPF
IVGLWLKPSSGSERILLKAANTHSSAKPCGQQSIHLGGVFELQPGASVFVNVTDPSQVSH
GTGFTSFGLLKL

Certain nucleotide sequences encoding the chimeric CD40-L of SEQ ID NOs: 7 to 18 are disclosed in U.S. Pat. Nos. 7,495,090, 7,928,213, and 8,138,310. These nucleotide sequences are incorporated herein by reference and use of such nucleotide sequences is envisioned herein.

The oncolytic viruses of the invention can comprise a nucleic acid encoding IFN-α, wherein the IFN-α comprises the human wild-type IFN-α (SEQ ID NO: 1) or a IFN-α having between 80.00% and, up to, including 99.99% (e.g., 80, 81, 82, 83, 84, 85, 86, 87, 88, 89, 90, 91, 92, 93, 94, 95, 96, 97, 98, or 99%) sequence identity to the human wild-type IFN-α, for example, IFN-α of SEQ ID NO: 1.

The oncolytic viruses of the invention can comprise a nucleic acid encoding IFN-β, wherein the IFN-β comprises the human wild-type IFN-β (SEQ ID NO: 2) or a IFN-β having between 80.00% and, up to, including 99.99% (e.g., 80, 81, 82, 83, 84, 85, 86, 87, 88, 89, 90, 91, 92, 93, 94, 95, 96, 97, 98, or 99%) sequence identity to the human wild-type IFN-β, for example, IFN-β of SEQ ID NO: 2. In a specific embodiment, the nucleotide sequence encodes for a IFN-β that lacks the first 22 amino acids of SEQ ID NO: 2 and optionally, further has a substitution of cysteine 17 of the resultant 165 amino acid peptide with serine.

The oncolytic viruses of the invention can comprise a nucleic acid encoding IFN-ε, wherein the IFN-ε comprises the human wild-type IFN-ε (SEQ ID NO: 3) or a IFN-ε having between 80.00% and, up to, including 99.99% (e.g., 80, 81, 82, 83, 84, 85, 86, 87, 88, 89, 90, 91, 92, 93, 94, 95, 96, 97, 98, or 99%) sequence identity to the human wild-type IFN-ε, for example, IFN-ε of SEQ ID NO: 3.

The oncolytic viruses of the invention can comprise a nucleic acid encoding IFN-κ, wherein the IFN-κ comprises the human wild-type IFN-κ (SEQ ID NO: 4) or a IFN-κ having between 80.00% and, up to, including 99.99% (e.g., 80, 81, 82, 83, 84, 85, 86, 87, 88, 89, 90, 91, 92, 93, 94, 95, 96, 97, 98, or 99%) sequence identity to the human wild-type IFN-κ, for example, IFN-κ of SEQ ID NO: 4.

The oncolytic viruses of the invention can comprise a nucleic acid encoding IFN-ω, wherein the IFN-ω comprises the human wild-type IFN-ω (SEQ ID NO: 5) or a IFN-ω having between 80.00% and, up to, including 99.99% (e.g., 80, 81, 82, 83, 84, 85, 86, 87, 88, 89, 90, 91, 92, 93, 94, 95, 96, 97, 98, or 99%) sequence identity to the human wild-type IFN-ω, for example, IFN-ω of SEQ ID NO: 5.

The oncolytic viruses of the invention can comprise a nucleic acid encoding CD40-L, wherein the CD40-L comprises the human wild-type CD40-L (SEQ ID NO: 6) or a CD40-L having between 80.00% and, up to, including 99.99% (e.g., 80, 81, 82, 83, 84, 85, 86, 87, 88, 89, 90, 91, 92, 93, 94, 95, 96, 97, 98, or 99%) sequence identity to the human wild-type CD40-L, for example, CD40-L of SEQ ID NO: 6. The oncolytic viruses of the invention can also comprise a nucleic acid encoding a chimeric CD40-L, wherein the chimeric CD40-L has a sequence selected from SEQ ID NOs: 7 to 18 or a CD40-L having between 80.00% and, up to, including 99.99% (e.g., 80, 81, 82, 83, 84, 85, 86, 87, 88, 89, 90, 91, 92, 93, 94, 95, 96, 97, 98, or 99%) sequence identity to a chimeric CD40-L having a sequence selected from SEQ ID NOs: 7 to 18.

In preferred embodiments, the oncolytic virus of the invention can comprise a nucleic acid encoding MEM40. For example, in preferred embodiments, the oncolytic viruses of the invention comprise IFNβ comprising at least 80% sequence identity to the human IFNβ (SEQ ID NO: 2) and the CD40-L comprises at least 80% sequence identity to a chimeric CD40-L having a sequence of SEQ ID NO: 12.

One or more heterologous nucleic acid sequences encoding the combination of type I IFN and CD40-L can be in one or more viral constructs. Non-limiting examples of the viral constructs include an adenoviral construct, adeno-associated viral construct (AAV), poxvirus construct, lentiviral construct, alphaviral construct, herpesviral construct, retroviral construct, vaccinia viral construct, vesicular stomatitis viral construct, or herpes simplex viral construct.

In addition to nucleic acids encoding a type I interferon (e.g., IFNβ) and CD40-L (chimeric human/mouse CD40L), the oncolytic virus in accordance with the present invention can further include other modifications in its genome. For example, it can comprise additional DNA inserted into an already inactivated gene, or substituted for a deleted gene. The oncolytic virus may also have incorporated therein one or more promoters that impart to the virus an enhanced level of tumor cell specificity. In this way, the oncolytic virus may be targeted to specific cancer types using cancer cell-specific promoters. The term “tumor cell-specific promoter” or “tumor cell-specific transcriptional regulatory sequence” or “tumor-specific promoter” or “tumor-specific transcriptional regulatory sequence” indicates a transcriptional regulatory sequence, promoter and/or enhancer that is present at a higher level in the target cancer cell than in a normal cell. For example, the oncolytic virus for use in the invention may be under the control of an exogenously added regulator.

In preferred embodiments, the oncolytic virus is an adenovirus (Ad). Ad is a large (approximately 36 kb) DNA virus that infects humans, but which also display a broad host range. Physically, adenovirus is an icosahedral virus containing a double-stranded, linear DNA genome. There are approximately 50 serotypes of human adenoviruses, which are divided into six families based on molecular, immunological, and functional criteria. By adulthood, virtually every human has been infected with the more common adenovirus serotypes, the major effect being cold-like symptoms.

Adenoviral infection of host cells results in adenoviral DNA being maintained episomally, which reduces the potential genotoxicity associated with integrating vectors. In addition, adenoviruses are structurally stable, and no genome rearrangement has been detected after extensive amplification. Adenovirus can infect most epithelial cells regardless of their cell cycle stage. So far, adenoviral infection appears to be linked only to mild disease such as acute respiratory disease in humans.

The infectious cycle of the adenovirus takes place in 2 steps: the early phase which precedes initiation of the replication of the adenoviral genome, and which permits production of the regulatory proteins and proteins involved in the replication and transcription of the viral DNA, and the late phase which leads to the synthesis of the structural proteins. The early genes are distributed in 4 regions that are dispersed in the adenoviral genome, designated E1 to E4 (“E” denotes “early”). The early regions comprise at least-six transcription units, each of which possesses its own promoter. The expression of the early genes is itself regulated, some genes being expressed before others. Three regions, E1, E2, and E4 are essential to replication of the virus. Thus, if an adenovirus is defective for one of these functions this protein will have to be supplied in trans, or the virus cannot replicate.

The E1 early region is located at the 5′ end of the adenoviral genome, and contains 2 viral transcription units, E1A and E1B. This region encodes proteins that participate very early in the viral cycle and are essential to the expression of almost all the other genes of the adenovirus. In particular, the E1 A transcription unit codes for a protein that transactivates the transcription of the other viral genes, inducing transcription from the promoters of the E1B, E2A, E2B, E3, and E4 regions and the late genes.

The adenovirus enters the permissive host cell via a cell surface receptor, and it is then internalized. The viral DNA associated with certain viral proteins needed for the first steps of the replication cycle enters the nucleus of the infected cells, where transcription is initiated. Replication of the adenoviral DNA takes place in the nucleus of the infected cells and does not require cell replication. New viral particles or virions are assembled after which they are released from the infected cells, and can infect other permissive cells.

The adenovirus is an attractive delivery system. Embodiments of the disclosure can utilize manufacturing process that can be free of or essentially free of protein, serum, and animal derived components making it suitable for a broad range of both prophylactic and therapeutic vaccine products.

If an adenovirus has been mutated so that it is conditionally replicative (replication-competent under certain conditions), a helper cell may be required for viral replication. When required, helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, for example Vero cells or other monkey embryonic mesenchymal or epithelial cells. In certain aspects a helper cell line is 293. Various methods of culturing host and helper cells may be found in the art, for example (Racher, A. J., Fooks, A. R. & Griffiths, J. B. Biotechnol Tech (1995) 9: 169.)

Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### Method of Treating Cancer

The disclosed compositions can be used to treat any disease where uncontrolled cellular proliferation occurs such as cancers. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

In one aspect disclosed herein are methods of treating, reducing, inhibiting, decreasing, ameliorating, and/or preventing a cancer and/or metastasis (including abscopal tumors) in a subject comprising administering to a subject any of the expanded TILs or MILs disclosed herein. For example, disclosed herein are methods of treating, reducing, inhibiting, decreasing, ameliorating, and/or preventing a cancer and/or metastasis a cancer in a subject comprising: a) administering an oncolytic virus expressing one or more type 1 interferon (IFN) (such as, for example, IFN-α, IFN-β, IFN-κ, IFN-δ, IFN-ε, IFN-τ, IFN-ω, and/or IFN-ζ) and/or one or more exogenous immunostimulatory molecules (such as, for example, CD40-L, MEM40, B7-1(CD80)/B7-2(CD86), OX40L, 4-1BBL, CD70, GITRL, LIGHT, TIM-4, ICAM-1, CD58 and/or SLAMF6) into a tumor cell; b) harvesting the tumor infiltrating lymphocytes (TILs) and/or marrow infiltrating lymphocytes (MILs); c) expanding the harvested TILs and/or MILs ex vivo; and d) administering the expanded TILs and/or MILs to the subject. In some aspects, the cancerous or metastatic tumor being treated, reduced, inhibited, decreased, ameliorated, and/or prevented are abscopal to the tumor receiving any of the oncolytic viruses and/or TILs or MILs disclosed herein.

Alternatively, the TILs or MILs can be harvested first from the tumor and then expanded ex vivo in the presence of antigen presenting cells infected with oncolytic virus expressing one or more type 1 interferon (IFN) and/or one or more exogenous immunostimulatory molecules; and administering the expanded TILs and/or MILs to the subject. The expanded TILs or MILs can then in-turn be administered (adoptively transferred) to the subject with the cancer. Thus, in one aspect, disclosed herein are methods of treating, reducing, inhibiting, decreasing, ameliorating, and/or preventing a cancer and/or metastasis in a subject comprising a) harvesting tumor infiltrating lymphocytes (TILs) and/or marrow infiltrating lymphocytes (MILs) from a subject with a cancer; culturing the harvested TILs or MILs in the presence of antigen presenting cells infected with an oncolytic virus expressing one or more type 1 interferon (IFN) (such as, for example, IFN-α, IFN-β, IFN-κ, IFN-δ, IFN-ε, IFN-τ, IFN-ω, and/or IFN-ζ) and/or one or more exogenous immunostimulatory molecules (such as, for example, CD40-L, MEM40, B7-1(CD80)/B7-2(CD86), OX40L, 4-1BBL, CD70, GITRL, LIGHT, TIM-4, ICAM-1, CD58 and/or SLAMF6); and administering the expanded TILs and/or MILs to the subject.

In one aspect, it is understood and herein contemplated that successful treatment of a cancer in a subject is important and doing so may include the administration of additional treatments. Accordingly, it is intended herein that the disclosed methods of treating, inhibiting, reducing, and/or preventing cancer can augmented with any therapeutic treatment of a cancer including, but not limited surgical, radiological, and/or pharmaceutical treatments of a cancer. Thus, the disclosed treatments can include and/or further include any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositunomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil-Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil-Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Also contemplated herein are chemotherapeutics that are PD1/PDL1 blockade inhibitors (such as, for example, lambrolizumab, nivolumab, pembrolizumab, pidilizumab, BMS-936559, Atezolizumab, Durvalumab, or Avelumab).

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Immunostimulatory Activity of Human IFNβ in a Mouse Melanoma Model

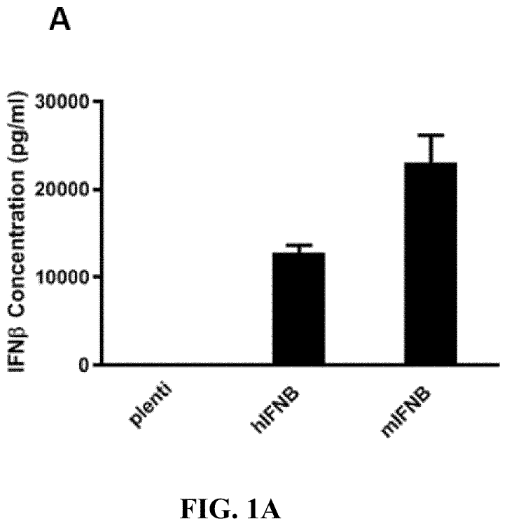

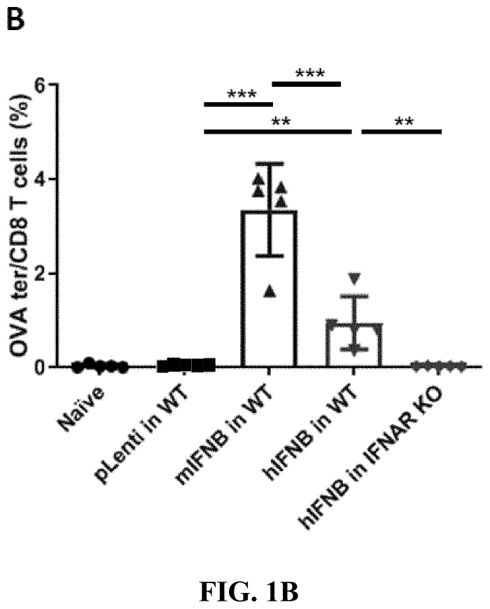

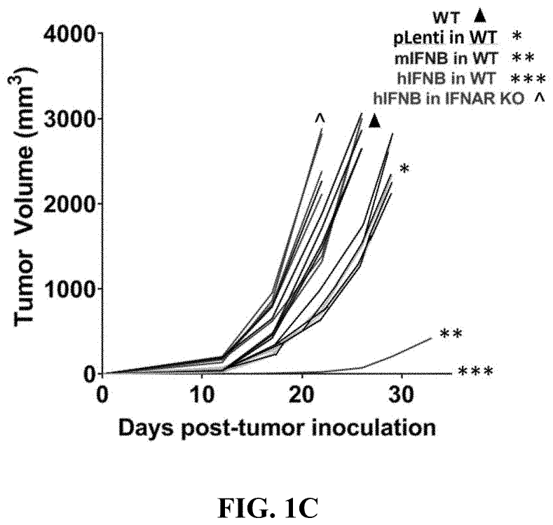

It is crucial to perform animal studies using the same agent as tested in humans. This however represents a problem for biologics, e.g. for the use of cytokines cross-reactivity varies widely between different human and mouse cytokines. As discussed above, we wished to develop an OV that expresses CD40L and human IFNβ as an anti-cancer therapeutic; IFNβ triggers signaling in mice albeit less proficiently than mouse IFNβ. Prior to OV development, it was first determined whether human IFNβ could trigger an antitumor T cell response in mice as that is the primary focus of this study. To this end, we utilized a recently developed assay where we found that irradiated B16-F10 or B16-OVA melanoma expressing mouse IFNβ functions as a powerful prophylactic vaccine, the protective effect of which is dependent on both CD8 and CD4 T cells (unpublished results). This approach is functionally analogous to GM-CSF expressing tumor cells (GVAX). B16-OVA cells were transduced with a pLenti-Puro control lentivirus or lentivirus expressing mouse or human IFNβ followed by puromycin selection. Equal numbers of cells were plated 2 days after which supernatant was collected for ELISA to detect human and mouse IFNβ. Both human and mouse IFNβ were expressed at high levels but higher levels of mouse IFNβ were detected (FIG. 1A). Notably, we found that both mouse and human IFNβ were highly effective in inducing a T cell response against a tumor-encoded model antigen although mouse IFNβ induced a higher response (FIG. 1B). It is possible that the higher response to mouse IFNβ is due in part to higher expression (FIG. 1A). Furthermore, this effect of human IFNβ was strictly dependent on the presence of type 1 IFN receptor α 1 (IFNαR1) in the host (FIG. 1B; similar results were obtained is separate experiments with mouse IFNβ). Functionally, both mouse and human IFNβ were highly effective in preventing tumor growth after challenge with live B16-OVA (FIG. 1C). These results indicate that in the setting of OV usage, human IFNβ are also capable of inducing an antitumor T cell response in mice.

### Example 2: Generation and In Vitro Testing of MEM-288

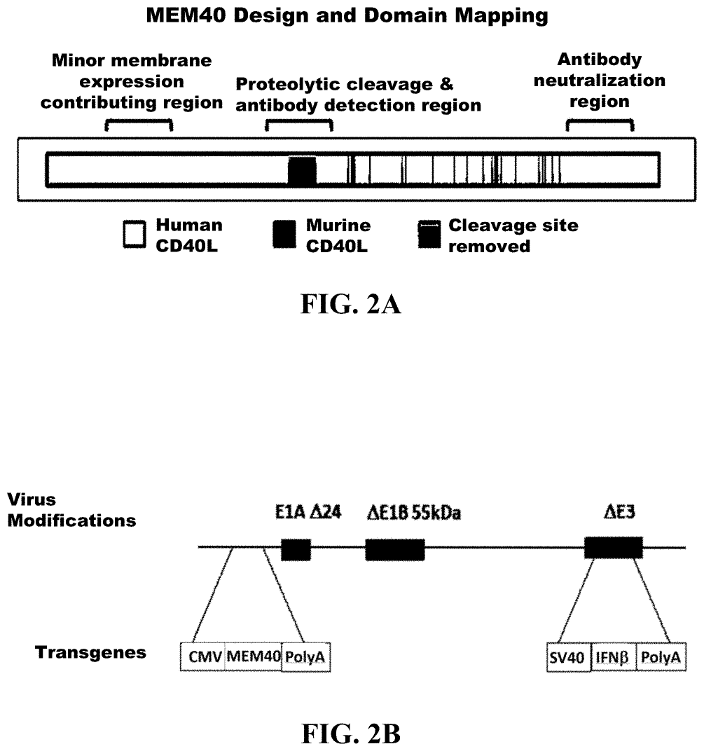

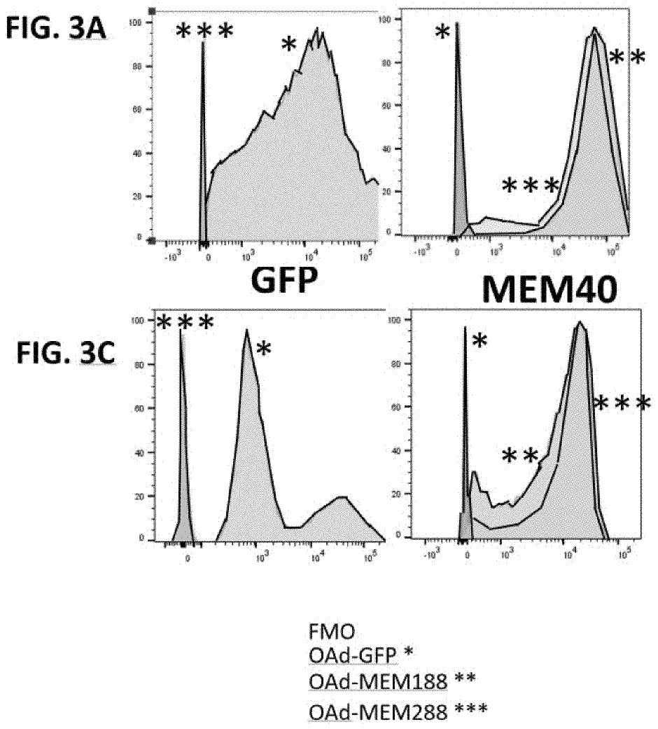

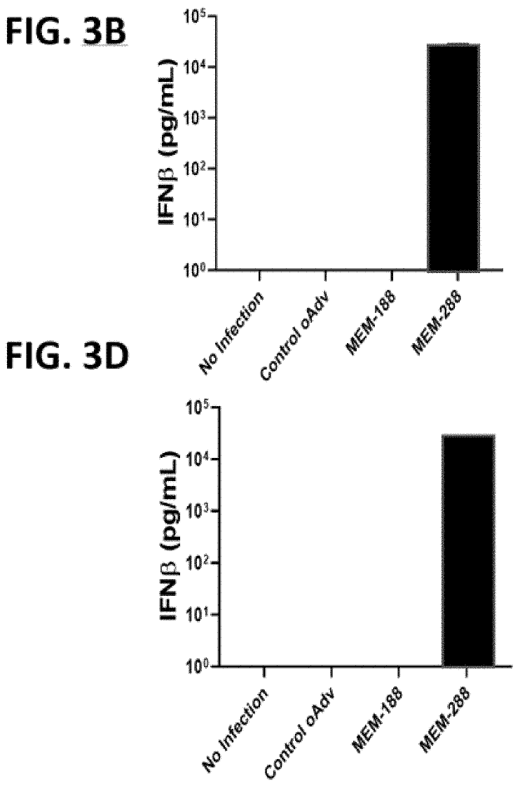

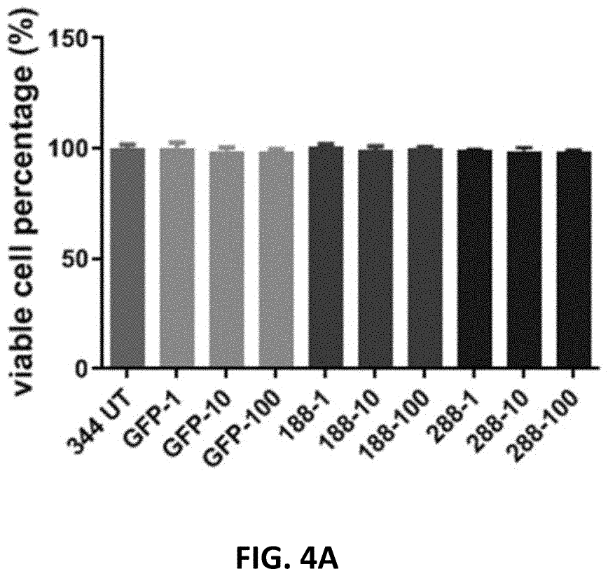

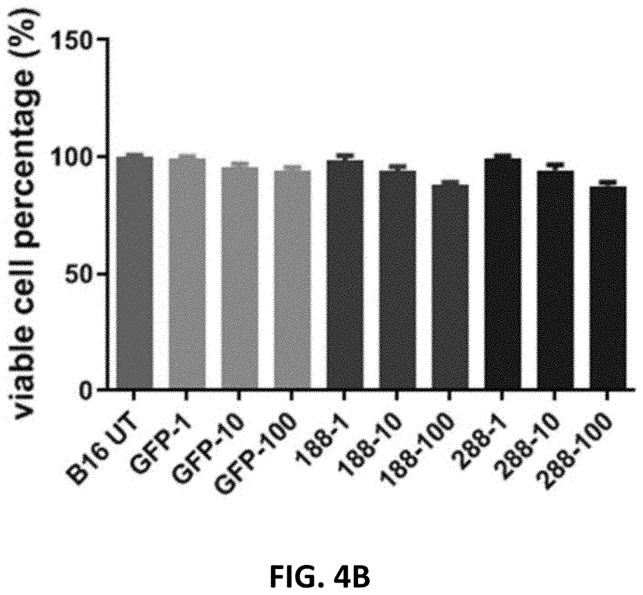

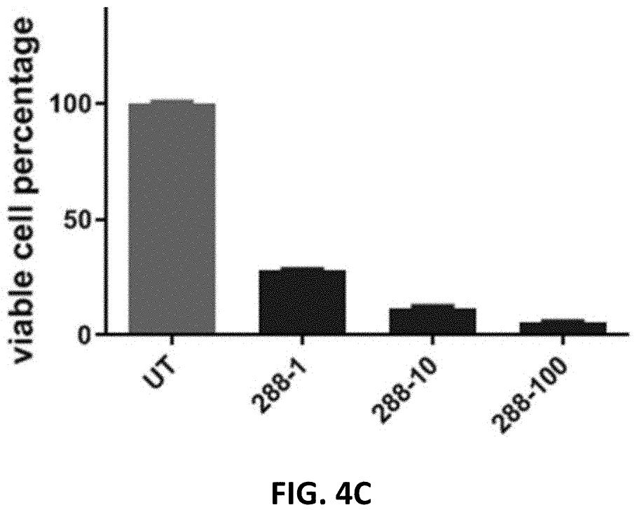

We generated an oncolytic adenovirus type 5 with CMV promoter driven expression of chimeric CD40L (FIG. 2A) with addition of an expression cassette to drive human IFNβ expression (MEM-288)(FIG. 2B). As controls, we used adenoviruses that express GFP (Ad-GFP) and chimeric CD40L only (MEM-188). All 3 viruses used contain the E1A D24 deletion to allow specific replication and lysis of cancer cells (see FIG. 4 below). Infection of human A549 lung cancer line with either MEM-188 or MEM-288 induced high CD40L expression as determined by flow cytometry, while infection with Ad-GFP led to high GFP expression (FIG. 3A). High-level secretion of IFNβ into the culture supernatant was seen only following MEM-288 infection (FIG. 3B). Similar results were obtained in multiple additional human cell lines. The studies with the B16-F10 melanoma cell line also demonstrated high transgene expression (FIG. 3C) and secretion of IFNβ into the culture supernatant after MEM-288 infection (FIG. 3D). Cell death by OVs is due to virus replication in infected cancer cells that leads to cytolysis. Type 1 IFN does not impair adenovirus replication. Notably, mouse cells do not support adenovirus replication, consistent with which we found that A24 deletion viruses did not induce lysis of mouse B16-F10 and 344SQ cell lines (FIG. 4) while human A549 cells were readily lysed (FIG. 4; MEM-288 shown). These results indicate that in in-vivo mouse studies, OV injection induced antitumor responses are more likely due to immune stimulatory effects rather than oncolytic effects. Despite lack of oncolytic effect in mouse cells, we refer to these viruses as OVs to indicate their application in humans.

### Example 3: Studies in a Mouse Melanoma Model

A main premise of use of intralesional approaches is the potential for generation of a systemic antitumor immune response that can target non-injected lesions, i.e. trigger an abscopal effect. As such, a main goal of the studies has been to determine whether combined activation of CD40 and type 1 IFN signaling can generate a strong antitumor T cell response that is capable of curtailing growth of both injected and uninjected tumors. Studies with a non-oncolytic replication-incompetent adenovirus expressing MEM40/CD40L showed activity in the B16-F10 melanoma model. However, 4 injections of this virus were needed to observe activity as a single agent or in combination with ICI.

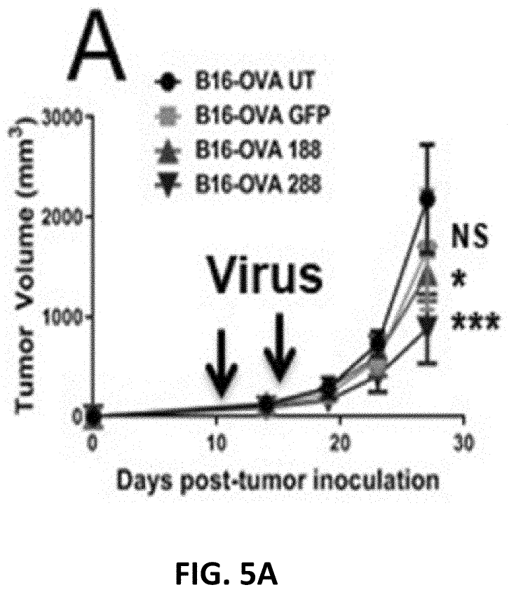

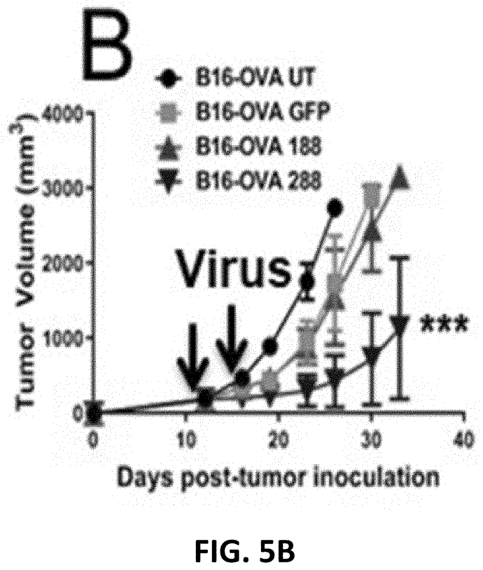

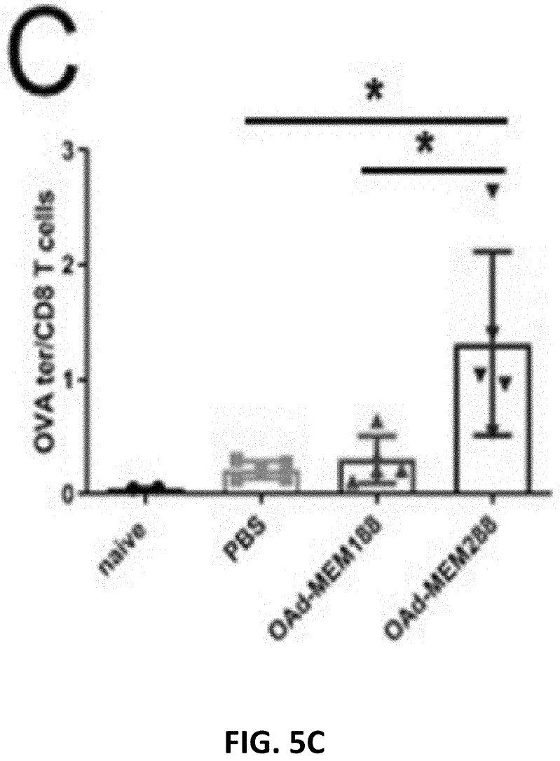

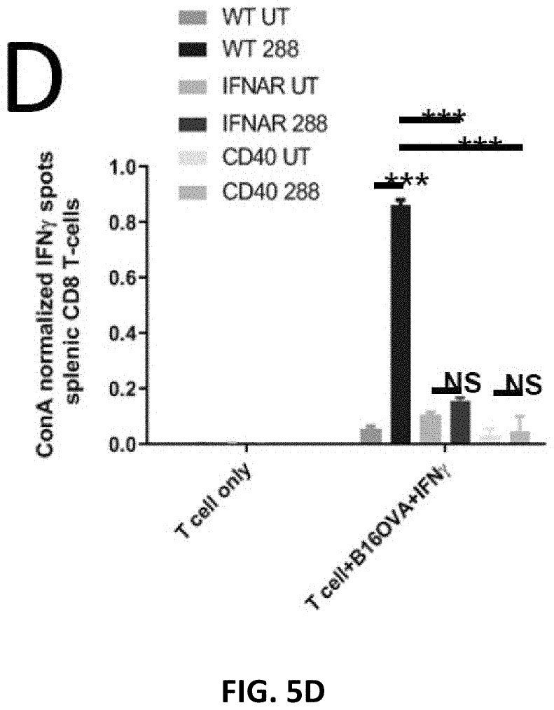

We first performed studies in B16-OVA melanoma to allow tracking of OVA-reactive CD8 T cells. Typical doses of adenovirus that have been used in mice to determine antitumor therapeutic effects are 10e9-10 infectious units (IU). A lower dose of 1×10⁸ of Ad-GFP, MEM-188 and MEM-288 was used for intratumoral injection in the first experiment (FIG. 5A) Interestingly, MEM-288 showed a more significant reduction in tumor growth compared to Ad-GFP and MEM-188 (FIG. 5A). In the next experiment, we used a 10-fold higher 10e9 IU dose of all 3 viruses where MEM-288 injection led to the most significant decrease in tumor growth (FIG. 5B). The above-mentioned study showed notable activity after 4 injections with MEM40/CD40L expressing adenovirus. However, we did not see similar activity after 2 injections with MEM-188, indicating that more sustained CD40L expression may be required through multiple virus injections, MEM-288 also led to an increase in circulating OVA-specific CD8 T cells while MEM-188 did not (FIG. 5C).

### MEM-288 Impact on Systemic T Cell Responses: Roles of CD40 and IFNαR1.

For these studies, we only used MEM-288 in comparison to a PBS control, MEM-288 administration into B16-OVA tumors led to a dramatic increase in the number of tumor-reactive CD8 T cells in mouse spleens as determined by IFNγ secretion (ELISPOT assay) (FIG. 5D). To assess the role of CD40 and type 1 IFN signaling in this, we used CD40 and IFNαR1 KO mice bearing B16-OVA tumors. Importantly, the CD8 T cell response was significantly reduced in both IFNαR1 and CD40 KO mice (FIG. 5D) demonstrating that both pathways are independently required for MEM-288 activity. Studies proposed in this application determine precisely how these 2 pathways function in activating a T cell response by investigating regulation of both APCs and T cells.

MEM-288 Activity with ICI Treatment.

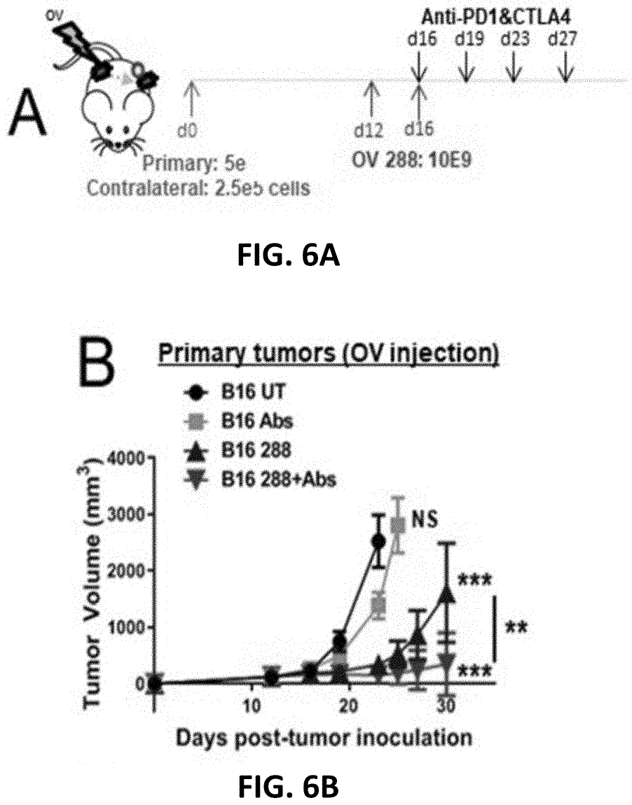

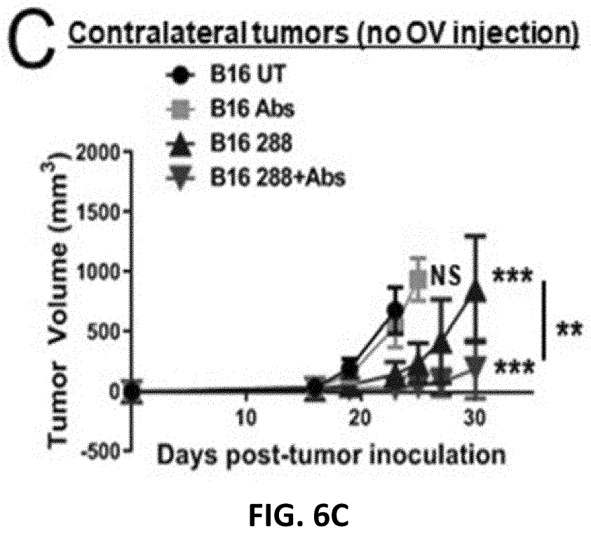

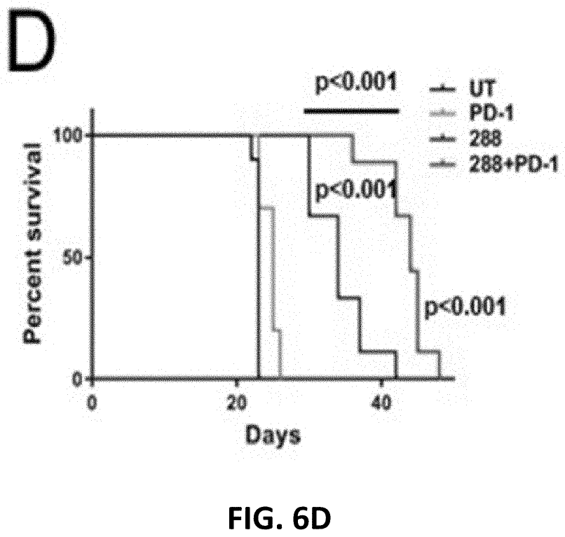

We wished to determine the effect of intratumoral injection of MEM-288 on the growth of both injected and non-injected contralateral tumors (FIG. 6A). Importantly, MEM-288 suppressed growth of both injected and contralateral tumors (FIG. 6B-C). In contrast, as is known for this tumor model, ICIs anti-CTLA4 and anti-PD-1 therapy minimally impacted tumor growth (FIG. 6B-C). These results indicate that MEM-288 has superior efficacy in this tumor model in comparison with anti-CTLA4+PD-1 treatment. Importantly, the combination of anti-CTLA4+PD-1 and MEM-288 significantly reduced tumor growth compared to MEM-288 alone indicating that ICI benefit is more evident when combined with MEM-288 (FIG. 6B-C). Finally, MEM-288 alone increased mouse survival which was further enhanced when combined with ICI (FIG. 6D). These results therefore indicate that (a) localized MEM-288 administration induces strong abscopal activity as a single agent, and (b) benefit from ICI therapy is manifested when combined with MEM-288 in an otherwise ICI resistant tumor model.

### Example 4: Studies in a Lung Metastatic Model

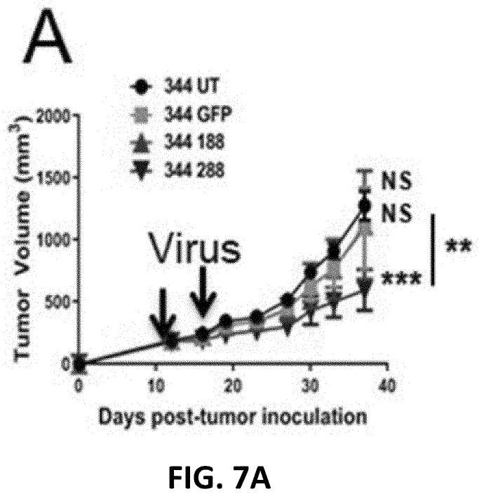

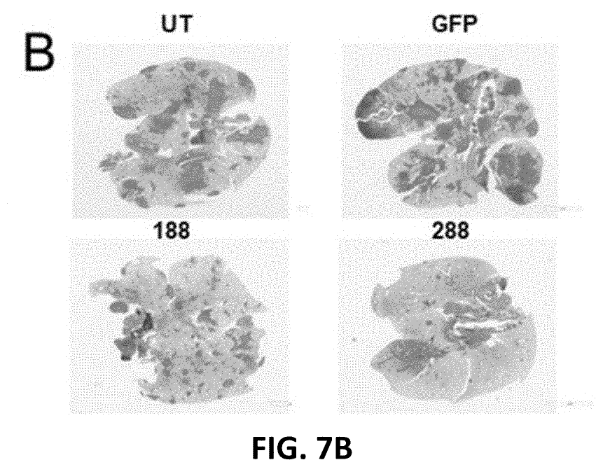

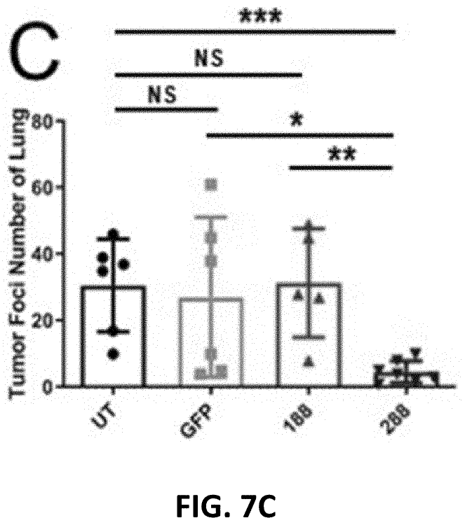

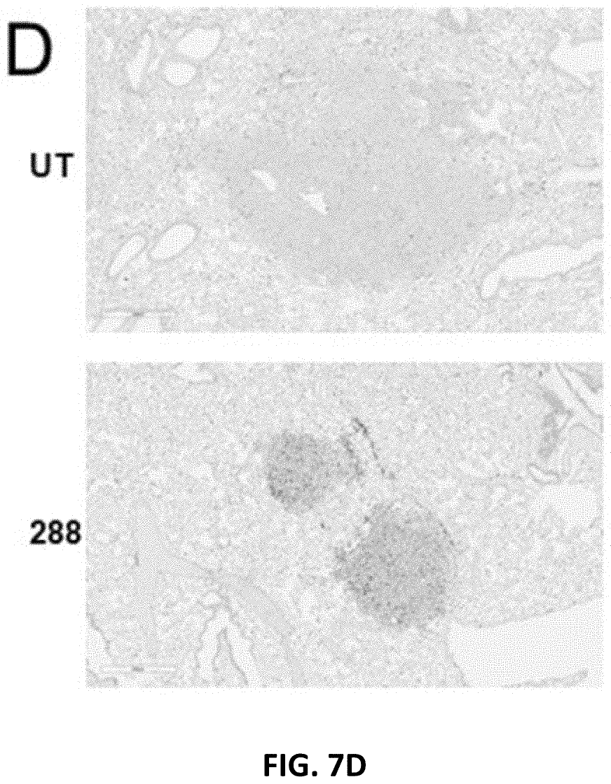

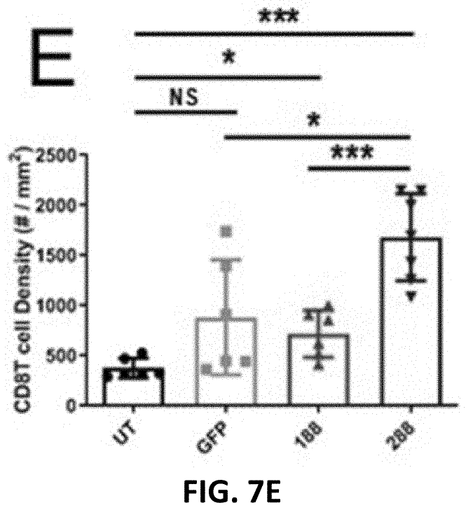

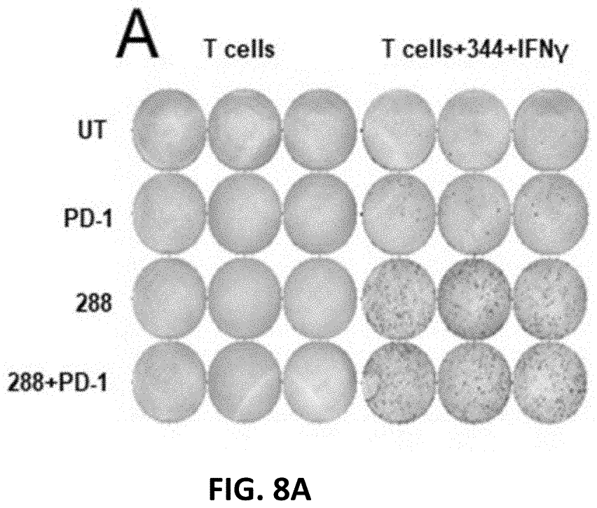

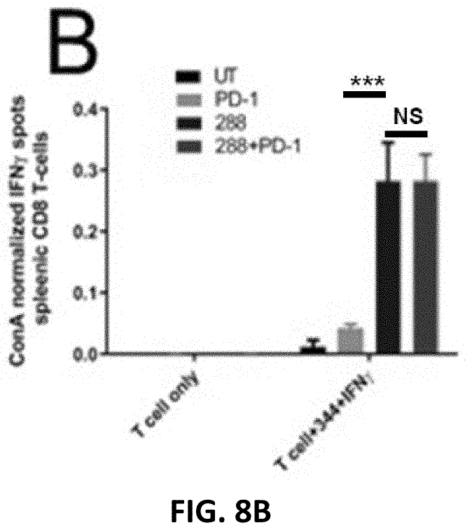

KRAS and TP53 mutant murine 344SQ (344) lung tumor cells injected subcutaneously form tumors at the primary site of injection followed by metastatic dissemination to lungs and others sites. We found that this model is resistant to PD-1 checkpoint blockade. A key question we wanted to address is the effectiveness of MEM-288 as a single agent in PD-1 refractory tumors. We show herein whether MEM-288 induced T cell activation and expansion prevents metastatic dissemination and/or curtail primary tumor growth. Two doses of 10e9 IU of Ad-GFP, MEM-188 and MEM-288 were administered intratumorally 12 days after inoculation. Only MEM-288 administration led to a significant decrease in s.c. tumor growth (FIG. 7A). Tumor lesions in lungs were determined 38 days after tumor inoculation. As expected, mice in which PBS were injected in s.c. tumors had high number of lungs mets, as did Ad-GFP and MEM-188 injected mice (FIG. 7B-C). In marked contrast, lung tumor lesions in MEM-288 injected mice were significantly reduced (FIG. 7B-C). Furthermore, tumor lesion size also appeared to be substantially smaller in MEM-288 as well as MEM-188 injected mice, CD8 IHC further revealed significantly higher TIL density in lung tumors of MEM-288 and to a smaller extent MEM-188 treated mice (FIG. 7D-E). These results indicate that the addition of IFNβ to CD40L likely enhances immune stimulatory function in this model. We next determined whether this is indeed the case and additionally investigated MEM-288 efficacy side-by-side with anti-PD-1. Importantly, we found a robust increase in tumor cell reactive CD8 T cells determined by IFNγ ELISPOT in spleens of MEM-288 injected mice (FIG. 8A-B). Anti-PD-1 on the other hand did not substantially increase tumor reactive T cells. We conclude that localized MEM-288 injection has the potential for triggering a strong systemic T cell response likely capable of controlling tumor growth as a single agent in PD-1 resistant tumors. In additional ongoing studies, we determine the synergy in antitumor activity when MEM-288 and ICI (anti-PD-1+/−anti-CTLA4) are combined in this model.

### Example 5: Investigating Immunostimulatory Mechanisms of CD40L and IFNβ in the Tumor Microenvironment

The results show that a virus expressing CD40L and IFNβ (MEM-288) is capable of inducing a potent systemic antitumor T cell response which can control the growth of abscopal tumor lesions. In addition to the viruses described above, we can also utilize a virus that expresses human IFNβ alone for the studies proposed here. We can determine the impact of CD40L and IFNβ expression in the TME on multiple immune cells types but especially DCs. Macrophages represent another major target of CD40L activity and can lead to enhancement of their antitumor activity. The studies include in-depth assessment of OV impact on DCs, macrophages and T cells with a key goal of defining the unique and synergistic contributions of CD40L and IFNβ. In addition to the above mentioned IFNαR1 and CD40 KO mice we can also use BATF3 KO mice, which lack DC1 (see below). We showed above that human IFNβ triggers T cell activation in mice in an IFNαR1-dependent manner (FIG. 1). However, it is possible that human IFNβ can trigger a more substantive response in the setting of human IFNαR1/2 receptors in mouse cells. Important, Harari et al generated a transgenic mouse strain named HyBNαR (Hybrid IFNαR) with human IFNαR1/2 extracellular ligand binding domain and mouse IFNαR1/2 receptor signaling domains. It was shown in this study that while human IFNβ induces signaling in wild-type mice, consistent with the results, this response is enhanced in HyBNαR mice.

The main goal of here is to show the impact of intralesional OV administration on the TME in B16 melanoma and 344 lung tumor models. We define here the unique and complementary effects of engagement of CD40L and IFNβ receptors on immune cells in the TME and the underlying mechanisms can also be investigated through use of mice lacking CD40 or IFNαR1. We believe DCs represent a major target of MEM-288 activity, to which end we can perform the most in-depth studies to investigate OV impact on the functionality of DC1 and DC2 subsets.

### Impact of OVs on the TME: Delineating the Specific Roles of CD40L and IFNβ

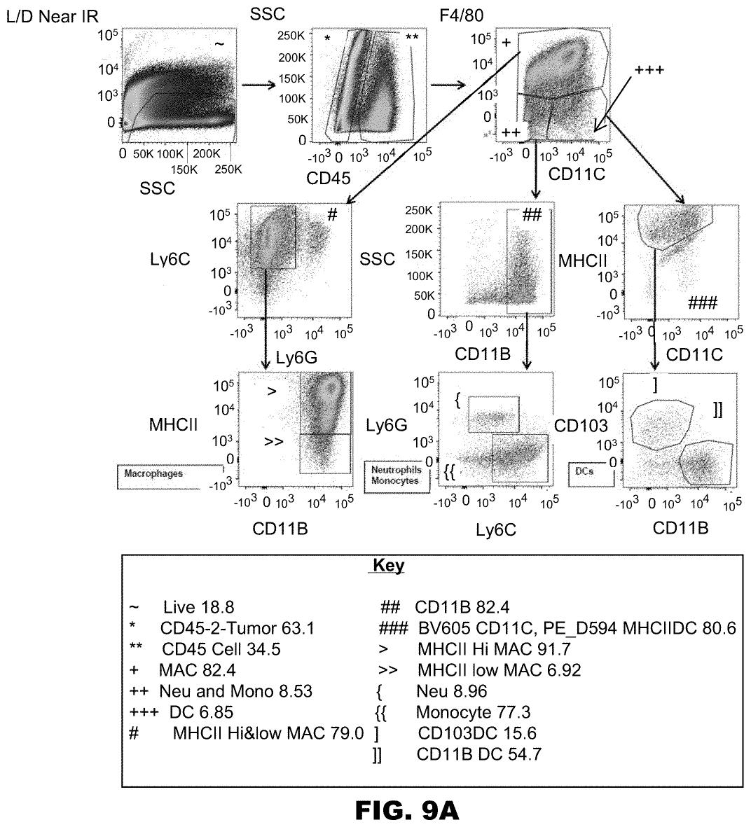

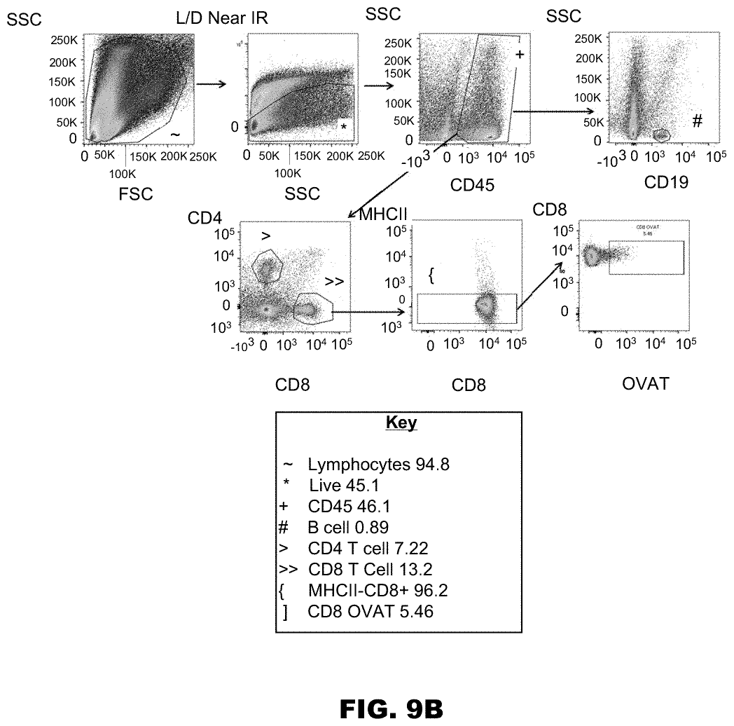

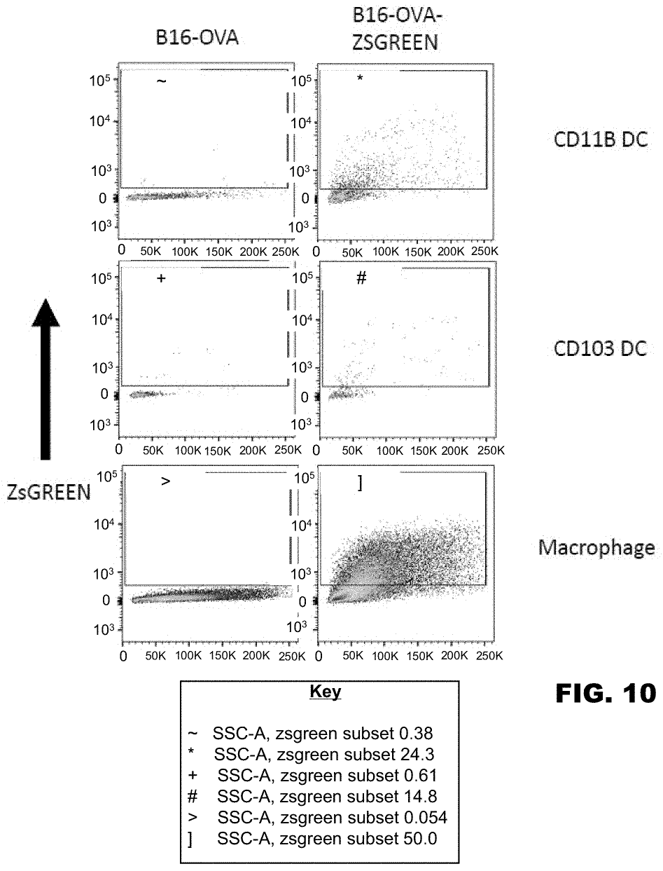

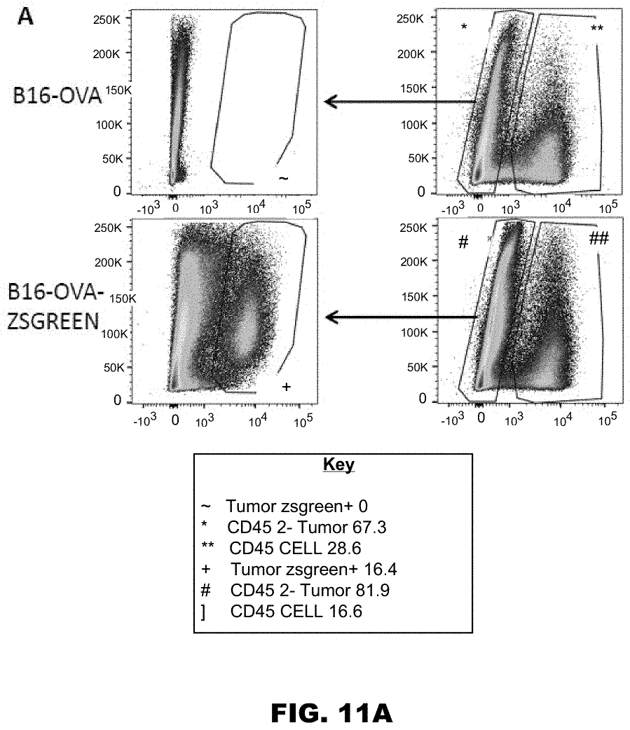

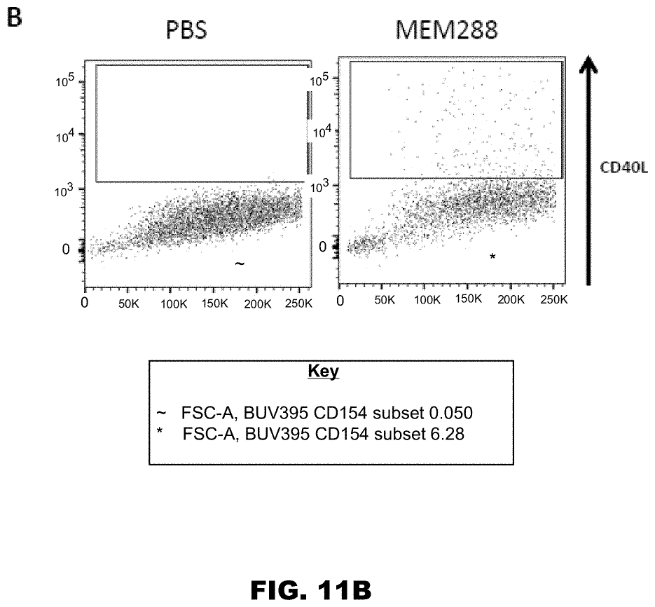

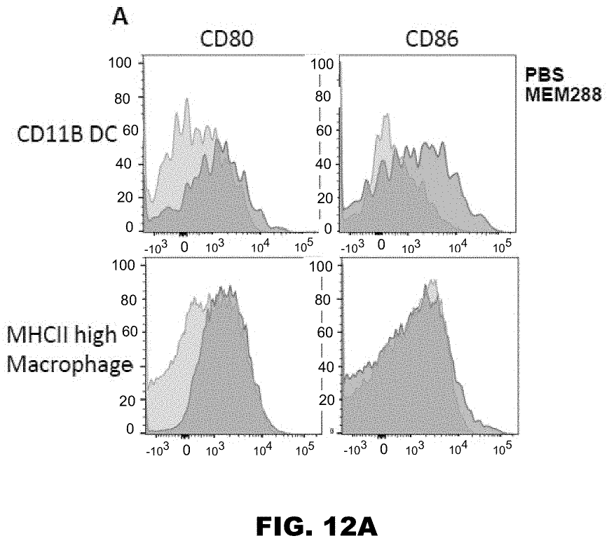

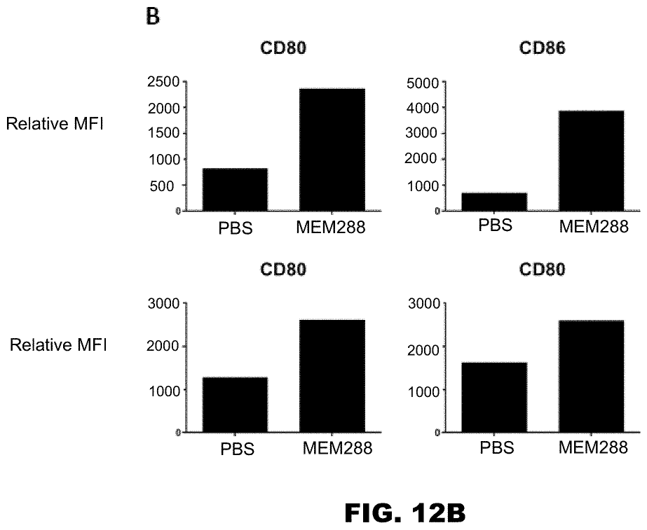

The primary goal is to determine mechanisms of action of CD40L and IFNβ in the TME for which we can use Ad-GFP, Ad-IFNβ, MEM-188 and MEM-288 OVs. These exploratory studies can help assess broad impact of above OVs on major populations of myeloid cells, DCs, T and B cells. As shown in FIG. 9A, we readily detected macrophages (F4/80⁺ Ly6C⁺ CD11b⁺ MHC-II⁺), monocytes and neutrophils (F4/80⁻ CD11b⁺ Ly6C⁺ or Ly6G⁺) and DCs including DC1 (CD11c⁺ CD11b⁻ MHC-II⁺ CD103⁺) and DC2 (CD11c⁺ CD11b⁺ MHC-II⁺ CD103⁻) by flow cytometry analysis of B16-OVA tumors. We also detected B cells, CD4⁺ T cells, CD8⁺ T cells and OVA-specific CD8 T cells (FIG. 9B). A major focus can be to determine OV effect on activation of DCs in tumors. Tumor antigens are readily taken up by resident macrophages and DCs, which can be detected by using tumors that express GFP or ZsGreen. Recent studies have also demonstrated that antigen uptake functionally reprograms DCs. We generated B16-OVA and B16-F10 expressing ZsGreen to track antigen uptake by tumor resident APCs (FIG. 10). As shown, in FIG. 10, ZsGreen⁺ populations of macrophages and DCs were readily detected in ZsGreen expressing tumors compared to parental non-expressing tumors. ZsGreen expression was used to identify tumor cells (ZsGreen⁺ CD45⁻) wherein we could also detect CD40L expression 2 days after a single MEM-288 injection (FIG. 11A-B). Importantly, we also found increase in CD80/CD86 in DCs and MHC-II^{high} macrophages in the same tumor after MEM-288 injection (FIG. 12A-B). While these results indicate that MEM-288 induces the activation of both macrophages and DCs, we show them to demonstrate feasibility of detection of CD40L expression and activation markers in APCs rather than as an actual finding. We are presently further determining the kinetics of CD40L transgene expression by testing tumors at 2, 4 and 6 days after a single injection in both B16 and 344 models. We can choose the time point with highest expression for studies below.

### Impact of OVs on the TME.

Herein we determine the impact of different OVs on the numbers and phenotypes of tumor infiltrating CD45⁺ immune cells in B16-OVA, B16-F10 and 344 models. Enumeration of each cell type can be performed by determining percentage of CD45⁺ cells and cell numbers per gram of tumor. The main populations can be as shown in FIG. 9 but with the addition of NK cell detection with NK1.1 Ab as NK cells are known targets of both CD40L and type 1 IFNs, and Treg (with intracellular FoxP3 expression) as they also play a role in modulating T cell responses after OV injections. Groups of mice (also see statistical plan) bearing ZsGreen expressing tumors can be treated with PBS or the above 4 OVs. We can use 5 mice per treatment in 2 independent experiments. 2 days after treatment (or a different time point based on highest CD40L expression from above studies), tumors can be digested to perform flow cytometry to determine differences in immune cells after injection of different OVs. A specific focus can be on tumor antigen-specific CD8 T cells in B16-OVA tumors. In B16-OVA implanted mice that were treated with MEM-288, we readily detected increase in antitumor T cells in blood (FIG. 5C) and spleen (FIG. 5D). We can determine here whether MEM-288 treatment results in the highest levels of total and OVA-specific CD8 T cells in tumors (detected as in FIG. 9B). We next determine how the different OVs impact ZsGreen uptake by macrophages and DCs. In key studies, expression of MHC-II and activation markers CD80, CD86 and CD40 in DC1 and DC2 can be determined. We can also determine activation state in both ZsGreen⁺ and ZsGreen⁻ cells. In addition, we can determine treatment effect on CCR7 expression in DCs as it plays a crucial role in DLN trafficking of DCs. IL-12 p70 is a critical Th1-response promoting cytokine secreted by DCs, and a known target of CD40L. Furthermore, expression of IL-12 p70 by tumor DCs is crucial for T cell antitumor immune response. We can determine expression of IL-12p40 subunit by intracellular flow in DCs after administration of brefeldin A in mice as described. Together, these studies can help determine how the different transgenes, i.e., CD40L, IFNβ and CD40L+IFNβ impact overall numbers and activation state of DC1 and DC2. For example, these studies can help determine whether increase in CD80/CD86 expression we observed after MEM-288 injection (FIG. 12) is due to CD40L, IFNβ or both. Statistical tests: For two group comparisons, Student's t-test can be used to determine differences in means. Multiple treatment group differences in means (e.g. of specific cell types or activation marker expression) can be determined by Tukey's method.

### Defining Specific Roles of CD40L and IFNβ Using Receptor KO Mice.

OVs expressing different transgenes have varying effects on the broader TME and on T cell/DC activation. Furthermore, MEM-288 has the most robust stimulatory effects because of the combined expression of CD40L and IFNβ. Using a complementary approach, we can determine the individual roles of CD40L and IFNβ on key phenotypes observed after MEM-288 injection by using mice lacking CD40 and IFNαR1. While the above studies have shown that CD40 and IFNαR1 are important for T cell responses (FIG. 5D), we determine here the role of DCs in this response. This approach can help provide additional insights into the unique and overlapping functions of these two pathways in the context of MEM-288 treatment. These studies can be performed in B16-F10 and B16-OVA models to match the C57BL/6 genetic background of CD40 and IFNαR1 KOs.

### Defining Impact of OVs on DC Trafficking to DLN and T Cell Stimulatory Ability

As shown in FIG. 10, both macrophages and DCs readily take up ZsGreen. However, DCs have the unique ability to traffic from tumors to DLN where they present tumor antigens to naive to T cells to trigger their activation. Shown herein is how the different OVs impact DC migration to DLN and their ability to prime T cells. For these studies, we can track DC1 and DC2 migration to DLNs by ZsGreen, as shown in a recent study, and also assess their ability to activate T cells. These studies help determine how the different transgenes impact DC migration and T cell priming.

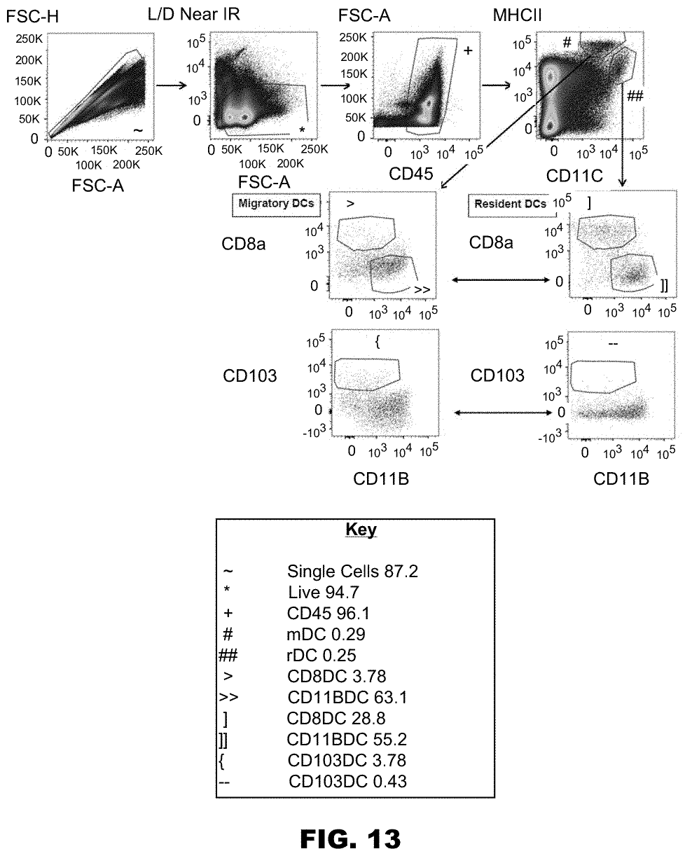

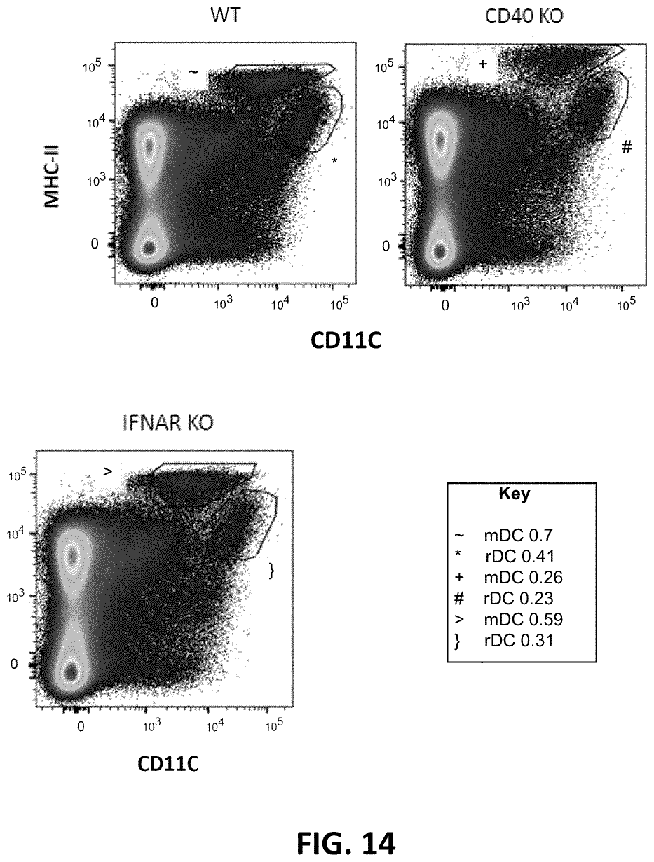

As shown in FIG. 13, 2 main populations of MHC-II⁺ CD11c⁺ cells are detected in inguinal DLNs. The MHC^{high} population is thought to comprise of migratory DC1 and DC2, which as expected has the higher percentage of CD103⁺ DCs as well as CD11b⁺ DCs. A CD11c⁺ population with intermediate levels of MHC-II (MHC^{int}) population comprises of resident DC1 and DC2 and has a lower percentage of CD103⁺ DCs. The majority of ZsGreen+DCs reside in the MHC^{high} population. We can determine here the impact of different OVs on the distribution and activation phenotype of these distinct populations, MEM-288 administration can result in the highest DLN trafficking and that these DCs have the strongest activation phenotype. We can also determine the ability of MEM-288 to induce DC migration in CD40 and IFNαR1 KO mice. As shown in FIG. 14, LN populations of DCs do not appear to be impacted by absence of CD40 and IFNαR1. Nonetheless, it is not known whether CD40 and IFNαR1 absence impacts DC migration from tumor to DLNs.

We can next determine the ability of LN DC to activate T cells. While the studies have already shown that T cell priming is greatly enhanced by MEM-288, these studies can specifically determine whether this is due, at least in part, to DC activation. To this end, we can use mice bearing B16-OVA tumors for injection with all 4 OVs. Next, we can use DC1 and DC2 sorted from DLN cells for co-culture with CFSE labeled naïve OT-1 CD8 T cells to perform CFSE-dilution assays as described herein. These studies can determine whether MEM-288 induced DC activation results in the highest levels of T cell proliferation. In addition, we can perform ELISPOT assays by culturing DCs with OT-1 CD8 T cells to determine IFNγ secretion. Using MEM-288, we can next use CD40 and IFNαR1 KO mice to determine the specific roles the 2 transgenes play in DC ability to activate T cells. Finally, using BATF3 KO mice, we can determine the role of DC1 for MEM-288 induced increase in T cell proliferation and IFNγ secretion.

### Example 6: Oncolytic Virus Treatment of Tumors

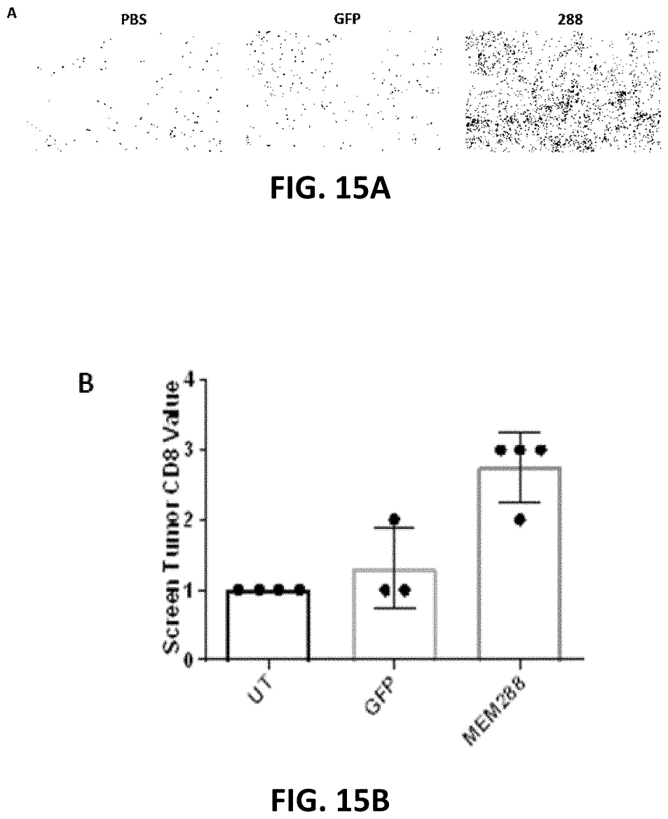

To show the effect of the oncolytic virus on tumors in vivo, C57BL/6 mice were inoculated s.c. with 5e5 B16-OVA cells on the flank. Tumors were subjected to PBS, Adenovirus-GFP, or MEM-288, injection at 10⁹ IU on D12 and D16. On day 20, tumors were obtained to perform IHC to detect presence of CD8⁺ tumor infiltrating lymphocytes (TILs). Mice treated with PBS or oncolytic virus expressing GFP showed similar numbers of TILs. By contrast, MEM-288 vaccinated tumors had a near 3-fold increase in TILs (FIG. 15).

### SEQUENCE LISTING

The Sequence Listing is available in electronic form from the USPTO and was filed with the file name “15041500016US Sequence Listing” in XML format, which was created on Apr. 25, 2023. The XML file is 20,872 bytes.

### REFERENCES

Ahonen, C. L., Doxsee, C. L., McGurran, S. M., Riter, T. R., Wade, W. F., Barth, R. J., Vasilakos, J. P., Noelle, R. J., and Kedl, R. M. 2004. Combined TLR and CD40 triggering induces potent CD8+ T cell expansion with variable dependence on type I IFN. J Exp Med 199:775-784.
Aran, D., Looney, A. P., Liu, L., Wu, E., Fong, V., Hsu, A., Chak, S., Naikawadi, R. P., Wolters, P. J., Abate, A. R., Butte, A. J., and Bhattacharya, M. 2019. Reference-based analysis of lung single-cell sequencing reveals a transitional profibrotic macrophage. Nature Immunology 20:163-172.
Bevan, M. J. 2004. Helping the CD8(+) T-cell response. Nat Rev Immunol 4:595-602.
Billerbeck, E., Barry, W. T., Mu, K., Domer, M., Rice, C. M., and Ploss, A. 2011. Development of human CD4+FoxP3+ regulatory T cells in human stem cell factor-, granulocyte-macrophage colony-stimulating factor-, and interleukin-3-expressing NOD-SCID IL2Rgamma(null) humanized mice. Blood 117:3076-3086.
Billerbeck, E., Horwitz, J. A., Labitt, R. N., Donovan, B. M., Vega, K., Budell, W. C., Koo, G. C., Rice, C. M., and Ploss, A. 2013. Characterization of human antiviral adaptive immune responses during hepatotropic virus infection in HLA-transgenic human immune system mice. J Immunol 191:1753-1764.
Blair, G. E., Dixon, S. C., Griffiths, S. A., and Zajdel, M. E. 1989. Restricted replication of human adenovirus type 5 in mouse cell lines. Virus Res 14:339-346.
Blander, J. M. 2018. Regulation of the Cell Biology of Antigen Cross-Presentation. Annu Rev Immunol 36:717-753.
Bommareddy, P. K., Shettigar, M., and Kaufman, H. L, 2018. Integrating oncolytic viruses in combination cancer immunotherapy. Nat Rev Immunol 18:498-513.
Bommareddy, P. K., Silk, A. W., and Kaufman, H. L, 2017. Intratumoral Approaches for the Treatment of Melanoma. Cancer J 23:40-47.
Brahmer, J., Reckamp, K. L., Baas, P., Crino, L., Eberhardt, W. E., Poddubskaya, E., Antonia, S., Pluzanski, A., Vokes, E. E., Holgado, E., Waterhouse, D., Ready, N., Gainor, J., Aren Frontera, O., Havel, L., Steins, M., Garassino, M. C., Aerts, J. G., Domine, M., Paz-Ares, L., Reck, M., Baudelet, C., Harbison, C. T., Lestini, B., and Spigel, D. R. 2015. Nivolumab versus Docetaxel in Advanced Squamous-Cell Non-Small-Cell Lung Cancer. N Engl J Med 373:123-135.
Brahmer, J. R., Tykodi, S. S., Chow, L. Q., Hwu, W. J., Topalian, S. L., Hwu, P., Drake, C. G., Camacho, L. H., Kauh, J., Odunsi, K., Pitot, H. C., Hamid, O., Bhatia, S., Martins, R., Eaton, K., Chen, S., Salay, T. M., Alaparthy, S., Grosso, J. F., Korman, A. J., Parker, S. M., Agrawal, S., Goldberg, S. M., Pardoll, D. M., Gupta, A., and Wigginton, J. M. 2012. Safety and activity of anti-PD-L1 antibody in patients with advanced cancer. N Engl J Med 366:2455-2465.
Chen, D. S., and Mellman, I. 2017. Elements of cancer immunity and the cancer-immune set point. Nature 541:321-330.
Cho, H. I., and Celis, E. 2009. Optimized peptide vaccines eliciting extensive CD8 T-cell responses with therapeutic antitumor effects. Cancer research 69:9012-9019.
Curtsinger, J. M., Valenzuela, J. O., Agarwal, P., Lins, D., and Mescher, M. F. 2005. Type I IFNs provide a third signal to CD8 T cells to stimulate clonal expansion and differentiation. J Immunol 174:4465-4469.
de Mingo Pulido, A., Gardner, A., Hiebler, S., Soliman, H., Rugo, H. S., Krummel, M. F., Coussens, L. M., and Ruffell, B. 2018, TIM-3 Regulates CD103(+) Dendritic Cell Function and Response to Chemotherapy in Breast Cancer. Cancer Cell 33:60-74 e66.
Diamond, M. S., Kinder, M., Matsushita, H., Mashayekhi, M., Dunn, G. P., Archambault, J. M., Lee, H., Arthur, C. D., White, J. M., Kalinke, U., Murphy, K. M., and Schreiber, R. D. 2011. Type I interferon is selectively required by dendritic cells for immune rejection of tumors. The Journal of experimental medicine 208:1989-2003.
Dobin, A., Davis, C. A., Schlesinger, F., Drenkow, J., Zaleski, C., Jha, S., Batut, P., Chaisson, M., and Gingeras, T. R. 2013. STAR: ultrafast universal RNA-seq aligner. Bioinformatics 29:15-21.
Ferris, S. T., Durai, V., Wu, R., Theisen, D. J., Ward, J. P., Bern, M. D., Davidson, J. T. t., Bagadia, P., Liu, T., Briseno, C. G., Li, L., Gillanders, W. E., Wu, G. F., Yokoyama, W. M., Murphy, T. L., Schreiber, R. D., and Murphy, K. M. 2020. cDC1 prime and are licensed by CD4(+) T cells to induce anti-tumour immunity. Nature 584:624-629.
Flood, B. A., Higgs, E. F., Li, S., Luke, J. J., and Gajewski, T. F. 2019. STING pathway agonism as a cancer therapeutic. Immunol Rev 290:24-38.
Fuertes, M. B., Kacha, A. K., Kline, J., Woo, S. R., Kranz, D. M., Murphy, K. M., and Gajewski, T. F. 2011. Host type I IFN signals are required for antitumor CD8+ T cell responses through CD8{alpha}+dendritic cells. The Journal of experimental medicine 208:2005-2016.
Gardner, A., and Ruffell, B. 2016. Dendritic Cells and Cancer Immunity. Trends Immunol.
Gibbons, D. L., Lin, W., Creighton, C. J., Rizvi, Z. H., Gregory, P. A., Goodall, G. J., Thilaganathan, N., Du, L., Zhang, Y., Pertsemlidis, A., and Kurie, J. M. 2009. Contextual extracellular cues promote tumor cell EMT and metastasis by regulating miR-200 family expression. Genes & development 23:2140-2151.
Hanzelmann, S., Castelo, R., and Guinney, J. 2013. GSVA: gene set variation analysis for microarray and RNA-Seq data. BMC Bioinformatics 14:7.
Harari, D., Abramovich, R., Zozulya, A., Smith, P., Pouly, S., Koster, M., Hauser, H., and Schreiber, G. 2014. Bridging the species divide: transgenic mice humanized for type-I interferon response. PLoS One 9:e84259.
Herbst, R. S., Soria, J. C., Kowanetz, M., Fine, G. D., Hamid, O., Gordon, M. S., Sosman, J. A., McDermott, D. F., Powderly, J. D., Gettinger, S. N., Kohrt, H. E., Hom, L., Lawrence, D. P., Rost, S., Leabman, M., Xiao, Y., Mokatrin, A., Koeppen, H., Hegde, P. S., Mellman, I., Chen, D. S., and Hodi, F. S. 2014. Predictive correlates of response to the anti-PD-L1 antibody MPDL3280A in cancer patients. Nature 515:563-567.
Hildner, K., Edelson, B. T., Purtha, W. E., Diamond, M., Matsushita, H., Kohyama, M., Calderon, B., Schraml, B. U., Unanue, E. R., Diamond, M. S., Schreiber, R. D., Murphy, T. L., and Murphy, K. M. 2008. Batf3 deficiency reveals a critical role for CD8alpha+ dendritic cells in cytotoxic T cell immunity. Science 322:1097-1100.
Huarte, E., Larrea, E., Hemandez-Alcoceba, R., Alfaro, C., Murillo, O., Arina, A., Tirapu, I., Azpilicueta, A., Hervas-Stubbs, S., Bortolanza, S., Perez-Gracia, J. L., Civeira, M. P., Prieto, J., Riezu-Boj, J. I., and Melero, I. 2006. Recombinant adenoviral vectors turn on the type I interferon system without inhibition of transgene expression and viral replication. Mol Ther 14:129-138.
Joyce, J. A., and Fearon, D. T. 2015. T cell exclusion, immune privilege, and the tumor microenvironment. Science 348:74-80.
Kolumam, G. A., Thomas, S., Thompson, L. J., Sprent, J., and Murali-Krishna, K. 2005. Type I interferons act directly on CD8 T cells to allow clonal expansion and memory formation in response to viral infection. J Exp Med 202:637-650.
Macosko, E. Z., Basu, A., Satija, R., Nemesh, J., Shekhar, K., Goldman, M., Tirosh, I., Bialas, A. R., Kamitaki, N., Martersteck, E. M., Trombetta, J. J., Weitz, D. A., Sanes, J. R., Shalek, A. K., Regev, A., and McCarroll, S. A. 2015. Highly parallel genome-wide expression profiling of individual cells using nanoliter droplets. Cell 161:1202-1214.
Maier, B., Leader, A. M., Chen, S. T., Tung, N., Chang, C., LeBerichel, J., Chudnovskiy, A., Maskey, S., Walker, L., Finnigan, J. P., Kirkling, M. E., Reizis, B., Ghosh, S., D'Amore, N. R., Bhardwaj, N., Rothlin, C. V., Wolf, A., Flores, R., Marron, T., Rahman, A. H., Kenigsberg, E., Brown, B. D., and Merad, M. 2020. A conserved dendritic-cell regulatory program limits antitumour immunity. Nature 580:257-262.
Melo-Cardenas, J., Urquiza, M., Kipps, T. J., and Castro, J. E. 2012. Intratumoral delivery of CD154 homolog (Ad-ISF35) induces tumor regression: analysis of vector biodistribution, persistence and gene expression. Cancer Gene Ther 19:336-344.
O'Hara, M. H. 2019. A Phase Ib study of CD40 agonistic monoclonal antibody APX005M together with gemcitabine (Gem) and nab-paclitaxel (NP) with or without nivolumab (Nivo) in untreated metastatic ductal pancreatic adenocarcinoma (PDAC) patients. AACR Annual Meeting.
Ribas, A., Dummer, R., Puzanov, I., VanderWalde, A., Andtbacka, R. H. I., Michielin, O., Olszanski, A. J., Malvehy, J., Cebon, J., Fernandez, E., Kirkwood, J. M., Gajewski, T. F., Chen, L., Gorski, K. S., Anderson, A. A., Diede, S. J., Lassman, M. E., Gansert, J., Hodi, F. S., and Long, G. V. 2017. Oncolytic Virotherapy Promotes Intratumoral T Cell Infiltration and Improves Anti-PD-1 Immunotherapy. Cell 170:1109-1119 e1110.
Ribas, A., Medina, T., Kummar, S., Amin, A., Kalbasi, A., Drabick, J. J., Barve, M., Daniels, G. A., Wong, D. J., Schmidt, E. V., Candia, A. F., Coffman, R. L., Leung, A. C. F., and Janssen, R. S. 2018. SD-101 in Combination with Pembrolizumab in Advanced Melanoma: Results of a Phase Ib, Multicenter Study. Cancer Discov 8:1250-1257.
Roberts, E. W., Broz, M. L., Binnewies, M., Headley, M. B., Nelson, A. E., Wolf, D. M., Kaisho, T., Bogunovic, D., Bhardwaj, N., and Krummel, M. F. 2016. Critical Role for CD103(+)/CD141(+) Dendritic Cells Bearing CCR7 for Tumor Antigen Trafficking and Priming of T Cell Immunity in Melanoma. Cancer Cell 30:324-336.
Ruffell, B., and Coussens, L. M. 2015. Macrophages and therapeutic resistance in cancer. Cancer Cell 27:462-472.
Ruffell, B., Chang-Strachan, D., Chan, V., Rosenbusch, A., Ho, C. M., Pryer, N., Daniel, D., Hwang, E. S., Rugo, H. S., and Coussens, L. M. 2014. Macrophage IL-10 blocks CD8+ T cell-dependent responses to chemotherapy by suppressing IL-12 expression in intratumoral dendritic cells. Cancer Cell 26:623-637.
Sanchez, P. J., McWilliams, J. A., Haluszczak, C., Yagita, H., and Kedl, R. M. 2007. Combined TLR/CD40 stimulation mediates potent cellular immunity by regulating dendritic cell expression of CD70 in vivo. J Immunol 178:1564-1572.
Scarlett, U.K., Cubillos-Ruiz, J. R., Nesbeth, Y. C., Martinez, D. G., Engle, X., Gewirtz, A. T., Ahonen, C. L., and Conejo-Garcia, J. R. 2009. In situ stimulation of CD40 and Toll-like receptor 3 transforms ovarian cancer-infiltrating dendritic cells from immunosuppressive to immunostimulatory cells. Cancer Res 69:7329-7337.
Schoenberger, S. P., Toes, R. E., van der Voort, E. I., Offringa, R., and Melief, C. J. 1998. T-cell help for cytotoxic T lymphocytes is mediated by CD40-CD40L interactions. Nature 393:480-483.
Seymour, L. W., and Fisher, K. D. 2016. Oncolytic viruses: finally delivering. Br J Cancer 114:357-361.
Sharma, P., Hu-Lieskovan, S., Wargo, J. A., and Ribas, A. 2017. Primary, Adaptive, and Acquired Resistance to Cancer Immunotherapy. Cell 168:707-723.
Singh, M., Vianden, C., Cantwell, M. J., Dai, Z., Xiao, Z., Sharma, M., Khong, H., Jaiswal, A. R., Faak, F., Hailemichael, Y., Janssen, L. M. E., Bharadwaj, U., Curran, M. A., Diab, A., Bassett, R. L., Tweardy, D. J., Hwu, P., and Overwijk, W. W. 2017. Intratumoral CD40 activation and checkpoint blockade induces T cell-mediated eradication of melanoma in the brain. Nat Commun 8:1447.
Subramanian, A., Tamayo, P., Mootha, V. K., Mukherjee, S., Ebert, B. L., Gillette, M. A., Paulovich, A., Pomeroy, S. L., Golub, T. R., Lander, E. S., and Mesirov, J. P. 2005. Gene set enrichment analysis: A knowledge-based approach for interpreting genome-wide expression profiles. Proceedings of the National Academy of Sciences 102:15545-15550.
Tirosh, I., Izar, B., Prakadan, S. M., Wadsworth, M. H., 2nd, Treacy, D., Trombetta, J. J., Rotem, A., Rodman, C., Lian, C., Murphy, G., Fallahi-Sichani, M., Dutton-Regester, K., Lin, J.-R., Cohen, O., Shah, P., Lu, D., Genshaft, A. S., Hughes, T. K., Ziegler, C. G. K., Kazer, S. W., Gaillard, A., Kolb, K. E., Villani, A.-C., Johannessen, C. M., Andreev, A. Y., Van Allen, E. M., Bertagnolli, M., Sorger, P. K., Sullivan, R. J., Flaherty, K. T., Frederick, D. T., Jand-Valbuena, J., Yoon, C. H., Rozenblatt-Rosen, O., Shalek, A. K., Regev, A., and Garraway, L. A. 2016. Dissecting the multicellular ecosystem of metastatic melanoma by single-cell RNA-seq. Science (New York, N.Y.) 352:189-196.
Topalian, S. L., Hodi, F. S., Brahmer, J. R., Gettinger, S. N., Smith, D. C., McDermott, D. F., Powderly, J. D., Carvajal, R. D., Sosman, J. A., Atkins, M. B., Leming, P. D., Spigel, D. R., Antonia, S. J., Hom, L., Drake, C. G., Pardoll, D. M., Chen, L., Sharfman, W. H., Anders, R. A., Taube, J. M., McMiller, T. L., Xu, H., Korman, A. J., Jure-Kunkel, M., Agrawal, S., McDonald, D., Kollia, G. D., Gupta, A., Wigginton, J. M., and Sznol, M. 2012. Safety, activity, and immune correlates of anti-PD-1 antibody in cancer. The New England journal of medicine 366:2443-2454.
Tumeh, P. C., Harview, C. L., Yearley, J. H., Shintaku, I. P., Taylor, E. J., Robert, L., Chmielowski, B., Spasic, M., Henry, G., Ciobanu, V., West, A. N., Carmona, M., Kivork, C., Seja, E., Cherry, G., Gutierrez, A. J., Grogan, T. R, Mateus, C., Tomasic, G., Glaspy, J. A., Emerson, RO., Robins, H., Pierce, R H., Elashoff, D. A., Robert, C., and Ribas, A. 2014. PD-1 blockade induces responses by inhibiting adaptive immune resistance. Nature 515:568-571.
Urquiza, M., Melo-Cardenas, J., Aguillon, R, Kipps, T. J., and Castro, J. E. 2015. Intratumoral injection of Ad-ISF35 (Chimeric CD154) breaks tolerance and induces lymphoma tumor regression. Hum Gene Ther 26:14-25.
Valenzuela, J. O., Iclozan, C., Hossain, M. S., Prlic, M., Hopewell, E., Bronk, C. C., Wang, J., Celis, E., Engelman, R W., Blazar, B. R., Bevan, M. J., Waller, E. K., Yu, X. Z., and Beg, A. A. 2009. PKCtheta is required for alloreactivity and GVHD but not for immune responses toward leukemia and infection in mice. J Clin Invest 119:3774-3786.
Valenzuela, J. O., Iclozan, C., Hossain, M. S., Prlic, M., Hopewell, E., Bronk, C. C., Wang, J., Celis, E., Engelman, R W., Blazar, B. R., Bevan, M. J., Waller, E. K., Yu, X. Z., and Beg, A. A. 2009. PKCtheta is required for alloreactivity and GVHD but not for immune responses toward leukemia and infection in mice. The Journal of clinical investigation 119:3774-3786.
Vonderheide, R. H. 2018. The Immune Revolution: A Case for Priming, Not Checkpoint. Cancer Cell 33:563-569.
Vonderheide, R. H. 2020, CD40 Agonist Antibodies in Cancer Immunotherapy. Annu Rev Med 71:47-58.
Wculek, S. K., Cueto, F. J., Mujal, A. M., Melero, I., Krummel, M. F., and Sancho, D. 2020. Dendritic cells in cancer immunology and immunotherapy. Nat Rev Immunol 20:7-24.
Xie, M., Zheng, H., Madan-Lala, R, Dai, W., Gimbrone, N. T., Chen, Z., Kinose, F., Blackstone, S. A., Smalley, K. S. M., Cress, W. D., Haura, E. B., Rix, U., and Beg, A. A. 2019. MEK Inhibition Modulates Cytokine Response to Mediate Therapeutic Efficacy in Lung Cancer. Cancer Res 79:5812-5825.
Zheng, H., Zhao, W., Yan, C., Watson, C. C., Messengill, M., Xie, M., Messengill, C., Noyes, D., Martinez, G. V., Afzal, R, Chen, Z., Ren, X., Antonia, S. J., Haura, E. B., Ruffell, B., and Beg, A. A. 2016. HDAC inhibitors enhance T cell chemokine expression and augment response to PD-1 immunotherapy in lung adenocarcinoma. Clin Cancer Res 22:4119-4132.

SEQUENCES
is the amino acid sequence of the human wild-type
interferon-a (UniProtKB Reference No. P05014):
SEQ ID NO: 1
MALSFSLLMAVLVLSYKSICSLGCDLPQTHSLGNRRALILLAQMGRISHFSCLKDRHDF
GFPEEEFDGHQFQKAQAISVLHEMIQQTFNLFSTEDSSAAWEQSLLEKFSTELYQQLND
LEACVIQEVGVEETPLMNEDSILAVRKYFQRITLYLTEKKYSPCAWEVVRAEIMRSLSF
STNLQKRLRRKD
is the amino acid sequence of the human wild-type
interferon-b(UniProtKB Reference No. P01574):
SEQ ID NO: 2
MTNKCLLQIALLLCFSTTALSMSYNLLGFLQRSSNFQCQKLLWQLNGRLEYCLKDRMNF
DIPEEIKQLQQFQKEDAALTIYEMLQNIFAIFRQDSSSTGWNETIVENLLANVYHQINH
LKTVLEEKLEKEDFTRGKLMSSLHLKRYYGRILHYLKAKEYSHCAWTIVRVEILRNFYF
INRLTGYLRN
is the amino acid sequence of the human wild-type
interferon-e (UniProtKB Reference No. Q9P0W0):
SEQ ID NO: 3
MSTKPDMIQKCLWLEILMGIFIAGTLSLDCNLLNVHLRRVTWQNLRHLSSMSNSFPVEC
LRENIAFELPQEFLQYTQPMKRDIKKAFYEMSLQAFNIFSQHTFKYWKERHLKQIQIGL
DQQAEYLNQCLEEDKNENEDMKEMKENEMKPSEARVPQLSSLELRRYFHRIDNFLKEKK
YSDCAWEIVRVEIRRCLYYFYKFTALFRRK
is the amino acid sequence of the human wild-type
interferon-k:
SEQ ID NO: 4
MSTKPDMIQKCLWLEILMGIFIAGTLSLDCNLLNVHLRRVTWQNLRHLSSMSNSFPVEC
LRENIAFELPQEFLQYTQPMKRDIKKAFYEMSLQAFNIFSQHTFKYWKERHLKQIQIGL
DQQAEYLNOCLEEDKNENEDMKEMKENEMKPSEARVPQLSSLELRRYFHRIDNFLKEKK
YSDCAWEIVRVEIRRCLYYFYKFTALFRRK
is the amino acid sequence of the human wild-type
interferon-w (UniProtKB Reference No. P05000):
SEQ ID NO: 5
MALLFPLLAALVMTSYSPVGSLGCDLPQNHGLLSRNTLVLLHQMRRISPFLCLKDRRDF
RFPQEMVKGSQLQKAHVMSVLHEMLQQIFSLFHTERSSAAWNMTLLDQLHTGLHQQLQH
LETCLLQVVGEGESAGAISSPALTLRRYFQGIRVYLKEKKYSDCAWEVVRMEIMKSLFL
STNMQERLRSKDRDLGSS
is the amino acid sequence of the human CD40-L
(ETniProtKB Reference No. P29965):
SEQ ID NO: 6
MIETYNQTSPRSAATGLPISMKIFMYLLTVFLITQMIGSALFAVYLHRRLDKIEDERNL
HEDFVFMKTIQRCNTGERSLSLLNCEEIKSQFEGFVKDIMLNKEETKKENSFEMQKGDQ
NPQIAAHVISEASSKTTSVLQWAEKGYYTMSNNLVTLENGKQLTVKRQGLYYIYAQVTF
CSNREASSQAPFIASLCLKSPGRFERILLRAANTHSSAKPCGQQSIHLGGVFELQPGAS
VFVNVTDPSQVSHGTGFTSFGLLKL
is the amino acid sequence for a chimeric
CD40-L:
SEQ ID NO: 7
MIETYSQPSPRSVATGLPASMKIFMYLLTVFLITQMIGSVLFAVYLHRRLDKVEEEVNL
HEDFVFIKKLKRCNKGEGSLSLLNCEEMRRQFEDLVKDITLNKEEKKENSFEMQRGDED
PQIAAHVVSEANSNAASVLQWAKKGYYTMKSNLVTLENGKQLTVKRQGLYYIYAQVTFC
SNREPSSQRPFIVGLWLKPSSGSERILLKAANTHSSSQLCEQQSVHLGGVFELQPGASV
FVNVTDPSQVSHGTGFTSFGLLKL
is the amino acid sequence for a chimeric CD40-L:
SEQ ID NO: 8
MIETYNQTSPRSAATGLPISMKIFMYLLTVFLITQMIGSALFAVYLHRRLDKIEDERNL
HEDFVFMKTIQRCNTGERSLSLLNCEEIKSQFEGFVKDIMLNKEETKKDEDPQIAAHVV
SEANSNAASVLQWAKKGYYTMKSNLVTLENGKQLTVKRQGLYYIYAQVTFCSNREPSSQ
RPFIVGLWLKPSSGSERILLKAANTHSSSQLCEQQSVHLGGVFELQPGASVFVNVTDPS
QVSHGTGFTSFGLLKL
is the amino acid sequence for a chimeric CD40-L:
SEQ ID NO: 9
MIETYSQPSPRSVATGLPASMKIFMYLLTVFLITQMIGSVLFAVYLHRRLDKVEEEVNL
HEDFVFIKKLKRCNKGEGSLSLLNCEEMRRQFEDLVKDITLNKEEKKENSFEMQRGDED
PQIAAHVVSEANSNAASVLQWAKKGYYTMKSNLVTLENGKQLTVKRQGLYYIYAQVTFC
SNREASSQAPFIVGLWLKPSSGSERILLKAANTHSSSQLCEQQSVHLGGVFELQPGASV
FVNVTDPSQVSHGTGFTSFGLLKL
is the amino acid sequence for a chimeric CD40-L:
SEQ ID NO: 10
MIETYNQTSPRSAATGLPISMKIFMYLLTVFLITQMIGSALFAVYLHRRLDKIEDERNL
HEDFVFMKTIQRCNTGERSLSLLNCEEIKSQFEGFVKDIMLNKEETKKDEDPQIAAHVV
SEANSNAASVLQWAKKGYYTMKSNLVTLENGKQLTVKRQGLYYIYAQVTFCSNREASSQ
APFIVGLWLKPSSGSERILLKAANTHSSSQLCEQQSVHLGGVFELQPGASVFVNVTDPS
QVSHGTGFTSFGLLKL
is the amino acid sequence for a chimeric CD40-L:
SEQ ID NO: 11
MIETYSQPSPRSVATGLPASMKIFMYLLTVFLITQMIGSVLFAVYLHRRLDKVEEEVNL
HEDFVFIKKLKRCNKGEGSLSLLNCEEMRRQFEDLVKDITLNKEEKKENSFEMQRGDED
PQIAAHVVSEANSNAASVLQWAKKGYYTMKSNLVTLENGKQLTVKRQGLYYIYAQVTFC
SNREASSQAPFIVGLWLKPSSGSERILLKAANTHSSSQLCEQQSIHLGGVFELQPGASV
FVNVTDPSQVSHGTGFTSFGLLKL
is the amino acid sequence for a chimeric CD40-L:
SEQ ID NO: 12
MIETYNQTSPRSAATGLPISMKIFMYLLTVFLITQMIGSALFAVYLHRRLDKIEDERNL
HEDFVFMKTIQRCNTGERSLSLLNCEEIKSQFEGFVKDIMLNKEETKKDEDPQIAAHVV
SEANSNAASVLQWAKKGYYTMKSNLVTLENGKQLTVKRQGLYYIYAQVTFCSNREASSQ
APFIVGLWLKPSSGSERILLKAANTHSSSQLCEQQSIHLGGVFELQPGASVFVNVTDPS
QVSHGTGFTSFGLLKL
is the amino acid sequence for a chimeric CD40-L:
SEQ ID NO: 13
MIETYSQPSPRSVATGLPASMKIFMYLLTVFLITQMIGSVLFAVYLHRRLDKVEEEVNL
HEDFVFIKKLKRCNKGEGSLSLLNCEEMRRQFEDLVKDITLNKEEKKENSFEMQRGDED
PQIAAHVVSEANSNAASVLQWAKKGYYTMKSNLVTLENGKQLTVKRQGLYYIYAQVTFC
SNREASSQAPFIVGLWLKPSSGSERILLKAANTHSSAKPCGQQSIHLGGVFELQPGASC
FVNVTDPSQVSHGTGFTSFGLLKL
is the amino acid sequence for a chimeric CD40-L:
SEQ ID NO: 14
MIETYNQTSPRSAATGLPISMKIFMYLLTVFLITQMIGSALFAVYLHRRLDKIEDERNL
HEDFVFMKTIQRCNTGERSLSLLNCEEIKSQFEGFVKDIMLNKEETKKDEDPQIAAHVV
SEANSNAASVLQWAKKGYYTMKSNLVTLENGKQLTVKRQGLYYIYAQVTFCSNREASSQ
APFIVGLWLKPSSGSERILLKAANTHSSAKPCGQQSIHLGGVFELQPGASVFVNVTDPS
QVSHGTGFTSFGLLKL
is the amino acid sequence for a chimeric CD40-L:
SEQ ID NO: 15
MIETYSQPSPRSVATGLPASMKIFMYLLTVFLITQMIGSVLFAVYLHRRLDKVEEEVNL
HEDFVFIKKLKRCNKGEGSLSLLNCEEMRRQFEDLVKDITLNKEEKKENSFEMQRGDED
PQIAAHVVSEANSNAASVLQWAKKGYYTMKSNLVTLENGKQLTVKRQGLYYIYAQVTFC
SNREPSSQRPFIVGLWLKPSSGSERILLKAANTHSSSQLCEQQSIHLGGVFELQPGASV
FVNVTDPSQVSHGTGFTSFGLLKL
is the amino acid sequence for a chimeric CD40-L:
SEQ ID NO: 16
MIETYNQTSPRSAATGLPISMKIFMYLLTVFLITQMIGSALFAVYLHRRLDKIEDERNL
HEDFVFMKTIQRCNTGERSLSLLNCEEIKSQFEGFVKDIMLNKEETKKDEDPQIAAHVV
SEANSNAASVLQWAKKGYYTMKSNLVTLENGKQLTVKRQGLYYIYAQVTFCSNREPSSQ
RPFIVGLWLKPSSGSERILLKAANTHSSSQLCEQQSIHLGGVFELQPGASVFVNVTDPS
QVSHGTGFTSFGLLKL
is the amino acid sequence for a chimeric CD40-L:
SEQ ID NO: 17
MIETYSQPSPRSVATGLPASMKIFMYLLTVFLITQMIGSVLFAVYLHRRLDKVEEEVNL
HEDFVFIKKLKRCNKGEGSLSLLNCEEMRRQFEDLVKDITLNKEEKKENSFEMQRGDED
PQIAAHVVSEANSNAASVLQWAKKGYYTMKSNLVTLENGKQLTVKRQGLYYIYAQVTFC
SNREPSSQRPFIVGLWLKPSSGSERILLKAANTHSSAKPCGQQSIHLGGVFELQPGASV
FVNVTDPSQVSHGTGFTSFGLLKL
is the amino acid sequence for a chimeric CD40-L:
SEQ ID NO: 18
MIETYNQTSPRSAATGLPISMKIFMYLLTVFLITQMIGSALFAVYLHRRLDKIEDERNL
HEDFVFMKTIQRCNTGERSLSLLNCEEIKSQFEGFVKDIMLNKEETKKDEDPQIAAHVV
SEANSNAASVLQWAKKGYYTMKSNLVTLENGKQLTVKRQGLYYIYAQVTFCSNREPSSQ
RPFIVGLWLKPSSGSERILLKAANTHSSAKPCGQQSIHLGGVFELQPGASVFVNVTDPS
QVSHGTGFTSFGLLKL

What is claimed is:

1. A method of generating tumor infiltrating lymphocytes comprising
a. administering an oncolytic virus expressing one or more exogenous immunostimulatory molecules into a tumor cell; and
b. harvesting the tumor infiltrating lymphocytes.

2. The method of generating tumor infiltrating lymphocytes of claim 1, wherein the oncolytic virus further expresses one or more type 1 interferons.

3. The method of generating tumor infiltrating lymphocytes of claim 2, wherein the one or more type 1 interferons comprises IFN-α, IFN-β, IFN-κ, IFN-δ, IFN-ε, IFN-τ, IFN-ω, and/or IFN-ζ.

4. The method of generating tumor infiltrating lymphocytes of any of claims 1-3, wherein the one or more immunostimulatory molecule comprises CD40-L, MEM40, B7-1(CD80)/B7-2(CD86), OX40L, 4-1BBL, CD70, GITRL, LIGHT, TIM-4, ICAM-1, CD58 and/or SLAMF6.

5. The method of generating tumor infiltrating lymphocytes of any of claims 1-4, wherein the oncolytic virus is MEM288.

6. The method of generating tumor infiltrating lymphocytes of any of claims 1-5, wherein the oncolytic virus is administered via intra tumoral injection.

7. The method of generating tumor infiltrating lymphocytes of any of claims 1-6, further comprising expanding the harvested TILs ex vivo.

8. A method of expanding a population of tumor infiltrating lymphocytes (TILs) or marrow infiltrating lymphocytes (MILs) comprising:
a. harvesting TILs or MILs from a subject with a cancer;
b. culturing the harvested TILs or MILs in the presence of antigen presenting cells infected with an oncolytic virus expressing one or more exogenous immunostimulatory molecules.

9. The method of expanding a population of tumor infiltrating lymphocytes (TILs) or marrow infiltrating lymphocytes (MILs) of claim 8, wherein oncolytic virus further expresses one or more type 1 interferons.

10. The method of expanding a population of tumor infiltrating lymphocytes (TILs) or marrow infiltrating lymphocytes (MILs) of claim 9, wherein the one or more type 1 interferons comprises IFN-α, IFN-β, IFN-κ, IFN-δ, IFN-ε, IFN-τ, IFN-ω, and/or IFN-ζ.

11. The method of expanding a population of tumor infiltrating lymphocytes (TILs) or marrow infiltrating lymphocytes (MILs) of any of claims 8-10, wherein the one or more immunostimulatory molecule comprises CD40-L, MEM40, B7-1(CD80)/B7-2(CD86), OX40L, 4-1BBL, CD70, GITRL, LIGHT, TIM-4, ICAM-1, CD58 and/or SLAMF6.

12. The method of expanding a population of tumor infiltrating lymphocytes (TILs) or marrow infiltrating lymphocytes (MILs) of any of claims 8-11, wherein the oncolytic virus is MEM-288.

13. A method of treating a cancer in a subject comprising administering to a subject the TILs or MILs of any of claims 1-12.

14. A method of treating a cancer in a subject comprising:
a. administering an oncolytic virus expressing one or more immunostimulatory molecule into a tumor cell;
b. harvesting the tumor infiltrating lymphocytes (TILs) and/or marrow infiltrating lymphocytes (MILs);
c. expanding the harvested TILs and/or MILs ex vivo; and
d. administering the expanded TILs and/or MILs to the subject.

15. A method of treating a cancer in a subject comprising:
a. harvesting tumor infiltrating lymphocytes (TILs) and/or marrow infiltrating lymphocytes (MILs) from a subject with a cancer;
b. culturing the harvested TILs or MILs in the presence of antigen presenting cells infected with an oncolytic virus expressing one or more exogenous immunostimulatory molecules; and
c. administering the expanded TILs and/or MILs to the subject.

16. The method of treating a cancer in a subject of claim 14 or 15, wherein the oncolytic virus further expresses one or more type 1 interferons.

17. The method of treating a cancer of claim 16, wherein the one or more type 1 interferons comprises IFN-α, IFN-β, IFN-κ, IFN-δ, IFN-ε, IFN-τ, IFN-ω, and/or IFN-ζ.

18. The method of treating a cancer of any of claims 14-17, wherein the one or more immunostimulatory molecules comprises CD40-L, MEM40, B7-1(CD80)/B7-2(CD86), OX40L, 4-1BBL, CD70, GITRL, LIGHT, TIM-4, ICAM-1, CD58 and/or SLAMF6.

19. The method of treating a cancer of any of claims 14-18, wherein the oncolytic virus is MEM-288.

20. The method of treating a cancer of any of claims 13-19, further comprising administering to the subject an anticancer agent.
